(19) United States
(12) Patent Application Publication
(10) Pub. No.: US 2006/0121043 A1
(43) Pub. Date: Jun. 8, 2006

(54) Use of modulators of EphA2 and EphrinA1 for the treatment and prevention of infections

(75) Inventors: Michael S. Kinch, Laytonsville, MD (US); Kelly Carles-Kinch, Laytonsville, MD (US)

Correspondence Address: JOHNATHAN KLEIN-EVANS, ONE MEDIMMUNE WAY, GAITHERSBURG, MD, 20878, US

(73) Assignee: MEDIMMUNE, INC., Gaithersburg, MD (US)

(21) Appl. No.: 11/259,266

(22) Filed: Oct. 27, 2005

Related U.S. Application Data
(60) Provisional application No. 60/622,489, filed on Oct. 27, 2004.
(60) Provisional application No. 60/705,705, filed on Aug. 3, 2005.

(51) Int. Cl. A61K 39/395

(52) U.S. Cl. 424/155.1

(57) ABSTRACT

The present invention provides methods and compositions designed for the treatment, management, and/or amelioration of an infection, in particular an intracellular pathogen infection, such as a viral, bacterial, protozoa or fungal infection. In particular, the present invention provides methods for treating, managing, preventing and/or ameliorating an infection where the expression of EphA2 is upregulated in infected cells (e.g., infected epithelial cells), said methods comprising administering to a subject an effective amount of one or more EphA2/EphrinA1 Modulators. In accordance with the present invention, such methods may also comprise the administration of one or more therapies other than an EphA2/EphrinA1 Modulator. The present invention also provides pharmaceutical compositions comprising EphA2/EphrinA1 Modulators, and optionally, one or more prophylactic or therapeutic agents other than an EphA2/EphrinA1 Modulator, and the use of such compositions in the treating, management, prevention and/or amelioration of an infection. Further provided by the invention are articles of manufacture and kits comprising an EphA2/EphrinA1 Modulator of the invention, and, optionally, other prophylactic or therapeutic agents (e.g., immunomodulatory agents, anti-viral agents, anti-inflammatory agents, anti-bacterial agents, anti-fungal agents, etc.).

This application claims priority to U.S. Provisional Application Ser. No. 60/622,489, filed Oct. 27, 2004 and U.S. Provisional Application Ser. No. 60/705,705, filed Aug. 3, 2005, each of which is incorporated by reference herein in its entirety.

### 1. FIELD OF THE INVENTION

The present invention provides methods and compositions designed for the treatment, management, and/or amelioration of a pathogen infection such as a viral, bacterial, protozoa or fungal infection. In particular, the present invention provides methods for treating, managing, preventing and/or ameliorating an infection where the expression of EphA2 is upregulated in infected cells (e.g., infected epithelial cells), said methods comprising administering to a subject an effective amount of one or more EphA2/EphrinA1 Modulators that modulate the expression and/or activity of EphA2 and/or its endogenous ligand, EphrinA1. In accordance with the present invention, such methods may also comprise the administration of one or more therapies other than an EphA2/EphrinA1 Modulator. The present invention also provides pharmaceutical compositions comprising EphA2/EphrinA1 Modulators, and optionally, one or more prophylactic or therapeutic agents other than an EphA2/EphrinA1 Modulator, and the use of such compositions in the treatment, management, prevention and/or amelioration of an infection. Also provided by the invention are methods of detecting, diagnosing and/or prognosing a pathogen infection and/or monitoring the efficacy of a therapy in the treatment, prevention, management or amelioration of a pathogen infection. Further provided by the invention are articles of manufacture and kits comprising an EphA2/EphrinA1 Modulator of the invention, and, optionally, other prophylactic or therapeutic agents (e.g., immunomodulatory agents, anti-viral agents, anti-inflammatory agents, anti-bacterial agents, anti-fungal agents, etc.).

### 2. BACKGROUND OF THE INVENTION

### 2.1 EphA2

EphA2 (epithelial cell kinase) is a 130 kDa member of the Eph family of receptor tyrosine kinases (Zantek et al, 1999, Cell Growth Differ. 10:629-38; Lindberg et al., 1990, Mol. Cell. Biol. 10:6316-24). The function of EphA2 is not known, but it has been suggested to regulate proliferation, differentiation, and barrier function of colonic epitnelium (Rosenberg et al., 1997, Am. J. Physiol. 273:G824-32), vascular network assembly, endothelial migration, capillary morphogenesis, and angiogenesis (Stein et al., 1998, Genes Dev. 12:667-78), nervous system segmentation and axon pathfinding (Bovenkamp and Greer, 2001, DNA Cell Biol. 20:203-13), tumor neovascularization (Ogawa K. et al., 2000, Oncogene 19:6043-52), and cancer metastasis (International Patent Publication Nos. WO 01/9411020, WO 96/36713, WO 01/12840, WO 01/12172).

The natural ligand of EphA2 is EphrinA1 (Eph Nomenclature Committee, 1997, Cell 90(3):403-404; Gale, et al., 1997, Cell Tissue Res. 290(2): 227-41). The and EphrinA1 interaction is thought to help anchor cells on the surface of an organ and also down regulate epithelial and/or endothelial cell proliferation by decreasing EphA2 expression through EphA2 autophosphorylation (Lindberg et al., 1990, Mol. Cell. Biol. 10:6316-24). Under natural conditions, the interaction helps maintain an epithelial cell barrier that protects the organ and helps regulate over proliferation and growth of epithelial cells. However, there are disease states that prevent epithelial cells from forming a protective barrier or cause the destruction and/or shedding of epithelial and/or endothelial cells and thus prevent proper healing from occurring.

### 2.2 Infections

Although the development of antimicrobial drugs to treat infections has advanced rapidly in the past several years, such agents can act against only certain groups of microbes and are associated with increasing rates of resistance (Rachakonda and Sartee, 2004, Curr. Med. Chem. 1 1(6):775-93). Thus, the treatment of infections remains an important clinical focus and challenge. Current therapies for infections involve the administration of anti-viral agents, anti-bacterial, and anti-fungal agents for the treatment, prevention, or amelioration of viral, bacterial, and fungal infections, respectively. Unfortunately, in regard to certain infections, there are no therapies available, infections have been proven to be refractory to therapies, or the occurrence of side effects outweighs the benefits of the administration of a therapy to a subject. For example, the administration of anti-fungal agents may cause renal failure or bone marrow dysfunction and may not be effective against fungal infection in patients with suppressed immune systems. Additionally, the infection causing microorganism (e.g., virus, bacterium, or fungus) may be resistant or develop resistance to the administered therapy agent or combination of therapies. In fact, microorganisms that develop resistance to administered therapies often develop pleiotropic drug or multidrug resistance, that is, resistance to therapies that act by mechanisms different from the mechanisms of the administered therapies. Thus, as a result of drug resistance, many infections prove refractory to a wide array of standard treatment protocols. Therefore, new therapies with unique mechanisms of action for the treatment, prevention, and amelioration of infections and symptoms thereof are needed.

### 2.2.1 Viral Infections

All viruses are parasitic by nature and require the survival of the host in order to survive and replicate. Viruses can be subdivided, depending on their genome, into RNA and DNA viruses. RNA viruses can be single- or double-stranded. DNA viruses are also either single- or double-stranded. RNA viruses can be further classified into segmented and nonsegmented viruses, and both RNA and DNA viruses are distinguished into those that are enveloped and those that are not. The taxonomy of viruses includes orders, families and subfamilies, and genera and species. Non-limiting examples of important viruses that are pathogenic in humans and the diseases that they cause include: Hepatitis A virus (acute hepatitis); HIV (AIDS); Severe Acute Respiratory Syndrome Virus (respiratory infections); Poliomyelitis virus (mild febrile symptoms, aseptic meningitis, paralysis); Rubella virus (rash, low-grade fever, arthralgia, hearing loss, congenital heart disease); West Nile Fever virus (headache, fever, encephalitis in elderly patients); Rabies virus (encephalitis, paralysis, coma); Ebola virus Zaire (fever, hemorrhagic shock); Mumps virus (parotitis, meningoencephalitis, orchitis); Measles virus (fever, rash, pneumonitis, lymphopenia); Hantavirus (fever, capillary leakage, pulmonary edema); Lassa fever virus (fever, sore throat, capillary leakage); Rotavirus (diarrhea); Cytomegalovirus (mononucleosis; in infant, microcephaly, hearing loss, optic atrophy); Hepatitis B virus (hepatitis; acute and chronic hepatocarcinoma); Parainfluenza virus (“PIV”) (in infants, respiratory tract disease); Respiratory syncytial virus (“RSV”) (in infants, lower respiratory tract disease; in adults, upper respiratory tract disease); and Avian & Human Metapneumovirus (upper respiratory tract disease, severe bronchiolitis, pneumonia). See, e.g., Ertl, H. C., Viral Immunology, in: Fundamental Immunology, 5^{th} ed. (Paul, ed.) Lippincott Williams & Wilkins (Philadelphia, 2003).

Most viruses infect their hosts through the mucosal surfaces of the airways, the conjunctivae, the gastrointestinal tract, or the urogenital tract. Others invade through the skin or through direct inoculation into a tissue. In an active viral infection, the virus, upon entering the host cell or tissue, begins to replicate its genetic material and viral proteins.

Much of the damage resulting from a viral infection is due to death of the host cells during viral replication. The host has many early immune defense mechanisms against a viral infection. For example, natural killer (NK) cells become activated in the absence of class I MHC molecules that on normal cells bind to inhibitory receptors. Once activated, NK cells secrete cytokines, e.g., Interferon (IFN)-γ or perforin. Marginal zone B cells and B1 cells, upon activation, secrete immunoglobulin M (IgM) antibodies with low affinity to an array of pathogens. Such antibodies can bind and neutralize a circulating virus in the early stages of the infection. Following an early immune response, the host immune system begins induction of the antigen-specific (adaptive) immune response, which involves CD4+ and CD8+ T cells and B cells, which takes at least 4 to 5 days post-infection. The adaptive immune response involves the presentation of processed viral antigens to the immune system as well as the activation of B cells to produce antigen-specific antibodies which recognize specific viral antigens.

In the case of some infections, some viruses may escape the host immune system by shutting off viral protein synthesis and by entering a state of latency (latent infection). In such a state, the host immune system remains ignorant of latently infected cells that do not express viral antigens. This allows the virus to evade complete destruction during the height of an acute immune response. Once the immune system assumes a more relaxed stage of memory, the virus can reactivate and replicate unhindered for a few days until T cells convert from memory cells back to effector cells. These short bursts of viral replication may be sufficient to produce ample amounts of virus to allow its spread to other organisms.

Although modern medicine with its vaccines and drugs has dramatically reduced the impact of viral infections on human health, new viruses emerge constantly, and increased global travel has increased the spread of viruses. Thus, new therapies that take advantage of the pathogenic mechanisms of viral infections are needed.

### 2.2.1.1 Parainfluenza Virus Infections

Parainfluenza viral (“PIV”) infection results in serious respiratory tract disease in infants and children. (Tao et al., 1999, Vaccine 17: 1100-08). Infectious parainfluenza viral infections account for approximately 20% of all hospitalizations of pediatric patients suffering from respiratory tract infections worldwide. Id.

PIV is a member of the paramyxovirus genus of the paramyxoviridae family. PIV is made up of two structural modules: (1) an internal ribonucleoprotein core or nucleocapsid, containing the viral genome, and (2) an outer, roughly spherical lipoprotein envelope. Its genome is a single strand of negative sense RNA, approximately 15,456 nucleotides in length, encoding at least eight polypeptides. These proteins include, but are not limited to, the nucleocapsid structural protein (NP, NC, or N depending on the genera), the phosphoprotein (P), the matrix protein (M), the fusion glycoprotein (F), the hemagglutinin-neuraminidase glycoprotein (HN), the large polymerase protein (L), and the C and D proteins of unknown function. Id.

The parainfluenza nucleocapsid protein (NP, NC, or N) consists of two domains within each protein unit including an amino-terminal domain, comprising about two-thirds of the molecule, which interacts directly with the RNA, and a carboxyl-terminal domain, which lies on the surface of the assembled nucleocapsid. A hinge is thought to exist at the junction of these two domains thereby imparting some flexibility to this protein (see Fields et al. (ed.), 1991, Fundamental Virology, 2nd ed., Raven Press, New York, incorporated by reference herein in its entirety). The matrix protein (M), is apparently involved with viral assembly and interacts with both the viral membrane as well as the nucleocapsid proteins. The phosphoprotein (P), which is subject to phosphorylation, is thought to play a regulatory role in transcription and may also be involved in methylation, phosphorylation and polyadenylation. The fusion glycoprotein (F) interacts with the viral membrane and is first produced as an inactive precursor then cleaved post-translationally to produce two disulfide linked polypeptides. The active F protein is also involved in penetration of the parainfluenza virion into host cells by facilitating fusion of the viral envelope with the host cell plasma membrane. Id. The glycoprotein, hemagglutinin-neuraminidase (HN), protrudes from the envelope allowing the virus to contain both hemagglutinin and neuraminidase activities. HN is strongly hydrophobic at its amino terminal which functions to anchor the HN protein into the lipid bilayer. Id. Finally, the large polymerase protein (L) plays an important role in both transcription and replication. Id.

Currently, therapies for PIV comprises treatment of specific symptoms. In most cases rest, fluids, and a comfortable environment are sufficient therapy for a PIV infection. In cases in which fever is high, acetaminophen is recommended over aspirin, especially in children to avoid the risk of Reye's syndrome with influenza. For croup associated with PIV infection, therapies such as humidified air, oxygen, aerosolized racemic epinephrine, and oral dexamethasone (a steroid) are recommended to decrease upper airway swelling and intravenous fluids are administered for dehydration. Therapy for bronchiolitis associated with PIV infection include supportive therapy (e.g., oxygen, humidified air, chest clapping, and postural drainage to remove secretions, rest, and clear fluids) and administration of albuterol or steroids. Antibiotic, anti-viral, and/or anitfungal agents may be administered to prevent secondary respiratory infections. See Merck Manual of Diagnosis and Therapy (17th ed., 1999).

### 2.2.1.2 Respiratory Syncytial Virus Infections

Respiratory syncytial virus (“RSV”) is the leading cause of serious lower respiratory tract disease in infants and children (Feigen et al., eds., 1987, Textbook of Pediatric Infections, W B Saunders, Philadelphia at pages 1653-1675; New Vaccine Development, Establishing Priorities, Vol. 1, 1985, National Academy Press, Washington D.C. at pages 397-409; and Ruuskanen et al., 1993, Curr. Probl. Pediatr. 23:50-79). The yearly epidemic nature of RSV infection is evident worldwide, but the incidence and severity of RSV disease in a given season vary by region (Hall, C. B., 1993, Contemp. Pediatr. 10:92-110). In temperate regions of the northern hemisphere, it usually begins in late fall and ends in late spring. Primary RSV infection occurs most often in children from 6 weeks to 2 years of age and uncommonly in the first 4 weeks of life during nosocomial epidemics (Hall et al., 1979, New Engl. J. Med. 300:393-396). Children at increased risk from RSV infection include, but are not limited to, preterm infants (Hall et al., 1979, New Engl. J. Med. 300:393-396) and children with bronchopulmonary dysplasia (Groothuis et al., 1988, Pediatrics 82:199-203), congenital heart disease (MacDonald et al, New Engl. J. Med. 307:397-400), congenital or acquired immunodeficiency (Ogra et al., 1988, Pediatr. Infect. Dis. J. 7:246-249; and Pohl et al., 1992, J. Infect. Dis. 165:166-169), and cystic fibrosis (Abman et al., 1988, J. Pediatr. 113:826-830). The fatality rate in infants with heart or lung disease who are hospitalized with RSV infection is 3%-4% (Navas et al., 1992, J. Pediatr. 121:348-354).

RSV infects adults as well as infants and children. In healthy adults, RSV causes predominantly upper respiratory tract disease. It has recently become evident that some adults, especially the elderly, have symptomatic RSV infections more frequently than had been previously reported (Evans, A. S., eds., 1989, Viral Infections of Humans Epidemiology and Control, 3rd ed., Plenum Medical Book, New York at pages 525-544). Several epidemics also have been reported among nursing home patients and institutionalized young adults (Falsey. A. R., 1991, Infect. Control Hosp. Epidemiol. 12:602-608; and Garvie et al., 1980, Br. Med. J. 281:1253-1254). Finally, RSV may cause serious disease in immunosuppressed persons, particularly bone marrow transplant patients (Hertz et al., 1989, Medicine 68:269-281).

Therapies available for the treatment of established RSV disease are limited. Severe RSV disease of the lower respiratory tract often requires considerable supportive care, including administration of humidified oxygen and respiratory assistance (Fields et al., eds, 1990, Fields Virology, 2nd ed., Vol. 1, Raven Press, New York at pages 1045-1072).

While a vaccine might prevent RSV infection, no vaccine is yet licensed for this indication. A major obstacle to vaccine development is safety. A formalin-inactivated vaccine, though immunogenic, unexpectedly caused a higher and more severe incidence of lower respiratory tract disease due to RSV in immunized infants than in infants immunized with a similarly prepared trivalent parainfluenza vaccine (Kim et al., 1969, Am. J. Epidemiol. 89:422-434; and Kapikian et al., 1969, Am. J. Epidemiol. 89:405-421). Several candidate RSV vaccines have been abandoned and others are under development (Murphy et al., 1994, Virus Res. 32:13-36), but even if safety issues are resolved, vaccine efficacy must also be improved. A number of problems remain to be solved. Immunization would be required in the immediate neonatal period since the peak incidence of lower respiratory tract disease occurs at 2-5 months of age. The immaturity of the neonatal immune response together with high titers of maternally acquired RSV antibody may be expected to reduce vaccine immunogenicity in the neonatal period (Murphy et al., 1988, J. Virol. 62:3907-3910; and Murphy et al., 1991, Vaccine 9:185-189). Finally, primary RSV infection and disease do not protect well against subsequent RSV disease (Henderson et al., 1979, New Engl. J. Med. 300:530-534).

Currently, the only approved approach to prophylaxis of RSV disease is passive immunization. Initial evidence suggesting a protective role for IgG was obtained from observations involving maternal antibody in ferrets (Prince, G. A., Ph.D. diss., University of California, Los Angeles, 1975) and humans (Lambrecht et al., 1976, J. Infect. Dis. 134:211-217; and Glezen et al., 1981, J. Pediatr. 98:708-715). Hemming et al. (Morell et al., eds., 1986, Clinical Use of Intravenous Immunoglobulins, Academic Press, London at pages 285-294) recognized the possible utility of RSV antibody in treatment or prevention of RSV infection during studies involving the pharmacokinetics of an intravenous immune globulin (IVIG) in newborns suspected of having neonatal sepsis. They noted that one infant, whose respiratory secretions yielded RSV, recovered rapidly after IVIG infusion. Subsequent analysis of the IVIG lot revealed an unusually high titer of RSV neutralizing antibody. This same group of investigators then examined the ability of hyperimmune serum or immune globulin, enriched for RSV neutralizing antibody, to protect cotton rats and primates against RSV infection (Prince et al., 1985, Virus Res. 3:193-206; Prince et al., 1990, J. Virol. 64:3091-3092; Hemming et al., 1985, J. Infect. Dis. 152:1083-1087; Prince et al., 1983, Infect. Immun. 42:81-87; and Prince et al., 1985, J. Virol. 55:517-520). Results of these studies suggested that RSV neutralizing antibody given prophylactically inhibited respiratory tract replication of RSV in cotton rats. When given therapeutically, RSV antibody reduced pulmonary viral replication both in cotton rats and in a nonhuman primate model. Furthermore, passive infusion of immune serum or immune globulin did not produce enhanced pulmonary pathology in cotton rats subsequently challenged with RSV.

Recent clinical studies have demonstrated the ability of this passively administered RSV hyperimmune globulin (RSV IVIG) to protect at-risk children from severe lower respiratory infection by RSV (Groothius et al., 1993, New Engl. J. Med. 329:1524-1530; and The PREVENT Study Group, 1997, Pediatrics 99:93-99). While this is a major advance in preventing RSV infection, this therapy poses certain limitations in its widespread use. First, RSV IVIG must be infused intravenously over several hours to achieve an effective dose. Second, the concentrations of active material in hyperimmune globulins are insufficient to treat adults at risk or most children with comprised cardiopulmonary function. Third, intravenous infusion necessitates monthly hospital visits during the RSV season. Finally, it may prove difficult to select sufficient donors to produce a hyperimmune globulin for RSV to meet the demand for this product. Currently, only approximately 8% of normal donors have RSV neutralizing antibody titers high enough to qualify for the production of hyperimmune globulin.

One way to improve the specific activity of the immunoglobulin would be to develop one or more highly potent RSV neutralizing monoclonal antibodies (MAbs). Such MAbs should be human or humanized in order to retain favorable pharmacokinetics and to avoid generating a human anti-mouse antibody response, as repeat dosing would be required throughout the RSV season. Two glycoproteins, F and G, on the surface of RSV have been shown to be targets of neutralizing antibodies (Fields et al., 1990, supra; and Murphy et al., 1994, supra). These two proteins are also primarily responsible for viral recognition and entry into target cells; G protein binds to a specific cellular receptor and the F protein promotes fusion of the virus with the cell. The F protein is also expressed on the surface of infected cells and is responsible for subsequent fusion with other cells leading to syncytia formation. Thus, antibodies to the F protein may directly neutralize virus or block entry of the virus into the cell or prevent syncytia formation. Although antigenic and structural differences between A and B subtypes have been described for both the G and F proteins, the more significant antigenic differences reside on the G glycoprotein, where amino acid sequences are only 53% homologous and antigenic relatedness is 5% (Walsh et aL, 1987, J. Infect. Dis. 155:1198-1204; and Johnson et aL., 1987, Proc. Natl. Acad. Sci. USA 84:5625-5629). Conversely, antibodies raised to the F protein show a high degree of cross-reactivity among subtype A and B viruses. Comparison of biological and biochemical properties of 18 different murine MAbs directed to the RSV F protein resulted in the identification of three distinct antigenic sites that are designated A, B, and C. (Beeler and Coelingh, 1989, J. Virol. 7:2941-2950). Neutralization studies were performed against a panel of RSV strains isolated from 1956 to 1985 that demonstrated that epitopes within antigenic sites A and C are highly conserved, while the epitopes of antigenic site B are variable.

A humanized antibody directed to an epitope in the A antigenic site of the F protein of RSV, palivizumab (SYNAGIS®), is approved for intramuscular administration to pediatric patients for prevention of serious lower respiratory tract disease caused by RSV at recommended monthly doses of 15 mg/kg of body weight throughout the RSV season (November through April in the northern hemisphere). Palivizumab (SYNAGIS®) is a composite of human (95%) and murine (5%) antibody sequences. See, Johnson et al., 1997, J. Infect. Diseases 176:1215-1224 and U.S. Pat. No. 5,824,307, the entire contents of which are incorporated herein by reference. The human heavy chain sequence was derived from the constant domains of human IgG₁ and the variable framework regions of the VH genes of Cor (Press et al., 1970, Biochem. J. 117:641-660) and Cess (Takashi et al., 1984, Proc. Natl. Acad. Sci. USA 81:194-198). The human light chain sequence was derived from the constant domain of CK and the variable framework regions of the VL gene K104 with Jκ-4 (Bentley et al., 1980, Nature 288:5194-5198). The murine sequences derived from a murine monoclonal antibody, Mab 1129 (Beeler et al., 1989, J. Virology 63:2941-2950), in a process which involved the grafting of the murine complementarity determining regions into the human antibody frameworks.

### 2.2.1.3 Avian & Human Metapneumovirus

Recently, a new member of the Paramyxoviridae family has been isolated from 28 children with clinical symptoms reminiscent of those caused by human respiratory syncytial virus (“hRSV”) infection, ranging from mild upper respiratory tract disease to severe bronchiolitis and pneumonia (Van Den Hoogen et al., 2001, Nature Medicine 7:719-724). The new virus was named human metapneumovirus (hMPV) based on sequence homology and gene constellation. The study further showed that by the age of five years virtually all children in the Netherlands have been exposed to hMPV and that the virus has been circulating in humans for at least half a century.

The genomic organization of human metapneumovirus is described in van den Hoogen et al., 2002, Virology 295:119-132. Human metapneumovirus has recently been isolated from patients in North America (Peret et al., 2002, J. Infect. Diseases 185:1660-1663).

Human metapneumovirus is related to avian metapneumovirus. For example, the F protein of hMPV is highly homologous to the F protein of avian pneumonovirus (“APV”). Alignment of the human metapneumoviral F protein with the F protein of an avian pneumovirus isolated from Mallard Duck shows 85.6% identity in the ectodomain. Alignment of the human metapneumoviral F protein with the F protein of an avian pneumovirus isolated from Turkey (subgroup B) shows 75% identity in the ectodomain. See, e.g., co-owned and co-pending Provisional Application No. 60/358,934, entitled “Recombinant Parainfluenza Virus Expression Systems and Vaccines Comprising Heterologous Antigens Derived from Metapneumovirus,” filed on Feb. 21, 2002, by Haller and Tang, which is incorporated herein by reference in its entirety.

Respiratory disease caused by an APV was first described in South Africa in the late 1970s (Buys et al., 1980, Turkey 28:36-46) where it had a devastating effect on the turkey industry. The disease in turkeys was characterized by sinusitis and rhinitis and was called turkey rhinotracheitis (TRT). The European isolates of APV have also been strongly implicated as factors in swollen head syndrome (SHS) in chickens (O'Brien, 1985, Vet. Rec. 117:619-620). Originally, the disease appeared in broiler chicken flocks infected with Newcastle disease virus (NDV) and was assumed to be a secondary problem associated with Newcastle disease (ND). Antibody against European APV was detected in affected chickens after the onset of SHS (Cook et al., 1988, Avian Pathol. 17:403-410), thus implicating APV as the cause.

The avian pneumovirus is a single stranded, non-segmented RNA virus that belongs to the sub-family Pneumovirinae of the family Paramyxoviridae, genus metapneumovirus (Cavanagh and Barrett, 1988, Virus Res. 11:241-256; Ling et al., 1992, J. Gen. Virol. 73:1709-1715; Yu et al., 1992, J. Gen. Virol. 73:1355-1363). The Paramyxoviridae family is divided into two sub-families: the Paramyxovirinae and Pneumovirinae. The subfamily Paramyxovirinae includes, but is not limited to, the genera: Paramyxovirus, Rubulavirus, and Morbillivirus. Recently, the sub-family Pneumovirinae was divided into two genera based on gene order, i.e., pneumovirus and metapneumovirus (Naylor et al., 1998, J. Gen. Virol., 79:1393-1398; Pringle, 1998, Arch. Virol. 143:1449-1159). The pneumovirus genus includes, but is not limited to, human respiratory syncytial virus (hRSV), bovine respiratory syncytial virus (bRSV), ovine respiratory syncytial virus, and mouse pneumovirus. The metapneumovirus genus includes, but is not limited to, European avian pneumovirus (subgroups A and B), which is distinguished from HRSV, the type species for the genus pneumovirus (Naylor et al., 1998, J. Gen. Virol., 79:1393-1398; Pringle, 1998, Arch. Virol. 143:1449-1159). The US isolate of APV represents a third subgroup (subgroup C) within metapneumovirus genus because it has been found to be antigenically and genetically different from European isolates (Seal, 1998, Virus Res. 58:45-52; Senne et al., 1998, In: Proc. 47th WPDC, California, pp. 67-68).

Electron microscopic examination of negatively stained APV reveals pleomorphic, sometimes spherical, virions ranging from 80 to 200 nm in diameter with long filaments ranging from 1000 to 2000 nm in length (Collins and Gough, 1988, J. Gen. Virol. 69:909-916). The envelope is made of a membrane studded with spikes 13 to 15 nm in length. The nucleocapsid is helical, 14 nm in diameter and has 7 nm pitch. The nucleocapsid diameter is smaller than that of the genera Paramyxovirus and Morbillivirus, which usually have diameters of about 18 mn.

Avian pneumovirus infection is an emerging disease in the USA despite its presence elsewhere in the world in poultry for many years. In May 1996, a highly contagious respiratory disease of turkeys appeared in Colorado, and an APV was subsequently isolated at the National Veterinary Services Laboratory (NVSL) in Ames, Iowa (Senne et al., 1997, Proc. 134th Ann. Mtg., AVMA, pp. 190). Prior to this time, the United States and Canada were considered free of avian pneumovirus (Pearson et al., 1993, In: Newly Emerging and Re-emerging Avian Diseases: Applied Research and Practical Applications for Diagnosis and Control, pp. 78-83; Hecker and Myers, 1993, Vet. Rec. 132:172). Early in 1997, the presence of APV was detected serologically in turkeys in Minnesota. By the time the first confirmed diagnosis was made, APV infections had already spread to many farms. The disease is associated with clinical signs in the upper respiratory tract: foamy eyes, nasal discharge and swelling of the sinuses. It is exacerbated by secondary infections. Morbidity in infected birds can be as high as 100%. The mortality can range from 1 to 90% and is highest in six to twelve week old poults.

Avian pneumovirus is transmitted by contact. Nasal discharge, movement of affected birds, contaminated water, contaminated equipment; contaminated feed trucks and load-out activities can contribute to the transmission of the virus. Recovered turkeys are thought to be carriers. Because the virus is shown to infect the epithelium of the oviduct of laying turkeys and because APV has been detected in young poults, egg transmission is considered a possibility.

Based upon the recent work with hMPV, hMPV likewise appears to be a significant factor in human, particularly, juvenile respiratory disease.

Thus, theses three viruses, RSV, hMPV, and PIV, cause a significant portion of human respiratory disease. Accordingly, a broad spectrum therapy is needed to reduce the incidence of viral respiratory disease caused by these viruses.

### 2.2.1.4 Severe Acute Respiratory Syndome Virus

A new coronavirus has been found in patients with Severe Acute Respiratory Syndrome (SARS) and has been identified as the probable cause of SARS (SARS; Drosten et al., 2003, N Engl J Med 348:1967-76). SARS is an infection with a high potential for transmission to close contacts. Symptoms of SARS include fever (>38° Celsius), dry cough, shortness of breath or breathing difficulties, and changes in chest X-rays indicative of pneumonia. Other symptoms include headache, muscular stiffness, loss of appetite, malaise, confusion, rash and diarrhea. At present, there is no specific therapy available for the prevention or treatment of a SARS-associated coronavirus infection. Given the potential for spread of SARS-associated coronavirus and the lethality of SARS, there is a need for prophylactic and therapeutic therapies for the prevention, treatment and/or amelioration of SARS-associated coronavirus infection.

### 2.2.1.5 Hepatitis B Virus

Hepatitis B virus (“HPV”) is present in bodily fluids such as blood and semen, and can be transmitted by inoculating these fluids through the skin or mucous membranes. The highest concentrations of HBV are found in blood and serous fluids.

In order to reach the liver, HBV must gain access to the blood circulation by crossing the skin or mucous membranes. In addition to being a highly infectious virus, HBV is stable on environmental surfaces for up to 7 days, and so may be inoculated indirectly from inanimate objects. Four major modes of transmission are recognized: perinatal (vertical), parenteral/percutaneous, sexual, and horizontal (Physical contact).

Two distinct patterns of transmission are observed in areas where infection is highly prevalent. In Asia, perinatal infections account for at least 25 percent of chronic HBV infections in the adult population. In these regions, 5-12 percent of pregnant females are HBsAg-positive and up to half of these women are viraemic. Maternal serum HBV DNA is the most important determinant of infection outcome in the infant. Perinatal transmission rates can be as high as 90 percent. It is not clear whether HBV is transmitted vertically from mother to child in utero or during birth. In Africa and the Middle East, perinatal transmission is less frequent but horizontal transmission within the family or from sources outside the family is more important. All young children have a high risk of acquiring chronic infection during their first 5 years of life. The precise routes of horizontal transmission are uncertain.

In areas with intermediate prevalence, transmission occurs in all age groups from newborn to adult. Early childhood infection may be responsible for most of the chronic infections, but higher rates of acute infection are thought to occur among older children, adolescents and young adults. Such infections are less likely to become chronic. HBV may be transmitted sexually or through acupuncture or ritual practices where the skin is cut.

In countries where there is a low prevalence of HBV infection, transmission occurs primarily among adults in defined risk groups whose life-style places them at risk of infection. The two groups with the highest risk are intravenous drug abusers, who share needles, and heterosexuals or homosexuals with multiple partners. Incidence is also elevated among immigrants from endemic regions. In the USA, at least 30 percent of cases of hepatitis B occur among people without an identifiable source of infection.

Other epidemiological studies have shown that the risk of HBV infection is higher in the following groups: individuals with multiple sexual partners and a history of other sexually-transmitted diseases; household contacts of individuals with hepatitis B; healthcare workers who are exposed to blood and body fluids or who may have needle stick injuries; staff and residents in prisons and mental institutions; recipients of contaminated blood transfusions or blood products; parenteral drug abusers are exposed to the additional threat of delta hepatitis (HDV), an infection which increases the severity of both acute and chronic hepatitis B. Outbreaks have occurred among parenteral drug abusers in the USA. Like HBV, HDV, the causative agent, is transmitted through blood. HCV and HIV co-infections may also be acquired through sharing needles.

### 2.2.1.6 Human Immunodeficiency Virus

HFV infection is a viral infection caused by the human immunodeficiency syndrome virus (“HIV”) that gradually destroys the immune system, resulting infections that the body cannot fight. Acute HIV infection may be associated with symptoms resembling mononucleosis or the flu within 2 to 4 weeks of exposure. HIV seroconversion (converting from HIV negative to HIV positive) usually occurs within 3 months of exposure to the virus. Humans who become infected with HIV may have no symptoms for up to 10 years, but they can still transmit the infection to others. Meanwhile, their immune system gradually weakens until they are diagnosed with Acquired Immune Deficiency Syndrome (“AIDS”). Most individuals infected with HIV will develop AIDS if not treated. The Centers for Disease and Control has defined AIDS as beginning when a person with HIV infection has a CD4 T cell count of below 200. It is also defined by numerous opportunistic infections and cancers that occur in the presence of HIV infection.

The HIV epidemic has occurred in multiple waves, depending on the timing of introduction of the virus into a population and the demographics of the population in question. In certain regions of the world, the incidence of infection has recently plateaued, while in other regions incidence rates continue to rise. In 16 African countries, the prevalence of HIV infection among adults aged 15-49 exceeds 10%; similar rates may be seen in the near future in regions of Asia where the epidemic is accelerating. In the United States, male-to-male sexual contact remains the most common mechanism of HIV transmission over the entire course of the epidemic; however, heterosexual transmission and injection drug use account for an increasing proportion of cases of HIV over the past few years. Transmission of HIV, which causes AIDS, occurs through sexual contact (e.g., oral vaginal and anal), through blood, (e.g., blood transfusions or needle sharing), and from mother to child. Other transmission methods are rare and include accidental needle injury, artificial insemination with donated semen, and through a donated organ.

Although many effective medicines are developed to fight the many symptoms of AIDS, there is currently no cure for AIDs. Thus, new therapies must be developed to treat this deadly disease.

### 2.2.2 Bacterial Infections

Bacterial infections are caused by the presence and growth of microorganisms that damage host tissue. The extent of infection is generally determined by how many organisms are present and the toxins they release. Worldwide, bacterial infections are responsible for more deaths than any other cause. Symptoms can include inflammational and swelling, pain, heat, redness, and loss of function. The most important risk factors are burns, severe trauma, low white blood cell counts, patients on immunotherapy treatment, and anyone with malnutrition or vitamin deficiency.

Bacteria are generally spread from an already infected person to the newly infected person. The most common invasion routes are inhalation of airborne bacteria, ingestion into the stomach from dirty hands or utensils, or through contaminated food or water, direct contact with an infected area of another person's body, contaminated blood, or by insect bite.

Pathogenic bacteria that cause human disease are diverse. On the basis of the pathogenesis of infection and the resulting immune response, these bacteria can be categorized into two general types: those causing intracellular infections and those causing extracellular infections. Most bacteria causing intracellular infections avoid being killed by phagocytosis by either interfering with phagosome-lysosome fusion or by escapting from the phagosome and into the cytoplasm. Cellular immunity is critical against intracellular bacteria. For a review of immune responses to intracellular bacteria, see, e.g., Kaufmann, Immunity to Intracellular Bacteria, in: Fundamental Immunology, 5^{th} ed., Paul (ed.) Philadelphia, pp. 1229-1283, 2003.

Intracellular bacteria comprise numerous pathogens. Of paramount significance for humans are Mycobacterium tuberculosis, Mycobacterium leprae, Salmonella enterica serovar Typhi, and Chlamydia trachomatis, the etiologic agents of tuberculosis, leprosy, typhoid, and trachoma, respectively, which together, afflict more than 600 million people. An association of Chlamydia peneumoniae with cardiovascular diseases has been claimed. Some opportunistic pathogens such as Mycobacterium avium/Mycobacterium intracellulare are gaining increasing significance with the growing number of immunodeficient patients, such as AIDS patients.

Intracellular bacteria can live inside host cells for most of their lives. Non-limiting examples of intracellular bacteria and the infections they cause in humans include: Mycobacterium tuberculosis (tuberculosis), Mycobacterium leprae (leprosy), Salmonella enterica serovar Typhi (typhoid fever), Brucella sp (Brucellosis), Legionella sp (Legionnaire's disease), Listeria monocytogenes (Listeriosis), Francisella tularensis (Tularemia), Rickettsia rickettsii (Rocky Mountain spotted fever); Rickettsia prowazekii (endemic typhus); Rickettsia typhi (typhus); Rickettsia tsutsugamushi (scrub typhus); Chlamydia trachoratis (urogenital infection, conjunctivitis, trachoma, lymphogranuloma venerum (different serovars)); Chlamydia psittaci (psittacosis); and Chlamydia pneumoniae (pneumonia, coronary heart disease).

The first of the body's three primary lines of defense includes naturally occurring chemicals such as the lysozymes found in tears, gastric acid of the stomach, pancreatic enzymes of the bowel, and fatty acids in the skin. The body's immune response becomes involved only if the infective organism manages to invade the body. Nonspecific immune response—the body's second line of defense—consists primarily of inflammation, whereas specific immune response—the third line of defense—relies on the activation of lymphocytes, which send T- and B-cells to try to recognize the specific type of organism involved. T-cells marshal cytotoxic cells, which are sent to destroy the organism, and B-cells produce the antibodies—immunoglobulins—that can destroy specific types of bacteria.

Acute bacterial infections require immediate conventional medical care. If FDA-approved antibiotics fail to work, European antibiotics, which are several years more advanced than American antibiotics, may be effective.

When antibiotics were discovered in the 1940s, they were incredibly effective in the treatment of many bacterial infections. Over time many antibiotics have lost their effectiveness against certain types of bacteria because resistant strains have developed, mostly through the expression of resistance genes.

There are several ways in which bacteria become resistant to antibiotic therapy. One way is that some bacteria have now developed “efflux” pumps. When the bacterium recognizes invasion by an antibiotic, the efflux pump simply pumps the antibiotic out of its cells. Resistance genes code for more than pumps, however. Some lead to the manufacture of enzymes that degrade or chemically alter (and therefore inactivate) the antibiotic. Where do these resistance genes come from? Usually, bacteria get them from other bacteria. In some cases they pick up a gene containing plasmid from a “donor” cell. Also, viruses have been shown to extract a resistance gene from one bacterium and inject it into a different one. Furthermore, some bacteria “scavenge” DNA from dead cells around them, and occasionally, scavenged genes are incorporated in a stable manner into the recipient cell's chromosome or into a plasmid and become part of the recipient bacterium. A few resistance genes develop through random mutations in the bacterium's DNA.

Thus, there is an increasing need to develop new therapies to treat bacterial infections, particularly intracellular bacterial infections.

### 2.2.2.1 Mycobacterium Tuberculosis

Mycobacterium tuberculosis infects 1.9 billion and the active disease, tuberculosis (“TB”) results in 1.9 million deaths around the world each year. (Dye et al., 1999, JAMA 282:677-686). After a century of steadily declining rates of TB cases in the United States, the downward trend was reversed in the late 1980s as a result of the emergence of a multidrug-resistant strain of M. tuberculosis, the HIV epidemic, and the influx of immigrants. (Navin et al., 2002, Emerg. Infect. Dis. 8:11).

M. tuberculosis is an obligate aerobe, nonmotile rod-shaped bacterium. In classic cases of tuberculosis, M. tuberculosis complexes are in the well-aerated upper lobes of the lungs. M. tuberculosis are classified as acid-fast bacteria due to the impermeability of the cell wall by certain dyes and stains. The cell wall of M. tuberculosis, composed of peptidoglycan and complex lipids, is responsible for the bacterium's resistance to many antibiotics, acidic and alkaline compounds, osmotic lysis, and lethal oxidations, and survival inside macrophages.

TB progresses in five stages. In the first stage, the subject inhales the droplet nuclei containing less than three bacilli. Although alveolar macrophages take up the M. tuberculosis, the macrophages are not activated and do not destroy the bacterium. Seven to 21 days after the initial infection, the M. tuberculosis multiples within the macrophages until the macrophages burst, which attracts additional macrophages to the site of infection that phagocytose the M. tuberculosis, but are not activated and thus do not destroy the M. tuberculosis. In stage 3, lymphocytes, particularly T-cells, are activated and cytokines, including IFN activate macrophages capable of destroying M. tuberculosis are produced. At this stage, the patient is tuberculin-positive and a cell mediated immune response, including activated macrophages releasing lytic enzymes and T cell secreting cytokines, is initiated. Although, some marcrophages are activated against the M. tuberculosis, the bacteria continue to multiply within inactivated macrophages and begin to grow tubercles which are characterized by semi-solid centers. In stage 4, tubercles may invade the bronchus, other parts of the lung, and the blood supply line and the patient may exhibit secondary lesions in other parts of the body, including the genitourinary system, bones, joints, lymph nodes, and peritoneum. In the final stage, the tubercles liquify inducing increased growth of M. tuberculosis. The large bacterium load causes the walls of nearby bronchi to rupture and form cavities that enables the infection to spread quickly to other parts of the lung.

Current therapies available for TB comprise an initial two month regime of multiple antibiotics, such as rifampein, isoniazid, pyranzinamide, ethambutol, or streptomycin. In the next four months, only rifampicin and isoniazid are administered to destroy persisting M. tuberculosis. Although proper prescription and patient compliance results in a cure in most cases, the number of deaths from TB has been on the rise as a result of the emergence of new M. tuberculosis strains resistant to current antibiotic therapies. (Rattan et al., 1998, Emerging Infections, 4(2):195-206). In addition, fatal and severe liver injury has been associated with treatment of latent TB with rifampcin and pyranzinamide. (CDC Morbidity and Mortality Weekly Report, 51(44):998-999).

### 2.2.3 Fungal Infections

The number of systemic invasive fungal infections rose sharply in the past decade due to the increase in the at-risk patient population as a result of organ transplants, oncology, human immunodeficiency virus, use of vascular catheters, and misuse of broad spectrum antibiotics. Dodds et al., 2000 Pharmacotherapy 20(11): 1335-1355. Seventy percent of fungal-related deaths are caused by Candida species, Aspergillus species, and Cryptococcus neoformans. Yasuda, Calif. Journal of Health-System Pharmacy, May/June 2001, pp. 4-11. Non-limiting examples of fungi that cause infections include Absidia species (e.g., Absidia corymbifera and Absidia ramosa), Aspergillus species, (e.g., Aspergillus flavus, Aspergillus fumigatus, Aspergillus nidulans, Aspergillus niger, and Aspergillus terreus), Basidiobolus ranarum, Blastomyces dermatitidis, Candida species (e.g., Candida albicans, Candida glabrata, Candida kerr, Candida krusei, Candida parapsilosis, Candida pseudotropicalis, Candida quillermondii, Candida rugosa, Candida stellatoidea, and Candida tropicalis), Coccidioides immitis, Conidiobolus species, Cryptococcus neoforms, Cunninghamella species, dermatophytes, Histoplasma capsulatum, Microsporum gypseum, Mucor pusillus, Paracoccidioides brasiliensis, Pseudallescheria boydii, Rhinosporidium seeberi, Pneumocystis carinii, Rhizopus species (e.g., Rhizopus arrhizus, Rhizopus oryzae, and Rhizopus microsporus), Saccharomyces species, Sporothrix schenckii, zygomycetes, and classes such as Zygomycetes, Ascomycetes, the Basidiomycetes, Deuteromycetes, and Oomycetes.

### 2.2.3.1 Systemic Candidiasis

80% of all major systemic fungal infections are due to Candida species. The Merk Manual of Diagnosis and Therapy, 17th ed., 1999. Invasive candidiasis is most often caused by Candida albicans, Candida troicalis, and Candida glabrata in immunosuppressd patients. Id. Candidiasis is a defining opportunistic infection of AIDS, infecting the esophagus, trachea, bronchi, and lungs. Id. In HIV-infected patients, candidiasis is usually mucocutaneous and infects the oropharynx, the esophagus, and the vagina. Ampel, April-June 1996, Emerg. Infect. Dis. 2(2): 109-116.

Candida species are commensals that colonize the normal gastrointestinal tract and skin. The Merk Manual of Diagnosis and Therapy, Berkow et al. (eds.), 17th ed., 1999. Thus, cultures of Candidia from sputum, the mouth, urine, stool, vagina, or skin does not necessarily indicate an invasive, progressive infection. Id. In most cases, diagnosis of candidiasis requires presentation of a characteristic clinical lesion, documentation of histopathologic evidence of tissue invasion, or the exclusion of other causes. Id. Symptoms of systemic candidiasis infection of the respiratory tract are typically nonspecific, including dysphagia, coughing, and fever. Id.

All forms of candidiasis are considered serious, progressive, and potentially fatal. Id. Therapies for the treatment of candidiasis typically include the administration of the combination of the anti-fungal agents amphotericin B and flucytosine. Id. Unfortunately, acute renal failure has been associated with amphotericin B therapy. Dodds, supra. Fluconazole is not as effective as amphotericin B in treating certain species of Candida, but is useful as initial therapy in high oral or intravenous doses while species identification is pending. The Merk Manual of Diagnosis and Therapy, 17th ed., 1999. Fluconazole, however, has led to increasing treatment failures and anti-fungal resistance. Ampel, supra. Thus, there is a need for novel therapies for the treatment of systemic candidiasis.

### 2.2.3.2 Asperillosis

Aspergillus includes 132 species and 18 variants among which Aspergillus fumigatus is involved in 80% of Aspergillus-related diseases. Kurp et aL, 1999, Medscape General Medicine 1(3). Aspergillus fumigatus is the most common cause of invasive pulmonary aspergillosis that extends rapidly, causing progressive, and ultimately fatal respiratory failure. The Merck Manual of Diagnosis and Therapy, 17th ed., 1999. Patients undergoing long-term high-dose corticosteroid therapy, organ transplant patients, patients with hereditary disorders of neutrophil function, and patients infected with AIDS are at risk for aspergillosis.

Clinical manifestations of invasive pulmonary infection by Aspergillus include fever, cough, and chest pain. Aspergillus colonize preexisting cavity pulmonary lesions in the form of aspergilloma (fungus ball) which is composed of tangled masses hyphae, fibrin exudate, and inflammatory cells encapsulated by fibrous tissue. Id. Aspergillomas usually form and enlarge in pulmonary cavities originally caused by bronchiectasis, neoplasm, TB, and other chronic pulmonary infections. Id. Most aspergillomas do not respond to or require systemic anti-fungal therapy. Id. However, invasive infections often progress rapidly and are fatal, thus aggressive therapy comprising IV amphotericin B or oral itraconazole is required. Id. Unfortunately, high-dose amphotericin B may cause renal failure and itraconazole is effective only in moderately severe cases. Id. Therefore, there is a need for new therapies for the treatment of aspergillosis.

### 2.2.3.3 Cryptococcosis

Cases of cryptococcosis were rare before the HIV epidemic. Ampel, supra. AIDS patients, patients with Hodgkin's or other lymphomas or sarcoidosis, and patients undergoing long-term corticosteroid therapy are at increased risk for cryptococcosis. The Merk Manual of Diagnosis and Therapy, 17th ed., 1999. In most cases, cryptococcal infections are self-limited, but AIDS-associated cryptococcal infection may be in the form of a severe, progressive pneumonia with acute dyspnea and primary lesions in the lungs. Id. In cases of progressive disseminated cryptococcosis affecting non-immunocompromised patients, chronic meningitis is most common without clinically evident pulmonary lesions. Id.

Immunocompetent patients do not always require the administration of a therapy to treat localized pulmonary cryptococcosis. However, when such patients are administered a therapy for the treatment of localized pulmonary cryptococcosis, it typically consists of the administration of amphotericin B with or without flucytosine. Id. AIDS patients are generally administered an initial therapy consisting of amphotericin B and flucytosine and then oral fluconazole thereafter to treat cryptococcosis. Id. Renal and hematologic function of all patients receiving ampotericin B with or without flucytosine must be evaluated before and during therapy since flucytosine blood levels must be monitored to limit toxicity and administration of flucytosine may not be safe for patients with preexisting renal failure or bone marrow dysfunction. Id. Thus, new therapies for the treatment of cryptococcosis are needed.

### 2.2.4 Protozoan Infections

Protozoa are one-celled animals found worldwide in most habitats. Most species are free-living, but all higher animals are infected with one or more species of protozoa. Infections range from asymptomatic to life-threatening, depending on the species and strain of the parasite and the resistance of the host. Protozoa are microscopic unicellular eukaryotes that have a relatively complex internal structure and carry out complex metabolic activities. Some protozoa have structures for propulsion or other types of movement. In terms of classification, most protozoa are classified on the basis of light and electron microscopic morphology. The protozoa are currently classified into six phyla, with the members of the Sacromastigophora and Apicomplexa phyla causing human disease.

Virtually all humans have protozoa living in or on their body at some time, and many persons are infected with one or more species throughout their life. Some species are considered commensals, i.e., normally not harmful, whereas others are pathogens and usually produce disease. Protozoan diseases range from very mild to life-threatening. Individuals whose defenses are able to control but not eliminate a parasitic infection become carriers and constitute a large source of infection for others.

Many protozoan infections that are inapparent or mild in normal individuals can be life-threatening in immunosuppressed patients, particularly in patients with acquired immune deficiency syndrome (“AIDS”). Evidence suggests that many healthy persons harbor low numbers of Pneumocystis carinii in their lungs. However, this parasite produces a frequently fatal pneumonia in immunosuppressed patients such as those with AIDS. Toxoplasma gondii, a very common protozoan parasite, usually causes a rather mild initial illness followed by a long-lasting latent infection. AIDS patients, however, can develop fatal toxoplasmic encephalitis. Cryptosporidium was described in the 19^{th} century, but widespread human infection has only recently been recognized. Cryptosporidium is another protozoan that can produce serious complications in patients with AIDS. Microsporidiosis in humans was reported in only a few instances prior to the appearance of AIDS. It has now become a more common infection in AIDS patients. As more thorough studies of patients with AIDS are made, it is likely that other rare or unusual protozoan infections will be diagnosed.

Non-limiting examples of the genera of parasitic protozoa and their associated diseases include: Leishmania (visceral, cutaneous and mucocutaneous infection); Trypanosoma (sleeping sickness, Chagas' disease); Giardia (diarrhea); Trichomonas (vaginitis); Entamoeba (dysentery, liver abscess); Dientamoeba (colitis); Naegleria and Acanthamoeba (central nervous system and corneal ulcers); Babesia (Babesiosis); Plasmodium (malaria); Isospora (diarrhea); Sarcocystis (diarrhea); Toxoplasma (toxoplasmosis); Enterocytozoon (diarrhea); Balantidium (dysentery); and Pneumocystis (pneumonia). For reviews of protozoan infections, see, e.g., Englund and Sher (eds): The Biology of Parasitism. A Molecular and humunological Approach. Alan R. Liss, New York, 1988; Goldsmith and Heyneman (eds): Tropical Medicine and Parasitology. Appleton and Lange, East Norwalk, Conn., 1989; Lee et al. (eds): An Illustrated Guide to the Protozoa. Society of Protozoologists, Lawrence, K S, 1985; Kotlar and Orenstein, 1994, J. Gastroenterol. 89:1998; and Neva and Brown, Basic Clinical Parasitology, 6^{th} ed., Appleton & Lange, Norwalk, Conn., 1994.

### 2.3 EphA2 and Infections

Many clinically important pathogens, including bacteria, initiate disease by invading the epithelial cell layers. Microbial entry into the epithelium is an active process that requires signaling from the invading pathogen to the host cell, although the specific signaling pathways involved differ for different types of pathogens (Finlay and Cossart, 1997, Science 276:718-725). Besides facilitating the invasion process, the interaction between an invading pathogen and a host cell leads to activation of a program of epithelial gene expression. This program encompasses genes involved in the inflammatory response and membrane-associated proteins. Recent studies using cDNA array expression analysis have revealed that a host of specific genes are upregulated or downregulated in response to an infection.

EphA2 (epithelial cell kinase) is a 130 kDa member of the Eph family of receptor tyrosine kinases (Zantek N. et al, 1999, Cell Growth Differ. 10:629-38; Lindberg R. et al., 1990, Mol. Cell. Biol. 10:6316-24). The function of EphA2 is not known, but it has been suggested to regulate proliferation, differentiation, and barrier function of colonic epithelium (Rosenberg et al., 1997, Am. J. Physiol. 273:G824-32), vascular network assembly, endothelial migration, capillary morphogenesis, and angiogenesis (Stein et al., 1998, Genes Dev. 12:667-78), nervous system segmentation and axon pathfinding (Bovenkamp D. and Greer P., 2001, DNA Cell Biol. 20:203-13), tumor neovascularization (Ogawa K. et al., 2000, Oncogene 19:6043-52), and cancer metastasis (International Patent Publication Nos. WO 01/9411020, WO 96/36713, WO 01/12840, WO 01/12172).

The natural ligand of EphA2 is EphrinA1 (Eph Nomenclature Committee, 1997, Cell 90(3):403-4; Gale, et al., 1997, Cell Tissue Res. 290(2): 227-41). The EphA2 and EphrinA1 interaction is thought to help anchor cells on the surface of an organ and also down regulate epithelial and/or endothelial cell proliferation by decreasing EphA2 expression through EphA2 autophosphorylation (Lindberg et al., 1990, supra). Under natural conditions, the interaction helps maintain an epithelial cell barrier that protects the organ and helps regulate over proliferation and growth of epithelial cells. However, there are disease states that prevent epithelial cells from forming a protective barrier or cause the destruction and/or shedding of epithelial and/or endothelial cells and thus prevent proper healing from occurring.

### 3. SUMMARY OF THE INVENTION

The present invention is based, in part, on the inventors' discovery that EphA2 is upregulated in epithelial cells infected with RSV. Without being bound to a particular theory or mechanism, the upregulation of EphA2 expression in pathogen-infected cells could promote unwanted cell survival. The invention thus provides methods and compositions designed for the treatment, management, prevention and/or amelioration of a pathogen infection, including, but not limited to, a viral infection, a bacterial infection, a fungal infection and a protozoan infection. In particular, the present invention provides methods for treating, managing, preventing, and/or ameliorating an infection where the expression of EphA2 is upregulated in infected cells (e.g., infected EphA2-expressing cells), said methods comprising administering to a subject in need thereof an effective amount of one or more EphA2/EphrinA1 Modulators, and optionally, an effective amount of a therapy other than an EphA2/EphrinA1 Modulator. In a preferred embodiment, the pathogen infections to be treated, prevented, managed and/or ameliorated in accordance with the methods of the invention are intracellular pathogen infections.

In a preferred embodiment, the bacterial infections to be treated, managed, prevented and/or ameliorated in accordance with the methods of the present invention are intracellular bacterial infections. Non-limiting examples of intracellular bacteria that cause and/or are associated with infections in humans include Mycobacterium tuberculosis, Mycobacterium leprae, Salmonella enterica serovar Typhi, Brucella sp, Legionella sp, Listeria monocytogenes, Francisella tularensis, Rickettsia rickettsii; Rickettsia prowazekii; Rickettsia typhi; Rickettsia tsutsugamushi; Chlamydia trachomatis; Chlamydia psittaci; and Chlamydia pneumoniae. In a specific embodiment, the invention provides a method of preventing, treating, managing and/or ameliorating an intracellular bacterial infection, the method comprising administering to a subject in need thereof an EphA2/EphrinA1 Modulator, and optionally, a therapy other than an EphA2/EphrinA1 Modulator. In a preferred embodiment, the intracellular bacterial infection that is prevented, treated, managed and/or ameliorated causes and/or is associated with an increase in EpbA2 expression in infected cells (e.g., infected epithelial cells). In a specific embodiment, cells infected with the intracellular bacteria have at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 98%, or at least 1 fold, at least 1.5 fold, at least 2 fold, at least 2.5 fold, at least 3 fold, at least 3.5 fold, at least 4 fold, at least 4.5 fold, at least 5 fold, at least 7 fold, or at least 10 fold higher level of expression of EphA2 than uninfected cells from a subject (e.g., the same subject) or a population of subjects as assessed by an assay described herein or known in the art (e.g., RT-PCR, Northern blot, FACS analysis, or an immunoassay such as ELISA). In another specific embodiment, the intracellular bacterial infection to be prevented, treated, managed, and/or ameliorated in accordance with the methods of the invention is active. In another embodiment, the intracellular bacterial infection to be prevented, treated, managed, and/or ameliorated in accordance with the methods of the invention is latent.

In certain embodiments, the intracellular bacterial infection to be prevented, treated, managed, and/or ameliorated in accordance with the methods of the invention is an infection caused by Mycobacterium tuberculosis, Mycobacterium leprae, Salmonella enterica serovar Typhi, Brucella sp, Legionella sp, Listeria monocytogenes, Francisella tularensis, Rickettsia rickettsii; Rickettsia prowazekii; Rickettsia typhi; Rickettsia tsutsugamushi; Chlamydia trachomatis; Chlamydia psittaci; and Chlamydia pneumoniae. In certain other embodiments, the intracellular bacterial infection to be prevented, treated, managed, and/or ameliorated in accordance with the methods of the invention is not an infection caused by one or more of the following intracellular bacteria: Mycobacterium tuberculosis, Mycobacterium leprae, Salmonella enterica serovar Typhi, Brucella sp, Legionella sp, Listeria monocytogenes, Francisella tularensis, Rickettsia rickettsii; Rickettsia prowazekii; Rickettsia typhi; Rickettsia tsutsugamushi; Chlamydia trachomatis; Chlamydia psittaci; and Chlamydia pneumoniae.

Non-limiting examples of viruses that cause and/or are associated with infections in humans include Hepatitis A virus; Hepatitis B virus; HIV; Severe Acute Respiratory Syndrome Virus; Poliomyelitis virus; Rubella virus; West Nile Fever virus; Rabies virus; Ebola virus Zaire; Mumps virus; Measles virus; Hantavirus; Lassa fever virus; Rotavirus; Cytomegalovirus; Parainfluenza virus; Respiratory syncytial virus (“RSV”); and Avian & Human Metapneumovirus. In a specific embodiment, the invention provides a method of preventing, treating, managing and/or ameliorating a viral infection, the method comprising administering to a subject in need thereof an EphA2/EphrinA1 Modulator, and optionally, a therapy other than an EphA2/EphrinA1 Modulator. In a preferred embodiment, the viral infection that is prevented, treated, managed and/or ameliorated causes and/or is associated with an increase in EphA2 expression in infected cells (e.g., infected epithelial cells). In a specific embodiment, cells infected with the virus have at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 98%, or at least 1 fold, at least 1.5 fold, at least 2 fold, at least 2.5 fold, at least 3 fold, at least 3.5 fold, at least 4 fold, at least 4.5 fold, at least 5 fold, at least 7 fold, or at least 10 fold higher level of expression of EphA2 than uninfected cells from a subject (e.g., the same subject) or a population of subjects as assessed by an assay described herein or known in the art (e.g., RT-PCR, Northern blot, FACS analysis, or an immunoassay such as ELISA). In another specific embodiment, the viral infection to be prevented, treated, managed, and/or ameliorated in accordance with the methods of the invention is active. In another embodiment, the viral infection to be prevented, treated, managed, and/or ameliorated in accordance with the methods of the invention is latent.

In certain embodiments, the viral infection to be prevented, treated, managed, and/or ameliorated in accordance with the methods of the invention is an infection caused by Human papilloma virus, Varicella Zoster virus, Dengue virus, Ebola virus, Herpes Simplex virus-2, Hantavirus, Hepatitis A virus, Hepatitis B virus, Hepatitis C virus, Hepatitis D virus, Hepatitis E virus, Influenza viruses A, B and C, Junin virus, Lassa virus, Machupo virus, Rubeola virus, Epstein Barr virus, Cytomegalovirus, Human coronavirus, Variola virus, Yellow fever virus, West Nile virus, Western EE virus, Adenovirus, Rotavirus, Semliki Forest virus, Vaccinia virus, Venezuelan EE virus, Lymphocytic choriomeningitis virus, Guanarito virus, Rift valley fever virus, Marburg virus, Tick borne encephalitis virus, Hendra virus, Nipah virus, Crimean-Congo hemorrhagic fever virus, Sabia virus, Parainfluenza virus, Respiratory syncytial virus, or Avian & Human Metapneumovirus. In certain other embodiments, the viral infection to be prevented, treated, managed, and/or ameliorated in accordance with the methods of the invention is not an infection caused by one or more of the following viruses: Human papilloma virus, Varicella Zoster virus, Dengue virus, Ebola virus, Herpes Simplex virus-2, Hantavirus, Hepatitis A virus, Hepatitis C virus, Hepatitis D virus, Hepatitis E virus, Influenza viruses A, B and C, Junin virus, Lassa virus, Machupo virus, Rubeola virus, Epstein Barr virus, Cytomegalovirus, Human coronavirus, Variola virus, Yellow fever virus, West Nile virus, Western EE virus, Adenovirus, Rotavirus, Semliki Forest virus, Vaccinia virus, Venezuelan EE vias, Lymphocytic choriomeningitis virus, Guanarito virus, Rift valley fever virus, Marburg virus, Tick borne encephalitis virus, Hendra virus, Nipah virus, Crimean-Congo hemorrhagic fever virus, Sabia virus, Parainfluenza virus, Respiratory syncytial virus, or Avian & Human Metapneumovirus. In a specific embodiment, a viral infection to be prevented, treated, managed and/or ameliorated by the methods and compositions of the invention is not a respiratory viral infection. In a specific embodiment, the viral infection to be prevented, treated, managed and/or ameliorated by the methods and compositions of the invention is not a RSV infection.

Non-limiting examples of protozoa that cause and/or are associated with infections in humans include Leishmania; Trypanosoma; Giardia; Trichomonas; Entamoeba; Dientamoeba; Naegleria and Acanthamoeba; Babesia; Plasmodium; Isospora; Sarcocystis; Toxoplasma; Enterocytozoon; Balantidium; and Pneumocystis. In a specific embodiment, the invention provides a method of preventing, treating, managing and/or ameliorating an intracellular protozoan infection, the method comprising administering to a subject in need thereof an EphA2/EphrinA1 Modulator, and optionally, a therapy other than an EphA2/EphrinA1 Modulator. In a preferred embodiment, the protozoan infection that is prevented, treated, managed and/or ameliorated causes and/or is associated with an increase in EphA2 expression in infected cells (e.g., infected epithelial cells). In a specific embodiment, cells infected with the protozoan have at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 98%, or at least 1 fold, at least 1.5 fold, at least 2 fold, at least 2.5 fold, at least 3 fold, at least 3.5 fold, at least 4 fold, at least 4.5 fold, at least 5 fold, at least 7 fold, or at least 10 fold higher level of expression of EphA2 than uninfected cells from a subject (e.g., the same subject) or a population of subjects as assessed by an assay described herein or known in the art (e.g., RT-PCR, Northern blot, FACS analysis, or an immunoassay such as ELISA). In another specific embodiment, the protozoan infection to be prevented, treated, managed, and/or ameliorated in accordance with the methods of the invention is active. In another embodiment, the protozoan infection to be prevented, treated, managed, and/or ameliorated in accordance with the methods of the invention is latent.

In certain embodiments, the protozoan infection to be prevented, treated, managed, and/or ameliorated in accordance with the methods of the invention is an infection caused by Leishmania; Trypanosoma; Giardia; Trichomonas; Entamoeba; Dientamoeba; Naegleria and Acanthamoeba; Babesia; Plasmodium; Isospora; Sarcocystis; Toxoplasma; Enterocytozoon; Balantidium; and Pneumocystis. In certain other embodiments, the protozoan infection to be prevented, treated, managed, and/or ameliorated in accordance with the methods of the invention is not an infection caused by one or more of the following intracellular protozoa: Leishmania; Trypanosoma; Giardia; Trichomonas; Entamoeba; Dientamoeba; Naegleria and Acanthamoeba; Babesia; Plasmodium; Isospora; Sarcocystis; Toxoplasma; Enterocytozoon; Balantidium; and Pneumocystis.

Non-limiting examples of fungi that cause and/or are associated with infections in humans include Absidia species (e.g., Absidia corymbifera and Absidia ramosa), Aspergillus species, (e.g., Aspergillus flavus, Aspergillus fumigatus, Aspergillus nidulans, Aspergillus niger, and Aspergillus terreus), Basidiobolus ranarum, Blastomyces dermatitidis, Candida species (e.g., Candida albicans, Candida glabrata, Candida kerr, Candida krusei, Candida parapsilosis, Candida pseudotropicalis, Candida quillermondii, Candida rugosa, Candida stellatoidea, and Candida tropicalis), Coccidioides immitis, Conidiobolus species, Cryptococcus neoforms, Cunninghamella species, dermatophytes, Histoplasma capsulatum, Microsporum gypseum, Mucor pusillus, Paracoccidioides brasiliensis, Pseudallescheria boydii, Rhinosporidium seeberi, Pneumocystis carinii, Rhizopus species (e.g., Rhizopus arrhizus, Rhizopus oryzae, and Rhizopus microsporus), Saccharomyces species, Sporothrix schenckii, zygomycetes, and classes such as Zygomycetes, Ascomycetes, the Basidiomycetes, Deuteromycetes, and Oomycetes. In a specific embodiment, the invention provides a method of preventing, treating, managing and/or ameliorating a fungal infection, the method comprising administering to a subject in need thereof an EphA2/EphrinA1 Modulator, and optionally, a therapy other than an EphA2/EphrinA1 Modulator. In a preferred embodiment, the fungal infection that is prevented, treated, managed and/or ameliorated causes and/or is associated with an increase in EphA2 expression in infected cells (e.g., infected epithelial cells). In a specific embodiment, cells infected with the fungi have at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 98%, or at least 1 fold, at least 1.5 fold, at least 2 fold, at least 2.5 fold, at least 3 fold, at least 3.5 fold, at least 4 fold, at least 4.5 fold, at least 5 fold, at least 7 fold, or at least 10 fold higher level of expression of EphA2 than uninfected cells from a subject (e.g., the same subject) or a population of subjects as assessed by an assay described herein or known in the art (e.g., RT-PCR, Northern blot, FACS analysis, or an immunoassay such as ELISA). In another specific embodiment, the fungal infection to be prevented, treated, managed, and/or ameliorated in accordance with the methods of the invention is active. In another embodiment, the fungal infection to be prevented, treated, managed, and/or ameliorated in accordance with the methods of the invention is latent.

In certain embodiments, the fungal infection to be prevented, treated, managed, and/or ameliorated in accordance with the methods of the invention is an infection caused by by Candida species, Aspergillus species, and Cryptococcus neoformans. In certain other embodiments, the fungal infection to be prevented, treated, managed, and/or ameliorated in accordance with the methods of the invention is not an infection caused by one or more of the following fungus species: Absidia species (e.g., Absidia corymbifera and Absidia ramosa), Aspergillus species, (e.g., Aspergillus flavus, Aspergillus fumigatus, Aspergillus nidulans, Aspergillus niger, and Aspergillus terreus), Basidiobolus ranarum, Blastomyces dermatitidis, Candida species (e.g., Candida albicans, Candida glabrata, Candida kerr, Candida krusei, Candida parapsilosis, Candida pseudotropicalis, Candida quillermondii, Candida rugosa, Candida stellatoidea, and Candida tropicalis), Coccidioides immitis, Conidiobolus species, Cryptococcus neoforms, Cunninghamella species, dermatophytes, Histoplasma capsulatum, Microsporum gypseum, Mucor pusillus, Paracoccidioides brasiliensis, Pseudallescheria boydii, Rhinosporidium seeberi, Pneumocystis carinii, Rhizopus species (e.g., Rhizopus arrhizus, Rhizopus oryzae, and Rhizopus microsporus), Saccharomyces species, Sporothrix schenckii, zygomycetes, and classes such as Zygomycetes, Ascomycetes, the Basidiomycetes, Deuteromycetes, and Oomycetes.

EphA2/EphrinA1 Modulators are agents that confer a biological effect by modulating (directly or indirectly): (i) the expression of EphA2 and/or an endogenous ligand(s) of EphA2 (preferably, EphrinA1), at, e.g., the transcriptional, post-transcriptional, translational or post-translation level; and/or (ii) an activity(ies) of EphA2 and/or EphrinA1. Examples of EphA2/EphrinA1 Modulators include, but are not limited to, agents that inhibit or reduce the interaction between EphA2 and an endogenous ligand(s) of EphA2, preferably, EphrinA (hereinafter “EphA2/EphrinA1 Interaction Inhibitors”). Non-limiting examples of EphA2/EphrinA1 Interaction Inhibitors include: (i) agents that bind to EphA2, prevent or reduce the interaction between EphA2 and EphrinA1, and induce EphA2 signal transduction (e.g., soluble forms of EphrinA1 (e.g., an EphrinA1-Fc in monomeric or multimeric form), and antibodies that bind to EphA2, induce signaling and phosphorylation of EphA2 (i.e., an EphA2 agonistic antibody)); (ii) agents that bind to EphA2, prevent or reduce the interaction between the EphA2 and EphrinA1 and prevent or induce very low to negligible levels of EphA2 signal transduction (e.g., EphA2 antagonistic antibodies and dominant negative forms of EphrinA1); (iii) agents that bind to EphrinA1, prevent or reduce the interaction between EphA2 and EphrinA1, and induce EphrinA1 signal transduction (e.g., soluble forms of EphA2 (e.g., EphA2-Fc) and antibodies that bind to EphrinA1 and induce EphrinA1 signal transduction); and (iv) agents that bind to EphrinA1, prevent or reduce the interaction between an EphA2 and EphrinA1, and prevent or induce very low to negligible levels of EphrinA1 signal transduction (e.g., dominant negative forms of an EphA2 and anti-EphrinA1 antibodies).

EphA2/EphrinA1 Modulators also include, but are not limited to, agents that modulate the expression of EphA2. Such agents can decrease/downregulate EphA2 expression (e.g., EphA2 antisense molecules, RNAi and ribozymes) or increase/upregulate EphA2 expression such that the amount of EphA2 on the cell surface exceeds the amount of endogenous ligand (preferably, EphrinA1) available for binding, and thus, increases the amount of unbound EphA2 (e.g., nucleic acids encoding an EphA2)).

In certain embodiments, EphA2/EphrinA1 Modulators are agents that modulate the expression of EphrinA1. Such agents can decrease/downregulate EphrinA1 expression (e.g., EphrinA1 antisense molecules, RNAi and ribozymes) or increase/upregulate Ephrin expression (e.g., nucleic acids encoding EphrinA1)).

In yet other embodiments, EphA2/EphrinA1 Modulators of the invention include, but are not limited to, agents that modulate the protein stability or protein accumulation of EphA2 or EphrinA1.

In further embodiments, EphA2/EphrinA1 Modulators of the invention are agents that promote kinase activity (e.g., of EphA2, EphrinA1 or of a heterologous protein known to associate with EphA2 or EphrinA1 at the cell membrane).

In yet further embodiments, EphA2/EphrinA1 Modulators include, but are not limited to, agents that bind to EphA2 and prevent or reduce EphA2 signal transduction but do not inhibit or reduce the interaction between EphA2 and EphrinA1 (e.g., an EphA2 intrabody); and agents that bind to EphrinA1 and prevent or reduce EphrinA1 signal transduction but do not inhibit or reduce the interaction between EphrinA1 and EphA2 (e.g., an EphrinA1 antibody).

In specific embodiments of the invention, an EphA2/EphrinA1 Modulator does one or more of the following: (i) decreases EphA2 expression and/or activity; (ii) causes apoptosis and/or necrosis of EphA2-expressing cells infected with a pathogen; and (iii) causes EphA2 ligand-induced phosphorylation (e.g., autophosphorylation) and degradation. In other specific embodiments, an EphA2/EphrinA1 Modulator is one of the following: (i) a soluble EphrinA1 molecule (e.g., EphrinA1-Fc); (ii) an EphA2 antisense nucleic acid molecule; (iii) an EphA2 agonistic antibody that induces EphA2 phosphorylation and degradation; (iv) an EphA2 vaccine; (v) an anti-EphrinA1 or anti-EphA2 antibody conjugated to a cytotoxic agent; (vi) a multispecific antibody (e.g., bispecific antibody (such as a BiTE molecule) that targets, e.g., EphA2 and a pathogen antigen or cell marker.

The EphA2/EphrinA1 Modulator can be an antibody, preferably a monoclonal antibody, which may have a low K_{off} rate (e.g., K_{off} less than 3×10⁻³s⁻¹). In one embodiment, the antibodies used in the methods of the invention are Eph099B-102.147, Eph099B-208.261, Eph099B-210.248, B233, EA2 or EA5. In a more preferred embodiment, the antibodies used in the methods of the invention are human, humanized or chimerized Eph099B-102.147, Eph099B-208.261, Eph099B-210.248, B233, EA2 or EA5. In a specific embodiment, an EphA2/EphrinA1 Modulator is not Eph099B-102.147, Eph099B-208.261, Eph099B-210.248, B233, EA2 or EA5.

In a specific embodiment, an EphA2/EphrinA1 Modulator of the invention is not an agent or compound disclosed in U.S. Patent Publication No. US 2004/0180823A1 or International Publication No. WO 2004/028551 A1.

The present invention provides pharmaceutical compositions comprising EphA2/EphrinA1 Modulators, and optionally, therapeutic or prophylactic agents (e.g., immunomodulatory agents, anti-viral agents, anti-inflammatory agents, anti-bacterial agents, anti-fungal agents, etc.) other than an EphA2/EphrinA1 Modulator. The present invention also provides methods of detecting, diagnosing and/or prognosing an infection, in particular an intracellular pathogen infection, and/or methods monitoring the efficacy of a therapy for the prevention, treatment, management and/or amelioration of an infection using the EphA2/EphrinA1 Modulators of the invention. Such methods may be used in combination with other methods for detecting, diagnosing, monitoring or prognosing an infection. In a preferred embodiment, the infection causes and/or is associated with EphA2 overexpression. In specific embodiments, the invention provides methods for detecting, diagnosing, monitoring or prognosing latent infections.

The invention further provides articles of manufacture and kits comprising an EphA2/EphrinA1 Modulator of the invention, and optionally, one or more therapeutic or prophylactic agents (e.g., immunomodulatory agents, anti-viral agents, anti-inflammatory agents, anti-bacterial agents, anti-fungal agents, etc.) other than an EphA2/EphrinA1 Modulator. In specific embodiments, the articles of manufacture and kits include instructions for dosage and administration of the EphA2/EphrinA1 Modulator and, optional other therapy.

### 3.1 DEFINITIONS

As used herein, the term “agent” refers to a molecule that has a desired biological effect. Agents include, but are not limited to, proteinaceous molecules, including, but not limited to, peptides, polypeptides, proteins, post-translationally modified proteins, antibodies etc.; vaccines (e.g., Listeria-based vaccines) small molecules (less than 1000 daltons), inorganic or organic compounds; and nucleic acid molecules including, but not limited to, double-stranded or single-stranded DNA, or double-stranded or single-stranded RNA (e.g., antisense, RNAi, etc.), aptamers, as well as triple helix nucleic acid molecules. Agents can be derived or obtained from any known organism (including, but not limited to, animals (e.g., mammals (human and non-human mammals)), plants, bacteria, fungi, and protista, or viruses) or from a library of synthetic molecules. Agents that are EphA2/EphrinA1 Modulators modulate (directly or indirectly): (i) the expression of EphA2 and/or an endogenous ligand(s) of EphA2, preferably, EphrinA1, at, e.g., the transcriptional, post-transcriptional, translational or post-translation level; and/or (ii) an activity(ies) of EphA2 and/or an endogenous ligand(s) of EphA2, preferably, EphrinA1.

As used herein, the term “analog” in the context of a proteinaceous agent (e.g., a peptide, polypeptide, protein or antibody) refers to a proteinaceous agent that possesses a similar or identical function as a second proteinaceous agent (e.g., an EphA2 polypeptide or an EphrinA1 polypeptide) but does not necessarily comprise a similar or identical amino acid sequence or structure of the second proteinaceous agent. A proteinaceous agent that has a similar amino acid sequence refers to a proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second proteinaceous agent; (b) a proteinaceous agent encoded by a nucleotide sequence that hybridizes under stringent conditions to a nucleotide sequence encoding a second proteinaceous agent of at least 20 amino acid residues, at least 30 amino acid residues, at least 40 amino acid residues, at least 50 amino acid residues, at least 60 amino residues, at least 70 amino acid residues, at least 80 amino acid residues, at lcast 90 amino acid residues, at least 100 amino acid residues, at least 125 amino acid residues, or at least 150 amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second proteinaceous agent. A proteinaceous agent with similar structure to a second proteinaceous agent refers to a proteinaceous agent that has a similar secondary, tertiary or quaternary structure of the second proteinaceous agent. The structure of a proteinaceous agent can be determined by methods known to those skilled in the art, including but not limited to, X-ray crystallography, nuclear magnetic resonance, and crystallographic electron microscopy. Preferably, the proteinaceous agent has EphA2 or EphrinA1 activity.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87: 2264-2268, modified as in Karlin and Altschul, 1993, Proc. Natl. Acad. Sci. U.S.A. 90: 5873-5877. Such an algorithm is incorporated into the NBLAST and XBLAST programs of Altschul et al., 1990, J. Mol. Biol. 215: 403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25: 3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4: 11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “analog” in the context of a non-proteinaceous analog refers to a second organic or inorganic molecule which possesses a similar or identical function as a first organic or inorganic molecule and is structurally similar to the first organic or inorganic molecule.

As used herein, the term “antibodies that immunospecifically bind to EphA2” and analogous terms refer to antibodies that specifically bind to an EphA2 polypeptide or a fragment of an EphA2 polypeptide, and do not specifically bind to non-EphA2 polypeptides. Preferably, antibodies that immunospecifically bind to an EphA2 polypeptide or a fragment thereof do not cross-react with other non-related antigens. In certain embodiments, antibodies or fragments that immunospecifically bind to EphA2 may be cross-reactive with related antigens (e.g., other types Eph receptors from the A or B family of Eph receptors). Antibodies that immunospecifically bind to an EphA2 polypeptide or a fragment thereof can be identified, for example, by immunoassays or other techniques known to those of skill in the art. Preferably, antibodies that immunospecifically bind to an EphA2 polypeptide or a fragment thereof only modulate an EphA2 activity(ies) and do not significantly affect other activities. Antibodies that immunospecifically bind to an EphA2 polypeptide or fragment thereof are preferably monoclonal antibodies, which may have a low K_{off} rate (e.g., K_{off} less than 3×10⁻³s⁻¹). In one embodiment, the antibodies used in the methods of the invention are Eph099B-102.147, Eph099B-208.261, Eph099B-210.248, B233, EA2 or EA5. In a more preferred embodiment, the antibodies used in the methods of the invention are human or hummanized Eph099B-102.147, Eph099B-208.261, Eph099B-210.248, B233, EA2 or EA5. In a specific embodiment, an EphA2/EphrinA1 Modulator is not Eph099B-102.147, Eph099B-208.261, Eph099B-210.248, B233, EA2 or EA5.

As used herein, the term “antibodies that immunospecifically bind to EphrinA1 ” and analogous terms refer to antibodies that specifically bind to an EphrinA1 polypeptide or a fragment of an EphrinA1 polypeptide, and do not specifically bind to non-EphrinA1 polypeptides. Preferably, antibodies that immunospecifically bind to an EphrinA1 polypeptide or a fragment thereof do not cross-react with other non-related antigens. In certain embodiments, antibodies or fragments that immunospecifically bind to EphrinA1 may be cross-reactive with related antigens (e.g., other types Ephrins from the A or B family of Ephrin ligands). Antibodies that immunospecifically bind to an EphrinA1 polypeptide or a fragment thereof can be identified, for example, by immunoassays or other techniques known to those of skill in the art. Preferably, antibodies that immunospecifically bind to an EphrinA1 polypeptide or a fragment thereof only modulate an EphrinA1 activity(ies) and do not significantly affect other activities.

Antibodies of the invention include, but are not limited to, synthetic antibodies, monoclonal antibodies, recombinantly produced antibodies, multispecific antibodies (including bi-specific antibodies), human antibodies, humanized antibodies, chimeric antibodies, intrabodies, single-chain Fvs (scFv) (e.g., including monospecific and bi-specific, etc.), Fab fragments, F(ab′) fragments, disulfide-linked Fvs (sdFv), anti-idiotypic (anti-Id) antibodies, and epitope-binding fragments of any of the above. In particular, antibodies of the present invention include immunoglobulin molecules and immunologically active portions of immunoglobulin molecules, i.e., molecules that contain an antigen-binding site that immunospecifically binds to an EphA2 antigen or an EphrinA1 antigen (e.g., one or more complementarity determining regions (CDRs) of an anti-EphA2 antibody or of an anti-EphrinA1 antibody). The antibodies of the invention can be of any type (e.g., IgG, IgE, IgM, IgD, IgA and IgY), class (e.g., IgG₁, IgG₂, IgG₃, IgG₄, IgA₁ and IgA2) or subclass of immunoglobulin molecule.

As used herein, the term “derivative” in the context of a proteinaceous agent (e.g., proteins, polypeptides, peptides, and antibodies) refers to a proteinaceous agent that comprises the amino acid sequence which has been altered by the introduction of amino acid residue substitutions, deletions, and/or additions. The term “derivative” as used herein also refers to a proteinaceous agent which has been modified, i.e., by the covalent attachment of a type of molecule to the proteinaceous agent. For example, but not by way of limitation, a derivative of a proteinaceous agent may be produced, e.g., by glycosylation, acetylation. pegylation, phosphorylation, amidation, derivatization by known protecting/blocking groups, proteolytic cleavage, linkage to a cellular ligand or other protein, etc. A derivative of a proteinaceous agent may also be produced by chemical modifications using techniques known to those of skill in the art, including, but not limited to specific chemical cleavage, acetylation, formylation, metabolic synthesis of tunicamycin, etc. Further, a derivative of a proteinaceous agent may contain one or more non-classical amino acids. A derivative of a proteinaceous agent possesses an identical function(s) as the proteinaceous agent from which it was derived. In a specific embodiment, a derivative of a proteinaceous agent is a derivative an EphA2 polypeptide, an EphrinA1 polypeptide, a fragment of an EphA2 polypeptide or EphrinA1 polypeptide, an antibody that immunospecifically binds to an EphA2 polypeptide or fragment thereof, or an antibody that immunospecifically binds to an EphrinA1 polypeptide or fragment thereof. In one embodiment, a derivative of an EphA2 polypeptide, an EphrinA1 polypeptide, a fragment of an EphA2 polypeptide or EphrinA1 polypeptide, an antibody that immunospecifically binds to an EphA2 polypeptide or fragment thereof, or an antibody that immunospecifically binds to an EphrinA1 polypeptide or fragment thereof possesses a similar or identical function as an EphA2 polypeptide, an EphrinA1 polypeptide, a fragment of an EphA2 polypeptide or EphrinA1 polypeptide, an antibody that immunospecifically binds to an EphA2 polypeptide or fragment thereof, or an antibody that immunospecifically binds to an EphrinA1 polypeptide or fragment thereof. In another embodiment, a derivative of an EphA2 polypeptide, an EphrinA1 polypeptide, a fragment of an EphA2 polypeptide or EphrinA1 polypeptide, an antibody that immunospecifically binds to an EphA2 polypeptide or fragment thereof, or an antibody that immunospecifically binds to an EphrinA1 polypeptide or fragment thereof has an altered activity when compared to an unaltered polypeptide. For example, a derivative antibody or fragment thereof can bind to its epitope more tightly or be more resistant to proteolysis.

As used herein, the term “derivative” in the context of a non-proteinaceous derivative refers to a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, carboxyl, nitryl, or amine group. An organic molecule may also, for example, be esterified, alkylated and/or phosphorylated.

As used herein, the term “effective amount” refers to the amount of a therapy (e.g., a prophylactic or therapeutic agent) which is sufficient to reduce and/or ameliorate the severity and/or duration of an ian ion, symptom thereof, prevent the advancement of said infection, cause regression of said infection, prevent the recurrence, development, or onset of one or more symptoms associated with said infection, or enhance or improve the prophylactic or therapeutic effect(s) of another therapy (e.g., prophylactic or therapeutic agent). Non-limiting examples of effective amounts of EphA2/EphrinA1 Modulators are provided in Section 5.4, infra.

As used herein, the term “endogenous ligand” or “natural ligand” refers to a molecule that normally binds a particular receptor in vivo. For example, EphrinA1 is an endogenous ligand of EphA2.

As used herein, the term “EphA2/EphrinA1 Modulator” refers to an agent(s) that confers a biological effect by modulating (directly or indirectly): (i) the expression of EphA2 and/or an endogenous ligand(s) of EphA2, preferably, EphrinA1, at, e.g., the transcriptional, post-transcriptional, translational or post-translation level; and/or (ii) an activity(ies) of EphA2 and/or an endogenous ligand(s) of EphA2, preferably, EphrinA1.

Examples of EphA2/EphrinA1 Modulators include, but are not limited to, agents that inhibit or reduce the interaction between EphA2 and an endogenous ligand(s) of EphA2, preferably, EphrinA1 (hereinafter “EphA2/EphrinA1 Interaction Inhibitors”). Non-limiting examples of EphA2/EphrinA1 Interaction Inhibitors include: (i) agents that bind to EphA2, prevent or reduce the interaction between EphA2 and EphrinA1, and induce EphA2 signal transduction (e.g., soluble forms of EphrinA1 (e.g., an EphrinA1-Fc in monomeric or multimeric form), and antibodies that bind to EphA2, induce signaling and phosphorylation of EphA2 (i.e., an EphA2 agonistic antibody)); (ii) agents that bind to EphA2, prevent or reduce the interaction between the EphA2 and EphrinA1, and prevent or induce very low to negligible levels of EphA2 signal transduction (e.g., EphA2 antagonistic antibodies and dominant negative forms of EphrinA1); (iii) agents that bind to EphrinA1, prevent or reduce the interaction between EphA2 and EphrinA1, and induce EphrinA1 signal transduction (e.g., soluble forms of EphA2 (e.g., EphA2-Fc) and antibodies that bind to EphrinA1 and induce EphrinA1 signal transduction); and (iv) agents that bind to EphrinA1, prevent or reduce the interaction between an EphA2 and EphrinA1, and prevent or induce very low to negligible levels of EphrinA1 signal transduction (e.g., dominant negative forms of an EphA2 and anti-EphrinA1 antibodies).

In further embodiments, EphA2/EphrinA1 Modulators include, but are not limited to, agents that modulate the expression of EphA2. Such agents can decrease/downregulate EphA2 expression (e.g., EphA2 antitsense molecules, RNAi and ribozymes) or increase/upregulate EphA2 expression such that the amount of EphA2 on the cell surface exceeds the amount of endogenous ligand (preferably, EphrinA1) available for binding, and thus, increases the amount of unbound EphA2 (e.g., nucleic acids encoding an EphA2)).

In other embodiments, EphA2/EphrinA1 Modulators are agents that modulate the expression of EphrinA1. Such agents can decrease/downregulate EphrinA1 expression (e.g., EphrinA1 antisense molecules, RNAi and ribozymes) or increase/upregulate Ephrin expression (e.g., nucleic acids encoding EphrinA1)).

In yet other embodiments, EphA2/EphrinA1 Modulators of the invention include, but are not limited to, agents that modulate the protein stability or protein accumulation of EphA2 or EphrinA1.

In further embodiments, EphA2/EphrinA1 Modulators of the invention are agents that promote kinase activity (e.g., of EphA2, EphrinA1 or of a heterologous protein known to associate with EphA2 or EphrinA1 at the cell membrane).

In yet further embodiments, EphA2/EphrinA1 Modulators include, but are not limited to, agents that bind to EphA2 and prevent or reduce EphA2 signal transduction but do not inhibit or reduce the interaction between EphA2 and EphrinA1 (e.g., an EphA2 intrabody); and agents that bind to EphrinA1 and prevent or reduce EphrinA1 signal transduction but do not inhibit or reduce the interaction between EphrinA1 and EphA2 (e.g., an EphrinA1 antibody).

In a specific embodiment, an EphA2/EphrinA1 Modulator has one, two or all of the following cellular effects: (i) increase EphA2 cytoplasmic tail phosphorylation; (ii) increase EphA2 autophosphorylation; and (iii) increase EphA2 degradation.

As used herein, the term “EphA2 polypeptide” refers to EphA2, an analog, derivative or a fragment thereof, or a fusion protein comprising EphA2, an analog, derivative or a fragment thereof. The EphA2 polypeptide may be from any species. In certain embodiments, the term “EphA2 polypeptide” refers to the mature, processed form of EphA2. In other embodiments, the term “EphA2 polypeptide” refers to an immature form of EphA2. In accordance with this embodiment, the antibodies of the invention immunospecifically bind to the portion of the immature form of EphA2 that corresponds to the mature, processed form of EphA2.

The nucleotide and/or amino acid sequences of EphA2 polypeptides can be found in the literature or public databases, or the nucleotide and/or amino acid sequences can be determined using cloning and sequencing techniques known to one of skill in the art. For example, the nucleotide sequence of human EphA2 can be found in the GenBank database (see, e.g., Accession Nos. BC037166, M59371 and M36395). The amino acid sequence of human EphA2 can be found in the GenBank database (see, e.g., Accession Nos. AAH37166 and AAA53375). Additional non-limiting examples of amino acid sequences of EphA2 are listed in Table 1, infra.

TABLE 1

| Species | GenBank Accession No. |
|---|---|
| Mouse | NP_034269, AAH06954 |
| Rat | XP_345597 |

In a specific embodiment, a EphA2 polypeptide is EphA2 from any species. In a preferred embodiment, an EphA2 polypeptide is human EphA2. 101161 As used herein, the term “EphrinA1 polypeptide” refers to EphrinA1, an analog, derivative or a fragment thereof, or a fusion protein comprising EphrinA1, an analog, derivative or a fragment thereof. The EphrinA1 polypeptide may be from any species. In certain embodiments, the term “EphrinA1 polypeptide” refers to the mature, processed form of EphrinA1. In other embodiments, the term “EphrinA1 polypeptide” refers to an immature form of EphrinA1. In accordance with this embodiment, the antibodies of the invention immunospecifically bind to the portion of the immature form of EphrinA1 that corresponds to the mature, processed form of EphrinA1.

The nucleotide and/or amino acid sequences of EphrinA1 polypeptides can be found in the literature or public databases, or the nucleotide and/or amino acid sequences can be determined using cloning and sequencing techniques known to one of skill in the art. For example, the nucleotide sequence of human EphrinA1 can be found in the GenBank database (see, e.g., Accession No. BC032698). The amino acid sequence of human EphrinA1 can be found in the GenBank database (see, e.g., Accession No. AAH32698). Additional non-limiting examples of amino acid sequences of EphrinA1 are listed in Table 2, infra.

TABLE 2

| Species | GenBank Accession No. |
|---|---|
| Mouse | NP_034237 |
| Rat | NP_446051 |

In a specific embodiment, a EphrinA1 polypeptide is EphrinA1 from any species. In a preferred embodiment, an EphrinA1 polypeptide is human EphrinA1.

As used herein, the term “epitope” refers to sites or fragments of a polypeptide or protein having antigenic or immunogenic activity in an animal, preferably in a mammal, and most preferably in a human. In specific embodiments, the term “epitope” refers to a portion of an EphA2 polypeptide or an EphrinA1 polypeptide having antigenic or immunogenic activity in an animal, preferably in a mammal, and most preferably in a human. An epitope having immunogenic activity is a site or fragment of a polypeptide or protein that elicits an antibody response in an animal. In specific embodiments, an epitope having immunogenic activity is a portion of an EphA2 polypeptide or an EphrinA1 polypeptide that elicits an antibody response in an animal. An epitope having antigenic activity is a site or fragment of a polypeptide or protein to which an antibody immunospecifically binds as determined by any method well-known to one of skill in the art, for example by immunoassays. In specific embodiments, an epitope having antigenic activity is a portion of an EphA2 polypeptide or an EphrinA1 polypeptide to which an antibody immunospecifically binds as determined by any method well known in the art, for example, by immunoassays. Antigenic epitopes need not necessarily be immunogenic.

As used herein, the term “fragment” in the context of a proteinaceous agent refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 30 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of another polypeptide or protein. In a specific embodiment, a fragment is a fragment of an EphA2 or EphrinA1 polypeptide, or an antibody that immunospecifically binds to an EphA2 or EphrinA1 polypeptide. In an embodiment, a fragment of a protein or polypeptide retains at least one function of the protein or polypeptide. In another embodiment, a fragment of a polypeptide or protein retains at least two, three, four, or five functions of the polypeptide or protein. Preferably, a fragment of an antibody that immunospecifically binds to an EphA2 polypeptide or fragment thereof, or an EphrinA1 polypeptide or fragment thereof retains the ability to immunospecifically bind to an EphA2 polypeptide or fragment thereof, or an EphrinA1 polypeptide or fragment thereof, respectively. Preferably, antibody fragments are epitope-binding fragments.

As used herein, the term “fusion protein” refers to a polypeptide or protein that comprises the amino acid sequence of a first polypeptide or protein or fragment, analog or derivative thereof, and the amino acid sequence of a heterologous polypeptide or protein (i.e., a second polypeptide or protein or fragment, analog or derivative thereof different than the first polypeptide or protein or fragment, analog or derivative thereof, or not normally part of the first polypeptide or protein or fragment, analog or derivative thereof). In one embodiment, a fusion protein comprises a prophylactic or therapeutic agent fused to a heterologous protein, polypeptide or peptide. In accordance with this embodiment, the heterologous protein, polypeptide or peptide may or may not be a different type of prophylactic or therapeutic agent. For example, two different proteins, polypeptides, or peptides with immunomodulatory activity may be fused together to form a fusion protein. In a preferred embodiment, fusion proteins retain or have improved activity relative to the activity of the original polypeptide or protein prior to being fused to a heterologous protein, polypeptide, or peptide.

As used herein, the term “humanized antibody” refers to forms of non-human (e.g., murine) antibodies, preferably chimeric antibodies, which contain minimal sequence derived from non-human immunoglobulin. For the most part, humanized antibodies are human immunoglobulins (recipient antibody) in which hypervariable region or complementarity determining (CDR) residues of the recipient are replaced by hypervariable region residues or CDR residues from an antibody from a non-human species (donor antibody) such as mouse, rat, rabbit or non-human primate having the desired specificity, affinity, and capacity. In some instances, one or more Framework Region (FR) residues of the human immunoglobulin are replaced by corresponding non-human residues or other residues based upon structural modeling, e.g., to improve affinity of the humanized antibody. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., 1986, Nature 321:522-525; Reichmann et al., 1988, Nature 332:323-329; Presta, 1992, Curr. Op. Struct. Biol. 2:593-596; and Queen et al., U.S. Pat. No. 5,585,089.

As used herein, the term “hybridizes under stringent conditions” describes conditions for hybridization and washing under which nucleotide sequences at least 30% (preferably, 35%, 40%, 45%, 50%, 55%, 60%, 65%, 70%, 75%, 80%, 85%, 90%, 95% and 98%) identical to each other typically remain hybridized to each other. Such stringent conditions are known to those skilled in the art and can be found in Current Protocols in Molecular Biology, John Wiley & Sons, N.Y. (1989), 6.3.1-6.3.6.

Generally, stringent conditions are selected to be about 5 to 10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (for example, 10 to 50 nucleotides) and at least about 60° C. for long probes (for example, greater than 50 nucleotides). Stringent conditions may also be achieved with the addition of destabilizing agents, for example, formamide. For selective or specific hybridization, a positive signal is at least two times background, preferably 10 times background hybridization.

In one, non-limiting example stringent hybridization conditions are hybridization at 6× sodium chloride/sodium citrate (SSC) at about 45° C., followed by one or more washes in 0.1×SSC, 0.2% SDS at about 68° C. In a preferred, non-limiting example stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

As used herein, the term “hypervariable region” refers to the amino acid residues of an antibody which are responsible for antigen binding. The hypervariable region comprises amino acid residues from a “Complementarity Determining Region” or “CDR” (i.e. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop”(i.e. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917). “Framework Region” or “FR” residues are those variable domain residues other than the hypervariable region residues as herein defined.

As used herein, the term “immunomodulatory agent” refers to an agent that modulates a subject's immune system. In particular, an immunomodulatory agent is an agent that alters the ability of a subject's immune system to respond to one or more foreign antigens. In a specific embodiment, an immunomodulatory agent is an agent that shifts one aspect of a subject's immune response. In a preferred embodiment of the invention, an immunomodulatory agent is an agent that inhibits or reduces a subject's immune response (i.e., an immunosuppressant agent). Preferably, an immunomodulatory agent that inhibits or reduces a subject's immune response inhibits or reduces the ability of a subject's immune system to respond to one or more foreign antigens. In certain embodiments, antibodies that immunospecifically bind IL-9 are immunomodulatory agents.

As used herein, the term “immunospecifically binds to EphA2” and analogous terms refers to peptides, polypeptides, proteins, fusion proteins, and antibodies or fragments thereof that specifically bind to an EphA2 receptor or one or more fragments thereof and do not specifically bind to other receptors or fragments thereof. The terms “immunospecifically binds to EphrinA1” and analogous terms refer to peptides, polypeptides, proteins, fusion proteins, and antibodies or fragments thereof that specifically bind to EphrinA1 or one or more fragments thereof and do not specifically bind to other ligands or fragments thereof. A peptide, polypeptide, protein, or antibody that immunospecifically binds to EphA2 or EphrinA1, or fragments thereof, may bind to other peptides, polypeptides, or proteins with lower affinity as determined by, e.g., immunoassays or other assays known in the art to detect binding affinity. Antibodies or fragments that immunospecifically bind to EphA2 or EphrinA1 may be cross-reactive with related antigens. Preferably, antibodies or fragments thereof that immunospecifically bind to EphA2 or EphrinA1 can be identified, for example, by immunoassays or other techniques known to those of skill in the art. An antibody or fragment thereof binds specifically to EphA2 or EphrinA1 when it binds to EphA2 or EphrinA1 with higher affinity than to any cross-reactive antigen as determined using experimental techniques, such as radioimmunoassays (RIAs) and enzyme-linked immunosorbent assays (ELISAs). See, e.g., Paul, ed., 1989, Fundamental Immunology, 2^{nd} ed., Raven Press, New York at pages 332-336 for a discussion regarding antibody specificity. To a preferred embodiment, an antibody that immunospecifically binds to EphA2 or EphrinA1 does not bind or cross-react with other antigens. In another embodiment, an antibody that binds to EphA2 or EphrinA1 that is a fusion protein specifically binds to the portion of the fusion protein that is EphA2 or EphrinA1.

As used herein, the term “in combination” refers to the use of more than one therapy. The use of the term “in combination” does not restrict the order in which therapies are administered to a subject with an infection. A first therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject which had, has, or is susceptible to an infection. Any additional therapy can be administered in any order with the other additional therapies. In certain embodiments, EphA2/EphrinA1 Modulators of the invention can be administered in combination with one or more therapies (e.g., non-EphA2/EphrinA1 Modulators currently administered to treat, prevent, manage and/or ameliorate the infection, analgesic agents, anesthetic agents, antibiotics, or immunomodulatory agents).

As used herein, the term “infection” refers to all stages of a pathogen's life cycle in a host (including, but not limited to the invasion by and replication of a pathogen in a cell or body tissue), and the pathological state resulting from the invasion by and replication of a pathogen. The invasion by and multiplication of a virus includes, but is not limited to, the following steps: the docking of the virus particle to a cell, the introduction of viral genetic information into a cell, the expression of viral proteins, the production of new virus particles and the release of virus particles from a cell. In a specific embodiment, an infection is caused by an intracellular pathogen (e.g., a virus, a bacteria, a protozoan, or a fungus). In a preferred embodiment, the infection by the intracellular pathogen requires invasion of the pathogen into an infected cell. In a preferred embodiment, the infection caused by a pathogen causes and/or is associated with an increase in EphA2 expression in the infected cells. In a specific embodiment, the level of EphA2 expression in the cells (e.g., epithelial cells) of a subject infected with a pathogen is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% or at least 1.5 fold, at least 2 fold, at least 2.5 fold, at least 3 fold, at least 3.5 fold, at least 4 fold, at least 4.5, at least 5 fold, at least 7 fold or at least 10 fold relative to the level of EphA2 expression in the uninfected cells of said subject, cells of a normal, healthy subject and/or a population of normal, healthy cells.

As used herein, the term “increased” with respect to EphA2 expression refers to an increase in the expression of EphA2 in the cells (e.g., epithelial cells ) of a subject infected with a pathogen, for example, by a bacteria, virus, fungi or protozoan, relative to the level of EphA2 expression in uninfected cells of said subject, cells of a normal, healthy subject and/or a population of normal, healthy cells. In a specific embodiment, the level of EphA2 expression in the cells (e.g., epithelial cells) of a subject infected with a pathogen is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% or at least 1.5 fold, at least 2 fold, at least 2.5 fold, at least 3 fold, at least 3.5 fold, at least 4 fold, at least 4.5, at least 5 fold, at least 7 fold or at least 10 fold relative to the level of EphA2 expression in the uninfected cells of said subject, cells of a normal, healthy subject and/or a population of normal, healthy cells.

As used herein, the term “isolated” in the context of an organic or inorganic molecule (whether it be a small or large molecule), other than a proteinaceous agent or a nucleic acid, refers to an organic or inorganic molecule substantially free of a different organic or inorganic molecule. Preferably, an organic or inorganic molecule is 60%, 65%, 70%, 75%, 80%, 85%, 90%, 95%, or 99% free of a second, different organic or inorganic molecule. In a preferred embodiment, an organic and/or inorganic molecule is isolated.

As used herein, the term “isolated” in the context of a proteinaceous agent (e.g., a peptide, polypeptide, fusion protein, or antibody) refers to a proteinaceous agent which is substantially free of cellular material or contaminating proteins from the cell or tissue source from which it is derived, or substantially free of chemical precursors or other chemicals when chemically synthesized. The language “substantially free of cellular material” includes preparations of a proteinaceous agent in which the proteinaceous agent is separated from cellular components of the cells from which it is isolated or recombinantly produced. Thus, a proteinaceous agent that is substantially free of cellular material includes preparations of a proteinaceous agent having less than about 30%, 20%, 10%, or 5% (by dry weight) of heterologous protein, polypeptide, peptide, or antibody (also referred to as a “contaminating protein”). When the proteinaceous agent is recombinantly produced, it is also preferably substantially free of culture medium, i.e., culture medium represents less than about 20%, 10%, or 5% of the volume of the proteinaceous agent preparation. When the proteinaceous agent is produced by chemical synthesis, it is preferably substantially free of chemical precursors or other chemicals, i.e., it is separated from chemical precursors or other chemicals which are involved in the synthesis of the proteinaceous agent. Accordingly, such preparations of a proteinaceous agent have less than about 30%, 20%, 10%, 5% (by dry weight) of chemical precursors or compounds other than the proteinaceous agent of interest. In a specific embodiment, proteinaceous agents disclosed herein are isolated. In a preferred embodiment, an antibody of the invention is isolated.

As used herein, the term “isolated” in the context of nucleic acid molecules refers to a nucleic acid molecule which is separated from other nucleic acid molecules which are present in the natural source of the nucleic acid molecule. Moreover, an “isolated” nucleic acid molecule, such as a cDNA molecule, is preferably substantially free of other cellular material, or culture medium when produced by recombinant techniques, or substantially free of chemical precursors or other chemicals when chemically synthesized. In a specific embodiment, nucleic acid molecules are isolated. In a preferred embodiment, a nucleic acid molecule encoding an antibody of the invention is isolated.

As used herein, the term “low tolerance” refers to a state in which the patient suffers from side effects from treatment so that the patient does not benefit from and/or will not continue therapy because of the adverse effects and/or the harm from side effects outweighs the benefit of the treatment.

As used herein, the terms “manage”, “managing” and “management” refer to the beneficial effects that a subject derives from a therapy, which does not result in a cure of the infection. In certain embodiments, a subject is administered one or more therapies to “manage” a infection so as to prevent the progression or worsening of the disorder (i.e., hold disease progress).

As used herein, the term “pathology-causing cell phenotype” refers to a function that an infected cell performs that causes or contributes to the pathological state of an infection. Pathology-causing cell phenotypes include, but are not limited to, increased EphA2 expression, decreased cell/cell intraction, increased extracellular matrix deposition, increased migration, increased cell survival and/or proliferation of a cell infected (e.g., an epithelial cell) by an infectious pathogen/agent (e.g., bacteria, virus, fungus or protozoan). One or more of these pathology-causing cell phenotypes causes or contributes to symptoms in a patient suffering from an infection.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the term “potentiate” refers to an improvement in the efficacy of a therapy at its common or approved dose.

As used herein, the terms “prevent,” “preventing,” and “prevention” refer to the inhibition of the development or onset of an infection in a subject resulting from the administration of a therapy (e.g., a prophylactic or therapeutic agent), or the administration of a combination of therapies (e.g., a combination of prophylactic or therapeutic agents).

As used herein, the term “prophylactic agent” refers to any agent that can prevent the recurrence, spread or onset of an infection, or a symptom thereof. In certain embodiments, the term “prophylactic agent” refers to an EphA2/EphrinA1 Modulator. In certain other embodiments, the term “prophylactic agent” refers to an agent other than an EphA2/EphrinA1 Modulator. Preferably, a prophylactic agent is an agent which is known to be useful to or has been or is currently being used to the prevent or impede the onset, development, progression and/or severity of an infection or one or more symptoms thereof.

As used herein, a “prophylactically effective amount” refers to that amount of a therapy (e.g., a prophylactic agent) sufficient to result in the prevention of the recurrence, spread or onset of an infection or a symptom thereof. A prophylactically effective amount may refer to the amount of a therapy (e.g., a prophylactic agent) sufficient to prevent the occurrence, spread or recurrence of an infection, for example those having previously suffered from such an infection, or those who are immunocompromised or immunosuppressed, or are genetically predisposed to such an infection. A prophylactically effective amount may also refer to the amount of a therapy (e.g., a prophylactic agent) that provides a prophylactic benefit in the prevention of an infection. Further, a prophylactically effective amount with respect to a therapy (e.g., a prophylactic agent of the invention) means that amount of the therapy (e.g., prophylactic agent) alone, or in combination with one or more other therapies (e.g., non-EphA2/EphrinA1 Modulators currently administered to prevent the infection, analgesic agents, anesthetic agents, antibiotics, or immunomodulatory agents) that provides a prophylactic benefit in the prevention of an infection. Used in connection with an amount of an EphA2/EphrinA1 Modulator of the invention, the term can encompass an amount that improves overall prophylaxis or enhances the prophylactic efficacy of or synergies with another therapy, (e.g., a prophylactic agent).

A used herein, a “protocol” includes dosing schedules and dosing regimens.

As used herein, the term “refractory” refers to an infection, that is not responsive to one or more therapies (e.g., currently available therapies). In a certain embodiment, that an infection is refractory to a therapy means that at least some significant portion of the symptoms associated with said infection are not eliminated or lessened by that therapy. The determination of whether an infection, is refractory can be made either in vivo or in vitro by any method known in the art for assaying the effectiveness of therapy for an infection.

As used herein, the phrase “side effects” encompasses unwanted and adverse effects of a therapy (e.g., a prophylactic or therapeutic agent). Adverse effects are always unwanted, but unwanted effects are not necessarily adverse. An adverse effect from a therapy (e.g., a prophylactic or therapeutic agent) might be harmful or uncomfortable or risky. Examples of side effects include, but are not limited to, nausea, vomiting, anorexia, abdominal cramping, fever, pain, loss of body weight, dehydration, alopecia, dyspnea, insomnia, dizziness, mucositis, nerve and muscle effects, fatigue, dry mouth, and loss of appetite, rashes or swellings at the site of administration, flu-like symptoms such as fever, chills and fatigue, digestive tract problems and allergic reactions. Additional undesired effects experienced by patients are numerous and known in the art. Many are described in the Physicians' Desk Reference (59^{th} ed., 2005).

As used herein, the term “single-chain Fv” or “scFv” refers to antibody fragments comprising the V_{H} and V_{L} domains of antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv see Pluckthun in The Pharmacology of Monoclonal Antibodies, vol. 113, Rosenburg and Moore eds. Springer-Verlag, New York, pp. 269-315 (1994).

As used herein, the terms “subject” and “patient” are used interchangeably. As used herein, a subject is preferably a mammal such as a non-primate (e.g., cows, pigs, horses, cats, dogs, rats etc.) and a primate (e.g., monkey and human), most preferably a human. In one embodiment, the subject is a mammal, preferably a human, with an infection. In another embodiment, the subject is a farm animal (e.g., a horse, pig, or cow), a pet (e.g., a guinea pig, dog or cat), or a laboratory animal (e.g., an animal model) with an infection. In another embodiment, the subject is a mammal, preferably a human, at risk of developing an intracellular pathogen infection (e.g., an immunocompromised or immunosuppressed mammal, or a genetically predisposed mammal). In another embodiment, the subject is not an immunocompromised or immunosuppressed mammal, preferably a human. In another embodiment, the subject is a mammal, preferably a human, with a lymphocyte count that is not under approximately 500 cells/mm³.

As used herein, the term “synergistic” refers to a combination of therapies (e.g., prophylactic or therapeutic agents) which is more effective than the additive effects of any two or more single therapies (e.g., one or more prophylactic or therapeutic agents). A synergistic effect of a combination of therapies (e.g., a combination of prophylactic or therapeutic agents) permits the use of lower dosages of one or more of therapies (e.g., one or more prophylactic or therapeutic agents) and/or less frequent administration of said therapies to a subject with an infection. The ability to utilize lower dosages of therapies (e.g., prophylactic or therapeutic agents) and/or to administer said therapies less frequently reduces the toxicity associated with the administration of said therapies to a subject without reducing the efficacy of said therapies in the prevention or treatment of an infection. In addition, a synergistic effect can result in improved efficacy of therapies (e.g., prophylactic or therapeutic agents) in the prevention or treatment of an infection. Finally, synergistic effect of a combination of therapies (e.g., prophylactic or therapeutic agents) may avoid or reduce adverse or unwanted side effects associated with the use of any single therapy.

As used herein, the term “therapeutic agent” refers to any agent that can be used in the treatment, management, prevention, or symptom reduction of an infection. In certain embodiments, the term “therapeutic agent” refers to an EphA2/EphrinA1 Modulator. In certain other embodiments, the term “therapeutic agent” refers an agent other than an EphA2/EphrinA1 Modulator. Preferably, a therapeutic agent is an agent which is known to be useful for, or has been or is currently being used for the prevention, treatment, management, or amelioration of an intracellular pathogen infection or one or more symptoms thereof.

As used herein, a “therapeutically effective amount” refers to that amount of a therapy (e.g., a therapeutic agent) sufficient to reduce the severity of an infection, reduce the duration of an infection, ameliorate one or more symptoms of an infection, prevent the advancement of an infection, cause regression of an infection, or to enhance or improve the therapeutic effect(s) of another therapeutic agent. With respect to the treatment of an infection, a therapeutically effective amount refers to the amount of a therapeutic agent sufficient to reduce or inhibit the replication of a pathogen, inhibit or reduce the infection of a cell with the pathogen, inhibit or reduce the production of pathogen proteins, inhibit or reduce the release of pathogen, inhibit or reduce the spread of the pathogen to other tissues or subjects, or ameliorate one or more symptoms associated with the infection. Preferably, a therapeutically effective amount of a therapeutic agent reduces the replication or spread of a pathogen by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to a control (e.g., a negative control such as phosphate buffered saline) in an assay known in the art or described herein.

As used herein, the term “therapy” refers to any protocol, method and/or agent that can be used in the prevention, treatment or management of an infection. In certain embodiments, the terms “therapies” and “therapy” refer to a biological therapy, supportive therapy, and/or other therapies useful the in treatment, management, prevention, or amelioration of an infection or one or more symptoms thereof known to one of skill in the art such as medical personnel.

As used herein, the terms “treat”, “treatment” and “treating” to the reduction or amelioration of the progression, severity, and/or duration of an infection or the amelioration of one or more symptoms thereof resulting from the administration of one or more therapies (including, but not limited to, the administration of one or more prophylactic or therapeutic agents). In specific embodiments, such terms refer to the reduction or inhibition of the replication of a pathogen, the inhibition or reduction in the spread of a pathogen to other tissues or subjects, the inhibition or reduction of infection of a cell with a pathogen, or the amelioration of one or more symptoms associated with an infection.

### 4. DESCRIPTION OF THE FIGURES

FIG. 1. Western blot analysis of total EphA2 protein isolated from RSV-infected BEAS-2B cells 24 and 48 hours post-infection (at a high multiplicity of infection (MOI)).

FIG. 2. FACS analysis of RSV-F protein present on BEAS-2B cells infected with RSV 1 and 2 days post infection.

FIG. 3. FACS analysis of EphA2 protein present on BEAS-2B cells infected with RSV 1 and 2 days post infection.

FIG. 4. EphA2 expression in BEAS-2B cells following RSV infection (1 and 2 days) as determined by RT-PCR.

FIG. 5. Western blot analysis of total EphA2 protein isolated from RSV-infected NHBE cells (24 hrs).

FIG. 6. Detection of RSV-F protein present on the surface of NHBE cells infected and uninfected with RSV using FACS analysis.

FIG. 7. Detection of EphA2 protein present on the surface of NHBE cells infected and uninfected with RSV using FACS analysis.

FIG. 8. Detection of EphA2 on NHBE cells infected with RSV at a MOI of 0.1 using FACS quadrant analysis.

FIG. 9. Detection of EphA2 on BEAS-2B cells infected with RSV at a MOI of 0.1 using FACS quadrant analysis.

FIG. 10. Detection of RSV-F protein expressed on NHBE cells infected with RSV±UV irradiation (MOI=1).

FIG. 11. Detection of EphA2 protein expressed on NHBE cells infected with RSV±UV irradiation (MOI=1).

FIG. 12. Detection of RSV-F protein expressed on NHBE cells infected with RSV±UV irradiation (MOI=0.1).

FIG. 13. Detection of EphA2 protein expressed on NHBE cells infected with RSV±UV irradiation (MOI=0.1).

FIG. 14. Detection of RSV-F protein expressed on BEAS-2B cells infected with RSV±UV irradiation (MOI=1).

FIG. 15. Detection of EphA2 protein expressed on BEAS-2B cells infected with RSV±UV irradiation (MOI=1).

FIG. 16. Detection of RSV-F protein expressed on BEAS-2B cells infected with RSV±UV irradiation (MOI=0.1).

FIG. 17. Detection of EphA2 protein expressed on BEAS-2B cells infected with RSV±UV irradiation (MOI=0.1).

FIG. 18. Detection of EphA2 in A549 and Hep2 cells as determined by FACS analysis.

FIG. 19. Imnunohistochemistry for EphA2 in normal murine lung tissue.

FIG. 20. Immunohistochemistry staining for EphA2 in RSV-infected murine lung tissue.

FIG. 21. Immunohistochemistry staining for EphA2 in bleomycin-treated murine lung tissue.

### 5. DETAILED DESCRIPTION OF THE INVENTION

The present invention is based, in part, on the inventors' discovery that EphA2 is upregulated in epithelial cells infected with RSV. Without being bound to a particular theory or mechanism, the upregulation of EphA2 expression in pathogen-infected cells could promote unwanted cell survival. The invention thus provides methods and compositions designed for the treatment, management, prevention and/or amelioration of a pathogen infection, including, but not limited to, a viral infection, a bacterial infection, a fungal infection and a protozoan infection. In particular, the present invention provides methods for treating, managing, preventing, and/or ameliorating an infection where the expression of EphA2 is upregulated in infected cells (e.g., infected EphA2-expressing cells), said methods comprising administering to a subject in need thereof an effective amount of one or more EphA2/EphrinA1 Modulators, and optionally, an effective amount of a therapy other than an EphA2/EphrinA1 Modulator. In a preferred embodiment, the viral, bacterial, fungal and protozoan infections to be treated, managed, prevented and/or ameliorated in accordance with the methods of the present invention are intracellular infections.

The present invention provides pharmaceutical compositions comprising EphA2/EphrinA1 Modulators, and optionally, therapeutic or prophylactic agents (e.g., immunomodulatory agents, anti-viral agents, anti-inflammatory agents, anti-bacterial agents, anti-fungal agents, etc.) other than an EphA2/EphrinA1 Modulator. The present invention also provides methods of detecting, diagnosing and/or prognosing an infection and/or methods for monitoring the efficacy of a therapy for the prevention, treatment, management and/or amelioration of an infection. Such methods may be used in combination with other methods for detecting, diagnosing, monitoring or prognosing an infection. In specific embodiments, the invention provides methods for detecting, diagnosing, monitoring or prognosing latent infections.

The invention further provides articles of manufacture and kits comprising an EphA2/EphrinA1 Modulator of the invention, and optionally, one or more therapeutic or prophylactic agents (e.g., immunomodulatory agents, anti-viral agents, anti-inflammatory agents, anti-bacterial agents, anti-fungal agents, etc.) other than an EphA2/EphrinA1 Modulator. In specific embodiments, the articles of manufacture and kits include instructions for dosage and administration of the EphA2/EphrinA1 Modulatory, and optional a therapy other than an EphA2/EphrinA1 Modulator.

### 5.1 EphA2/EphrinA1 Modulators

The invention provides modulators of EphA2 and/or EphrinA1 (“EphA2/EphrinA1 Modulators”). EphA2/EphrinA1 Modulators are therapies that confer a biological effect by modulating (directly or indirectly): (i) the expression of EphA2 and/or an endogenous ligand(s) of EphA2 (preferably, EphrinA1), at, e.g., the transcriptional, post-transcriptional, translational or post-translation level; and/or (ii) an activity(ies) of EphA2 and/or EphrinA1.

Examples of EphA2/EphrinA1 Modulators include, but are not limited to, agents that inhibit or reduce the interaction between EphA2 and an endogenous ligand(s) of EphA2, preferably, EphrinA1 (hereinafter “EphA2/EphrinA1 Interaction Inhibitors”). Non-limiting examples of EphA2/EphrinA1 Interaction Inhibitors include: (i) agents that bind to EphA2, prevent or reduce the interaction between EphA2 and EphrinA1, and induce EphA2 signal transduction (e.g., soluble forms of EphrinA1 (e.g., an EphrinA1-Fc in monomeric or multimeric form), and antibodies that bind to EphA2, induce signaling and phosphorylation of EphA2 (i.e., an EphA2 agonistic antibody)); (ii) agents that bind to EphA2, prevent or reduce the interaction between the EphA2 and EphrinA1, and prevent or induce very low to negligible levels of EphA2 signal transduction (e.g., EphA2 antagonistic antibodies and dominant negative forms of EphrinA1); (iii) agents that bind to EphrinA1, prevent or reduce the interaction between EphA2 and EphrinA1, and induce EphrinA1 signal transduction (e.g., soluble forms of EphA2 (e.g., EphA2-Fc) and antibodies that bind to EphrinA1 and induce EphrinA1 signal transduction); and (iv) agents that bind to EphrinA1, prevent or reduce the interaction between an EphA2 and EphrinA1, and prevent or induce very low to negligible levels of EphrinA1 signal transduction (e.g., dominant negative forms of an EphA2 and anti-EphrinA1 antibodies).

In further embodiments, EphA2/EphrinA1 Modulators include, but are not limited to, agents that modulate the expression of EphA2. Such agents can decrease/downregulate EphA2 expression (e.g., EphA2 antisense molecules, RNAi and ribozymes) or increase/upregulate EphA2 expression such that the amount of EphA2 on the cell surface exceeds the amount of endogenous ligand (preferably, EphrinA1) available for binding, and thus, increases the amount of unbound EphA2 (e.g., nucleic acids encoding an EphA2)).

In other embodiments, EphA2/EphrinA1 Modulators are agents that modulate the expression of EphrinA1. Such agents can decrease/downregulate EphrinA1 expression (e.g., EphrinA1 antisense molecules, RNAi and rihozymes) or increase/upregulate Ephrin expression (e.g., nucleic acids encoding EphrinA1)).

In yet other embodiments, EphA2/EphrinA1 Modulators of the invention include, but are not limited to, agents that modulate the protein stability or protein accumulation of EphA2 or EphrinA1.

In further embodiments, EphA2/EphrinA1 Modulators of the invention are agents that promote kinase activity (e.g., of EphA2, EphrinA1 or of a heterologous protein known to associate with EphA2 or EphrinA1 at the cell membrane).

In yet further embodiments, EphA2/EphrinA1 Modulators include, but are not limited to, agents that bind to EphA2 and prevent or reduce EphA2 signal transduction but do not inhibit or reduce the interaction between EphA2 and EphrinA1 (e.g., an EphA2 intrabody); and agents that bind to EphrinA1 and prevent or reduce EphrinA1 signal transduction but do not inhibit or reduce the interaction between EphrinA1 and EphA2 (e.g., an EphrinA1 antibody).

In a specific embodiment, an EphA2/EphrinA1 Modulator is not an agent that inhibits or reduces the interaction between EphA2 and an endogenous ligand, preferably, EphrinA1. In a further embodiment, an EphA2/EphrinA1 Modulator is not an EphA2 agonistic antibody. In a further embodiment, an EphA2/EphrinA1 Modulator is not an Eph receptor antisense molecule or EphrinA1 antisense molecule. In yet a further embodiment, an EphA2/EphrinA1 Modulator is not a soluble form of an Eph receptor (e.g., Eph-Fc) or is not a soluble form of EphrinA1 (e.g., Ephrin-Fc).

In specific embodiments of the invention, an EphA2/EphrinA1 Modulator does one or more of the following: (i) decreases EphA2 expression and/or activity; (ii) causes apoptosis and/or necrosis of EphA2-expressing cells infected with a pathogen; and (iii) causes EphA2 ligand-induced phosphorylation (e.g., autophosphorylation) and degradation. In other specific embodiments, an EphA2/EphrinA1 Modulator is one of the following: (i) a soluble EphrinA1 molecule (e.g., EphrinA1-Fc); (ii) an EphA2 antisense nucleic acid molecule; (iii) an EphA2 agonistic antibody that induces EphA2 phosphorylation and degradation; (iv) an EphA2 vaccine; (v) an anti-EphrinA1 or anti-EphA2 antibody conjugated to a cytotoxic agent; (vi) a multispecific antibody (e.g., bispecific antibody (such as a BiTE molecule) that targets, e.g., EphA2 and a pathogen antigen or cell marker.

In a specific embodiment, an EphA2/EphrinA1 Modulator is an agent that decreases or downregulates EphA2 expression (e.g., EphA2 antisense molecules, RNAi and ribozymes). In a particular embodiment, the EphA2/EphrinA1 Modulator decreases or downregulates EphA2 expression by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline) in an assay described herein or known in the art (e.g., RT-PCR, a Northern blot or an immunoassay such as an ELISA).

In a specific embodiment, an EphA2/EphrinA1 Modulator is an agent that reduces the protein stability and/or protein accumulation of EphA2 by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline or a control IgG) in an assay described herein or known in the art (e.g., an immunoassay).

In a specific embodiment, an EphA2/EphrinA1 Modulator is an agent that inhibits or decreases the expression of EphrinA1 (e.g., EphrinA1 antisense molecules, RNAi and ribozymes). In a particular embodiment, the EphA2/EphrinA1 Modulator decreases the expression of EphrinA1 by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline or a control IgG) in an assay described herein or known in the art (e.g., RT-PCR, a Northern blot or an immunoassay such as an ELISA).

In another embodiment, an EphA2/EphrinA1 Modulator is an agent that binds to EphA2 and prevents or reduces EphA2 signal transduction but does not inhibit or reduce the interaction between EphA2 and an endogenous ligand(s) of EphA2, preferably, EphrinA1 (e.g., an EphA2 intrabody). In a particular embodiment, the EphA2/EphrinA1 Modulator reduces EphA2 signal transduction by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline or a control IgG) in an assay described herein or known in the art (e.g., an immunoassay). In accordance with this embodiment, the EphA2/EphrinA1 Modulator does not reduce or only reduces the interaction between EphA2 and an endogenous ligand(s) of EphA2 (preferably, EphrinA1) by 40% or less, 35% or less, 30% or less, 25% or less, 20% or less, 15% or less, 10% or less, or 5% or less relative to a control (e.g., phosphate buffered saline) in an assay described herein or known in the art.

In another embodiment, an EphA2/EphrinA1 Modulator is an agent that binds to EphrinA1 and prevents or reduces EphrinA1 signal transduction but does not inhibit or reduce the interaction between EphrinA1 and EphA2 (e.g., an EphrinA1 antibody). In a particular embodiment, the EphA2/EphrinA1 Modulator reduces EphrinA1 signal transduction by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline or a control IgG) in an assay described herein or known in the art (e.g., an immunoassay). In accordance with this embodiment, the EphA2/EphrinA1 Modulator does not reduce or only reduces the interaction between EphA2 and an endogenous ligand(s) of EphA2 (preferably, EphrinA1) by 40% or less, 35% or less, 30% or less, 25% or less, 20% or less, 15% or less, 10% or less, or 5% or less or less, or 2 fold or less, 1.5 fold or less or 1 fold or less relative to a control (e.g., phosphate buffered saline or a control IgG) in an assay described herein or known in the art.

In a specific embodiment, an EphA2/EphrinA1 Modulator is an EphA2/EphrinA1 Interaction Inhibitor. In one embodiment, an EphA2/EphrinA1 Interaction Inhibitor is an agent that binds to EphA2, prevents or reduces the interaction between EphA2 and an endogenous ligand of EphA2, preferably, EphrinA1, and induces EphA2 signal transduction (e.g., soluble forms of EphrinA1 (EphrinA1-Fc) and antibodies that bind to EphA2, induce signaling and phosphorylation of EphA2 (i.e., an agonistic antibody)). In a particular embodiment, such an EphA2/EphrinA1 Interaction Inhibitor reduces the interaction between EphA2 and an endogenous ligand of EphA2 (preferably, EphrinA1) by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline or a control IgG) in an assay described herein or known in the art. In accordance with this embodiment, the EphA2/EphrinA1 Interaction Inhibitor induces EphA2 signal transduction by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline or a control IgG) in an assay described herein or known in the art (e.g., an immunoassay).

In another embodiment, an EphA2/EphrinA1 Interaction Inhibitor is an agent that binds to EphA2, prevents or reduces the interaction between EphA2 and an endogenous ligand of EphA2, preferably, EphrinA1, and prevents or induces very low to negligible levels of EphA2 signal transduction (e.g., antibodies). In a particular embodiment, such an EphA2/EphrinA1 Interaction Inhibitor reduces the interaction between EphA2 and an endogenous ligand of EphA2 (preferably, EphrinA1 ) by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline or a control IgG) in an assay described herein or known in the art. In accordance with this embodiment, the EphA2/EphrinA1 Interaction Inhibitor induces EphA2 signal transduction by 5% or less, 10% or less, 15% or less, 20% or less, 25% or less, 30% or less, 35% or less, 40% or less, or 2 fold or less, 1.5 fold or less or 1 fold or less relative to a control (e.g., phosphate buffered saline) in an assay described herein or known in the art (e.g., an immunoassay).

In another embodiment, an EphA2/EphrinA1 Interaction Inhibitor is an agent that binds to EphrinA1, prevents or reduces the interaction between EphA2 and EphrinA1 and induces EphrinA1 signal transduction (e.g., soluble forms of EphA2, dominant negative forms of EphA2, and antibodies that bind to EphrinA1 and induce EphrinA1 signal transduction). In a particular embodiment, such an EphA2/EphrinA1 Interaction Inhibitor reduces the interaction between EphA2 and EphrinA1 by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline or a control IgG) in an assay described herein or known in the art In accordance with this embodiment, the EphA2/EphrinA1 Interaction Inhibitor induces EphrinA1 signal transduction by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline a control IgG) in an assay described herein or known in the art (e.g., an immunoassay).

In another embodiment, an EphA2/EphrinA1 Interaction Inhibitor is an agent that binds to EphrinA1, prevents or reduces the interaction between EphA2 and EphrinA1, and prevents or induces very low to negligible levels of EphrinA1 signal transduction (e.g., antibodies). In a particular embodiment, such an EphA2/EphrinA1 Interaction Inhibitor reduces the interaction between EphA2 and EphrinA1 by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline a control IgG) in an assay described herein or known in the art. In accordance with this embodiment, the EphA2/EphrinA1 Interaction Inhibitor induces EphrinA1 signal transduction by 5% or less, 10% or less, 15% or less, 20% or less, 25% or less, 30% or less, 35% or less, 40% or less, or 2 fold or less, 1.5 fold or less or 1 fold or less relative to a control (e.g., phosphate buffered saline) in an assay described herein or known in the art (e.g., an immunoassay).

In a specific embodiment, an EphA2/EphrinA1 Modulator has one, two or all of the following cellular effects: (i) increase EphA2 cytoplasmic tail phosphorylation; (ii) increase EphA2 autophosphorylation; and (iii) increase EphA2 degradation.

EphA2/EphrinA1 Modulators of the invention include, but are not limited to, proteinaceous molecules (including, but not limited to, peptides, polypeptides, proteins, post-translationally modified proteins, antibodies, EphA2 vaccines, etc.), small molecules (less than 1000 daltons), inorganic or organic compounds, nucleic acid molecules (including, but not limited to, double-stranded, single-stranded DNA, double-stranded or single-stranded RNA (e.g., antisense, mediates RNAi, etc.), and triple helix nucleic acid molecules), aptamers, and derivatives of any of the above.

### 5.1.1 Polypeptides As EphA2/EphrinA1 Modulators

Methods of the present invention encompass EphA2/EphrinA1 Modulators that are polypeptides. In specific embodiment, a polypeptide EphA2/EphrinA1 Modulator prevents, reduces or slows the progression of an intracellular pathogen infection. In a preferred embodiment, the cells infected with the intracellular pathogen have increased EphA2 expression.

In one embodiment, a polypeptide EphA2/EphrinA1 Modulator is an antibody, preferably, a monoclonal antibody. In another embodiment, a polypeptide EphA2/EphrinA1 Modulator is a soluble form of EphA2 (e.g., EphA2-Fc). In another embodiment, a polypeptide EphA2/EphrinA1 Modulator is a dominant negative form of EphA2.

In one embodiment, a polypeptide EphA2/EphrinA1 Modulator is an EphA2/EphrinA1 Interaction Inhibitor. In a specific embodiment, an EphA2/EphrinA1 Modulator is an EphA2 antibody that immunospecifically binds EphA2, prevents or reduces the interaction between EphA2 and an endogenous ligand of EphA2, preferably, EphrinA1, and induces EphA2 signal transduction (including, but not limited to, EphA2 autophosphorylation). In another embodiment, an EphA2/EphrinA1 Modulator is an EphA2 antibody that immunospecifically binds to EphA2, prevents or reduces the interaction between EphA2 and an endogenous ligand of EphA2, preferably, EphrinA1, and prevents or induces very low to negligible levels of EphA2 signal transduction (including, but not limited to, autophosphorylation of EphA2). In certain embodiments, a polypeptide EphA2/EphrinA1 Modulator is not an EphA2 antibody that immunospecifically binds to EphA2, prevents or reduces the interaction between EphA2 and EphrinA1, and induces EphA2 signal transduction.

In a specific embodiment, a polypeptide EphA2/EphrinA1 Modulator is an EphrinA1 antibody that immunospecifically binds to EphrinA1, prevents or reduces the interaction between EphAl and EphrinA1, and induces EphrinA1 signal transduction. In another embodiment, an EphA2/EphrinA1 Modulator is an EphrinA1 antibody that immunospecifically binds EphrinA1, prevents or reduces the interaction between EphA2 and EphrinA1, and prevents or induces very low to negligible levels of EphrinA1 signal transduction.

In a specific embodiment, an EphA2/EphrinA1 Modulator is a soluble form of EphrinA1 or a fragment of EphrinA1 that binds EphA2 (e.g., EphrinA1-Fc), prevents or reduces the interaction between EphA2 and EphrinA1, and induces EphA2 signal transduction (including, but not limited to, autophosphorylation). In another embodiment, an EphA2/EphrinA1 Modulator is a soluble form of EphrinA1 or a fragment of EphrinA1 that binds to EphA2, prevents or reduces the interaction between EphA2 and EphrinA1, and prevents or induces very low to negligible levels of EphA2 signal transduction (including, but not limited to, autophosphorylation of EphA2).

In a specific embodiment, an EphA2/EphrinA1 Modulator is a soluble form of EphA2 or a fragment of EphA2 that binds to an endogenous ligand of EphA2 (preferably, EphrinA1), prevents or reduces the interaction between EphA2 and an endogenous ligand of EphA2 (preferably, EphrinA1), and induces EphrinA1 signal transduction. In another embodiment, an EphA2/EphrinA1 Modulator is a soluble form of EphA2 or a fragment of EphA2 that binds to an endogenous ligand of EphA2 (preferably, EphrinA1), prevents or reduces the interaction between EphA2 and an endogenous ligand of EphA2 (preferably, EphrinA1), and prevents or induces very low to negligible levels of EphrinA1 signal transduction.

In a specific embodiment, an EphA2/EphrinA1 Modulator is a dominant negative form of EphA2 that binds to an endogenous ligand of EphA2 (preferably, EphrinA1), prevents or reduces the interaction between EphA2 and an endogenous ligand of EphA2 (preferably, EphrinA1), and induces EphrinA1 signal transduction. In another embodiment, an EphA2/EphrinA1 Modulator is a dominant negative form of EphA2 that binds to an endogenous ligand of EphA2 (preferably, EphrinA1), prevents or reduces the interaction between EphA2 and an endogenous ligand of EphA2 (preferably, EphrinA1), and prevents or induces very low to negligible levels of EphrinA1 signal transduction.

In a specific embodiment, an EphA2/EphrinA1 Modulator is a fusion protein comprising EphA2 or a fragment thereof (e.g., the extracellular domain of EphA2) fused or conjugated to a heterologous protein, polypeptide or peptide. In a preferred embodiment, the fusion protein comprises EphA2 or a fragment thereof fused or conjugated to the Fc portion of an antibody (e.g., the Fc portion of an IgG antibody). In accordance with the invention, EphA2 or a fragment thereof can be conjugated or fused to an agent described in Section 5.1.1.3, infra. The agents and techniques discussed in Section 5.1.1.3 can be used to produce fusion proteins comprising EphA2 or a fragment thereof.

In a specific embodiment, an Eph2/EphrinA1 Modulator is a fusion protein comprising EphrinA1 or a fragment thereof (e.g., the extracellular domain of EphrinA1) fused or conjugated to a heterologous protein, polypeptide or peptide. In a preferred embodiment, the fusion protein comprises EphrinA1 or a fragment thereof fused or conjugated to the Fc portion of an antibody (e.g., the Fc portion of an IgG antibody). In accordance with the invention, EphrinA1 or a fragment thereof can be conjugated or fused to an agent described in Section 5.1.1.3, infra. The agents and techniques discussed in Section 5.1.1.3 can be used to produce fusion proteins comprising EphrinA1 or a fragment thereof.

### 5.1.1.1 Antibodies As EphA2/EphrinA1 Modulators

In one embodiment, an EphA2/EphrinA1 Modulator is an antibody, preferably a monoclonal antibody. More preferably, the antibody is humanized. Antibody EphA2/EphrinA1 Modulators of the invention immunospecifically bind EphA2 or EphrinA1 and modulate the activity and/or expression of EphA2 and/or EphrinA1. In a specific embodiment, an EphA2/EphrinA1 Modulator antibody which may have a low K_{off} rate (e.g., K_{off} less than 3×10⁻³s⁻¹). In one embodiment, the antibodies used in the methods of the invention are Eph099B-102.147, Eph099B-208.261, Eph099B-210.248, B233, EA2 or EA5. In a more preferred embodiment, the antibodies used in the methods of the invention are human or humanized Eph099B-102.147, Eph099B-208.261, Eph099B-210.248, B233, EA2 or EA5. In a specific embodiment, an EphA2/EphrinA1 Modulator is not Eph099B-102.147, Eph099B-208.261, Eph099B-210.248, B233, EA2 or EA5. In a preferred embodiment, antibody prevents, reduces or slows the progression of an infection.

In a specific embodiment, an antibody of the invention immunospecifically binds to the extracellular domain of EphA2 (e.g., at an epitope either within or outside of the EphA2 ligand binding site) and decreases EphA2 cytoplasmic tail phosphorylation without causing EphA2 degradation. In another specific embodiment, the antibody binds to the extracellular domain of EphA2 (e.g., at an epitope either within or outside of the EphA2 ligand binding site) and inhibits or reduces the extent of EphA2-ligand interaction. In another specific embodiment, an antibody of the invention immunospecifically binds to the extracellular domain of EphA2 (e.g., at an epitope either within or outside of the EphA2 ligand binding site) and decreases EphA2 signal transduction (including, but not limited to, EphA2 autophosphorylation). In yet another embodiment, an antibody of the invention immunospecifically binds to the extracellular domain of EphA2 (e.g., at an epitope either within or outside of the EphA2 ligand binding site), decreases EphA2 signal transduction (including, but not limited to, EphA2 autophosphorylation) and inhibits or reduces the extent of EphA2-ligand interaction. In a specific embodiment, an antibody of the invention immunospecifically binds to the ligand binding domain of human EphA2 (e.g., at amino acid residues 28 to 201) as disclosed in the GenBank database (Genbank accession no. NP_{—}004422.2).

In one embodiment, an antibody of the invention immunospecifically binds to EphrinA1 (e.g., at an epitope either within or outside of the EphA2 binding site) and prevents or reduces the binding to EphA2. In another embodiment, the EphrinA1 antibody of the invention immunospecifically binds to EphrinA1 (e.g., at an epitope either within or outside of the EphA2 binding site) and modulates (induces or inhibits) EphrinA1 signaling in an EphrinA1 expressing cell. In another specific embodiment, an antibody of the invention immunospecifically binds to EphrinA1 (e.g., at an epitope either within or outside of the EphA2 binding site), decreases EphrinA1 signal transduction and inhibits or reduces the extent of EphA2-EphrinA1 interaction. In another specific embodiment, an antibody of the invention immunospecifically binds to EphrinA1 (e.g., at an epitope either within or outside of the EphA2 binding site), induces EphrinA1 signal transduction and inhibits or reduces the extent of EphA2-EphrinA1 interaction. In a further embodiment, an antibody of the invention immunospecifically binds to EphrinA1 (e.g., at an epitope involved in EphrinA1 clustering), inhibits or reduces EphrinA1 interaction with other molecules such as the Src family kinases (e.g., Fyn,), and inhibits or reduces EphrinA1 signal transduction.

Antibodies of the invention include, but are not limited to, synthetic antibodies, monoclonal antibodies, recombinantly produced antibodies, multispecific antibodies (including bi-specific antibodies), human antibodies, humanized antibodies, chimeric antibodies, intrabodies, single-chain Fvs (scFv) (e.g., including monospecific and bi-specific, etc.), Fab fragments, F(ab′) fragments, disulfide-linked Fvs (sdFv), anti-idiotypic (anti-Id) antibodies, and epitope-binding fragments of any of the above. In particular, antibodies of the present invention include immunoglobulin molecules and immunologically active portions of immunoglobulin molecules, i.e., molecules that contain an antigen-binding site that immunospecifically binds to an EphA2 antigen or an EphrinA1 antigen (e.g., one or more complementarity determining regions (CDRs) of an anti-EphA2 antibody or of an anti-EphrinA1 antibody). The antibodies of the invention can be of any type (e.g., IgG₁ IgE, IgM, IgD, IgA and IgY), class (e.g., IgG₁, IgG₂, IgG₃, IgG₄, IgA₁ and IgA₂) or subclass of immunoglobulin molecule.

The present invention encompasses agonistic antibodies that immunospecifically bind to EphA2 and agonize EphA2, i.e., elicit EphA2 signaling and decrease EphA2 expression. Agonistic EphA2 antibodies may induce EphA2 autophosphorylation, thereby causing subsequent EphA2 degradation to down-regulate EphA2 expression and inhibit EphA2 interaction with its endogenous ligand (e.g., EphrinA1). Such antibodies are disclosed in U.S. Patent Pub. Nos. US 2004/0091486 A1 (May 13, 2004), and US 2004/0028685 A1 (Feb. 12, 2004), which are incorporated by reference herein in their entireties. In a specific embodiment, an EphA2/EphrinA1 Modulator antibody may have a low K_{off} rate (e.g., K_{off} less than 3×10⁻³s⁻¹). In another embodiment, the antibodies used in the methods of the invention are Eph099B-102.147, Eph099B-208.261, Eph099B-210.248, B233, EA2 or EA5. In a more preferred embodiment, the antibodies used in the methods of the invention are human or humanized Eph099B-102.147, Eph099B-208.261, Eph099B-210.248, B233, EA2 or EA5. In a specific embodiment, an EphA2/EphrinA1 Modulator is not Eph099B-102.147, Eph099B-208.261, Eph099B-210.248, B233, EA2 or EA5.

The present invention also encompasses single domain antibodies, including camelized single domain antibodies (see, e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Patent Publication Nos. WO 94/04678 and WO 94/25591; U.S. Pat. No. 6,005,079; which are incorporated herein by reference in their entireties). In one embodiment, the present invention provides single domain antibodies comprising two V_{H} domains having the amino acid sequence of a V_{H} domain(s) of any EphA2 or EphrinA1 antibody(ies) with modifications such that single domain antibodies are formed. In another embodiment, the present invention also provides single domain antibodies comprising two V_{H} domains comprising one or more of the V_{H} CDRs of any EphA2 or EphrinA1 antibody(ies).

Antibodies of the invention include EphA2 or EphrinA1 intrabodies (see Section 5.1.1.1.2, infra). Antibody EphA2/EphrinA1 Modulators of the invention that are intrabodies immunospecifically bind EphA2 or EphrinA1 and modulate (increase or decrease) the expression and/or activity of EphA2 or EphrinA1 . In a specific embodiment, an intrabody of the invention immunospecifically binds to the intracellular domain of EphA2 and decreases EphA2 cytoplasmic tail phosphorylation without causing EphA2 degradation. In another embodiment, an intrabody of the invention immunospecifically binds to EphA2 and prevents or reduces EphA2 signal transduction (including, but not limited to EphA2 autophosphorylation) but does not inhibit or reduce the interaction between EphA2 and an endogenous ligand(s) of EphA2, preferably, EphrinA1.

The antibodies used in the methods of the invention may be from any animal origin including birds and mammals (e.g., human, murine, donkey, sheep, rabbit, goat, guinea pig, camel, horse, or chicken). In a most preferred embodiment, the antibody is human or has been humanized. As used herein, “human” antibodies include antibodies having the amino acid sequence of a human immunoglobulin and include antibodies isolated from human immunoglobulin libraries or from mice that express antibodies from human genes.

The antibodies used in the methods of the present invention may be monospecific, bispecific, trispecific or of greater multi specificity. Multispecific antibodies may immunospecifically bind to different epitopes of an EphA2 polypeptide or an EphrinA1 polypeptide or may immunospecifically bind to both an EphA2 polypeptide or an EphrinA1 polypeptide as well a heterologous epitope, such as a heterologous polypeptide or solid support material. See, e.g., International Patent Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553.

### 5.1.1.1.1 BiTE Molecules

In a specific embodiment, antibodies for use in the methods of the invention are bispecific T cell engagers (BiTEs). Bispecific T cell engagers (BiTE) are bispecific antibodies that can redirect T cells for antigen-specific elimination of targets. A BiTE molecule has an antigen-binding domain that binds to a T cell antigen (e.g. CD3) at one end of the molecule and an antigen binding domain that will bind to an antigen on the target cell. A BiTE molecule was described in International Publication No. WO 99/54440, which is herein incorporated by reference. This publication describes a novel single-chain multifunctional polypeptide that comprises binding sites for the CD19 and CD3 antigens (CD19×CD3). This molecule was derived from two antibodies, one that binds to CD19 on the B cell and an antibody that binds to CD3 on the T cells. The variable regions of these different antibodies are linked by a polypeptide sequence, thus creating a single molecule. Also described, is the linking of the heavy chain (V_{H}) and light chain (V_{L}) variable domains with a flexible linker to create a single chain, bispecific antibody.

In an embodiment of this invention, an antibody or ligand that immunospecifically binds a polypeptide of interest (e.g., EphA2 and/or EphrinA1) will comprise a portion of the BiTE molecule. For example, the V_{H} and/or V_{L} (preferably a scFv) of an antibody that binds a polypeptide of interest (e.g., an Eph receptor and/or an Ephrin) can be fused to an anti-CD3 binding portion such as that of the molecule described above, thus creating a BiTE molecule that targets the polypeptide of interest (e.g., EphA2 and/or EphrinA1). In addition to the heavy and/or light chain variable domains of antibody against a polypeptide of interest (e.g., EphA2 and/or EphrinA1), other molecules that bind the polypeptide of interest (e.g., EphA2 and/or EphrinA1) can comprise the BiTE molecule, for example receptors (e.g., EphA2 and/or EphrinA1). In another embodiment, the BiTE molecule can comprise a molecule that binds to other T cell antigens (other than CD3). For example, ligands and/or antibodies that immunospecifically bind to T-cell antigens like CD2, CD4, CD8, CD11a, TCR, and CD28 are contemplated to be part of this invention. This list is not meant to be exhaustive but only to illustrate that other molecules that can immunospecifically bind to a T cell antigen can be used as part of a BiTE molecule. These molecules can include the VH and/or VL portions of the antibody or natural ligands (for example LFA3 whose natural ligand is CD3).

### 5.1.1.1.2 Intrabodies

In certain embodiments, the antibody to be used with the invention binds to an intracellular epitope, i.e., is an intrabody. In a specific embodiment, an intrabody of the invention binds to the cytoplasmic domain of EphA2 and prevents EphA2 signaling (e.g., autophosphorylation). An intrabody comprises at least a portion of an antibody that is capable of immunospecifically binding an antigen and preferably does not contain sequences coding for its secretion. Such antibodies will bind antigen intracellularly. In one embodiment, the intrabody comprises a single-chain Fv (“scFv”). scFvs are antibody fragments comprising the V_{H} and V_{L} domains of antibody, wherein these domains are present in a single polypeptide chain. Generally, the scFv polypeptide further comprises a polypeptide linker between the V_{H} and V_{L} domains which enables the scFv to form the desired structure for antigen binding. For a review of scFvs see Pluckthun in The Pharmacology of Monoclonal Antibodies, vol. 1 13, Rosenburg and Moore eds. Springer-Verlag, New York, pp. 269-315 (1994). In a further embodiment, the intrabody preferably does not encode an operable secretory sequence and thus remains within the cell (see generally Marasco, Wash., 1998, “Intrabodies: Basic Research and Clinical Gene Therapy Applications” Springer:New York).

Generation of intrabodies is well-known to the skilled artisan and is described, for example, in U.S. Pat. Nos. 6,004,940; 6,072,036; 5,965,371, which are incorporated by reference in their entireties herein. Further, the construction of intrabodies is discussed in Ohage and Steipe, 1999, J. Mol. Biol. 291:1119-1128; Ohage et al., 1999, J. Mol. Biol. 291:1129-1134; and Wirtz and Steipe, 1999, Protein Science 8:2245-2250. which references are incorporated herein by reference in their entireties. Recombinant molecular biological techniques such as those described for recombinant production of antibodies may also be used in the generation of intrabodies.

In one embodiment, intrabodies of the invention retain at least about 75% of the binding effectiveness of the complete antibody (i.e., having the entire constant domain as well as the variable regions) to the antigen. More preferably, the intrabody retains at least 85% of the binding effectiveness of the complete antibody. Still more preferably, the intrabody retains at least 90% of the binding effectiveness of the complete antibody. Even more preferably, the intrabody retains at least 95% of the binding effectiveness of the complete antibody.

In producing intrabodies, polynucleotides encoding variable region for both the V_{H} and V_{L} chains of interest can be cloned by using, for example, hybridoma mRNA or splenic mRNA as a template for PCR amplification of such domains (Huse et al., 1989, Science 246:1276). In one preferred embodiment, the polynucleotides encoding the V_{H} and V_{L} domains are joined by a polynucleotide sequence encoding a linker to make a single chain antibody (scFv). The scFv typically comprises a single peptide with the sequence V_{H}-linker-V_{L} or V_{L}-linker-V_{H}. The linker is chosen to permit the heavy chain and light chain to bind together in their proper conformational orientation (see for example, Huston et al., 1991, Methods in Enzym. 203:46-121, which is incorporated herein by reference). In a further embodiment, the linker can span the distance between its points of fusion to each of the variable domains (e.g., 3.5 nm) to minimize distortion of the native Fv conformation. In such an embodiment, the linker is a polypeptide of at least 5 amino acid residues, at least 10 amino acid residues, at least 15 amino acid residues, or greater. In a further embodiment, the linker should not cause a steric interference with the V_{H} and V_{L} domains of the combining site. In such an embodiment, the linker is 35 amino acids or less, 30 amino acids or less, or 25 amino acids or less. Thus, in a most preferred embodiment, the linker is between 15-25 amino acid residues in length. In a further embodiment, the linker is hydrophilic and sufficiently flexible such that the V_{H} and V_{L} domains can adopt the conformation necessary to detect antigen. Intrabodies can be generated with different linker sequences inserted between identical V_{H} and V_{L} domains. A linker with the appropriate properties for a particular pair of V_{H} and V_{L} domains can be determined empirically by assessing the degree of antigen binding for each. Examples of linkers include, but are not limited to, those sequences disclosed in Table 3, infra.

TABLE 3

| | |
|---|---|
| Sequence | SEQ ID NO. |

| | |
|---|---|
| (Gly Gly Gly Gly Ser)₃ | SEQ ID NO: 1 |
| Glu Ser Gly Arg Ser Gly Gly Gly Gly Ser | SEQ ID NO:2 |
| Gly Gly Gly Gly Ser | |
| Glu Gly Lys Ser Ser Gly Ser Gly Ser Glu | SEQ ID NO:3 |
| Ser Lys Ser Thr | |
| Glu Gly Lys Ser Ser Gly Ser Gly Ser Glu | SEQ ID NO:4 |
| Ser Lys Ser Thr Gln | |
| Glu Gly Lys Ser Ser Gly Ser Gly Ser Glu | SEQ ID NO:5 |
| Ser Lys Val Asp | |
| Gly Ser Thr Ser Gly Ser Gly Lys Ser Ser | SEQ ID NO:6 |
| Glu Gly Lys Gly | |
| Lys Glu Ser Gly Ser Val Ser Ser Glu Gln | SEQ ID NO:7 |
| Leu Ala Gln Phe Arg Ser Leu Asp | |
| Glu Ser Gly Ser Val Ser Ser Glu Glu Leu | SEQ ID NO:8 |
| Ala Phe Arg Ser Leu Asp | |

In one embodiment, intrabodies are expressed in the cytoplasm. In other embodiments, the intrabodies are localized to various intracellular locations. In such embodiments, specific localization sequences can be attached to the intrabody polypeptide to direct the intrabody to a specific location. Intrabodies can be localized, for example, to the following intracellular locations: endoplasmic reticulum (Munro et al., 1987, Cell 48:899-907; Hangejorden et al., 1991, J. Biol. Chem. 266:6015); nucleus (Lanford et al., 1986, Cell 46:575; Stanton et al.,1986, PNAS 83:1772; Harlow et al., 1985, Mol. Cell Biol. 5:1605; Pap et al., 2002, Exp. Cell Res. 265:288-93); nucleolar region (Seomi et al., 1990, J. Virology 64:1803; Kubota et al., 1989, Biochem. Biophys. Res. Comm. 162:963; Siomi et al., 1998, Cell 55:197); endosomal compartment (Bakke et al., 1990, Cell 63:707-716); mitochondrial matrix (Pugsley, A. P., 1989, “Protein Targeting”, Academic Press, Inc.); Golgi apparatus (Tang et al., 1992, J. Bio. Chem. 267:10122-6); liposomes (Letoumeur et al., 1992, Cell 69:1183); peroxisome (Pap et al., 2002, Exp. Cell Res. 265:288-93); trans Golgi network (Pap et al., 2002, Exp. Cell Res. 265:288-93); and plasma membrane (Marchildon et al., 1984, PNAS 81:7679-82; Henderson et al., 1987, PNAS 89:339-43; Rhee et al., 1987, J. Virol. 61:1045-53; Schultz et al., 1984, J. Virol. 133:431-7; Ootsuyarna et al., 1985, Jpn. J. Can. Res. 76:1132-5; Ratner et al., 1985, Nature 313:277-84). Examples of localization signals include, but are not limited to, those sequences disclosed in Table 4, infra.

TABLE 4

| | | |
|---|---|---|
| Localization | Sequence | SEQ ID NO. |

| | | |
|---|---|---|
| endoplasmic reticulum | Lys Asp Glu Leu | SEQ ID NO: 9 |
| endoplasmic reticulum | Asp Asp Glu Leu | SEQ ID NO: 10 |
| endoplasmic reticulum | Asp Glu Glu Leu | SEQ ID NO: 11 |
| endoplasmic reticulum | Gln Glu Asp Leu | SEQ ID NO: 12 |
| endoplasmic reticulum | Arg Asp Glu Leu | SEQ ID NO: 13 |
| Nucleus | Pro Lys Lys Lys Arg Lys Val | SEQ ID NO: 14 |
| Nucleus | Pro Gln Lys Lys Ile Lys Ser | SEQ ID NO: 15 |
| Nucleus | Gln Pro Lys Lys Pro | SEQ ID NO: 16 |
| Nucleus | Arg Lys Lys Arg | SEQ ID NO: 17 |
| Nucleus | Lys Lys Lys Arg Lys | SEQ ID NO: 18 |
| nucleolar region | Arg Lys Lys Arg Arg Gln Arg Arg Arg Ala | SEQ ID NO: 19 |
| | His Gln | |
| nucleolar region | Arg Gln Ala Arg Arg Asn Arg Arg Arg Arg | SEQ ID NO: 20 |
| | Trp Arg Glu Arg Gln Arg | |
| nucleolar region | Met Pro Leu Thr Arg Arg Arg Pro Ala Ala | SEQ ID NO: 21 |
| | Ser Gln Ala Leu Ala Pro Pro Thr Pro | |
| endosomal compartment | Met Asp Asp Gln Arg Asp Leu Ile Ser Asn | SEQ ID NO: 22 |
| | Asn Glu Gln Leu Pro | |
| mitochondrial matrix | Met Leu Phe Asn Leu Arg Xaa Xaa Leu Asn | SEQ ID NO: 23 |
| | Asn Ala Ala Phe Arg His Gly His Asn Phe | |
| | Met Val Arg Asn Phe Arg Cys Gly Gln Pro | |
| | Leu Xaa | |
| Peroxisome | Ala Lys Leu | SEQ ID NO: 24 |
| trans Golgi network | Ser Asp Tyr Gln Arg Leu | SEQ ID NO: 25 |
| plasma membrane | Gly Cys Val Cys Ser Ser Asn Pro | SEQ ID NO: 26 |
| plasma membrane | Gly Gln Thr Val Thr Thr Pro Leu | SEQ ID NO: 27 |
| plasma membrane | Gly Gln Glu Leu Ser Gln His Glu | SEQ ID NO: 28 |
| plasma membrane | Gly Asn Ser Pro Ser Tyr Asn Pro | SEQ ID NO: 29 |
| plasma membrane | Gly Val Ser Gly Ser Lys Gly Gln | SEQ ID NO: 30 |
| plasma membrane | Gly Gln Thr Ile Thr Thr Pro Leu | SEQ ID NO: 31 |
| plasma membrane | Gly Gln Thr Leu Thr Thr Pro Leu | SEQ ID NO: 32 |
| plasma membrane | Gly Gln Ile Phe Ser Arg Ser Ala | SEQ ID NO: 33 |
| plasma membrane | Gly Gln Ile His Gly Leu Ser Pro | SEQ ID NO: 34 |
| plasma membrane | Gly Ala Arg Ala Ser Val Leu Ser | SEQ ID NO: 35 |
| plasma membrane | Gly Cys Thr Leu Ser Ala Glu Glu | SEQ ID NO: 36 |

V_{H} and V_{L} domains are made up of the immunoglobulin domains that generally have a conserved structural disulfide bond. In embodiments where the intrabodies are expressed in a reducing environment (e.g., the cytoplasm), such a structural feature cannot exist. Mutations can be made to the intrabody polypeptide sequence to compensate for the decreased stability of the immunoglobulin structure resulting from the absence of disulfide bond formation. In one embodiment, the V_{H} and/or V_{L} domains of the intrabodies contain one or more point mutations such that their expression is stabilized in reducing environments (see Steipe et al., 1994, J. Mol. Biol. 240:188-92; Wirtz and Steipe, 1999, Protein Science 8:2245-50; Ohage and Steipe, 1999, J. Mol. Biol. 291:1119-28; Ohage et al., 1999, J. Mol Biol. 291:1129-34).

Intrabody Proteins as Therapeutics

In one embodiment, the recombinantly expressed intrabody protein is administered to a patient. Such an intrabody polypeptide must be intracellular to mediate a prophylactic or therapeutic effect. In this embodiment of the invention, the intrabody polypeptide is associated with a “membrane permeable sequence”. Membrane permeable sequences are polypeptides capable of penetrating through the cell membrane from outside of the cell to the interior of the cell. When linked to another polypeptide, membrane permeable sequences can also direct the translocation of that polypeptide across the cell membrane as well.

In one embodiment, the membrane permeable sequence is the hydrophobic region of a signal peptide (see, e.g., Hawiger, 1999, Curr. Opin. Chem. Biol. 3:89-94; Hawiger, 1997, Curr. Opin. Immunol. 9:189-94; U.S. Pat. Nos. 5,807,746 and 6,043,339, which are incorporated herein by reference in their entireties). The sequence of a membrane permeable sequence can be based on the hydrophobic region of any signal peptide. The signal peptides can be selected, e.g., from the SIGPEP database (see e.g., von Heijne, 1987, Prot. Seq. Data Anal. 1:41-2; von Heijne and Abrahmsen, 1989, FEBS Lett. 224:439-46). When a specific cell type is to be targeted for insertion of an intrabody polypeptide, the membrane permeable sequence is preferably based on a signal peptide endogenous to that cell type. In another embodiment, the membrane permeable sequence is a viral protein (e.g., Herpes Virus Protein VP22) or fragment thereof (see e.g., Phelan et al., 1998, Nat. Biotechnol. 16:440-3). A membrane permeable sequence with the appropriate properties for a particular intrabody and/or a particular target cell type can be determined empirically by assessing the ability of each membrane permeable sequence to direct the translocation of the intrabody across the cell membrane. Examples of membrane permeable sequences include, but are not limited to, those sequences disclosed in Table 5, infra.

TABLE 5

| | |
|---|---|
| Sequence | SEQ ID NO. |

| | |
|---|---|
| Ala Ala Val Ala Leu Leu Pro Ala Val | SEQ ID NO:37 |
| Leu Leu Ala Leu Leu Ala Pro | |
| Ala Ala Val Leu Leu Pro Val Leu Leu | SEQ ID NO:38 |
| Ala Ala Pro | |
| Val Thr Val Leu Ala Leu Gly Ala Leu | SEQ ID NO:39 |
| Ala Gly Val Gly Val Gly | |

In another embodiment, the membrane permeable sequence can be a derivative. In this embodiment, the amino acid sequence of a membrane permeable sequence has been altered by the introduction of amino acid residue substitutions, deletions, additions, and/or modifications. For example, but not by way of limitation, a polypeptide may be modified, e.g., by glycosylation, acetylation, pegylation, phosphorylation, amidation, derivatization by known protecting/blocking groups, proteolytic cleavage, linkage to a cellular ligand or other protein, etc. A derivative of a membrane permeable sequence polypeptide may be modified by chemical modifications using techniques known to those of skill in the art, including, but not limited to specific chemical cleavage, acetylation, formylation, metabolic synthesis of tunicamycin, etc. Further, a derivative of a membrane permeable sequence polypeptide may contain one or more non-classical amino acids. In one embodiment, a polypeptide derivative possesses a similar or identical function as an unaltered polypeptide. In another embodiment, a derivative of a membrane permeable sequence polypeptide has an altered activity when compared to an unaltered polypeptide. For example, a derivative membrane permeable sequence polypeptide can translocate through the cell membrane more efficiently or be more resistant to proteolysis.

The membrane permeable sequence can be attached to the intrabody in a number of ways. In one embodiment, the membrane permeable sequence and the intrabody are expressed as a fusion protein. To this embodiment, the nucleic acid encoding the membrane permeable sequence is attached to the nucleic acid encoding the intrabody using standard recombinant DNA techniques (see e.g., Rojas et al., 1998, Nat. Biotechnol. 16:370-5). In a further embodiment, there is a nucleic acid sequence encoding a spacer peptide placed in between the nucleic acids encoding the membrane permeable sequence and the intrabody. In another embodiment, the membrane permeable sequence polypeptide is attached to the intrabody polypeptide after each is separately expressed recombinantly (see e.g., Zhang et al., 1998, PNAS 95:9184-9). In this embodiment, the polypeptides can be linked by a peptide bond or a non-peptide bond (e.g. with a crosslinking reagent such as glutaraldehyde or a thiazolidino linkage see e.g., Hawiger, 1999, Curr. Opin. Chem. Biol. 3:89-94) by methods standard in the art.

The administration of the membrane permeable sequence-intrabody polypeptide can be by parenteral administration, e.g., by intravenous injection including regional perfusion through a blood vessel supplying the tissues(s) or organ(s) having the target cell(s), or by inhalation of an aerosol, subcutaneous or intramuscular injection, topical administration such as to skin wounds and lesions, direct transfection into, e.g., bone marrow cells prepared for transplantation and subsequent transplantation into the subject, and direct transfection into an organ that is subsequently transplanted into the subject. Further administration methods include oral administration, particularly when the complex is encapsulated, or rectal administration, particularly when the complex is in suppository form. A pharmaceutically acceptable carrier includes any material that is not biologically or otherwise undesirable, i.e., the material may be administered to an individual along with the selected complex without causing any undesirable biological effects or interacting in a deleterious manner with any of the other components of the pharmaceutical composition in which it is contained.

Conditions for the administration of the membrane permeable sequence-intrabody polypeptide can be readily be determined, given the teachings in the art (see e.g., Remington's Pharmaceutical Sciences, 18^{th} Ed., E. W. Martin (ed.), Mack Publishing Co., Easton, Pa. (1990)). If a particular cell type in vivo is to be targeted, for example, by regional perfusion of an organ or tumor, cells from the target tissue can be biopsied and optimal dosages for import of the complex into that tissue can be determined in vitro to optimize the in vivo dosage, including concentration and time length. Alternatively, culture cells of the same cell type can also be used to optimize the dosage for the target cells in vivo.

Intrabody Gene Therapy as Therapeutic

In another embodiment, a polynucleotide encoding an intrabody is administered to a patient (e.g., as in gene therapy). In this embodiment, methods as described in Section 5.3.1, infra can be used to administer the polynucleotide of the invention.

### 5.1.1.1.3 Methods of Producing Antibodies

The antibodies that immunospecifically bind to an antigen can be produced by any method known in the art for the synthesis of antibodies, in particular, by chemical synthesis or preferably, by recombinant expression techniques.

Polyclonal antibodies immunospecific for an antigen can be produced by various procedures well-known in the art. For example, a human antigen can be administered to various host animals including, but not limited to, rabbits, mice, rats, etc. to induce the production of sera containing polyclonal antibodies specific for the human antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T Cell Hybridomas 563 681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a non-murine antigen and once an immune response is detected, e.g., antibodies specific for the antigen are detected in the mouse serum, the mouse spleen is harvested and splenocytes isolateu. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

The present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with a non-murine antigen with myeloma cells and then screening the hybridomas resulting from the fusion for hybridoma clones that secrete an antibody able to bind to the antigen.

Antibody fragments which recognize specific particular epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; International application No. PCT/GB91/O1 134; International publication Nos. WO 90/02809, WO 91/10737; WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. Preferably, the vectors for expressing the VH or VL domains comprise an EF-1α promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use humanized antibodies or chimeric antibodies. Completely human antibodies and humanized antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International publication Nos. WO 98/46645, WO 98/50433, WO 98/24893, WO98/16654, WO 96/34096, WO 96/33735 and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, 1995, Int. Rev. Immunol. 13:65 93. For a detailed discussion of this technology for producing human antibodies and human monoclonal antibodies and protocols for producing such antibodies, see, e.g., International publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Fremont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,311,415, which are incorporated herein by reference in their entireties.

Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions (see, e.g., U.S. Pat. No. 5,585,089; and Riechmann et al., 1988, Nature 332:323, which are incorporated herein by reference in their entireties).

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immuoglobulin. A humanized antibody comprises substantially all of at least one, and typically two, variable domains (Fab, Fab′, F(ab′)₂, Fabc, Fv) in which all or substantially all of the CDR regions correspond to those of a non human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG₁, IgG₂, IgG₃ and IgG₄. Usually the constant domain is a complement fixing constant domain where it is desired that the humanized antibody exhibit cytotoxic activity, and the class is typically IgG1. Where such cytotoxic activity is not desirable, the constant domain may be of the IgG2 class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework and CDR sequences, more often 90%, and most preferably greater than 95%. A humanized antibody can be produced using variety of techniques known in the art, including but not limited to, CDR grafting (see e.g., European Pat. No. EP 239,400; International Publication No. WO 91/09967; and U.S. Pat. Nos. 5,225,539, 5,530,101, and 5,585,089, each of which is incorporated herein in its entirety by reference), veneering or resurfacing (see e.g., European Pat. Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973, each of which is incorporated herein by its entirety by reference), chain shuffling (see e.g., U.S. Pat. No. 5,565,332, which is incorporated herein in its entirety by reference), and techniques disclosed in, e.g., U.S. Pat. No. 6,407,213, U.S. Pat. No. 5,766,886, International Publication No. WO 9317105, Tan et al., J. Immunol. 169:1119 25 (2002), Caldas et al., Protein Eng. 13(5):353 60 (2000), Morea et al., Methods 20(3):267 79 (2000), Baca et al., J. Biol. Chem. 272(16):10678 84 (1997), Roguska et al., Protein Eng. 9(10):895 904 (1996), Couto et al., Cancer Res. 55 (23 Supp):5973s-5977s (1995), Couto et al., Cancer Res. 55(8):1717 22 (1995), Sandhu J S, Gene 150(2):40910 (1994), and Pedersen et al., J. Mol. Biol. 235(3):959 73 (1994), each of which is incorporated herein in its entirety by reference. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323, which are incorporated herein by reference in their entireties.)

Further, the antibodies that immunospecifically bind to EphA2 or EphrinA1 or fragments thereof can, in turn, be utilized to generate anti-idiotype antibodies that “mimic” an antigen using techniques well known to those skilled in the art. (See, e.g., Greenspan & Bona, 1989, FASEB J .7(5):437-444; and Nissinoff, 1991, J. Immunol. 147(8):2429-2438)

### 5.1.1.2 EphA2 Fragments and EphrinA1 Fragments as EphA2/EphrinA1 Modulators

In one embodiment, an EphA2/EphrinA1 Modulator of the invention is an EphA2 polypeptide. In a specific embodiment, an EphA2/Ephrin Modulator is a fragment of EphA2 (“EphA2 Fragments”). In accordance with this embodiment, the EphA2 Fragment preferably retains the ability to bind to EphrinA1. In a preferred embodiment, the EphA2 Fragment retains the ability to bind to EphrinA1 and inhibits or reduces binding of endogenous EphA2 to an endogenous ligand of EphA2, preferably EphrinA1. In a specific embodiment, an EphA2/Ephrin Modulator is an EphA2 Fragment that specifically binds to EphrinA1 or fragments thereof and does not bind to other Ephrin molecules or fragments thereof.

Non-limiting examples of EphA2 Fragments include, but are not limited to, EphA2 Fragments comprising the ligand binding domain of human EphA2 (amino acid residues 28 to 201) and any one or more of the following domains: the first fibronectin Type III domain (amino acid residues 332 to 424); the second fibronectin Type III domain (amino acid residues 439 to 519); the tyrosine kinase catalytic domain (amino acid residues 607 to 874); and/or the sterile alpha motif “SAM” domain (amino acid residues 902 to 968), the sequences of which may be found in the GenBank database (e.g., GenBank Accession No. NP_{—}004422.2 for human EphA2). In a specific embodiment, an EphA2 Fragment is soluble (i.e., not membrane-bound). In another specific embodiment, an EphA2 Fragment of the invention lacks the transmembrane domain of EphA2 (e.g., from amino acid residues 520 to 606) and is not membrane-bound. In certain embodiments, an EphA2 Fragment of the invention comprises the extracellular domain or a fragment thereof of EphA2. In other embodiments, an EphA2 Fragment of the invention comprises the extracellular domain or a fragment thereof and lacks the transmembrane domain or a portion thereof such that the EphA2 is not membrane-bound. In other embodiments, an EphA2 Fragment of the invention comprises the cytoplasmic domain or a fragment thereof of EphA2. In further embodiments, an EphA2 Fragment of the invention comprises the cytoplasmic domain or a fragment of the cytoplasmic domain of EphA2 and lacks the transmembrane domain or a fragment thereof such that the EphA2 is not membrane-bound. In yet further embodiments, an EphA2 Fragment of the invention comprises the extracellular domain or a fragment thereof of EphA2 and the cytoplasmic domain or a fragment thereof. Such an EphA2 Fragment preferably lacks the transmembrane domain.

In a specific embodiment, an EphA2 Fragment comprises only the extracellular domain of EphA2. In another specific embodiment, an EphA2 Fragment comprises only the ligand binding domain (e.g., amino acid residues 28 to 201 of human EphA2 as disclosed in GenBank Accession No. NP_{—}004422.2). In specific embodiments, an EphA2 Fragment of the invention comprises specific fragments of the extracellular domain of human of EphA2 (e.g., amino acid residues 1 to 25, 1 to 50, 1 to 75, 1 to 100, 1 to 125, 1 to 150, 1 to 175, 1 to 200, 1 to 225, 1 to 250, 1 to 275, 1 to 300, 1 to 325, 1 to 350, 1 to 375, 1 to 400, 1 to 425, 1 to 450, 1 to 475, 1 to 500, or 1 to 525). In another specific embodiment, an EphA2 Fragment of the invention comprises the transmembrane domain or a fragment of the transmembrane domain. In accordance with this embodiment, the EphA2 Fragment may further comprise the extracellular domain of a fragment thereof of EphA2 and/or the cytoplasmic domain or a fragment thereof of EphA2.

The EphA2 Fragments include polypeptides that are 100%, 98%, 95%, 90%, 85%, 80%, 75%, 70%, 65%, 60%, 55%, 50%, 45%, 40% identical to endogenous EphA2 sequences. The determination of percent identity of two amino acid sequences can be determined by any method known to one skilled in the art, including BLAST protein searches. In specific embodiments, EphA2 Fragments of the invention can be analogs or derivatives of EphA2. For example, EphA2 Fragments of the invention include derivatives that are modified, i.e., by covalent attachment of any type of molecule to the polypeptide. For example, but not by way of limitation, the polypeptide derivatives (e.g., EphA2 polypeptide derivatives) include polypeptides that have been modified, e.g., by glycosylation, acetylation, pegylation, phosphorylation, amidation, derivatization by known protecting/blocking groups, proteolytic cleavage, linkage to a cellular ligand, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

In a specific embodiment, an EphA2/EphrinA1 Modulator of the invention is a dominant negative form of EphA2 which lacks the cytoplasmic domain or a fragment thereof required for signaling. In accordance with this embodiment, the dominant negative form of EphA2 comprises the transmembrane domain or a fragment thereof of EphA2 and is membrane-bound. In a specific embodiment, the dominant negative form of EphA2 retains the ability to bind EphrinA1 but is incapable of signaling, induces low to negligible signaling or does not induce all the signal transduction pathways activated upon ligand-receptor interaction. In specific embodiments, low to negligible signaling in the context of EphA2 refers to a decrease in any aspect of EphA2 signaling upon ligand binding by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control in an in vivo and/or an in vitro assay described herein or well known to one of skill in the art. In certain aspects of the invention, EphA2 signaling encompasses any one or more of the signaling pathways that are activated upon EphA2 binding to its endogenous ligand (e.g., EphrinA1). Non-limiting examples of such signaling pathways include but are not limited to, the mitogen-activated protein kinase (MAPK)/ERK pathway, the Ras pathway, and pathways involving the Src family of kinases (for other Eph receptor pathways, see, Cheng et al., 2002, Cytokine & Growth Factor Rev. 13:75-85; Kullander and Klein, 2002, Nature Rev. 3:475-486; Holder and Klein, 1999, Development 126:2033-2044; Zhou, 1998, Pharmacol. Ther. 77:151-181; and Nakamoto and Bergemann, 2002, Microscopy Res. & Technique 59:58-67, which are all incorporated by reference herein in their entireties).

Various assays known to one of skill in the art may be performed to measure EphA2 signaling. For example, EphA2 phosphorylation may be measured to determine whether EphA2 signaling is activated upon ligand binding by measuring the amount of phosphorylated EphA2 present in EphrinA1-treated cells relative to control cells that are not treated with EphrinA1. EphA2 may be isolated using any protein immunoprecipitation method known to one of skill in the art and an EphA2 antibody of the invention. Phosphorylated EphA2 may then be measured using anti-phosphotyrosine antibodies (Upstate Tiotechnology, Inc., Lake Placid, N.Y.) using any standard immunoblotting method known to one of skill in the art. See, e.g., Cheng et al., 2002, Cytokine & Growth Factor Rev. 13:75-85. In another embodiment, MAPK phosphorylation may be measured to determine whether EphA2 signaling is activated upon ligand binding by measuring the amount of phosphorylated MAPK present in EphrinA1-treated cells relative to control cells that are not treated with EphrinA1 using standard immunoprecipitation and immunoblotting assays known to one of skill in the art (see, e.g., Miao et al., 2003, J. Cell Biol. 7:1281-1292, which is incorporated by reference herein in its entirety).

In one embodiment, an EphA2/EphrinA1 Modulator is an EphrinA1 polypeptide. In a specific embodiment, an EphA2/EphrinA1 Modulator of the invention is a fragment of EphrinA1 (“EphrinA1 Fragment”). In accordance with this embodiment, the EphrinA1 Fragment preferably retains the ability to bind to EphA2. In a preferred embodiment, the EphrinA1 Fragment retains the ability to bind to EphA2 and inhibits or reduces binding of endogenous EphrinA1 to endogenous EphA2.

Non-limiting examples of EphrinA1 Fragments include, but are not limited to, any fragment of human EphrinA1 as disclosed in the GenBank database (e.g., GenBank Accession Nos. NP_{—}004419 (variant 1) and NP_{—}872626 (variant 2)). In a specific embodiment, an EphrinA1 Fragment is soluble (i.e., not membrane-bound). In a specific embodiment, an EphrinA1 Fragment of the invention comprises the extracellular domain of human EphrinA1 or a portion thereof. In further embodiments, an EphrinA1 Fragment of the invention comprises the extracellular domain of human EphrinA1 or a fragment thereof and is not membrane-bound. In specific embodiments, an EphrinA1 Fragment of the invention comprises specific fragments of the extracellular domain of human EphrinA1 variant 1 or a fragment thereof and is not membrane bound. In other specific embodiments, an EphrinA1 Fragment of the invention comprises specific fragments of the extracellular domain of human EphrinA1 variant 2 or a fragment thereof and is not membrane-bound.

The EphrinA1 Fragments include polypeptides that are 100%, 98%, 95%, 90%, 85%, 80%, 75%, 70%, 65%, 60%, 55%, 50%, 45%, 40% identical to endogenous EphrinA1 sequences. The determination of percent identity of two amino acid sequences can be determined by any method known to one skilled in the art, including BLAST protein searches. In specific embodiments, EphrinA1 Fragments of the invention can be analogs or derivatives of EphrinA1. For example, EphrinA1 Fragments of the invention include derivatives that are modified, i.e., by covalent attachment of any type of molecule to the polypeptide. For example, but not by way of limitation, the polypeptide derivatives (e.g., EphrinA1 polypeptide derivatives) include polypeptides that have been modified, e.g., by glycosylation, acetylation, pegylation, phosphorylation, amidation, derivatization by known protectingiblocking groups, proteolytic cleavage, linkage to a cellular ligand, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

In a specific embodiment, an EphA2/EphrinA1 Modulator is an EphA2 or EphrinA1 fusion protein. EphA2/EphrinA1 Modulators that are fusion proteins are discussed in further detail, for example, in Section 5.1.1.3, infra. In a preferred embodiment, an EphA2 or EphrinA1 fusion protein is soluble. Non-limiting examples of EphA2 fusion proteins include soluble forms of EphA2 such as EphA2-Fc (see, e.g., Cheng et al., 2002, Mol. Cancer Res. 1:2-11, which is incorporated by reference herein in its entirety). In a specific embodiment, an EphA2 fusion protein comprises EphA2 fused to the Fc portion of human immunoglobulin IgG1. In another embodiment, an EphA2 fusion protein comprises an EphA2 Fragment which retains its ability to bind EphrinA1 (e.g., the extracellular domain of EphA2) fused to the Fc portion of human immunoglobulin IgG1 (see, e.g., Carles-Kinch et al., 2002, Cancer Res. 62:2840-2847; and Cheng et al., 2002, Mol. Cancer Res. 1:2-11, which are incorporated by reference herein in their entireties). In yet a further embodiment, an EphA2 fusion protein comprises an EphA2 Fragment which retains its ability to bind EphrinA1 fused to a heterologous protein (e.g., human serum albumin).

Non-limiting examples of EphrinA1 fusion proteins include soluble forms of EphrinA1 such as EphrinA1-Fc (see, e.g., Duxbury et al., 2004, Biochem. & Biophys. Res. Comm. 320:1096-1102, which is incorporated by reference herein in its entirety). In a specific embodiment, an EphrinA1 fusion protein comprises EphrinA1 fused to an the Fc domain of human immunoglobulin IgG. In another embodiment, an EphrinA1 fusion protein comprises an EphrinA1 Fragment which retains its ability to bind EphA2 fused to the Fc domain of human immunoglobulin IgG. In yet a further embodiment, an EphrinA1 fusion protein comprises an EphrinA1 Fragment which retains its ability to bind EphA2 fused to a heterologous protein (e.g., human serum albumin).

Fragments of EphA2 or EphrinA1 can be made and assayed for the ability to bind EphrinA1 or EphA2, respectively, using biochemical, biophysical, genetic, and/or computational techniques for studying protein-protein interactions that are described herein or by any method known in the art. Non-limiting examples of methods for detecting protein binding (e.g., for detecting EphA2 binding to EphrinA1), qualitatively or quantitatively, in vitro or in vivo, include GST-affinity binding assays, far-Western Blot analysis, surface plasmon resonance (SRP), fluorescence resonance energy transfer (FRET), fluorescence polarization (FP), isothermal titration calorimetry (ITC), circular dichroism (CD), protein fragment complementation assays (PCA), various two-hybrid systems, and proteomics and bioinformatics-based approaches, such as the Scansite program for computational analysis (see, e.g., Fu, H., 2004, Protein-Protein Interactions: Methods and Applications (Humana Press, Totowa, N.J.); and Protein-Protein Interactions: A Molecular Cloning Manual, 2002, Golemis, ed. (Cold Spring Harbor Laboratory Press, Cold Spring Harbor, N.Y.) which are incorporated by reference herein in their entireties).

### 5.1.1.3 Conjugates/Fusion Proteins

The present invention encompasses the use of EphA2/EphrinA1 Modulators (e.g., EphA2 and/or EphrinA1 antibodies or fragments thereof that immunospecifically bind to EphA2 and/or EphrinA1) that are recombinantly fused or chemically conjugated (including both covalent and non-covalent conjugations) to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. For example, antibodies may be used to target heterologous polypeptides to particular cell types, either in vitro or in vivo, by fusing or conjugating the antibodies to antibodies specific for particular cell surface receptors. Antibodies fused or conjugated to heterologous polypeptides may also be used in in vitro immunoassays and purification methods using methods known in the art. See e.g., International Publication WO 93/21232; EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452, which are incorporated by reference in their entireties.

The present invention further includes compositions comprising heterologous polypeptides fused or conjugated to antibody fragments. For example, the heterologous polypeptides may be fused or conjugated to a Fab fragment, Fd fragment, Fv fragment, F(ab)₂ fragment, or portion thereof. Methods for fusing or conjugating proteins, polypeptides, or peptides to an antibody or an antibody fragment are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA89:11337-11341 (said references are incorporated herein by reference in their entireties).

Additional fusion proteins, e.g., of any of the EphA2 or EphrinA1 Modulators of the invention, may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16:76; Hansson, et al., 1999, J. Mol. Biol. 287:265; and Lorenzo and Blasco, 1998, BioTechniques 24:308 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment, which portions immunospecifically bind to EphA2 or EphrinA1 may be recombined with one or more components, motifs, sections, parts, domains, fragments, etc. of one or more heterologous molecules.

Moreover, the EphA2/EphrinA1 Modulators can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, PNAS 86:821, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In other embodiments, EphA2/EphrinA1 Modulators are conjugated to a diagnostic or detectable agent. Such modulators can be useful for monitoring or prognosing the development or progression of an infection as part of a clinical testing procedure, such as determining the efficacy of a particular therapy. Additionally, such modulators can be useful for monitoring or prognosing the development or progression of an infection.

Such diagnosis and detection can accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (131I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (201Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

The present invention further encompasses uses of EphA2/EphrinA1 Modulators conjugated to a prophylactic or therapeutic agent. An EphA2/EphrinA1 Modulator may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Therapeutic moieties include, but are not limited to, antimetabolites (e.g., methotrexate, 6-mercaptopurine, 6-thioguanine, cytarabine, 5-fluorouracil decarbazine); alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP), and cisplatin); anthracyclines (e.g., daunorubicin (formerly daunomycin) and doxorubicin); antibiotics (e.g., dactinomycin (formerly actinomycin), bleomycin, mithramycin, and anthramycin (AMC)); Auristatin molecules (e.g., auristatin PHE, bryostatin 1, and solastatin 10; see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999), all of which are incorporated herein by reference); hormones (e.g., glucocorticoids, progestins, androgens, and estrogens), DNA-repair enzyme inhibitors (e.g., etoposide or topotecan), kinase inhibitors (e.g., compound ST1571, imatinib mesylate (Kantarjian et al., Clin Cancer Res. 8(7):2167-76 (2002)); cytotoxic agents (e.g., paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucorticoids, procaine, tetracaine, lidocaine, propranolol, and puromycin and analogs or homologs thereof and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459); famesyl transferase inhibitors (e.g., R115777, BMS-214662, and those disclosed by, for example, U.S. Pat. Nos: 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305); topoisomerase inhibitors (e.g., camptothecin; irinotecan; SN-38; topotecan; 9-aminocamptothecin; GG-211 (GI 147211); DX-8951f; IST-622; rubitecan; pyrazoloacridine; XR-5000; saintopin; UCE6; UCE1022; TAN-1518A; TAN-1518B; KT6006; KT6528; ED-1 10; NB-506; ED-110; NB-506; and rebeccamycin); bulgarein; DNA minor groove binders such as Hoescht dye 33342 and Hoechst dye 33258; nitidine; fagaronine; epiberberine; coralyne; beta-lapachone; BC-4-1; bisphosphonates (e.g., alendronate, cimadronte, clodronate, tiludronate, etidronate, ibandronate, neridronate, olpandronate, risedronate, piridronate, pamidronate, zolendronate) HMG-CoA reductase inhibitors, (e.g., lovastatin, simvastatin, atorvastatin, pravastatin, fluvastatin, statin, cerivastatin, lescol, lupitor, rosuvastatin and atorvastatin); antisense oligonucleotides (e.g., those disclosed in the U.S. Pat. Nos. 6,277,832, 5,998,596, 5,885,834, 5,734,033, and 5,618,709); adenosine deaminase inhibitors (e.g., Fludarabine phosphate and 2-Chlorodeoxyadenosine); ibritumomab tiuxetan (Zevalin®); tositumomab (Bexxar®)) and pharmaceutically acceptable salts, solvates, clathrates, and prodrugs thereof.

Moreover, an EphA2/EphrinA1 Modulator can be conjugated to therapeutic moieties such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the antibody via a linker molecule. Such linker molecules are commonly known in the art and described in Denardo et al., 1998, Clin Cancer Res. 4:2483-90; Peterson et al., 1999, Bioconjug. Chem. 10:553; and Zimmerman et al., 1999, Nucl. Med. Biol. 26:943-50 each incorporated by reference in their entireties.

Further, an EphA2/EphrinA Modulator may be conjugated to a prophylactic or therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, 62 -interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567-1574), and VEGF (see, International Publication No. WO 99/23105); or a biological response modifier such as, for example, a lymphokine (e.g., interferon gamma (“TFN-γ”), interleukin-1 (“IL-1”), interleukin-2 (“IL-2”), interleukin-4 (“IL-4”), interleukin-5 (“IL-5”), interleukin-6 (“IL-6”), interleuking-7 (“IL-7”), interleukin-10 (“IL-10”), interleukin-12 (“IL-12”), interleukin-15 (“IL-15”), interleukin-23 (“IL-23”), granulocyte macrophage colony stimulating factor (“GM-CSF”), and granulocyte colony stimulating factor (“G-CSF”)), or a growth factor (e.g., growth hormone (“GH”)), or a coagulation agent (e.g., calcium, vitamin K, tissue factors, such as but not limited to, Hageman factor (factor XII), high-molecular-weight kininogen (HMWK), prekallikrein (PK), coagulation proteins-factors II (prothrombin), factor V, XIIa, VIII, XIIIa, XI, XIa, IX, IXa, X, phospholipid fibrinopeptides A and B from the α and β chains of fibrinogen, fibrin monomer).

Moreover, an EphA2/EphrinA1 Modulator can be conjugated to prophylactic or therapeutic moieties such as a radioactive metal ion, such as alpha-emitters such as ²¹³Bi or macrocyclic chelators useful for conjugating radiometal ions, including but not limited to, ¹³¹In, ¹³¹L, ¹³¹Y, ¹³¹Ho, ¹³¹Sm, to polypeptides or any of those listed supra. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

In another embodiment, EphA2/EphrinA1 Modulators can be fused or conjugated to liposomes, wherein the liposomes are used to encapsulate prophylactic or therapeutic agents (see e.g., Park et al., 1997, Can. Lett. 118:153-160; Lopes de Menezes et al., 1998, Can. Res. 58:3320-30; Tseng et al., 1999, Int. J. Can. 80:723-30; Crosasso et al., 1997, J. Pharm. Sci. 86:832-9). In a preferred embodiment, the pharmokinetics and clearance of liposomes are improved by incorporating lipid derivatives of PEG into liposome formulations (see, e.g., Allen et al., 1991, Biochem Biophys Acta 1068:133-41; Huwyler et al., 1997, J. Pharmacol. Exp. Ther. 282:1541-6).

Techniques for conjugating prophylactic or therapeutic moieties to proteins are well known. Moieties can be conjugated to proteins by any method known in the art, including, but not limited to aldehyde/Schiff linkage, sulphydryl linkage, acid-labile linkage, cis-aconityl linkage, hydrazone linkage, enzymatically degradable linkage (see generally Garnett, 2002, Adv. Drug Deliv. Rev. 53:171-216). Techniques for conjugating prophylactic or therapeutic moieties to antibodies are well known, see, e.g., Arnon et al., “Monoclonal Antibodies For Immunotargeting Of Drugs In Cancer Therapy,” in Monoclonal Antibodies And Cancer Therapy, Reisfeld et al. (eds.), pp. 243-56 (Alan R. Liss, Inc. 1985); Hellstrom et al., “Antibodies For Drug Delivery,” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review,” in Monoclonal Antibodies 184: Biological And Clinical Applications, Pinchera et al. (eds.), pp. 475-506 (1985); “Analysis, Results, And Future Prospective Of The Therapeutic Use Of Radiolabeled Antibody In Cancer Therapy,” in Monoclonal Antibodies For Cancer Detection And Therapy, Baldwin et al. (eds.), pp. 303-16 (Academic Press 1985), and Thorpe et al., 1982, Immunol. Rev. 62:119-58. Methods for fusing or conjugating antibodies to polypeptide moieties are known in the art. See, e.g., U.S. Pat. Nos. 5,336,603, 5,622,929, 5,359,046, 5,349,053, 5,447,851, and 5,112,946; EP 307,434; EP 367,166; International Publication Nos. WO 96/04388 and WO 91/06570; Ashkenazi et al., 1991, PNAS 88: 10535-10539; Zheng et al., 1995, J, Immunol. 154:5590-5600; and Vil et al., 1992, PNAS 89:11337-11341. The fusion of an antibody to a moiety does not necessarily need to be direct, but may occur through linker sequences. Such linker molecules are commonly known in the art and described in Denardo et al., 1998, Clin Cancer Res. 4:2483-90; Peterson et al., 1999, Bioconjug. Chem. 10:553; Zimmerman et al., 1999, Nucl. Med. Biol. 26:943-50; Garnett, 2002, Adv. Drug Deliv. Rev. 53:171-216, each of which is incorporated herein by reference in its entirety.

Alternatively, an antibody can be conjugated to a second antibody to form an antibody heteroconjugate as described by Segal in U.S. Pat. No. 4,676,980, which is incorporated herein by reference in its entirety.

A conjugated agent's relative efficacy in comparison to the free agent can depend on a number of factors. For example, rate of uptake of the antibody-agent into the cell (e.g., by endocytosis), rate/efficiency of release of the agent from the antibody, rate of export of the agent from the cell, etc. can all effect the action of the agent. Antibodies used for targeted delivery of agents can be assayed for the ability to be endocytosed by the relevant cell type (i.e., the cell type associated with the disorder to be treated) by any method known in the art. Additionally, the type of linkage used to conjugate an agent to an antibody should be assayed by any method known in the art such that the agent action within the target cell is not impeded.

The prophylactic or therapeutic moiety or drug conjugated to an EphA2/EphrinA1 Modulator of the invention (e.g., an EphA2 or EphrinA1 antibody that immunospecificaily binds to an EphA2 or EphrinA1 polypeptide or fragment thereof, respectively) should be chosen to achieve the desired prophylactic or therapeutic effect(s) for the treatment, management or prevention of an infection. A clinician or other medical personnel should consider the following when deciding on which therapeutic moiety or drug to conjugate to an EphA2/EphrinA1 Modulators: the nature of the disease, the severity of the disease, and the condition of the subject.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### 5.1.1.4 Polynucleotides Encoding Polypeptide EphA2/EphrinA1 Modulators

The EphA2/EphrinA1 Modulators of the invention include polypeptides produced from polynucleotides that hybridize to polynucleotides which encode polypeptides disclosed in sections 5.1.1 above. In one embodiment, antibodies of the invention include EphA2 or EphrinA1 monoclonal antibodies produced from polynucleotides that hybridize to polynucleotides encoding monoclonal antibodies that modulate the expression and/or activity EphA2 and/or EphrinA1 in an assay well known to the art or described herein. In another embodiment, EphA2 Fragments or EphrinA1 Fragments used in the methods of the invention include polypeptides produced from polynucleotides that hybridize to polynucleotides encoding a fragments of EphA2 or EphrinA1. Conditions for hybridization include, but are not limited to, stringent hybridization conditions such as hybridization to filter-bound DNA in 6× sodium chloride/sodium citrate (SSC) at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 60° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3).

The EphA2/EphrinA1 Modulators of the invention include polynucleotides encoding polypeptides described herein. The polynucleotides encoding the polypeptides described herein (e.g., the antibodies of the invention or the EphA2 Fragments and EphrinA1 Fragments) may be obtained and sequenced by any method known in the art. For example, a polynucleotide encoding a polypeptide EphA2/EphrinA1 Modulator used in the methods of the invention may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the polypeptide, annealing and ligating of those oligonucleotides, and then amplification of the ligated oligonucleotides by PCR.

Alternatively, a polynucleotide encoding polypeptide EphA2/EphrinA1 Modulator used in the methods of the invention may be generated from nucleic acid from a suitable source. If a clone containing a nucleic acid encoding a particular polypeptide is not available, but the sequence of the polypeptide is known, a nucleic acid encoding the polypeptide may be chemically synthesized or obtained from a suitable source (e.g., an antibody cDNA library, or a cDNA library generated from, or nucleic acid, preferably poly A+RNA, isolated from, any tissue or cells expressing the desired polypeptide, such as hybridoma cells selected to express an antibody of the invention or epithelial and/or endothelial cells that express EphA2 or EphrinA1) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the polypeptide EphA2/EphrinA1 Modulator. Amplified nucleic acids generated by PCR may then be cloned into replicable cloning vectors using any method well known in the art.

Once the nucleotide sequence of the polypeptide EphA2/EphrinA1 Modulator used in the methods of the invention is determined, the nucleotide sequence may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate polypeptides having a different amino acid sequence, for example to create amino acid substitutions, deletions, and/or insertions.

Standard techniques known to those skilled in the art can be used to introduce mutations in the nucleotide sequence encoding a polypeptide EphA2/EphrinA1 Modulator including, e.g., site-directed mutagenesis and PCR-mediated mutagenesis, which results in amino acid substitutions. Preferably, the derivatives include less than 15 amino acid substitutions, less than 10 amino acid substitutions, less than 5 amino acid substitutions, less than 4 amino acid substitutions, less than 3 amino acid substitutions, or less than 2 amino acid substitutions relative to the original EphA2/EphrinA1 Modulator. In a preferred embodiment, the derivatives have conservative amino acid substitutions made at one or more predicted non-essential amino acid residues.

The present invention also encompasses the use of antibodies or antibody fragments comprising the amino acid sequence of any EphA2 or EphrinA1 antibodies with mutations (e.g., one or more amino acid substitutions) in the framework or variable regions. Preferably, mutations in these antibodies maintain or enhance the avidity and/or affinity of the antibodies for the particular antigen(s) to which they immunospecifically bind. Standard techniques known to those skilled in the art (e.g., immunoassays or ELISA assays) can be used to assay the degree of binding between a polypeptide EphA2/EphrinA1 Modulator and its binding partner. In a specific embodiment, when a polypeptide EphA2/EphrinA1 Modulator is an antibody, an EphA2 Fragment, an EphrinA1 Fragment, an EphA2 fusion protein, an EphrinA1 fusion protein or a dominant negative form of EphA2, binding to EphA2 or EphrinA1, as appropriate, can be assessed.

### 5.1.2 Recombinant Production of Polypeptide EphA2/EphrinA1 Modulators

Recombinant expression of a polypeptide EphA2/EphrinA1 Modulator (including, but not limited to derivatives, analogs or fragments thereof) requires construction of an expression vector containing a polynucleotide that encodes the polypeptide. Once a polynucleotide encoding a polypeptide EphA2/EphrinA1 Modulator has been obtained, a vector for the production of the polypeptide EphA2/EphrinA1 Modulator may be produced by recombinant DNA technology using techniques well known in the art. Methods which are well known to those skilled in the art can be used to construct expression vectors containing polypeptide coding sequences and appropriate transcriptional and translational control signals. Thus, methods for preparing a protein by expressing a polynucleotide containing are described herein. These methods include, for example, in vitro recombinant DNA techniques, synthetic techniques, and in vivo genetic recombination. The invention, thus, provides replicable vectors comprising a nucleotide sequence encoding an polypeptide EphA2/EphrinA1 Modulator.

The expression vector is transferred to a host cell by conventional techniques and the transfected cells are then cultured by conventional techniques to produce a polypeptide EphA2/EphrinA1 Modulator. Thus, the invention includes host cells containing a polynucleotide encoding a polypeptide EphA2/EphrinA1 Modulator operably linked to a heterologous promoter.

A variety of host-expression vector systems may be utilized to express polypeptide EphA2/EphrinA1 Modulator (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express a polypeptide EphA2/EphrinA1 Modulator of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing polypeptide EphA2/EphrinA1 Modulator coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant polypeptide EphA2/EphrinA1 Modulator, are used for the expression of a polypeptide EphA2/EphrinA1 Modulator. For example, mammalian cells such as Chinese hamster ovary cells (CHO), in conjunction with a vector such as the major intermediate early gene promoter element from human cytomegalovirus is an effective expression system for polypeptide EphA2/EphrinA1 Modulators, especially antibody polypeptide EphA2/EphrinA1 Modulators (Foecking et al., 1986, Gene 45:101; and Cockett et al., 1990, BioTechnology 8:2). In a specific embodiment, the expression of nucleotide sequences encoding a polypeptide EphA2/EphrinA1 Modulator is regulated by a constitutive promoter, inducible promoter or tissue specific promoter.

In bacterial systems, a number of expression vectors may be advantageously selected depending upon the use intended for the polypeptide being expressed. For example, when a large quantity of such a protein is to be produced, or the generation or pharmaceutical compositions, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the polypeptide coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the polypeptide EphA2/EphrinA1 Modulator in infected hosts (e.g., see Logan & Shenk, 1984, PNAS 81:3655-3659). Specific initiation signals may also be required for efficient translation of inserted polypeptide coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 3T3, W138, BT483, Hs578T, HTB2, BT2O and T47D, NS0 (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the polypeptide EphA2/EphrinA1 Modulator. Such engineered cell lines may be particularly useful in screening and evaluation of compositions that interact directly or indirectly with the polypeptide EphA2/EphrinA1 Modulator.

A number of selection systems may be used, including but not limited to, the herpes simplex virus thymidine kinase (Wigler et al., 1977, Cell 11:223), glutamine synthetase, hypoxanthine guanine phosphoribosyltransferase (Szybalska & Szybalski, 1992, Proc. Natl. Acad. Sci. USA48:202), and adenine phosphoribosyltransferase (Lowy et al., 1980, Cell 22:8-17) genes can be employed in tk-, gs-, hgprt- or aprt-cells, respectively. Also, antimetabolite resistance can be used as the basis of selection for the following genes: dhfr, which confers resistance to methotrexate (Wigler et al., 1980, PNAS 77:357; O'Hare et al., 1981, PNAS 78:1527); gpt, which confers resistance to mycophenolic acid (Mulligan & Berg, 1981, PNAS 78:2072); neo, which confers resistance to the aminoglycoside G-418 (Wu and Wu, 1991, Biotherapy 3:87; Tolstoshev, 1993, Ann. Rev. Pharmacol. Toxicol. 32:573; Mulligan, 1993, Science 260:926; and Morgan and Anderson, 1993, Ann. Rev. Biochem. 62: 191; May, 1993, TIB TECH 11:155-); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150: 1, which are incorporated by reference herein in their entireties.

The expression levels of a polypeptide EphA2/EphrinA1 Modulator can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing polypeptide EphA2/EphrinA1 Modulator is amplifiable, increase in the level of inhibitor present in culture of host cell will increase the number of copies of the marker gene. Since the amplified region is associated with the polypeptide EphA2/EphrinA1 Modulator gene, production of the polypeptide EphA2/EphrinA1 Modulator will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, PNAS 77:2197). The coding sequences for the heavy and light chains may comprise cDNA or genomic DNA.

Once a polypeptide EphA2/EphrinA1 Modulator of the invention has been produced by recombinant expression, it may be purified by any method known in the art for purification of a polypeptide, for example, by chromatography (e.g. ion exchange, affinity, and sizing column chromatography), centrifugation, differential solubility, or by any other standard technique for the purification of proteins. Further, the polypeptide EphA2/EphrinA1 Modulators may be fused to heterologous polypeptide sequences described herein or otherwise known in the art to facilitate purification.

Polypeptide EphA2/EphrinA1 Modulators of the invention that are antibodies may be expressed using vectors which already include the nucleotide sequence encoding the constant region of the antibody molecule (see, e.g., U.S. Pat. Nos. 5,919,900; 5,747,296; 5,789,178; 5,591,639; 5,658,759; 5,849,522; 5,122,464; 5,770,359; 5,827,739; International Patent Publication Nos. WO 89/01036; WO 89/10404; Bebbington et al., 1992, BioTechnology 10:169). The variable domain of the antibody may be cloned into such a vector for expression of the entire heavy, the entire light chain, or both the entire heavy and light chains. In preferred embodiments for the expression of double-chained antibodies, vectors encoding both the heavy and light chains may be co-expressed in the host cell for expression of the entire immunoglobulin molecule.

In a specific embodiment, the expression of a polypeptide EphA2/EphrinA1 Modulator of the invention (e.g., an EphA2 or EphrinA1 peptide, polypeptide, protein or a fusion protein) is regulated by a constitutive promoter. In another embodiment, the expression of a polypeptide EphA2/EphrinA1 Modulator of the invention (e.g., an EphA2 or EphrinA1 peptide, polypeptide, protein or a fusion protein) is regulated by an inducible promoter. In another embodiment, the expression of a polypeptide EphA2/EphrinA1 Modulator of the invention (e.g., an EphA2 or EphrinA1 peptide, polypeptide, protein or a fusion protein) is regulated by a tissue-specific promoter. For example, EphA2 is regulated by Hoxal And Hoxbl Homeobox transcription factors (see, e.g., Chen et al., 1998, J. Biol. Chem. 273:24670-24675, which is incorporated by reference herein in its entirety, and EphrinA1 is regulated by the Homeobox transcription factor HoxB3 (see, e.g., Myers et al., 2000, J. Cell Biol. 148:343-351, which is incorporated by reference herein in its entirety).

In one embodiment, the method of the invention comprises administration of a composition comprising nucleic acids comprising a nucleotide sequence encoding and EphA2/EphrinA1 Modulator, said nucleic acids being part of an expression vector that expresses the EphA2/EphrinA1 Modulator.

### 5.1.3 Polynucleotide EphA2/EphrinA1 Modulators

In addition to the polypeptide EphA2/EphrinA1 Modulators of the invention, nucleic acid molecules can be used in methods of the invention. In one embodiment, a nucleic acid molecule EphA2/EphrinA1 Modulator can encode all or a fragment of EphA2 to increase EphA2 expression or availability for ligand (preferably, EphrinA1) binding. In another embodiment, a nucleic acid molecule EphA2/EphrinA1 Modulator can encode all or a fragment of EphrinA1 to increase the amount of EphrinA1 available for binding to EphA2. Any method known in the art can be used to increase expression of EphA2 or EphrinA1 using nucleic acid molecules. In a further embodiment, a nucleic acid EphA2/EphrinA1 Modulator reduces the amount of endogenous EphA2 available for ligand binding to EphrinA1. In yet a further embodiment, a nucleic acid molecule EphA2/EphrinA1 Modulator reduces the amount of EphrinA1 available for binding to EphA2. Any method known in the art to decrease expression of EphA2 or EphrinA1 can be used in the methods of the invention including, but not limited to, antisense and RNA interference technology. Thus, EphA2/EphrinA1 Modulators encompasses those agents that serve to increase or decrease EphrinA1 expression or availability for EphA2-binding, and those agents that serve to increase or decrease EphA2 expression or availability for binding to an endogenous EphA2 ligand (preferably, EphrinA1).

### 5.1.3.1 Antisense

The present invention encompasses EphA2 and EphrinA1 antisense nucleic acid molecules, i.e., molecules which are complementary to all or part of a sense nucleic acid encoding EphA2 or EphrinA1, molecules which are complementary to the coding strand of a double-stranded EphA2 or EphrinA1 cDNA molecule or molecules complementary to an EphA2 or EphrinA1 mRNA sequence. EphA2 and EphrinA1 antisense nucleic acid molecules can be produced by any method known to those skilled in the art, using the human EphA2 and EphrinA1 mRNA sequences disclosed, for example, in the GenBank database.

In a specific embodiment, an EphA2 antisense nucleic acid molecule may be produced using the human EphA2 mRNA sequence disclosed in GenBank Accession No. NM_{—}004431.2. Examples of EphA2 antisense nucleic acid molecules are also disclosed, e.g., in Cheng et al., 2002, Mol. Cancer Res. 1:2-11 and in Carles-Kinch et al., 2002, Cancer Res. 62:2840-2847, which are both incorporated by reference herein in their entireties. In a specific embodiment, an EphA2 antisense nucleic acid molecule can be complementary to any of the following regions (or a portion thereof) of human EphA2 as encoded by the coding strand or sense strand of human EphA2: the ligand binding domain, the transmembrane domain, the first fibronectin type III domain, the second fibronectin type III domain, the tyrosine kinase domain, or the SAM domain.

In a specific embodiment, an EphA2 antisense nucleic acid molecule is not 5′-CCAGCAGTACCACTTCCTTGCCCTGCGCCG-3′ (SEQ ID NO:40) and/or 5′-GCCGCGTCCCGTTCCTTCACCATGACGACC-3′ (SEQ ID NO:41). In another specific embodiment, an EphA2 antisense nucleic acid moleucle is not 5′-CCAGCAGTACCGCTTCCTTGCCCTGCGGCCG-3′ (SEQ ID NO:42) and/or 5′-GCCGCGTCCCGTTCCTTCACCATGACGACC-3′(SEQ ID NO:43). In certain embodiments, an EphA2/EphrinA1 Modulator of the invention is not an EphA2 antisense nucleic acid molecule.

In a preferred embodiment, an antisense EphA2/EphrinA1 Modulator of the invention is a human EphrinA1 antisense nucleic acid molecule. In a specific embodiment, a human EphrinA1 antisense nucleic acid molecule may be produced using the human EphrinA1 mRNA sequence disclosed in Genbank Accession No. BC032698. Examples of EphrinA1 antisense nucleic acid molecules are disclosed, e.g., in Potla et al., 2002, Cancer Lett. 175(2):187-95, which is incorporated by reference herein in its entirety. In a specific embodiment, an EphrinA1 antisense nucleic acid molecule of the invention is not the EphrinA1 antisense nucleic acid molecule(s) disclosed in Potla et al., 2002, Cancer Lett. 175(2):187-95. In certain embodiments, the EphA2/EphrinA1 Modulator of the invention is not an EphrinA1 antisense nucleic acid molecule.

An antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides (e.g., phosphorothioate-modified) designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, i.e., EphrinA1).

The antisense nucleic acid molecules of the invention are typically administered to a subject or generated in situ such that they hybridize with or bind to cellular mRNA and/or genomic DNA encoding a selected polypeptide of the invention to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An a-anomeric nucleic acid molecule forms specific double-stranded hybrids with complementary RNA in which, contrary to the usual β-units, the strands run parallel to each other (Gaultier et al., 1987, Nucleic Acids Res. 15:6625). The antisense nucleic acid molecule can also comprise a 2′-o-methylribonucleotide (Inoue et al., 1987, Nucleic Acids Res. 15:6131) or a chimeric RNA-DNA analogue (Inoue et al., 1987, FEBS Lett. 215:327).

### 5.1.3.2 RNA Interference

In certain embodiments, an RNA interference (RNAi) molecule is used to decrease EphA2 expression. In other embodiments, an RNAi molecule is used to decrease EphrinA1 expression. RNAi is defined as the ability of double-stranded RNA (dsRNA) to suppress the expression of a gene corresponding to its own sequence. RNAi is also called post-transcriptional gene silencing or PTGS. Since the only RNA molecules normally found in the cytoplasm of a cell are molecules of single-stranded mRNA, the cell has enzymes that recognize and cut dsRNA into fragments containing 21-25 base pairs (approximately two turns of a double helix). The antisense strand of the fragment separates enough from the sense strand so that it hybridizes with the complementary sense sequence on a molecule of endogenous cellular mRNA (e.g., human EphrinA1 mRNA sequence at Genbank Accession No. BC032698). This hybridization triggers cutting of the mRNA in the double-stranded region, thus destroying its ability to be translated into a polypeptide. Introducing dsRNA corresponding to a particular gene thus knocks out the cell's own expression of that gene in particular tissues and/or at a chosen time.

Double-stranded (ds) RNA can be used to interfere with gene expression in mammals (Wianny & Zernicka-Goetz, 2000, Nature Cell Biology 2: 70-75; incorporated herein by reference in its entirety). dsRNA is used as inhibitory RNA or RNAi of the function of EphrinA1 to produce a phenotype that is the same as that of a null mutant of EphrinA1 (Wianny & Zernicka-Goetz, 2000, Nature Cell Biology 2: 70-75). In certain embodiments, dsDNA encoding dsRNA (e.g., as hairpin structures) is used to express RNAi-mediating dsDNA in the cell.

In specific embodiments, EphA2 RNAi molecules may be generated using the EphA2 mRNA sequence as disclosed in the GenBank database (e.g., human EphA2 mRNA sequence at Genbank Accession No. NM_{—}004431.2). In other embodiments, EphrinA1 RNAi molecules may be generated using the EphrinA1 mRNA sequence as disclosed in the GenBank database (e.g., human EphrinA1 mRNA sequence at Genbank Accession No. BC032698).

### 5.1.3.3 Aptamers as EphA2/EphrinA1 Modulators

In specific embodiments, the invention provides aptamers of EphA2 and EphrinA1. As is known in the art, aptamers are macromolecules composed of nucleic acid (e.g., RNA, DNA) that bind tightly to a specific molecular target (e.g., EphA2 or EphrinA1 proteins, EphA2 or EphrinA1 polypeptides and/or EphA2 or EphrinA1 epitopes as described herein). A particular aptamer may be described by a linear nucleotide sequence and is typically about 15-60 nucleotides in length. The chain of nucleotides in an aptamer form intramolecular interactions that fold the molecule into a complex three-dimensional shape, and this three-dimensional shape allows the aptamer to bind tightly to the surface of its target molecule. Given the extraordinary diversity of molecular shapes that exist within the universe of all possible nucleotide sequences, aptamers may be obtained for a wide array of molecular targets, including proteins and small molecules. In addition to high specificity, aptamers have very high affinities for their targets (e.g., affinities in the picomolar to low nanomolar range for proteins). Aptamers are chemically stable and can be boiled or frozen without loss of activity. Because they are synthetic molecules, they are amenable to a variety of modifications, which can optimize their function for particular applications. For in vivo applications, aptamers can be modified to dramatically reduce their sensitivity to degradation by enzymes in the blood. In addition, modification of aptamers can also be used to alter their biodistribution or plasma residence time.

Selection of aptamers that can bind to EphA2 or EphrinA1 or a fragment thereof can be achieved through methods known in the art. For example, aptamers can be selected using the SELEX (Systematic Evolution of Ligands by Exponential Enrichment) method (Tuerk and Gold, 1990, Science 249:505-510, which is incorporated by reference herein in its entirety). In the SELEX method, a large library of nucleic acid molecules (e.g., 10¹⁵ different molecules) is produced and/or screened with the target molecule (e.g., EphA2 or EphrinA1 proteins, EphA2 or EphrinA1 polypeptides and/or EphA2 or EphrinA1 epitopes or fragments thereof as described herein). The target molecule is allowed to incubate with the library of nucleotide sequences for a period of time. Several methods can then be used to physically isolate the aptamer target molecules from the unbound molecules in the mixture and the unbound molecules can be discarded. The aptamers with the highest affinity for the target molecule can then be purified away from the target molecule and amplified enzymatically to produce a new library of molecules that is substantially enriched for aptamers that can bind the target molecule. The enriched library can then be used to initiate a new cycle of selection, partitioning, and amplification. After 5-15 cycles of this selection, partitioning and amplification process, the library is reduced to a small number of aptamers that bind tightly to the target molecule. Individual molecules in the mixture can then be isolated, their nucleotide sequences determined, and their properties with respect to binding affinity and specificity measured and compared. Isolated aptamers can then be further refined to eliminate any nucleotides that do not contribute to target binding and/or aptamer structure (i.e., aptamers truncated to their core binding domain). See, e.g., Jayasena, 1999, Clin. Chem. 45:1628-1650 for review of aptamer technology, the entire teachings of which are incorporated herein by reference).

In particular embodiments, the aptamers of the invention have the binding specificity and/or functional activity described herein for the antibodies of the invention. Thus, for example, in certain embodiments, the present invention is drawn to aptamers that have the same or similar binding specificity as described herein for the antibodies of the invention (e.g., binding specificity for EphA2 or EphrinA1 polypeptide, fragments of vertebrate EphA2 or EphrinA1 polypeptides, epitopic regions of vertebrate EphA2 or EphrinA1 polypeptides (e.g., epitopic regions of EphA2 or EphrinA1 that are bound by the antibodies of the invention). In particular embodiments, the aptamers of the invention can bind to an EphA2 or EphrinA1 polypeptide and inhibit one or more activities of the EphA2 or EphrinA1 polypeptide.

### 5.1.4 Vaccines as EphA2/EphrinA2 Modulators

In a specific embodiment, an EphA2/EphrinA1 Modulator is an EphA2 and/or an EphrinA1 vaccine. As used herein, the term “EphA2 vaccine” refers to any reagent that elicits or mediates an immune response against cells that overexpress EphA2. In certain embodiments, an EphA2 vaccine is an EphA2 antigenic peptide of the invention, an expression vehicle (e.g., a naked nucleic acid or a viral or bacterial vector or a cell) for an EphA2 antigenic peptide (e.g., which delivers the EphA2 antigenic peptide), or T cells or antigen presenting cells (e.g., dendritic cells or macrophages) that have been primed with the EphA2 antigenic peptide of the invention. As used herein, the terms “EphA2 antigenic peptide” and “EphA2 antigenic polypeptide” refer to an EphA2 polypeptide, or a fragment, analog, or derivative thereof comprising one or more B cell epitopes or T cell epitopes of EphA2. The EphA2 polypeptide may be from any species. In certain embodiments, an EphA2 polypeptide refers to the mature, processed form of EphA2. In other embodiments, an EphA2 polypeptide refers to an immature form of EphA2. For a description of EphA2 vaccines, see, e.g., U.S. Provisional Application Ser. No. 60/556,601, entitled “EphA2 Vaccines,” filed Mar. 26, 2004; U.S. Provisional Application Ser. No. 60/602,588, filed Aug. 18, 2004, entitled “EphA2 Vaccines” (Attorney Docket No. 10271-136-888); U.S. Provisional Application Ser. No. 60/615,548, filed Oct. 1, 2004, entitled “EphA2 Vaccines” (Attorney Docket No. 10271-143-888); U.S. Provisional Application Ser. No. 60/617,564, filed Oct. 7, 2004, entitled “EphA2 Vaccines” (Attorney Docket No. 10271-148-888), and International Application No. PCT/US04/34693, filed Oct. 15, 2004 entitled “EphA2 Vaccines” (Attorney Docket No. 10271-148-228) each of which is incorporated by reference herein in its entirety.

In a specific embodiment, an EphA-A2/EphrinA1. Modulator is an EphrinA1 Vaccine. As used herein, the term “EphrinA1 vaccine” refers to any reagent that elicits or mediates an immune response against EphrinA1 on EphrinA1-expressing cells. In certain embodiments, an EphrinA1 vaccine is an EphrinA1 antigenic peptide of the invention, an expression vehicle (e.g., a naked nucleic acid or a viral or bacterial vector or a cell) for an EphrinA1 antigenic peptide (e.g., which delivers the EphrinA1 antigenic peptide), or T cells or antigen presenting cells (e.g., dendritic cells or macrophages) that have been primed with the EphrinA1 antigenic peptide of the invention. As used herein, the terms “EphrinA1 antigenic peptide” and “EphrinA1 antigenic polypeptide” refer to an EphrinA1 polypeptide, or a fragment, analog, or derivative thereof comprising one or more B cell epitopes or T cell epitopes of EphrinA1. The EphrinA1 polypeptide may be from any species. In certain embodiments, an EphrinA1 polypeptide refers to the mature, processed form of EphrinA1. In other embodiments, an EphA2 polypeptide refers to an immature form of EphrinA1.

The present invention thus provides EphA2/EphrinA1 Modulators that are EphA2 vaccines. In a specific embodiment, an EphA2/Ephrin A1 Modulator is an EphA2- and/or EphrinA1 antigenic peptide expression vehicle expressing an EphA2 or an EphrinA1 antigenic peptide that can elicit or mediate a cellular immune response, a humoral response, or both, against cells that overexpress EphA2 or EphrinA1. Where the immune response is a cellular immune response, it can be a Tc, Th1 or a Th2 immune response. In a preferred embodiment, the immune response is a Th2 cellular immune response. In another preferred embodiment, an EphA2 or an EphrinA1 antigenic peptide expressed by an EphA2-/EphrinA1-antigenic peptide expression vehicle is an EphA2 or EphrinA1 antigenic peptide that is capable of eliciting an immune response against EphA2- and/or EphrinA1-expressing cells involved in an infection.

In a specific embodiment, the EphA2- and/or EphrinA1 antigenic expression vehicle is a microorganism expressing an EphA2 and/or an EphrinA1 antigenic peptide. In another specific embodiment, the EphA2- and/or EphrinA1 antigenic expression vehicle is an attenuated bacteria. Non-limiting examples of bacteria that can be utilized in accordance with the invention as an expression vehicle include Listeria monocytogenes, include but are not limited to Borrelia burgdorferi, Brucella melitensis, Escherichia coli, enteroinvasive Escherichia coli, Legionella pneumophila, Salmonella typhi, Salmonella typhimurium, Shigella spp., Streptococcus spp., Treponema pallidum, Yersinia enterocohtica, Listeria monocytogenes, Mycobacterium avium, Mycobacterium bovis, Mycobacterium tuberculosis, BCG, Mycoplasma hominis, Rickettsiae quintana, Cryptococcus neoformans, Histoplasma capsulatum, Pneumocystis carnii, Eimeria acervulina, Neospora caninum, Plasmodium falciparum, Sarcocystis suihominis, Toxoplasma gondii, Leishmania amazonensis, Leishmania major, Leishmania mexacana, Leptomonas karyophilus, Phytomonas spp., Trypanasoma cruzi, Encephahtozoon cuniculi, Nosema helminthorum, Unikaryon legeri. In a specific embodiment, an EphA2/EphrinA1 Modulator vaccine is Listeria-based vaccine expresses an EphA2 and/or an EphrinA1 antigenic peptide. In a further embodiment, the Listeria-based vaccine expressing an EphA2- and/or an EphrinA1 antigenic peptide is attenuated. In a specific embodiment, an EphA2/EphrinA1 Modulator vaccine is not Listeria-based or is not EphA2-based.

In another embodiment, the EphA2- and/or EphrinA1 antigenic peptide expression vehicle is a virus expressing an EphA2- and/or an EphrinA1 antigenic peptide. Non-limiting examples of viruses that can be utilized in accordance with the invention as an expression vehicle include RNA viruses (e.g., single stranded RNA viruses and double stranded RNA viruses), DNA viruses (e.g., double stranded DNA viruses), enveloped viruses, and non-enveloped viruses. Other non-limiting examples of viruses useful as EphA2- and/or EphrinA1 antigenic peptide expression vehicles include retroviruses (including but not limited to lentiviruses), adenoviruses, adeno-associated viruses, or herpes simplex viruses. Preferred viruses for administration to human subjects are attenuated viruses. A virus can be attenuated, for example, by exposing the virus to mutagens, such as ultraviolet irradiation or chemical mutagens, by multiple passages and/or passage in non-permissive hosts, and/or genetically altering the virus to reduce the virulence and pathogenicity of the virus.

Microorganisms can be produced by a number of techniques well known in the art. For example, antibiotic-sensitive strains of microorganisms can be selected, microorganisms can be mutated, and mutants that lack virulence factors can be selected, and new strains of microorganisms with altered cell wall lipopolysaccharides can be constructed. In certain embodiments, the microorganisms can be attenuated by the deletion or disruption of DNA sequences which encode for virulence factors which insure survival of the microorganisms in the host cell, especially macrophages and neutrophils, by, for example, homologous recombination techniques and chemical or transposon mutagenesis. Many, but not all, of these studied virulence factors are associated with survival in macrophages such that these factors are specifically expressed within macrophages due to stress, for example, acidification, or are used to induced specific host cell responses, for example, macropinocytosis, Fields et al., 1986, Proc. Natl. Acad. Sci. USA 83:5189-5193. Bacterial virulence factors include, for example: cytolysin; defensin resistance loci; DNA K; fimbriae; GroEL; inv loci; lipoprotein.; LPS; lysosomal fusion inhibition; macrophage survival loci; oxidative stress response loci; pho loci (e.g., PhoP and PhoQ); pho activated genes (pag; e.g., pagB and pagC); phoP and phoQ regulated genes (prg); porins; serum resistance peptide; virulence plasmids (such as spvB, traT and ty2).

Yet another method for the attenuation of the microorganisms is to modify substituents of the microorganism which are responsible for the toxicity of that microorganism. For example, lipopolysaccharide (LPS) or endotoxin is primarily responsible for the pathological effects of bacterial sepsis. The component of LPS which results in this response is lipid A (LA). Elimination or mitigation of the toxic effects of LA results in an attenuated bacteria since 1) the risk of septic shock in the patient would be reduced and 2) higher levels of the bacterial EphA2 or EphrinA1 antigenic peptide expression vehicle could be tolerated.

Rhodobacter (Rhodopseudomonas) sphaeroides and Rhodobacter capsulatus each possess a monophosphoryl lipid A (MLA) which does not elicit a septic shock response in experimental animals and, further, is an endotoxin antagonist. Loppnow et al., 1990, Infect. Immun. 58:3743-3750; Takayma et al., 1989, Infect. Immun. 57:1336-1338. Gram negative bacteria other than Rhodobacter can be genetically altered to produce MLA, thereby reducing its potential of inducing septic shock.

Yet another example for altering the LPS of bacteria involves the introduction of mutations in the LPS biosynthetic pathway. Several enzymatic steps in LPS biosynthesis and the genetic loci controlling them in a number of bacteria have been identified, and several mutant bacterial strains have been isolated with genetic and enzymatic lesions in the LPS pathway. In certain embodiments, the LPS pathway mutant is a firA mutant. firA is the gene that encodes the enzyme UDP-3-O(R-30 hydroxymyristoyl)-glycocyamine N-acyltransferase, which regulates the third step in endotoxin biosynthesis (Kelley et al., 1993, J. Biol. Chem. 268:19866-19874).

As a method of insuring the attenuated phenotype and to avoid reversion to the non-attenuated phenotype, the bacteria may be engineered such that it is attenuated in more than one manner, e.g., a mutation in the pathway for lipid A production and one or more mutations to auxotrophy for one or more nutrients or metabolites, such as uracil biosynthesis, purine biosynthesis, and arginine biosynthesis.

The EphA2 or EphrinA1 antigenic peptides are preferably expressed in a microorganism, such as bacteria, using a heterologous gene expression cassette. A heterologous gene expression cassette is typically comprised of the following ordered elements: (1) prokaryotic promoter; (2) Shine-Dalgarno sequence; (3) secretion signal (signal peptide); and, (4) heterologous gene. Optionally, the heterologous gene expression cassette may also contain a transcription termination sequence, in constructs for stable integration within the bacterial chromosome. While not required, inclusion of a transcription termination sequence as the final ordered element in a heterologous gene expression cassette may prevent polar effects on the regulation of expression of adjacent genes, due to read-through transcription.

The expression vectors introduced into the microorganism EphA2 or EphrinA1 vaccines are preferably designed such that microorganism-produced EphA2 or EphrinA1 peptides and, optionally, prodrug converting enzymes, are secreted by microorganism. A number of bacterial secretion signals are well known in the art and may be used in the compositions and methods of the present invention. In certain embodiments of the present invention, the bacterial EphA2 antigenic peptide expression vehicles are engineered to be more susceptible to an antibiotic and/or to undergo cell death upon administration of a compound. In other embodiments of the present invention, the bacterial EphA2 or EphrinA1 antigenic peptide expression vehicles are engineered to deliver suicide genes to the target EphA2- or EphrinA1-expressing cells. These suicide genes include pro-drug converting enzymes, such as Herpes simplex thymidine kinase (TK) and bacterial cytosine deaminase (CD). TK phosphorylates the non-toxic substrates acyclovir and ganciclovir, rendering them toxic via their incorporation into genomic DNA. CD converts the non-toxic 5-fluorocytosine (5-FC) into 5-fluorouracil (5-FU), which is toxic via its incorporation into RNA. Additional examples of pro-drug converting enzymes encompassed by the present invention include cytochrome p450 NADPH oxidoreductase which acts upon mitomycin C and porfiromycin (Murray et al., 1994, J. Pharmacol. Exp. Therapeut. 270:645-649). Other exemplary pro-drug converting enzymes that may be used include: carboxypeptidase; beta-glucuronidase; penicillin-V-amidase; penicillin-G-amidase; beta-lactamase; beta.-glucosidase; nitroreductase; and carboxypeptidase A.

Exemplary secretion signals that can be used with gram-positive microorganisms include SecA (Sadaie et al., 1991, Gene 98:101-105), SecY (Suh et al., 1990, Mol. Microbiol. 4:305-314), SecE (Jeong et al., 1993, Mol. Microbiol. 10:133-142), FtsY and FfH (PCT/NL 96/00278), and PrsA (International Publication No. WO 94/19471). Exemplary secretion signals that may be used with gram-negative microorganisms include those of soluble cytoplasmic proteins such as SecB and heat shock proteins; that of the peripheral membrane-associated protein SecA- and those of the integral membrane proteins SecY, SecE, SecD and SecF.

The promoters driving the expression of the EphA2 or EphrinA1 antigenic peptides and, optionally, pro-drug converting enzymes, may be either constitutive, in which the peptides or enzymes are continually expressed, inducible, in which the peptides or enzymes are expressed only upon the presence of an inducer molecule(s), or cell-type specific control, in which the peptides or enzymes are expressed only in certain cell types. For example, a suitable inducible promoter can be a promoter responsible for the bacterial “SOS” response (Friedberg et al., In: DNA Repair and Mutagenesis, pp. 407-455, Am. Soc. Microbiol. Press, 1995). Such a promoter is inducible by numerous agents including chemotherapeutic alkylating agents such as mitomycin (Oda et al., 1985, Mutation Research 147:219-229; Nakamura et al., 1987, Mutation Res. 192:239-246; Shimda et al., 1994, Carcinogenesis 15:2523-2529) which is approved for use in humans. Promoter elements which belong to this group include umuC, sulA and others (Shinagawa et al., 1983, Gene 23:167-174; Schnarr et al., 1991, Biochemie 73:423-431). The sulA promoter includes the ATG of the sulA gene and the following 27 nucleotides as well as 70 nucleotides upstream of the ATG (Cole, 1983, Mol. Gen. Genet. 189:400-404). Therefore, it is useful both in expressing foreign genes and in creating gene fusions for sequences lacking initiating codons.

In certain embodiments, an EphA2/EphrinA1 Modulator vaccine does not comprise a microorganism.

### 5.2 Prophylactic/Therapeutic Methods

The present invention provides methods for treating, managing, preventing and/or ameliorating an infection (in particular, an intracellular infection), said methods comprising administering to a subject in need thereof one or more EphA2/EphrinA1 Modulators of the invention. The present invention also provides methods for treating, managing, preventing, and/or ameliorating a pathogen infection (in particular, an intracellular infection) said methods comprising administering to a subject in need thereof one or more EphA2/EphrinA1 Modulators and one or more other therapies (see Section 5.2.6, infra, for examples of such therapies). Preferably, such other therapies are useful in the treatment, prevention, management and/or amelioration of a pathogen infection and are used in combination with the EphA2/EphrinA1 Modulators of the invention. Non-limiting examples of pathogens include viruses, bacteria, protozoa and fungi. In a preferred embodiment, the pathogen is an intracellular pathogen. In a preferred embodiment, the cells infected with the pathogens have increased EphA2 expression.

The dosage amounts and frequences of administration provided herein are encompassed by the terms “effective amount”, “therapeutically effective” and “prophylactically” effective. The dosage and frequency further will typically vary according to factors specific for each patient depending on the specific therapeutic or prophylactic agents administered, the severity and type of infection, the route of administration, as well as age, body weight, response, and the past medical history of the patient. Suitable regimens can be selected by one skilled in the art by considering such factors and by following, for example, dosages reported in the literature and recommended in the Physicians' Desk Reference (59^{th} ed., 2005). See Section 5.4 for specific dosage amounts and frequencies of administration of the prophylactic and therapeutic agents provided by the invention.

### 5.2.1 Patient Population

The present invention provides methods for treating, managing, preventing and/or ameliorating an infection (in particular, an intracellular infection), or a symptom thereof, the methods comprising administering one or more EphA2/EphrinA1 Modulators of the invention alone or in combination with therapies other than an EphA2/EphrinA1 Modulator. The subject is preferably a mammal such as non-primate (e.g., cows, pigs, horses, cats, dogs, rats, etc.) and a primate (e.g., monkey, such as a cynomolgous monkey or human). In a preferred embodiment, the subject is a human.

The methods of the invention comprise the administration of one or more EphA2/EphrinA1 Modulators of the invention to patients suffering from or expected to suffer from (e.g., patients with a genetic predisposition for or patients that have previously suffered from) an infection. Such patients may have been previously treated or are currently being treated for the infection, e.g., with a non-EphA2/EphrinA1 Modulator therapy. In a further embodiment, the methods of the invention comprise the administration of one or more EphA2/EphrinA1 Modulators of the invention to patients that are immunocompromised or immunosuppressed. In a certain embodiment, an EphA2/EphrinA1 Modulator is not administered to patients that are immunocompromised or immunosuppressed. In accordance with the invention, an EphA2/EphrinA1 Modulator may be used as any line of therapy, including, but not limited to, a first, second, third and fourth line of therapy. Further, in accordance with the invention, an EphA2/EphrinA1 Modulator can be used before any adverse effects or intolerance of the non-EphA2/EphrinA1 Modulator therapies occurs. The invention encompasses methods for administering one or more EphA2/EphrinA1 Modulators of the invention to prevent the onset or recurrence of an infection.

In one embodiment, the invention also provides methods of treatment, management, prevention and/or amelioration of an infection as alternatives to current therapies. In a specific embodiment, the current therapy has proven or may prove too toxic (i.e., results in unacceptable or unbearable side effects) for the patient. In another embodiment, an EphA2/EphrinA1 Modulator decreases the side effects as compared to the current therapy. In another embodiment, the patient has proven refractory to a current therapy. In such embodiments, the invention provides for the administration of one or more EphA2/EphrinA1 Modulators of the invention without any other anti-infection therapies. In certain embodiments, one or more EphA2/EphrinA1 Modulators of the invention can be administered to a patient in need thereof instead of another therapy to treat an infection. In one embodiment, the invention provides methods of treating, managing, preventing and/or ameliorating of an active infection. In another embodiment, the invention provides methods of treating, managing, preventing and/or ameliorating a latent infection. In another embodiment, the invention provides methods of preventing the recurrence of an acute infection. In yet another embodiment, the invention provides methods of treating, managing, preventing and/or ameliorating a chronic infection.

The present invention also encompasses methods for administering one or more EphA2/EphrinA1 Modulators of the invention to treat or ameliorate symptoms of infections in patients that are or have become refractory to non-EphA2/EphrinA1 Modulator therapies. The determination of whether the infection is refractory can be made either in vivo or in vitro by any method known in the art for assaying the effectiveness of a therapy on affected cells in the infection, particularly epithelial cells, or in patients that are or have become refractory to non-EphA2/EphrinA1 Modulator therapies.

### 5.2.2 Viral Infections

One or more EphA2/EphrinA1 Modulators of the invention and compositions comprising said EphA2/EphrinA1 Modulators can be administered to a subject to prevent, treat, manage, and/or ameliorate a viral infection or one or more symptoms thereof. In a preferred embodiment, the viral infection to be treated, managed, prevented and/or ameliorated in accordance with the methods of the present invention are intracellular viral infections. One or more EphA2/EphrinA1 Modulators of the invention and compositions comprising said antibodies may be administered in combination with one or more other therapies (e.g., one or more prophylactic or therapeutic agents) other than EphA2/EphrinA1 Modulators of the invention to a subject predisposed to or with a viral infection useful for the prevention, treatment, management, or amelioration of a viral infection. Non-limiting examples of such therapies include the agents described in Section 5.2.6, infra, and in particular, the immunomodulatory agents described in Section 5.2.6.1, the anti-inflammatory agents described in Section 5.2.6.2, the anti-viral agents described in Section 5.2.6.3, the anti-bacterial agents described in Section 5.2.6.4, the anti-fungal agents described in Section 5.2.6.5, and the anti-protozoan agents described in Section 5.2.6.6.

In a specific embodiment, the invention provides methods of preventing, treating, managing, and/or ameliorating a viral infection or one or more symptoms thereof, said method comprising administering to a subject in need thereof an effective amount of one or more EphA2/EphrinA1 Modulators of the invention. In another embodiment, the invention provides a method of preventing, treating, managing, and/or ameliorating a viral infection or one or more symptoms thereof, said method comprising administering to a subject in need thereof an effective amount of one or more EphA2/EphrinA1 Modulators of the invention and an effective amount of one or more therapies (e.g., one or more prophylactic or therapeutic agents) other than EphA2/EphrinA1 Modulators of the invention.

In certain embodiments, an effective amount of one or more EphA2/EphrinA1 Modulators of the invention is administered in combination with an effective amount of one or more therapies (e.g., one or more prophylactic or therapeutic agents) currently being used, have been used, or are known to be useful in the prevention, management, treatment, and/or amelioration of a viral infection or one or more symptoms thereof to a subject in need thereof. Therapies for a viral infection, include, but are not limited to, anti-viral agents such as acyclovir, amantadine, oseltamivir, ribaviran, palivizumab, and anamivir. In certain embodiments, an effective amount of one or more EphA2/EphrinA1 Modulators of the invention is administered in combination with one or more supportive measures to a subject in need thereof to prevent, manage, treat, and/or ameliorate a viral infection or one or more symptoms thereof. Non-limiting examples of supportive measures include humidification of the air by an ultrasonic nebulizer, aerolized racemic epinephrine, oral dexamethasone, intravenous fluids, intubation, fever reducers (e.g., ibuprofen, acetometaphin), and antibiotic and/or anti-fungal therapy (i.e., to prevent or treat secondary bacterial infections).

Any type of viral infection or condition resulting from or associated with a viral infection can be prevented, treated, managed, and/or ameliorated in accordance with the methods of the invention, said methods comprising administering an effective amount of one or more EphA2/EphrinA1 Modulators of the invention alone or in combination with an effective amount of another therapy (e.g., a prophylactic or therapeutic agent other than EphA2/EphrinA1 Modulators of the invention). Examples of viruses which cause viral infections include, but are not limited to, retroviruses (e.g., human T-cell lymphotrophic virus (HTLV) types I and II and human immunodeficiency virus (HIV, e.g., HIV-1 and HIV-2)), herpes viruses (e.g., herpes simplex virus (HSV) types I and II, Epstein-Barr virus, HHV6-HHV8, and cytomegalovirus), arenavirues (e.g., lassa fever virus), paramyxoviruses (e.g., morbillivirus virus, human respiratory syncytial virus, mumps, hMPV, and pneumovirus), adenoviruses, bunyaviruses (e.g., hantavirus), cornaviruses, filoviruses (e.g., Ebola virus), flaviviruses (e.g., hepatitis C virus (HCV), yellow fever virus, and Japanese encephalitis virus), hepadnaviruses (e.g., hepatitis B viruses (HBV)), orthomyoviruses (e.g., influenza viruses A, B and C and PIV), papovaviruses (e.g., papillomavirues), picornaviruses (e.g., rhinoviruses, enteroviruses and hepatitis A viruses), poxviruses, reoviruses (e.g., rotavirues), togaviruses (e.g., rubella virus), and rhabdoviruses (e.g., rabies virus). Biological responses to a viral infection include, but not limited to, elevated levels of IgE antibodies, increased proliferation and/or infiltration of T cells, increased proliferation and/or infiltration of B cells, epithelial hyperplasia, and mucin production. In a specific embodiment, the invention also provides methods of preventing, treating, managing, and/or ameliorating viral infections that are associated with or cause the common cold, viral pharyngitis, viral laryngitis, viral croup, viral bronchitis, influenza, parainfluenza viral diseases (“PIV”) diseases (e.g., croup, bronchiolitis, bronchitis, pneumonia), respiratory syncytial virus (“RSV”) diseases, metapneumavirus diseases, and adenovirus diseases (e.g., febrile respiratory disease, croup, bronchitis, pneumonia), said method comprising administering an effective amount of one or more EphA2/EphrinA1 Modulators of the invention alone or in combination with an effective amount of another therapy.

In a specific embodiment, influenza virus infections, PIV infections, hMPV infections, adenovirus infections, and/or RSV infections, or one or more of symptoms thereof are prevented, treated, managed, and/and/or ameliorated in accordance with the methods of the invention. In a specific embodiment, the invention provides methods for preventing, treating, managing, and/or ameliorating a RSV infection or one or more symptoms thereof, said methods comprising administering to a subject in need thereof an effective amount of one or more EphA2/EphrinA1 Modulators of the invention alone or in combination with one or more anti-viral agents such as, but not limited to, amantadine, rimantadine, oseltamivir, znamivir, ribaviran, RSV-IVIG (i.e., intravenous immune globulin infusion) (RESPIGAM™), and palivizumab and those antibodies disclosed in U.S. patent application Ser. Nos. 09/996,288 and 09/996,265, both entitled “Methods of Administering/Dosing Anti-RSV Antibodies For Prophylaxis and Treatment,” filed Nov. 28, 2001. In certain embodiments, the viral infection treated, managed, prevented or ameliorated in accordance with the methods of the invention is not a RSV infection.

In a specific embodiment, the invention provides methods for preventing, treating, managing, and/or ameliorating a PIV infection or one or more symptoms thereof, said methods comprising administering to a subject in need thereof an effective amount of one or more EphA2/EphrinA1 Modulators of the invention alone or in combination with an effective amount of one or more anti-viral agents such as, but not limited to, amantadine, rimantadine, oseltamivir, znamivir, ribaviran, and palivizumab. In another specific embodiment, the invention provides methods for preventing, treating, managing, and/or ameliorating a hMPV infection or one or more symptoms thereof, said methods comprising of administering an effective amount of one or more antibodies of the invention alone or in combination with an effective amount of one or more anti-viral agents, such as, but not limited to, amantadine, rimantadine, oseltamivir, znamivir, ribaviran, and palivizumab to a subject in need thereof. In another specific embodiment, the invention provides methods for preventing, treating, managing, and/or ameliorating influenza, said methods comprising administering an effective amount of one or more EphA2/EphrinA1 Modulators of the invention alone or in combination with an effective amount of an anti-viral agent such as, but not limited to zanamivir (RELENZA®), oseltamivir (TAMIFLU®), rimantadine, and amantadine (SYMADINE®; SYMMETREL®) to a subject in need thereof.

The invention provides methods for preventing the development of asthma in a subject who suffers from or had suffered from a viral respiratory infection, said methods comprising administering an effective amount of one or more EphA2/EphrinA1 Modulators of the invention alone or in combination with an effective amount of another therapy. In a specific embodiment, the subject is an elderly person (i.e., a person who is 65 years or older), an infant born prematurely, an infant, or a child. In another specific embodiment, the subject suffered from or suffers from RSV infection. In a specific embodiment, the infection is not a viral respiratory infection. In a further embodiment, the infection is not an RSV infection.

In a specific embodiment, the invention provides methods for preventing, treating, managing, and/or ameliorating one or more secondary responses to a primary viral infection, said methods comprising of administering an effective amount of one or more EphA2/EphrinA1 Modulators of the invention alone or in combination with an effective amount of other therapies (e.g., other prophylactic or therapeutic agents). Examples of secondary responses to a primary viral infection, particularly a primary viral respiratory infection, include, but are not limited to, asthma-like responsiveness to mucosal stimula, elevated total respiratory resistance, increased susceptibility to secondary viral, bacterial, fungal and protozoan infections, and development of such conditions such as, but not limited to, pneumonia, croup, and febrile bronchitis. In a specific embodiment, the invention provides methods for preventing, treating, managing, and/or ameliorating an acute viral infection. In a further embodiment, the invention provides methods for preventing, treating, managing, and/or ameliorating a latent viral infection. In yet further embodiments, the invention provides methods for preventing, treating, managing, and/or ameliorating an HIV infection or an HBV infection.

In a specific embodiment, the invention provides methods of preventing, treating, managing, and/or ameliorating a viral infection or one or more symptoms thereof, said methods comprising administering to a subject in need thereof an effective amount of one or more EphA2/EphrinA1 Modulators of the invention in combination with an effective amount of VITAXIN™ (MedImmune, Inc., International Publication No. WO 00/78815, International Publication No. WO 02/070007 A1, dated Sep. 12, 2002, entitled “Methods of Preventing or Treating Inflammatory or Autoimmune Disorders by Administering Integrin AlphaV Beta3 Antagonists,” International Publication No. WO 03/075957 A1, dated Sep. 18, 2003, entitled “The Prevention or Treatment of Cancer Using Integrin AlphaVBeta3 Antagonists in Combination With Other Agents,” U.S. Patent Pub. No. US 2002/0168360 A1, dated Nov. 14, 2002, entitled “Methods of Preventing or Treating Inflammatory or Autoimmune Disorders by Administering Integrin αᵥβ3 Antagonists in Combination With Other Prophylactic or Therapeutic Agents,” and International Publication No. WO 03/075741 A2, dated Sep. 18, 2003, entitled, “Methods of Preventing or Treating Disorders by Administering an Integrin αvβ3 Antagonist in Combination With an HMG-CoA Reductase Inhibitor or a Bisphosphonate,” each of which is incorporated herewith by reference in its entirety). In another specific embodiment, the invention provides methods for preventing, treating, managing, and/or ameliorating a viral infection or one or more symptoms thereof, said methods comprising administering to a subject in need thereof an effective amount of one or more EphA2/EphrinA1 Modulators of the invention in combination with an effective amount of siplizumab (MedImmune, Inc., International Pub. No. WO 02/069904, which is incorporated herein by reference in its entirety). In another embodiment, the invention provides methods of preventing, treating, managing and/or ameliorating a viral infection or one or more symptoms thereof, said methods comprising administering to a subject in need thereof an effective amount of one or more EphA2/EphrinA1 Modulators in combination with an effective amount of one or more anti-IL-9 antibodies such as those disclosed in U.S. Pat. Pub. No. 20050002934 (Jan. 6, 2005), which is incorporated herein by reference in its entirety. In yet another embodiment, the invention provides methods for preventing, treating, managing, and/or ameliorating a viral infection or one or more symptoms thereof, said methods comprising administering to a subject in need thereof an effective amount of one or more EphA2/EphrinA1 Modulators of the invention in combination with an effective amount of two or more of the following: VITAXIN™, an anti-IL-9 antibody and/or siplizumab.

In one embodiment, an effective amount of one or more EphA2/EphrinA1 Modulators of the invention is administered in combination with an effective amount of one or more anti-IgE antibodies to a subject to prevent, treat, manage, and/or ameliorate a viral infection or one or more symptoms thereof. In a specific embodiment, an effective amount of one or more antibodies of the invention is administered in combination with an effective amount of anti-IgE antibody TNX901 to a subject to prevent, treat, manage, and/or ameliorate a viral infection or one or more symptoms thereof. In a specific embodiment, an effective amount of one or more antibodies of the invention is administered in combination with an effective amount of anti-IgE antibody rhuMAb-E25 omalizumab to a subject to prevent, treat, manage, and/or ameliorate a viral infection or one or more symptoms thereof. In another embodiment, an effective amount of one or more EphA2/EphrinA1 Modulators of the invention is administered in combination with an effective amount of anti-IgE antibody HMK-12 to a subject to prevent, treat, manage, and/or ameliorate a viral infection or one or more symptoms thereof. In a specific embodiment, an effective amount of one or more EphA2/EphrinA1 Modulators of the invention is administered in combination with an effective amount of anti-IgE antibody 6HD5 to a subject to prevent, treat, manage, and/or ameliorate a viral infection or one or more symptoms thereof. In another embodiment, an effective amount of one or more antibodies of the invention is administered in combination with an effective amount of anti-IgE antibody MAb Hu-901 to a subject to prevent, treat, manage, and/or ameliorate a viral infection or one or more symptoms thereof.

The invention encompasses methods for preventing the development of viral infections, in a patient expected to suffer from a viral infection or at increased risk of such an infection, e.g., patients with suppressed immune systems (e.g, organ-transplant recipients, AIDS patients, patients undergoing chemotherapy, the elderly, infants born prematurely, infants, children, patients with carcinoma of the esophagus with obstruction, patients with tracheobronchial fistula, patients with neurological diseases (e.g., caused by stroke, amyotrophic lateral sclerosis, multiple sclerosis, and myopathies), and patients already suffering from a viral infection). The patients may or may not have been previously treated for a viral infection.

The EphA2/EphrinA1 Modulators of the invention, compositions, or combination therapies of the invention may be used as any line of therapy, including but not limited to, the first, second, third, fourth, or fifth line of therapy, to prevent, manage, treat, and/or ameliorate a viral infection or one or more symptom thereof. The invention also includes methods of preventing, treating, managing, and/or ameliorating a viral infection, or one or more symptoms thereof in a patient undergoing therapies for other diseases or disorders associated increased in EphA2 expression. The invention encompasses methods of preventing, managing, treating, and/or ameliorating a viral infection, or one or more symptoms thereof in a patient before any adverse effects or intolerance to therapies other than EphA2/EphrinA1 Modulators of the invention develops. The invention also encompasses methods of preventing, treating, managing, and/or ameliorating a viral infection or a symptom thereof in refractory patients. In certain embodiments, a patient with a viral infection, is refractory to a therapy when the infection has not significantly been eradicated and/or the symptoms have not been significantly alleviated. The determination of whether a patient is refractory can be made either in vivo or in vitro by any method known in the art for assaying the effectiveness of a treatment of infections, using art-accepted meanings of “refractory” in such a context. In various embodiments, a patient with a viral infection is refractory when viral replication has not decreased or has increased. The invention also encompasses methods of preventing the onset or reoccurrence of viral infections in patients at risk of developing such infections. The invention also encompasses methods of preventing, managing, treating, and/or ameliorating a viral infection or a symptom thereof in patients who are susceptible to adverse reactions to conventional therapies. The invention further encompasses methods for preventing, treating, managing, and/or ameliorating a viral infection for which no anti-viral therapy is available.

The invention encompasses methods for preventing, treating, managing, and/or ameliorating a viral infection or a symptom thereof in a patient who has proven refractory to therapies other than EphA2/EphrinA 1 Modulators of the invention but are no longer on these. therapies. In certain embodiments, the patients being managed or treated in accordance with the methods of this invention are patients already being treated with antibiotics, anti-virals, anti-fungals, or other biological therapy/immunotherapy. Among these patients are refractory patients, patients who are too young for conventional therapies, and patients with reoccurring viral infections despite management or treatment with existing therapies.

The present invention encompasses methods for preventing, treating, managing, and/or ameliorating a viral infection, or one or more symptoms thereof as an alternative to other conventional therapies. In specific embodiments, the patient being managed or treated in accordance with the methods of the invention is refractory to other therapies or is susceptible to adverse reactions from such therapies. The patient may be a person with a suppressed immune system (e.g., post-operative patients, chemotherapy patients, and patients with immunodeficiency disease), a person with impaired renal or liver function, the elderly, children, infants, infants born prematurely, persons with neuropsychiatric disorders or those who take psychotropic drugs, persons with histories of seizures, or persons on medication that would negatively interact with conventional agents used to prevent, manage, treat, and/or ameliorate a viral infection or one or more symptoms thereof.

Viral infection therapies and their dosages, routes of administration and recommended usage are known in the art and have been described in such literature as the Physicians' Desk Reference (59th ed., 2005).

### 5.2.3 Bacterial Infections

The invention provides a method of preventing, treating, managing, and/or ameliorating a bacterial infection, in particular an intracellular bacterial infection, or one or more symptoms thereof, said method comprising administering to a subject in need thereof an effective amount of one or more EphA2/EphrinA1 Modulators of the invention. Preferably, cells infected with the intracellular bacteria have increased EphA2 expression. In another embodiment, the invention provides a method of preventing, treating, managing, and/or ameliorating a bacterial infection or one or more symptoms thereof, said method comprising administering to a subject in need thereof an effective amount of a one or more EphA2/EphrinA1 Modulators of the invention and an effective amount of one or more therapies (e.g., one or more prophylactic or therapeutic agents), other than EphA2/EphrinA1 Modulators of the invention. In a preferred embodiment, the bacterial infections to be treated, managed, prevented and/or ameliorated in accordance with the methods of the present invention are intracellular bacterial infections.

Any type of intracellular bacterial infection or condition resulting from or associated with a bacterial infection (e.g., a respiratory infection) can be prevented, treated, managed, and/or ameliorated in accordance with the methods of invention. Examples of intracellular bacteria which cause infections include, but not limited to, Mycobacterium tuberculosis, Mycobacterium leprae, Salmonella enterica serovar Typhi, Brucella sp, Legionella sp, Listeria monocytogenes, Francisella tularensis, Rickettsia rickettsii; Rickettsia prowazekii; Rickettsia typhi; Rickettsia tsutsugamushi; Chlamydia trachomatis; Chlamydia psittaci; and Chlamydia pneumoniae. In certain embodiments, an intracellular bacterial infection prevented, treated, managed and/or ameliorated in accordance with the methods of the invention is not a respiratory bacterial infection. In other embodiments, an intracellular bacterial infection prevented, treated, managed and/or ameliorated in accordance with the methods of the invention is not a Salmonella species infection. In yet other embodiments, an intracellular bacterial infection prevented, treated, managed and/or ameliorated in accordance with the methods of the invention is not Salmonella dublin infection.

In a specific embodiment, the invention provides methods for preventing, treating, managing, and/or ameliorating an intracellular bacterial infection or one or more symptoms thereof, said method comprising administering to a subject in need thereof an effective amount of one or more EphA2/EphrinA1 Modulators of the invention. In another embodiment, the invention provides a method of preventing, treating, managing, and/or ameliorating an intracellular bacterial infection or one or more symptoms thereof, said method comprising administering to a subject in need thereof an effective amount of a one or more EphA2/EphrinA1 Modulators of the invention and an effective amount of one or more therapies (e.g., prophylactic or therapeutic agents), other than EphA2/EphrinA1 Modulators of the invention.

In certain embodiments, the invention provides methods to prevent, treat, manage, and/or ameliorate a bacterial infection or one or more of the symptoms, said methods comprising administering to a subject in need thereof one or more EphA2/EphrinA1 Modulators of the invention in combination with and effective amount of one or more therapies (e.g., one or more prophylactic or therapeutic agents), other than EphA2/EphrinA1 Modulators of the invention, used to prevent, treat, manage, and/or ameliorate bacterial infections. Therapies for bacterial infections, particularly, bacterial infections include, but are not limited to, anti-bacterial agents (e.g., aminoglycosides (e.g., gentamicin, tobramycin, amikacin, netilicin) aztreonam, celphalosporins (e.g., cefaclor, cefadroxil, cephalexin, cephazolin), clindamycin, erythromycin, penicillin (e.g., penicillin V, crystalline penicillin G, procaine penicillin G), spectinomycin, and tetracycline (e.g., chlortetracycline, doxycycline, oxytetracycine)) and supportive therapy, such as supplemental and mechanical ventilation. In certain embodiments, one or more EphA2/EphrinA1 Modulators of the invention are administered in combination with one or more supportive measures to a subject in need thereof to prevent, manage, treat, and/or ameliorate a bacterial infection or one or more symptoms thereof. Non-limiting examples of supportive measures include humidification of air by ultrasonic nebulizer, aerolized racemic epinephrine, oral dexamethasone, intravenous fluids, intubation, fever reducers (e.g., ibuprofen, acetometaphin), and more preferably, antibiotic or anti-viral therapy (i.e., to prevent or treat secondary infections).

The invention provides methods for preventing, managing, treating, and/or ameliorating a biological response to a bacterial infection, such as, but not limited to, elevated levels of IgE antibodies, mast cell proliferation, degranulation, and/or infiltration, increased proliferation and/or infiltration of B cells, and increased proliferation and/or infiltration of T cells, said methods comprising administering to a subject in need thereof an effective amount of one or more EphA2/EphrinA1 Modulators of the invention alone or in combination with an effective amount one or more therapies (e.g. a prophylactic or therapeutic agent) other than EphA2/EphrinA1 Modulators of the invention. The invention also provides methods of preventing, treating, managing, and/or ameliorating respiratory conditions caused by or associated with bacterial infections, such as, but not limited to, pneumonia, recurrent aspiration pneumonia, legionellosis, whooping cough, meningitis, or tuberculosis, said methods comprising administering to a subject in need thereof an effective amount of one or more EphA2/EphrinA1 Modulators of the invention alone or in combination with an effective amount of another therapy.

In a specific embodiment, the methods of the invention are utilized to prevent, treat, manage, and/or ameliorate a bacterial infection caused by Mycobacteria or one or more symptoms thereof, said method comprising administering to a subject in need thereof of an effective amount of one or more EphA2/EphrinA1 Modulators of the invention alone or in combination with an effective amount of one or more other therapies (e.g., one or more prophylactic or therapeutic agents) other than EphA2/EphrinA1 Modulators of the invention.

In a specific embodiment, the invention provides methods for preventing, treating, managing, and/or ameliorating one or more secondary conditions or responses to a primary bacterial infection, preferably a primacy bacterial infection, said method comprising administering to a subject in need thereof an effective amount of one or more EphA2/EphrinA1 Modulators of the invention alone or in combination with an effective amount of other therapies (e.g., other prophylactic or therapeutic agents). Examples of secondary conditions or responses to a primary bacterial infection, particularly a bacterial infection, include, but are not limited to, asthma-like responsiveness to mucosal stimula, elevated total resistance, increased susceptibility to secondary viral, bacterial, fungal and protozoan infections, and development of such conditions such as, but not limited to, pneumonia, croup, and febrile bronchitits.

In a specific embodiment, the methods of the invention are used to prevent, manage, treat, and/or ameliorate a bacterial infection, or one or more symptoms thereof, said methods comprising administering to a subject in need thereof an effective amount of one or more EphA2/EphrinA1 Modulators of the invention in combination with an effective amount of VITAXIN™ (MedImmune, Inc., International Publication No. WO 00/78815, International Publication No. WO 02/070007 A1, dated Sep. 12, 2002, entitled “Methods of Preventing or Treating Inflammatory or Autoimmune Disorders by Administering Integrin AlphaV Beta3 Antagonists,” International Publication No. WO 03/075957 A1, dated Sep. 18, 2003, entitled “The Prevention or Treatment of Cancer Using Integrin AlphaVBeta3 Antagonists in Combination With Other Agents,” U.S. Patent Pub. No. US 2002/0168360 A1, dated Nov. 14, 2002, entitled “Methods of Preventing or Treating Inflammatory or Autoimmune Disorders by Administering Integrin αᵥβ3 Antagonists in Combination With Other Prophylactic or Therapeutic Agents,” and International Publication No. WO 03/075741 A2, dated Sep. 18, 2003, entitled, “Methods of Preventing or Treating Disorders by Administering an Integrin αvβ3 Antagonist in Combination With an HMG-CoA Reductase Inhibitor or a Bisphosphonate,” each of which is incorporated herewith by reference in its entirety).

In another specific embodiment, the methods of the invention are used to prevent, manage, treat, and/or ameliorate a bacterial infection, or one or more symptoms thereof, said methods comprising administering to a subject in need thereof an effective amount of one or more EphA2/EphrinA1 Modulators of the invention in combination with an effective amount of siplizumab (MedImmune, Inc., International Pub. No. WO 02/069904). In another embodiment, the methods of the invention are used to prevent, manage, treat and/or ameliorate a bacterial infection or one or more symptoms thereof, said methods comprising administering to a subject in need thereof an effective amount of one or more EphA1/EphrinA1 Modulators in combination with an effective mount of one or more anti-Il-9 antibodies (e.g., one of the anti-IL-9 antibodies described in U.S. Pat. Pub. No. 20050002934 (Jan. 6, 2005)), which is incorporated herein by reference in its entirety). In yet another embodiment, the invention provides methods of preventing, treating, managing, and/or ameliorating a bacterial infection, or one or more symptoms thereof, said methods comprising administering an effective amount of one or more EphA2/EphrinA1 Modulators of the invention in combination with an effective amount of two or more of the following: VITAXIN™, siplizumab, and/or anti-II-9 antibodies.

The invention encompasses methods for preventing the development of bacterial infections, in a patient expected to suffer from a bacterial infection or at increased risk of such an infection, e.g., patients with suppressed immune systems (e.g., organ-transplant recipients, AIDS patients, patients undergoing chemotherapy, the elderly, infants born prematurely, infants, children, patients with carcinoma of the esophagus with obstruction, patients with tracheobronchial fistula, patients with neurological diseases (e.g., caused by stroke, amyotrophic lateral sclerosis, multiple sclerosis, and myopathies), and patients already suffering from an infection). The patients may or may not have been previously treated for an infection.

The EphA2/EphrinA1 Modulators of the invention or combination therapies of the invention may be used as any line of therapy, including but not limited to the first, second, third, fourth, or fifth line of therapy, to prevent, manage, treat, and/or ameliorate a bacterial infection, or one or more symptom thereof. The invention also includes methods of preventing, treating, managing, and/or ameliorating a bacterial infection, or one or more symptoms thereof in a patient undergoing therapies for other diseases or disorders. The invention encompasses methods of preventing, managing, treating, and/or ameliorating a bacterial infection, or one or more symptoms thereof in a patient before any adverse effects or intolerance to therapies other than EphA2/EphrinA1 Modulators of the invention develops. The invention also encompasses methods of preventing, treating, managing, and/or ameliorating a bacterial infection, or a symptom thereof in refractory patients. In certain embodiments, a patient with a bacterial infection is refractory to a therapy when the infection has not significantly been eradicated and/or the symptoms have not been significantly alleviated. The determination of whether a patient is refractory can be made either in vivo or in vitro by any method known in the art for assaying the effectiveness of a treatment of infections, using art-accepted meanings of “refractory” in such a context. In various embodiments, a patient with a bacterial infection is refractory when bacterial replication has not decreased or has increased. The invention also encompasses methods of preventing the onset or reoccurrence of a bacterial infection, in patients at risk of developing such infection. The invention also encompasses methods of preventing, managing, treating, and/or ameliorating a bacterial infection, or a symptom thereof in patients who are susceptible to adverse reactions to conventional therapies. The invention further encompasses methods for preventing, treating, managing, and/or ameliorating bacterial infections, for which no anti-bacterial therapy is available.

The invention encompasses methods for preventing, treating, managing, and/or ameliorating a bacterial infection, or a symptom thereof in a patient who has proven refractory to therapies other than EphA2/EphrinA1 Modulators of the invention, but are no longer on these therapies. In certain embodiments, the patients being managed or treated in accordance with the methods of this invention are patients already being treated with anti-inflammatory agents, antibiotics, anti-virals, anti-fungals, anti-protozoan agents, or other biological therapy/immunotherapy. Among these patients are refractory patients, patients who are too young for conventional therapies, and patients with reoccurring bacterial infections despite management or treatment with existing therapies.

The present invention encompasses methods for preventing, treating, managing, and/or ameliorating a bacterial infection, or one or more symptoms thereof as an alternative to other conventional therapies. In specific embodiments, the patient being managed or treated in accordance with the methods of the invention is refractory to other therapies or is susceptible to adverse reactions from such therapies. The patient may be a person with a suppressed immune system (e.g., post-operative patients, chemotherapy patients, and patients with immunodeficiency disease), a person with impaired renal or liver function, the elderly, children, infants, infants born prematurely, persons with neuropsychiatric disorders or those who take psychotropic drugs, persons with histories of seizures, or persons on medication that would negatively interact with conventional agents used to prevent, manage, treat, and/or ameliorate a bacterial infection, or one or more symptoms thereof.

Bacterial infection therapies and their dosages, routes of administration and recommended usage are known in the art and have been described in such literature as the Physicians' Desk Reference (59th ed., 2005).

### 5.2.4 Fungal Infections

One or more EphA2/EphrinA1 Modulators of the invention can be administered according to methods of the invention to a subject to prevent, treat, manage, and/or ameliorate a fungal infection or one or more symptoms thereof. In a preferred embodiment, cells infected by fungi have increased EphA2 expression. One or more EphA2/EphrinA1 Modulators of the invention may be also administered to a subject to treat, manage, and/or ameliorate a fungal infection and/or one or more symptoms thereof in combination with one or more other therapies (e.g., one or more prophylactic or therapeutic agents) other than EphA2/EphrinA1 Modulators of the invention which are useful for the prevention, treatment, management, or amelioration of a fungal infection or one or more symptoms thereof. In a preferred embodiment, the fungal infections to be treated, managed, prevented and/or ameliorated in accordance with the methods of the present invention are intracellular fungal infections.

Any type of fungal infection or condition resulting from or associated with a fungal infection can be prevented, treated, managed, and/or ameliorated in accordance with the methods of invention. Examples of fungus which cause fungal infections include, but not limited to, Absidia species (e.g., Absidia corymbifera and Absidia ramosa), Aspergillus species, (e.g., Aspergillus flavus, Aspergillus fumigatus, Aspergillus nidulans, Aspergillus niger, and Aspergillus terreus), Basidiobolus ranarum, Blastomyces dermatitidis, Candida species (e.g., Candida albicans, Candida glabrata, Candida kerr, Candida krusei, Candida parapsilosis, Candida pseudotropicalis, Candida quillermondii, Candida rugosa, Candida stellatoidea, and Candida tropicalis), Coccidioides immitis, Conidiobolus species, Cryptococcus neoforms, Cunninghamella species, dermatophytes, Histoplasma capsulatum, Microsporum gypseum, Mucor pusillus, Paracoccidioides brasiliensis, Pseudallescheria boydii, Rhinosporidium seeberi, Pneumocystis carinii, Rhizopus species (e.g., Rhizopus arrhizus, Rhizopus oryzae, and Rhizopus microsporus), Saccharomyces species, Sporothrix schenckii, zygomycetes, and classes such as Zygomycetes, Ascomycetes, the Basidiomycetes, Deuteromycetes, and Oomycetes. In a specific embodiment, a fungal infection is not a respiratory fungal infection.

In a specific embodiment, the invention provides a method of preventing, treating, managing, and/or ameliorating a fungal infection or one or more symptoms thereof, said method comprising administering to a subject in need thereof an effective amount of one or more EphA2/EphrinA1 Modulators of the invention. In another embodiment, the invention provides a method of preventing, treating, managing, and/or ameliorating a fungal infection or one or more symptoms thereof, said method comprising administering to a subject in need thereof an effective amount of one or more EphA2/EphrinA1 Modulators of the invention and an effective amount of one or more therapies (e.g., one or more prophylactic or therapeutic agents) other than EphA2/EphrinA1 Modulators of the invention.

In certain embodiments, an effective amount of one or more antibodies is administered in combination with an effective amount of one or more therapies (e.g., one or more prophylactic or therapeutic agents), other than EphA2/EphrinA1 Modulators of the invention, which are currently being used, have been used, or are known to be useful in the prevention, management, treatment, or amelioration of a fungal infection, preferably a fungal infection, to a subject in need thereof. Therapies for fungal infections include, but are not limited to, anti-fungal agents such as azole drugs e.g., miconazole, ketoconazole (NIZORAL®), caspofungin acetate (CANCIDAS®), imidazole, triazoles (e.g., fluconazole (DIFLUCAN®)), and itraconazole (SPORANOX®)), polyene (e.g., nystatin, amphotericin B colloidal dispersion (“ABCD”)(AMPHOTEC®), liposomal amphotericin B (AMBISONE®), postassium iodide (KI), pyrimidine (e.g., flucytosine (ANCOBON®)), and voriconazole (VFEND®). In certain embodiments, an effective amount of one or more EphA2/EphrinA1 Modulators of the invention are administered in combination with one or more supportive measures to a subject in need thereof to prevent, manage, treat, and/or ameliorate a fungal infection or one or more symptoms thereof. Non-limiting examples of supportive measures include humidification of the air by an ultrasonic nebulizer, aerolized racemic epinephrine, oral desamethasone, intravenous fluids, intubation, fever reducers (e.g., ibuprofen and acetometaphin), and anti-viral or anti-bacterial therapy (i.e., to prevent or treat secondary viral or bacterial infections).

The invention also provides methods for preventing, managing, treating and/or ameliorating a biological response to a fungal infection such as, but not limited to, elevated levels of IgE antibodies, elevated nerve growth factor (NGF) levels, mast cell proliferation, degranulation, and/or infiltration, increased proliferation and/or infiltration of B cells, and increased proliferation and/or infiltration of T cells, said methods comprising administration of an effective amount of one or more EphA2/EphrinA1 Modulators alone or in combination with one or more other therapies.

In a specific embodiment, the invention provides methods for preventing, treating, managing, and/or ameliorating one or more secondary conditions or responses to a primary fungal infection, preferably a primary fungal infection, said method comprising of administering to a subject in need thereof an effective amount of one or more EphA2/EphrinA1 Modulators of the invention alone or in combination with an effective amount of other therapies (e.g., other prophylactic or therapeutic agents) other than EphA2/EphrinA1 Modulators of the invention. Examples of secondary conditions or responses to a primary fungal infections, particularly primary fungal infection include, but are not limited to, asthma-like responsiveness to mucosal stimula, elevated total resistance, increased susceptibility to secondary viral, fungal, and fungal infections, and development of such conditions such as, but not limited to, pneumonia, croup, and febrile bronchitits.

In a specific embodiment, the invention provides methods to prevent, treat, manage, and/or ameliorate a fungal infection or one or more symptoms thereof, said methods comprising administering to a subject in need thereof an effective amount of one or more EphA2/EphrinA1 Modulators of the invention in combination with an effective amount of VITAXIN™ (MedImmune, Inc., International Publication No. WO 00/78815, International Publication No. WO 02/070007 Al, dated Sep. 12, 2002, entitled “Methods of Preventing or Treating Inflammatory or Autoimmune Disorders by Administering Integrin AlphaV Beta3 Antagonists,” International Publication No. WO 03/075957 A1, dated Sep. 18, 2003, entitled “The Prevention or Treatment of Cancer Using Integrin AlphaVBeta3 Antagonists in Combination With Other Agents,” U.S. Patent Pub. No. US 2002/0168360 A1, dated Nov. 14, 2002, entitled “Methods of Preventing or Treating Inflammatory or Autoimmune Disorders by Administering Integrin αᵥβ3 Antagonists in Combination With Other Prophylactic or Therapeutic Agents,” and International Publication No. WO 03/075741 A2, dated Sep. 18, 2003, entitled, “Methods of Preventing or Treating Disorders by Administering an Integrin αvβ3 Antagonist in Combination With an HMG-CoA Reductase Inhibitor or a Bisphosphonate,” each of which is incorporated herewith by reference in its entirety) to a subject in need thereof.

In another embodiment, the invention provides methods of preventing, treating, managing, and/or ameliorating a fungal infection or one or more symptoms thereof, said methods comprising administering to a subject in need thereof an effective amount of one or more EphA2/EphrinA1 Modulators of the invention in combination with an effective amount of siplizumab (MedImmune, Inc., International Pub. No. WO 02/069904) to a subject in need thereof. In another embodiment, the invention provides methods of preventing, treating, managing and/or ameliorating a fungal infection or one or more symptoms thereof, said methods comprising administering to a subject in need thereof an effective amount of one or more EphA2/EphrinA1 Modulators in combination with an effective amount of one or more anti-IL-9 antibodies (e.g., one or more of the anti-IL-9 antibodies described in U.S. Pat. Pub. No. 20050002934 (Jan. 6, 2005)), which is incorporated herein by reference in its entirety). In another embodiment, the invention provides methods of preventing, treating, managing and/or ameliorating a fungal infection or one or more symptoms thereof, said methods comprising administering to a subject in need thereof an effective amount of one or more EphA2/EphrinA1 Modulators in combination with an effective amount of two or more of the following: Vitaxin, Siplizumab and/or anti-IL-9 antibodies.

The invention encompasses methods for preventing the development of fungal infections in a patient expected to suffer from a fungal infection, or at increased risk of such an infection. Such subjects include, but are not limited to, patients with suppressed immune systems (e.g., patients organ-transplant recipients, AIDS patients, patients undergoing chemotherapy, patients with carcinoma of the esophagus with obstruction, patients with tracheobronchial fistula, patients with neurological diseases (e.g., caused by stroke, amyotorphic lateral sclerosis, multiple sclerosis, and myopathies), and patients already suffering from a condition, particularly a infection). In a specific embodiment, the patient suffers from bronchopulmonary dysplasia, congenital heart disease, cystic fibrosis, and/or acquired or congenital immunodeficiency. In another specific embodiment, the patient is an infant born prematurely, an infant, a child, an elderly human, or a human in a group home, nursing home, or some other type of institution. The invention also encompasses methods of preventing, managing, treating, and/or ameliorating a fungal infection or one or more symptoms thereof in patients who are susceptible to adverse reactions to conventional anti-fungal therapies for conditions for which no therapies are available.

The EphA2/EphrinA1 Modulators of the invention or combination therapies of the invention may be used as any line of therapy, including but not limited to the first, second, third, fourth, or fifth line of therapy, to prevent, manage, treat, and/or ameliorate a fungal infection or one or more symptom thereof. The invention also includes methods of preventing, treating, managing, and/or ameliorating a fungal infection or one or more symptoms thereof in a patient undergoing therapies for other disease or disorders. The invention encompasses methods of preventing, managing, treating, and/or ameliorating a fungal infection or one or more symptoms thereof in a patient before any adverse effects or intolerance to therapies other EphA2/EphrinA1 Modulators of the invention develops. The invention also encompasses methods of preventing, treating, managing, and/or ameliorating a fungal infection or a symptom thereof in refractory patients. In certain embodiments, a patient with a fungal infection, is refractory to a therapy when the infection has not significantly been eradicated and/or the symptoms have not been significantly alleviated. The determination of whether a patient is refractory can be made either in vivo or in vitro by any method known in the art for assaying the effectiveness of a treatment of infections, using art-accepted meanings of “refractory” in such a context. In various embodiments, a patient with a fungal infection, is refractory when fungal replication has not decreased or has increased. The invention also encompasses methods of preventing the onset or reoccurrence of fungal infections, in patients at risk of developing such infections. The invention also encompasses methods of preventing, managing, treating, and/or ameliorating a fungal infection or a symptom thereof in patients who are susceptible to adverse reactions to conventional therapies. The invention further encompasses methods for preventing, treating, managing, and/or ameliorating fungal infections, for which no anti-fungal therapy is available.

The invention encompasses methods for preventing, treating, managing, and/or ameliorating a fungal infection, or a symptom thereof in a patient who has proven refractory to therapies other than EphA2/EphrinA1 Modulators of the invention but are no longer on these therapies. In certain embodiments, the patients being managed or treated in accordance with the methods of this invention are patients already being treated with antibiotics, anti-virals, anti-fungals, or other biological therapy/immunotherapy. Among these patients are refractory patients, patients who are too young for conventional therapies, and patients with reoccurring fungal infections despite management or treatment with existing therapies.

The present invention provides methods for preventing, treating, managing, and/or ameliorating a fungal infection or one or more symptoms thereof as an alternative to other conventional therapies. In specific embodiments, the patient being managed or treated in accordance with the methods of the invention is refractory to other therapies or is susceptible to adverse reactions from such therapies. The patient may be a person with a suppressed immune system (e.g., post-operative patients, chemotherapy patients, and patients with immunodeficiency disease), a person with impaired renal or liver function, the elderly, children, infants, infants born prematurely, persons with neuropsychiatric disorders or those who take psychotropic drugs, persons with histories of seizures, or persons on medication that would negatively interact with conventional agents used to prevent, manage, treat, and/or ameliorate a fungal infection, or one or more symptoms thereof.

Fungal infection therapies and their dosages, routes of administration and recommended usage are known in the art and have been described in such literature as the Physicians' Desk Reference (59th ed., 2005).

### 5.2.5 Protozoan Infections

One or more EphA2/EphrinA1 Modulators of the invention can be administered according to methods of the invention to a subject to prevent, treat, manage, and/or ameliorate a protozoan infection or one or more symptoms thereof. In a preferred embodiment, cells infected by protozoa have increased EphA2 expression. One or more EphA2/EphrinA1 Modulators of the invention may be also administered to a subject to treat, manage, and/or ameliorate a protozoa infection or one or more symptoms thereof in combination with one or more other therapies (e.g., one or more prophylactic or therapeutic agents) other than EphA2/EphrinA1 Modulators of the invention which are useful for the prevention, treatment, management, or amelioration of a fungal infection or one or more symptoms thereof. In a preferred embodiment, the protozoan infections to be treated, managed, prevented and/or ameliorated in accordance with the methods of the present invention are intracellular protozoan infections.

Any type of protozoa infection or condition resulting from or associated with a protozoa infection can be prevented, treated, managed, and/or ameliorated in accordance with the methods of invention. Examples of protozoa which cause infections include, but not limited to, Leishmania; Trypanosoma; Giardia; Trichomonas; Entamoeba; Dientamoeba; Naegleria and Acanthamoeba; Babesia; Plasmodium; Isospora; Sarcocystis; Toxoplasma; Enterocytozoon; Balantidium; and Pneumocystis.

In a specific embodiment, the invention provides a method of preventing, treating, managing, and/or ameliorating a protozoa infection or one or more symptoms thereof, said method comprising administering to a subject in need thereof an effective amount of one or more EphA2/EphrinA1 Modulators of the invention. In another embodiment, the invention provides a method of preventing, treating, managing, and/or ameliorating a protozoa infection or one or more symptoms thereof, said method comprising administering to a subject in need thereof an effective amount of one or more EphA2/EphrinA1 Modulators of the invention and an effective amount of one or more therapies (e.g., one or more prophylactic or therapeutic agents) other than EphA2/EphrinA1 Modulators of the invention.

In certain embodiments, an effective amount of one or more EphA2/EphrinA1 Modulators is administered in combination with an effective amount of one or more therapies (e.g., one or more prophylactic or therapeutic agents), other than EphA-/EphrinA1 Modulators of the invention, which are currently being used, have been used, or are known to be useful in the prevention, management, treatment, or amelioration of a protozoa infection, to a subject in need thereof. In certain embodiments, an effective amount of one or more EphA2/EphrinA1 Modulators of the invention are administered in combination with one or more supportive measures to a subject in need thereof to prevent, manage, treat, and/or ameliorate a protozoa infection or one or more symptoms thereof. Non-limiting examples of supportive measures include humidification of the air by an ultrasonic nebulizer, aerolized racemic epinephrine, oral desamethasone, intravenous fluids, intubation, fever reducers (e.g., ibuprofen and acetometaphin), and anti-viral or anti-bacterial therapy (i.e., to prevent or treat secondary viral or bacterial infections).

The invention also provides methods for preventing, managing, treating and/or ameliorating a biological response to a protozoa infection such as, but not limited to, elevated levels of IgE antibodies, elevated nerve growth factor (NGF) levels, mast cell proliferation, degranulation, and/or infiltration, increased proliferation and/or infiltration of B cells, and increased proliferation and/or infiltration of T cells, said methods comprising administration of an effective amount of one or more EphA2/EphrinA1 Modulators alone or in combination with one or more other therapies.

In a specific embodiment, the invention provides methods for preventing, treating, managing, and/or ameliorating one or more secondary conditions or responses to a primary infection, preferably a primary protozoa infection, said method comprising of administering to a subject in need thereof an effective amount of one or more EphA2/EphrinA1 Modulators of the invention alone or in combination with an effective amount of other therapies (e.g., other prophylactic or therapeutic agents) other than EphA2/EphrinA1 Modulators of the invention.

In a specific embodiment, the invention provides methods to prevent, treat, manage, and/or ameliorate a protozoa infection or one or more symptoms thereof, said methods comprising administering to a subject in need thereof an effective amount of one or more EphA2/EphrinA1 Modulators of the invention in combination with an effective amount of VITAXIN™ (MedImmune, Inc., International Publication No. WO 00/78815, International Publication No. WO 02/070007 A1, dated Sep. 12, 2002, entitled “Methods of Preventing or Treating Inflammatory or Autoimmune Disorders by Administering Integrin AlphaV Beta3 Antagonists,” International Publication No. WO 03/075957 A1, dated Sep. 18, 2003, entitled “The Prevention or Treatment of Cancer Using Integrin AlphaVBeta3 Antagonists in Combination With Other Agents,” U.S. Patent Pub. No. US 2002/0168360 A1, dated Nov. 14, 2002, entitled “Methods of Preventing or Treating Inflammatory or Autoimmune Disorders by Administering Integrin αᵥβ3 Antagonists in Combination With Other Prophylactic or Therapeutic Agents,” and International Publication No. WO 03/075741 A2, dated Sep. 18, 2003, entitled, “Methods of Preventing or Treating Disorders by Administering an Integrin αvβ3 Antagonist in Combination With an HMG-CoA Reductase Inhibitor or a Bisphosphonate,” each of which is incorporated herewith by reference in its entirety) to a subject in need thereof.

In another embodiment, the invention provides methods of preventing, treating, managing, and/or ameliorating a protozoa infection or one or more symptoms thereof, said methods comprising administering to a subject in need thereof an effective amount of one or more EphA2/EphrinA1 Modulators of the invention in combination with an effective amount of siplizumab (MedImmune, Inc., International Pub. No. WO 02/069904) to a subject in need thereof. In another embodiment, the invention provides methods of preventing, treating, managing, and/or ameliorating a protozoa infection or one or more symptoms thereof, said methods comprising administering to a subject in need thereof an effective amount of one or more EphA2/EphrinA1 Modulators in combination with an effective amount of one or more anti-IL-9 antibodies (e.g., the anti-IL-9 antibodies described in U.S. Pat. Pub. No. 20050002934 (Jan. 6, 2005)), which is incorporated herein by reference in its entirety). In another embodiment, the invention provides methods of preventing, treating, managing, and/or ameliorating a protozoa infection or one or more symptoms thereof, said methods comprising administering to a subject in need thereof an effective amount of one or more EphA2/EphrinA1 Modulators in combination with an effective amount of two or more of the following: Vitaxin, siplizumab and/or anti-IL-9 antibodies.

The invention encompasses methods for preventing the development of protozoa infections in a patient expected to suffer from a protozoa infection, or at increased risk of such an infection. Such subjects include, but are not limited to, patients with suppressed immune systems (e.g., patients organ-transplant recipients, AIDS patients, patients undergoing chemotherapy, patients with cancer, patients with tracheobronchial fistula, patients with neurological diseases (e.g., caused by stroke, amyotorphic lateral sclerosis, multiple sclerosis, and myopathies), and patients already suffering from a condition, particularly a infection). In a specific embodiment, the patient suffers from bronchopulmonary dysplasia, congenital heart disease, cystic fibrosis, and/or acquired or congenital immunodeficiency. In another specific embodiment, the patient is an infant born prematurely, an infant, a child, an elderly human, or a human in a group home, nursing home, or some other type of institution. The invention also encompasses methods of preventing, managing, treating, and/or ameliorating a protozoa infection or one or more symptoms thereof in patients who are susceptible to adverse reactions to conventional anti-protozoa therapies for conditions for which no therapies are available.

The EphA2/EphrinA1 Modulators of the invention or combination therapies of the invention may be used as any line of therapy, including but not limited to the first, second, third, fourth, or fifth line of therapy, to prevent, manage, treat, and/or ameliorate a protozoa infection or one or more symptom thereof. The invention also includes methods of preventing, treating, managing, and/or ameliorating a protozoa infection or one or more symptoms thereof in a patient undergoing therapies for other disease or disorders. The invention encompasses methods of preventing, managing, treating, and/or ameliorating a protozoa infection, or one or more symptoms thereof in a patient before any adverse effects or intolerance to therapies other EphA2/EphrinA1 Modulators of the invention develops. The invention also encompasses methods of preventing, treating, managing, and/or ameliorating a protozoa infection, or a symptom thereof in refractory patients. In certain embodiments, a patient with a protozoa infection, is refractory to a therapy when the infection has not significantly been eradicated and/or the symptoms have not been significantly alleviated. The determination of whether a patient is refractory can be made either in vivo or in vitro by any method known in the art for assaying the effectiveness of a treatment of infections, using art-accepted meanings of “refractory” in such a context. In various embodiments, a patient with a protozoa infection is refractory when protozoa replication has not decreased or has increased. The invention also encompasses methods of preventing the onset or reoccurrence of protozoa infections, in patients at risk of developing such infections. The invention also encompasses methods of preventing, managing, treating, and/or ameliorating a protozoa infection or a symptom thereof in patients who are susceptible to adverse reactions to conventional therapies. The invention further encompasses methods for preventing, treating, managing, and/or ameliorating protozoa infections, for which no anti-protozoa therapy is available.

The invention encompasses methods for preventing, treating, managing, and/or ameliorating a protozoa infection or a symptom thereof in a patient who has proven refractory to therapies other than EphA2/EphrinA1 Modulators of the invention but are no longer on these therapies. In certain embodiments, the patients being managed or treated in accordance with the methods of this invention are patients already being treated with antibiotics, anti-virals, anti-protozoa, or other biological therapy/immunotherapy. Among these patients are refractory patients, patients who are too young for conventional therapies, and patients with reoccurring protozoa infections despite management or treatment with existing therapies.

The present invention provides methods for preventing, treating, managing, and/or ameliorating a protozoa infection or one or more symptoms thereof as an alternative to other conventional therapies. In specific embodiments, the patient being managed or treated in accordance with the methods of the invention is refractory to other therapies or is susceptible to adverse reactions from such therapies. The patient may be a person with a suppressed immune system (e.g., post-operative patients, chemotherapy patients, and patients with immunodeficiency disease), a person with impaired renal or liver function, the elderly, children, infants, infants born prematurely, persons with neuropsychiatric disorders or those who take psychotropic drugs, persons with histories of seizures, or persons on medication that would negatively interact with conventional agents used to prevent, manage, treat, and/or ameliorate a fungal infection or one or more symptoms thereof.

Protozoa infection therapies and their dosages, routes of administration and recommended usage are known in the art and have been described in such literature as the Physicians' Desk Reference (59th ed., 2005).

### 5.2.6 Other Therapies

The invention provides methods for treating, managing or preventing an infection, in particular, an intracellular pathogen infection, by administering one or more EphA2/EphrinA1 Modulators of the invention in combination with one or more therapies. Preferably, those other therapies are currently being used or are useful in the treatment, management or prevention of an infection. In a specific embodiment, the invention provides a method of treating, managing, preventing and/or ameliorating an infection, the method comprising administering to a subject in need thereof an effective amount of an EphA2/EphrinA1 Modulator and an effective amount of a therapy other than an EphA2/EphrinA1 Modulator. Any therapy (e.g., prophylactic or therapeutic agents) which is known to be useful, or which has been used or is currently being used for the prevention, management, treatment or amelioration of an infection or a symptom thereof can be used in combination with an EphA2/EphrinA1 Modulator in accordance with the invention described herein. See, e.g., Gilman et al., Goodman and Gilman's: The Pharmacological Basis of Therapeutics, Tenth Ed., McGraw-Hill, New York, 2001; The Physicians' Desk Reference (59^{th} ed., 2005); The Merck Manual of Diagnosis and Therapy, Berkow, M. D. et al. (eds.). 17^{th} Ed., Merck Sharp & Dohme Research Laboratories, Rahway, N.J., 1999; and Cecil Textbook of Medicine, 20^{th} Ed., Bennett and Plum (eds.), W. B. Saunders, Philadelphia, 1996, for information regarding therapies, in particular prophylactic or therapeutic agents, which have been or are currently being used for preventing, treating, managing, and/or ameliorating an infection or a symptom thereof. Therapeutic or prophylactic agents include, but are not limited to, small molecules, synthetic drugs, peptides, polypeptides, proteins, nucleic acids, (e.g., DNA and RNA nucleotides including, but not limited to, antisense nucleotide sequences, triple helices, RNAi, and nucleotide sequences encoding biologically active proteins, polypeptides or peptides) antibodies, synthetic or natural inorganic molecules, mimetic agents, and synthetic or natural organic molecules. Examples of prophylactic and therapeutic agents include, but are not limited to, immunomodulatory agents, anti-inflammatory agents (e.g., adrenocorticoids, corticosteroids, (e.g., beclomethasone, budesonide, flunisolide, fluticasone, triamcinolone, methylprednisolone, prednisolone, prednisone, hydrocortisone), glucocorticoids, steroids, and non-steroidal anti-inflammatory drugs (e.g., aspirin, ibuprofen, diclofenac, and COX-2 inhibitors), anticholinergic agents (e.g., ipratropium bromide and oxitropium bromide), sulphasalazine, penicillamine, dapsone, antihistamines, anti-malarial agents (e.g., hydroxychloroquine), anti-viral agents, and antibiotics (e.g., dactinomycin (formerly actinomycin), bleomycin, erythromycin, penicillin, mithramycin, and anthramycin (AMC)).

In other embodiments, an EphA2/EphrinA1 Modulator of the invention is administered to a subject in need thereof in combination with an anti-inflammatory agent, an anti-viral agent, an antibiotic, an anti-fungal agent, anti-protozoa agent and/or an immunomodulatory agent.

The therapies can be administered to a subject in need thereof sequentially or concurrently. In particular, the therapies should be administered to a subject at exactly the same time or in a sequence within a time interval such that the therapies can act together to provide an increased benefit than if they were administered otherwise. In a specific embodiment, the combination therapies of the invention comprise an effective amount of one or more EphA2/EphrinA1 Modulators of the invention and an effective amount of at least one other therapy which has the same mechanism of action as said EphA2/EphrinA1 Modulators of the invention. In a specific embodiment, the combination therapies of the invention comprise an effective amount of one or more EphA2/EphrinA1 Modulators of the invention and an effective amount of at least one other therapy (e.g., prophylactic or therapeutic agent) which has a different mechanism of action than said EphA2/EphrinA1 Modulators of the invention.

In certain embodiments, the combination therapies of the present invention improve the prophylactic or therapeutic effect of one or more other therapies other than EphA2/EphrinA1 Modulators by functioning together with the EphA2/EphrinA1 Modulators of the invention to have an additive or synergistic effect. In certain embodiments, the combination therapies of the present invention reduce the side effects associated with the prophylactic or therapeutic agents. In various embodiments, the therapies are administered to a patient less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, a about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more therapies are administered within the same patient visit.

The prophylactic or therapeutic agents of the combination therapies can be administered to a subject, preferably a human subject, in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

In a specific embodiment, a pharmaceutical composition comprising one or more EphA2/EphrinA1 Modulators of the invention described herein is administered to a subject, preferably a human, to prevent, treat, manage and/or ameliorate an infection or a symptom thereof. In accordance with the invention, pharmaceutical compositions of the invention may also comprise one or more therapies (e.g., prophylactic or therapeutic agents), other than the EphA2/EphrinA1 Modulators of the invention, which are currently being used, have been used, or are known to be useful in the prevention, treatment or amelioration of one or more symptoms associated with an infection.

### 5.2.6.1 Immunomodulatory Therapies

In certain embodiments, the present invention provides compositions comprising one or more EphA2/EphrinA1 Modulators of the invention and one or more immunomodulatory agents (i.e., agents which modulate the immune response in a subject), and methods for treating, managing, preventing and/or ameliorating an infection or a symptom thereof, in a subject comprising the administration of said compositions. The invention also provides methods for treating, managing, preventing and/or ameliorating an infection or a symptom thereof comprising the administration of an EphA2/EphrinA1 Modulator in combination with one or more immunomodulatory agents. In a specific embodiment of the invention, the immunomodulatory agent inhibits or suppresses the immune response in a human subject. Immunomodulatory agents are well-known to one skilled in the art and can be used in the methods and compositions of the invention.

Any immunomodulatory agent well-known to one of skill in the art may be used in the methods and compositions of the invention. Immunomodulatory agents can affect one or more or all aspects of the immune response in a subject. Aspects of the immune response include, but are not limited to, the inflammatory response, the complement cascade, leukocyte and lymphocyte differentiation, proliferation, and/or effector function, monocyte and/or basophil counts, and the cellular communication among cells of the immune system. In certain embodiments of the invention, an immunomodulatory agent modulates one aspect of the immune response. In other embodiments, an immunomodulatory agent modulates more than one aspect of the immune response. In a preferred embodiment of the invention, the administration of an immunomodulatory agent to a subject inhibits or reduces one or more aspects of the subject's immune response capabilities. In a specific embodiment of the invention, the immunomodulatory agent inhibits or suppresses the immune response in a subject. In accordance with the invention, an immunomodulatory agent is not an EphA2/EphrinA1 Modulator. In certain embodiments, an immunomodulatory agent is not an anti-inflammatory agent. In certain embodiments, an immunomodulatory agent is a chemotherapeutic agent. In certain embodiments, an immunomodulatory agent is not a chemotherapeutic agent.

Examples of immunomodulatory agents include, but are not limited to, proteinaceous agents such as cytokines, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules and triple helices), small molecules, organic compounds, and inorganic compounds. In particular, immunomodulatory agents include, but are not limited to, methotrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine. deoxyspergualin, brequinar, malononitriloamindes (e.g., leflunamide), T cell receptor modulators, cytokine receptor modulators, and modulators mast cell modulators.

In a specific embodiment, an immunomodulatory agent is a T cell receptor modulator. As used herein, the term “T cell receptor modulator” refers to an agent which modulates the phosphorylation of a T cell receptor, the activation of a signal transduction pathway associated with a T cell receptor and/or the expression of a particular protein associated with T cell receptor activity such as a cytokine. Such an agent may directly or indirectly modulate the phosphorylation of a T cell receptor, and/or the expression of a particular protein associated with T cell receptor activity such as a cytokine. Examples of T cell receptor modulators include, but are not limited to, anti-T cell receptor antibodies (e.g., anti-CD4 antibodies (e.g., cM-T412 (Boeringer), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies (e.g., siplizumab (MedImmune, Inc., International Publication Nos. WO 02/098370 and WO 02/069904)), anti-CD11a antibodies (e.g., Xanelim (Genentech)), and anti-B7 antibodies (e.g., IDEC-114) (IDEC))), CTLA4-immunoglobulin, and LFA-3TIP (Biogen, International Publication No. WO 93/08656 and U.S. Pat. No. 6,162,432).

In a specific embodiment, an immunomodulatory agent is a cytokine receptor modulator. As used herein, the term “cytokine receptor modulator” refers to an agent which modulates the phosphorylation of a cytokine receptor, the activation of a signal transduction pathway associated with a cytokine receptor, and/or the expression of a particular protein such as a cytokine or cytokine receptor. Such an agent may directly or indirectly modulate the phosphorylation of a cytokine receptor, the activation of a signal transduction pathway associated with a cytokine receptor, and/or the expression of a particular protein such as a cytokine. Examples of cytokine receptor modulators include, but are not limited to, soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof), cytokines or fragments thereof (e.g., interleukin IL-2, IL-3, IL-4, IL-5, IL-6, IL-7, IL-8, IL-9, IL-10, IL-11, IL-12, IL-13, IL-15, IL-23, TNF-α, TNF-β, interferon (IFN)-α, IFN-β, IFN-γ, and GM-CSF), anti-cytokine receptor antibodies (e.g., anti-IFN receptor antibodies, anti-IL-2 receptor antibodies (e.g., Zenapax (Protein Design Labs)), anti-IL-3 receptor antibodies, anti-IL-4 receptor antibodies, anti-IL-6 receptor antibodies, anti-IL-10 receptor antibodies, anti-IL-12 receptor antibodies, anti-IL-13 receptor antibodies, anti-IL-15 receptor antibodies, and anti-IL-23 receptor antibodies), anti-cytokine antibodies (e.g., anti-IFN antibodies, anti-TNF-α antibodies, anti-IL-1β antibodies, anti-IL-3 antibodies, anti-IL-6 antibodies, anti-IL-8 antibodies (e.g., ABX-IL-8 (Abgenix)), anti-IL-9 antibodies (e.g., those disclosed in U.S. Pat. Pub. No. 20050002934 (Jan. 6, 2005)), anti-IL-9 receptor antibodies, anti-IL-12 antibodies, anti-IL-13 antibodies, anti-IL-15 antibodies, and anti-IL-23 antibodies).

In a specific embodiment, a cytokine receptor modulator is IL-3, IL-4, IL-10, or a fragment thereof. In another embodiment, a cytokine receptor modulator is an anti-IL-1β antibody, anti-IL-6 antibody, anti-IL-12 receptor antibody, or anti-TNF-α antibody. In another embodiment, a cytokine receptor modulator is the extracellular domain of a TNF-α receptor or a fragment thereof. In certain embodiments, a cytokine receptor modulator is not a TNF-α antagonist.

In a preferred embodiment, the immunomodulatory agent decreases the amount of IL-9. In a more preferred embodiment, the immunomodulatory agent is an antibody (preferably a monoclonal antibody) or fragment thereof that immunospecifically binds to IL-9 (see, e.g., U.S. patent application Ser. No. 10/823,810, filed Apr. 12, 2004 entitled “Methods of Preventing or Treating Respiratory Conditions” by Reed (Attorney Docket No. 10271-113-999), U.S. Pat. Pub. No. 20050002934 (Jan. 6, 2005), and U.S. Provisional Application No. 60/561,845 filed Apr. 12, 2004 entitled “Anti-IL-9 Antibody Formulations and Uses Thereof” by Reed (Attorney Docket No. 10271-126-888), all of which are incorporated by reference herein in their entireties. Although not intending to be bound by a particular mechanism of action, the use of anti-IL-9 antibodies neutralize the ability of IL-9 to have a biological effect and thereby blocks or decreases inflammatory cell recruitment.

In one embodiment, a cytokine receptor modulator is a mast cell modulator. In an alternative embodiment, a cytokine receptor modulator is not a mast cell modulator. Examples of mast cell modulators include, but are not limited to stem cell factor (c-kit receptor ligand) inhibitors (e.g., mAb 7H6, mAb 8H7a, pAb 1337, FK506, CsA, dexamthasone, and fluconcinonide), c-kit receptor inhibitors (e.g., STI 571 (formerly known as CGP 57148B)), mast cell protease inhibitors (e.g., GW-45, GW-58, wortmannin, LY 294002, calphostin C, cytochalasin D, genistein, KT5926, staurosproine, and lactoferrin), relaxin (“RLX”), IgE antagonists (e.g., antibodies rhuMAb-E25 omalizumab, HMK-12 and 6HD5, and mAB Hu-901), IL-3 antagonists, IL-4 antagonists, IL-10 antagonists; and TGF-beta.

An immunomodulatory agent may be selected to bind to and/or target B cells. For example, an immunomodulatory agent may be an antibody that binds to a B cell marker.

An immunomodulatory agent may be selected to interfere with the interactions between the T helper subsets (TH1 or TH2) and B cells to inhibit neutralizing antibody formation. Antibodies that interfere with or block the interactions necessary for the activation of B cells by TH (T helper) cells, and thus block the production of neutralizing antibodies, are useful as immunomodulatory agents in the methods of the invention. For example, B cell activation by T cells requires certain interactions to occur (Durie et al., Immunol. Today, 15(9):406-410 (1994)), such as the binding of CD40 ligand on the T helper cell to the CD40 antigen on the B cell, and the binding of the CD28 and/or CTLA4 ligands on the T cell to the B7 antigen on the B cell. Without both interactions, the B cell cannot be activated to induce production of the neutralizing antibody.

The CD40 ligand (CD40L)-CD40 interaction is a desirable point to block the immune response because of its broad activity in both T helper cell activation and function as well as the absence of redundancy in its signaling pathway. Thus, in a specific embodiment of the invention, the interaction of CD40L with CD40 is transiently blocked at the time of administration of one or more of the immunomodulatory agents. This can be accomplished by treating with an agent which blocks the CD40 ligand on the TH cell and interferes with the normal binding of CD40 ligand on the T helper cell with the CD40 antigen on the B cell. An antibody to CD40 ligand (anti-CD40L) (available from Bristol-Myers Squibb Co; see, e.g., European patent application 555,880, published Aug. 18, 1993) or a soluble CD40 molecule can be selected and used as an immunomodulatory agent in accordance with the methods of the invention.

An immunomodulatory agent may be selected to inhibit the interaction between TH1 cells and cytotoxic T lymphocytes (“CTLs”) to reduce the occurrence of CTL-mediated killing. An immunomodulatory agent may be selected to alter (e.g., inhibit or suppress) the proliferation, differentiation, activity and/or function of the CD4⁺ and/or CD8⁺ T cells. For example, antibodies specific for T cells can be used as immunomodulatory agents to deplete, or alter the proliferation, differentiation, activity and/or function of CD4⁺ and/or CD8⁺ T cells.

In one embodiment of the invention, an immunomodulatory agent that reduces or depletes T cells, preferably memory T cells, is administered to a subject at risk of or with an infection in accordance with the methods of the invention. See, e.g., U.S. Pat. No. 4,658,019. In another embodiment of the invention, an immunomodulatory agent that inactivates CD8⁺ T cells is administered to a subject at risk of or with an intracellular pathogen infection in accordance with the methods of the invention. In a specific embodiment, anti-CD8 antibodies are used to reduce or deplete CD8⁺ T cells.

In another embodiment, an immunomodulatory agent which reduces or inhibits one or more biological activities (e.g., the differentiation, proliferation, and/or effector functions) of TH0, TH1, and/or TH2 subsets of CD4⁺ T helper cells is administered to a subject at risk of or with an intracellular pathogen infection in accordance with the methods of the invention. One example of such an immunomodulatory agent is IL-4. IL-4 enhances antigen-specific activity of TH2 cells at the expense of the TH1 cell function (see, e.g., Yokota et al, 1986, Proc. Natl. Acad. Sci., USA 83:5894-5898; and U.S. Pat. No. 5,017,691). Other examples of immunomodulatory agents that affect the biological activity (e.g., proliferation, differentiation, and/or effector functions) of T-helper cells (in particular, TH1 and/or TH2 cells) include, but are not limited to, IL-2, IL-4, IL-5, IL-6, IL-10, IL-12, IL-13, IL-15, IL-23, and interferon (IFN)-γ.

In another embodiment, an immunomodulatory agent administered to a subject at risk of or with an intracellular pathogen infection in accordance with the methods of the invention is a cytokine that prevents antigen presentation. In a specific embodiment, an immunomodulatory agent used in the methods of the invention is IL-10. IL-10 also reduces or inhibits macrophage action which involves bacterial elimination.

An immunomodulatory agent may be selected to reduce or inhibit the activation, degranulation, proliferation, and/or infiltration of mast cells. In certain embodiments, the immunomodulatory agent interferes with the interactions between mast cells and mast cell activating agents, including, but not limited to stem cell factors (c-kit ligands), IgE, IL-4, environmental irritants, and infectious agents. In a specific embodiment, the immunomodulatory agent reduces or inhibits the response of mast cells to environmental irritants such as, but not limited to pollen, dust mites, tobacco smoke, and/or pet dander. In another specific embodiment, the immunomodulatory agent reduces or inhibits the response of mast cells to infectious agents, such as viruses, bacteria, fungi and protozoa. Examples of mast cell modulators that reduce or inhibit the activation, degranulation, proliferation, and/or infiltration of mast cells include, but are not limited to, stem cell factor (c-kit receptor ligand) inhibitors (e.g., mAb 7H6, mAb 8H7a, and pAb 1337 (see Mendiaz et al., 1996, Eur J Biochem 293(3):842-849), FK506 and CsA (Ito et al., 1999 Arch Dermatol Res 291(5):275-283), dexamthasone and fluconcinonide (see Finooto et al., 1997, J. Clin. Invest. 99(7):1721-1728)), c-kit receptor inhibitors (e.g., STI 571 (formerly known as CGP 57148B) (see Heinrich et al., 2000 Blood 96(3):925-932)), mast cell protease inhibitors (e.g., GW-45 and GW-58 (see, Temkin et al., 2002, J Immunol 169(5):2662-2669), wortmannin, LY 294002, calphostin C, and cytochalasin D (see Vosseller et al., 1997, Mol Biol Cell 1997:909-922), genistein, KT5926, and staurosproine (see Nagai et al. 1995, Biochem Biophys Res Commun 208(2):576-581), and lactoferrin (see He et al., 2003 Biochem Pharmacol 65(6):1007-1015)), relaxin (“RLX”) (see Bani et al., 2002 Int Immunopharmacol 2(8):1195-1294), ), IgE antagonists (e.g., antibodies rhuMAb-E25 omalizumab (see Finn et al., 2003 J Allergy Clin Immuno 111(2):278-284; Corren et al., 2003 J Allergy Clin Immuno 111(1):87-90; Busse and Neaville, 2001 Curr Opin Allergy Clin Immunol. 1(1):105-108; and Tang and Powell, 2001, Eur J Pediatr 160(12): 696-704), HMK-12 and 6HD5 (see Miyajima et al., 2202 Int Arch Allergy Immuno 128(1):24-32), and mAB Hu-901 (see van Neerven et al., 2001 Int Arch Allergy Immuno 124(1-3):400), IL-3 antagonist, IL-4 antagonists, IL-10 antagonists, and TGF-beta (see Metcalfe et al., 1995, Exp Dermatol 4(4 Pt 2):227-230).

In accordance with the invention, one or more immunomodulatory agents are administered to a subject at risk of or with an infection prior to, subsequent to, or concomitantly with an antibody that immunospecifically binds to an EphA2 or EphrinA1 polypeptide. Preferably, one or more immunomodulatory agents are administered in combination with an antibody that immunospecifically binds to an EphA2 or EphrinA1 polypeptide to a subject at risk of or with an infection to reduce or inhibit one or more aspects of the immune response as deemed necessary by one of skill in the art. Any technique well-known to one skilled in the art can be used to measure one or more aspects of the immune response in a particular subject, and thereby determine when it is necessary to administer an immunomodulatory agent to said subject. In a preferred embodiment, a mean absolute lymphocyte count of approximately 500 cells/mm³, preferably 600 cells/mm³, 650 cells/mm³, 700 cells/mm³, 750 cells/mm³, 800 cells/mm³, 900 cells/mm³, 1000 cells/mm³, 1100 cells/mm³, or 1200 cells/mm³ is maintained in a subject. In another preferred embodiment, a subject at risk of or with an infection is not administered an immunomodulatory agent if their absolute lymphocyte count is 500 cells/mm³ or less, 550 cells/mm³ or less, 600 cells/mm³ or less, 650 cells/mm³ or less, 700 cells/mm³ or less, 750 cells/mm³ or less, or 800 cells/mm³ or less.

In a preferred embodiment, one or more immunomodulatory agents are administered in combination with an antibody that immunospecifically binds to an EphA2 or EphrinA1 polypeptide to a subject at risk of or with an infection so as to transiently reduce or inhibit one or more aspects of the immune response. Such a transient inhibition or reduction of one or more aspects of the immune system can last for hours, days, weeks, or months. Preferably, the transient inhibition or reduction in one or more aspects of the immune response lasts for a few hours (e.g., 2 hours, 4 hours, 6 hours, 8 hours, 12 hours, 14 hours, 16 hours, 18 hours, 24 hours, 36 hours, or 48 hours), a few days (e.g., 3 days, 4 days, 5 days, 6 days, 7 days, or 14 days), or a few weeks (e.g., 3 weeks, 4 weeks, 5 weeks or 6 weeks). The transient reduction or inhibition of one or more aspects of the immune response enhances the prophylactic and/or therapeutic effect(s) of an antibody that immunospecifically binds to an EphA2 or EphrinA1 polypeptide.

In a preferred embodiment, proteins, polypeptides or peptides (including antibodies) that are utilized as immunomodulatory agents are derived from the same species as the recipient of the proteins, polypeptides or peptides so as to reduce the likelihood of an immune response to those proteins, polypeptides or peptides. In another preferred embodiment, when the subject is a human, the proteins, polypeptides, or peptides that are utilized as immunomodulatory agents are human or humanized.

Nucleic acid molecules encoding proteins, polypeptides, or peptides with immunomodulatory activity or proteins, polypeptides, or peptides with immunomodulatory activity can be administered to a subject at risk of or with an infection in accordance with the methods of the invention. Further, nucleic acid molecules encoding derivatives, analogs, or fragments of proteins, polypeptides, or peptides with immunomodulatory activity, or derivatives, analogs, or fragments of proteins, polypeptides, or peptides with immunomodulatory activity can be administered to a subject at risk of or with an infection in accordance with the methods of the invention. Preferably, such derivatives, analogs, and fragments retain the immunomodulatory activity of the full-length, wild-type protein, polypeptide, or peptide.

The immunomodulator activity of an immunomodulatory agent can be determined in vitro and/or in vivo by any technique well-known to one skilled in the art, including, e.g., by CTL assays, proliferation assays, immunoassays (e.g. ELISAs) for the expression of particular proteins such as co-stimulatory molecules and cytokines, and FACS.

### 5.2.6.2 Anti-Inflammatory Therapies

Any anti-inflammatory agent, including agents useful in therapies for inflammatory disorders, well-known to one of skill in the art can be used in the compositions and methods of the invention. Non-limiting examples of anti-inflammatory agents include non-steroidal anti-inflammatory drugs (NSAIDs), steroidal anti-inflammatory drugs, anticholinergics (e.g., atropine sulfate, atropine methylnitrate, and ipratropium bromide (ATROVENT™)), beta2-agonists (e.g., abuterol (VENTOLIN™ and PROVENTIL™), bitolterol (TORNALATE™), levalbuterol (XOPONEX™), metaproterenol (ALUPENT™), pirbuterol (MAXAIR™), terbutlaine (BRETHAIRE™ and BRETHINE™), albuterol (PROVENTIL™, REPETABS™, and VOLMAX™), formoterol (FORADIL AEROLIZER™), and salmeterol (SEREVEN™ and SEREVENT DISKUS™)), and methylxanthines (e.g., theophylline (UNIPHYL™, THEO-DUR™, SLO-BID™, AND TEHO-42™)). Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), naburnentone (RELAFEN™), sulindac (CLINORIL™), tolmentin (TOLECTIN™), rofecoxib (VIOXX™), naproxen (ALEVE™, NAPROSYN™), ketoprofen (ACTRON™) and naburnetone (RELAFEN™). Such NSAIDs function by inhibiting a cyclooxgenase enzyme (e.g., COX-1 and/or COX-2). Examples of steroidal anti-inflammatory drugs include, but are not limited to, glucocorticoids, dexamethasone (DECADRON™), corticosteroids (e.g., methylprednisolone (MEDROL™)), cortisone, hydrocortisone, prednisone (PREDNISONE™ and DELTASONE™), prednisolone (PRELONE™ and PEDIAPRED™), triamcinolone, azulfidine, and inhibitors of eicosanoids (e.g., prostaglandins, thromboxanes, and leukotrienes (e.g., montelukast (SINGULAIR™), zafirlukast (ACCOLATE™), pranlukast (ONON™), or zileuton (ZYFLO™)).

Anti-inflammatory therapies and their dosages, routes of administration, and recommended usage are known in the art and have been described in such literature as the Physicians' Desk Reference (59th ed., 2005).

### 5.2.6.3 Anti-Viral Therapies

Any anti-viral agent well-known to one of skill in the art can be used in the compositions and the methods of the invention. Non-limiting examples of anti-viral agents include proteins, polypeptides, peptides, fusion proteins antibodies, nucleic acid molecules, organic molecules, inorganic molecules, and small molecules that inhibit and/or reduce the attachment of a virus to its receptor, the internalization of a virus into a cell, the replication of a virus, or release of virus from a cell. In particular, anti-viral agents include, but are not limited to, nucleoside analogs (e.g., zidovudine, acyclovir, gangcyclovir, vidarabine, idoxutidine, tifilulldinc, and ribavirn), foscarnet, amantadine, rimantadine, saquinavir, indinavir, ritonavir, alpha-interferons and other interferons, and AZT.

In specific embodiments, the anti-viral agent is an immunomodulatory agent that is immunospecific for a viral antigen. As used herein, the term “viral antigen” includes, but is not limited to, any viral peptide, polypeptide and protein (e.g., HIV gp120, HIV nef, RSV F glycoprotein, RSV G glycoprotein, influenza virus neuraminidase, influenza virus hemagglutinin, HTLV tax, herpes simplex virus glycoprotein (e.g., gB, gC, gD, and gE) and hepatitis B surface antigen) that is capable of eliciting an immune response. Antibodies useful in this invention for treatment of a viral infection include, but are not limited to, antibodies against antigens of pathogenic viruses, including as examples and not by limitation: adenovirdiae (e.g., mastadenovirus and aviadenovirus), herpesviridae (e.g., herpes simplex virus 1, herpes simplex virus 2, herpes simplex virus 5, and herpes simplex virus 6), leviviridae (e.g., levivirus, enterobacteria phase MS2, allolevirus), poxviridae (e.g., chordopoxvirinae, parapoxvirus, avipoxvirus, capripoxvirus, leporiipoxvirus, suipoxvirus, molluscipoxvirus, and entomopoxvirinae), papovaviridae (e.g., polyomavirus and papillomavirus), paramyxoviridae (e.g., paramyxovirus, parainfluenza virus 1, mobillivirus (e.g., measles virus), rubulavirus (e.g., mumps virus), pneumonovirinae (e.g., pneumovirus, human respiratory synctial virus), and metapneumovirus (e.g., avian pneumovirus and human metapneumovirus)), picornaviridae (e.g., enterovirus, rhinovirus, hepatovirus (e.g., human hepatits A virus), cardiovirus, and apthovirus), reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLV-HTLV retroviruses, lentivirus (e.g. human immunodeficiency virus 1 and human immunodeficiency virus 2), spumavirus), flaviviridae (e.g., hepatitis C virus), hepadnaviridae (e.g., hepatitis B virus), togaviridae (e.g., alphavirus (e.g., sindbis virus) and rubivirus (e.g., rubella virus)), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, and lassa virus), and coronaviridae (e.g., coronavirus and torovirus).

Specific examples of antibodies available useful for the treatment of a viral infection include, but are not limited to, PRO542 (Progenics) which is a CD4 fusion antibody useful for the treatment of HIV infection; Ostavir (Protein Design Labs, Inc., CA) which is a human antibody useful for the treatment of hepatitis B virus; and Protovir (Protein Design Labs, Inc., CA) which is a humanized IgG1 antibody useful for the treatment of cytomegalovirus (CMV); and palivizumab (SYNAGIS®; MedImmune, Inc.; International Publication No. WO 02/43660) which is a humanized antibody useful for treatment of RSV.

In a specific embodiment, the anti-viral agents used in the compositions and methods of the invention inhibit or reduce a virus infection, inhibit or reduce the replication of a virus that causes an infection, or inhibit or reduce the spread of a virus that causes an infection to other cells or subjects. In another specific embodiment, the anti-viral agents used in the compositions and methods of the invention inhibit or reduce infection by RSV, hMPV, or PIV, inhibit or reduce the replication of RSV, hMPV, or PIV, or inhibit or reduce the spread of RSV, hMPV, or PIV to other cells or subjects. Examples of such agents and methods of treatment of RSV, hMPV, and/or PIV infections include, but are not limited to, nucleoside analogs, such as zidovudine, acyclovir, gangcyclovir, vidarabine, idoxuridine, trifluridine, and ribavirin, as well as foscarnet, amantadine, rimantadine, saquinavir, indinavir, ritonavir, and the alpha-interferons. See U.S. Prov. Patent App. No. 60/398,475 filed Jul. 25, 2002, entitled “Methods of Treating and Preventing RSV, HMPV, and PIV Using Anti-RSV, Anti-HMPV, and Anti-PIV Antibodies,” and U.S. patent application Ser. No. 10/371,122 filed Feb. 21, 2003, which are incorporated herein by reference in its entirety.

In specific embodiments, the viral infection is RSV and the anti-viral antigen is an antibody that immunospecifically binds to an antigen of RSV. In certain embodiments, the anti-RSV-antigen antibody binds immunospecifically to an RSV antigen of the Group A of RSV. In other embodiments, the anti-RSV-antigen antibody binds immunospecifically to an RSV antigen of the Group B of RSV. In other embodiments, an antibody binds to an antigen of RSV of one Group and cross reacts with the analogous antigen of the other Group. In particular embodiments, the anti-RSV-antigen antibody binds immunospecifically to a RSV nucleoprotein, RSV phosphoprotein, RSV matrix protein, RSV small hydrophobic protein, RSV RNA-dependent RNA polymerase, RSV F protein, and/or RSV G protein. In additional specific embodiments, the anti-RSV-antigen antibody binds to allelic variants of a RSV nucleoprotein, a RSV nucleocapsid protein, a RSV phosphoprotein, a RSV matrix protein, a RSV attachment glycoprotein, a RSV fusion glycoprotein, a RSV nucleocapsid protein, a RSV matrix protein, a RSV small hydrophobic protein, a RSV RNA-dependent RNA polymerase, a RSV F protein, a RSV L protein, a RSV P protein, and/or a RSV G protein.

It should be recognized that antibodies that immunospecifically bind to a RSV antigen are known in the art. For example, palivizumab (SYNAGIS®)) is a humanized monoclonal antibody presently used for the prevention of RSV infection in pediatric patients. In a specific embodiment, an antibody to be used with the methods of the present invention is palivizumab or an antibody-binding fragment thereof (e.g., a fragment containing one or more complementarity determining regions (CDRs) and preferably, the variable domain of palivizumab). The amino acid sequence of palivizumab is disclosed, e.g., in Johnson et al., 1997, J. Infection 176:1215-1224, and U.S. Pat. No. 5,824,307 and International Application Publication No.: WO 02/43660, entitled “Methods of Administering/Dosing Anti-RSV Antibodies for Prophylaxis and Treatment”, by Young et al., which are incorporated herein by reference in their entireties.

One or more antibodies or antigen-binding fragments thereof that bind immunospecifically to a RSV antigen comprise a Fc domain with a higher affinity for the FcRn receptor than the Fc domain of palivizumab can also be used in accordance with the invention. Such antibodies are described in U.S. patent application No. 10/020,354, filed Dec. 12, 2001, which is incorporated herein by reference in its entireties. Further, one or more of the anti-RSV-antigen antibodies A4B4; P12f2 P12f4; P11d4; Ale9; A12a6; A13c4; A17d4; A4B4; 1X-493L1; FR H3-3F4; M3H9; Y10H6; DG; AFFF; AFFF(1); 6H8; L1-7E5; L2-15B10; A13a11; A1h5; A4B4(1);A4B4-F52S; or A4B4L1FR-S28R can be used in accordance with the invention. These antibodies are disclosed in International Application Publication No.: WO 02/43660, entitled “Methods of Administering/Dosing Anti-RSV Antibodies for Prophylaxis and Treatment”, by Young et al., and U.S. Provisional Patent Application 60/398,475 filed Jul. 25, 2002, entitled “Methods of Treating and Preventing RSV, HMPV, and PIV Using Anti-RSV, Anti-HMPV, and Anti-PIV Antibodies” which are incorporated herein by reference in their entireties.

In certain embodiments, the anti-RSV-antigen antibodies are the anti-RSV-antigen antibodies of or are prepared by the methods of U.S. application Ser. No: 09/724,531, filed Nov. 28, 2000; U.S. Ser. No. 09/996,288, filed Nov. 28, 2001; and U.S. Pat. Publication No. US2003/0091584 A1, published May 15, 2003, all entitled “Methods of Administering/Dosing Anti-RSV Antibodies for Prophylaxis and Treatment”, by Young et al., which are incorporated by reference herein in their entireties. Methods and composition for stabilized antibody formulations that can be used in the methods of the present invention are disclosed in U.S. Provisional Application Nos. 60/388,921, filed Jun. 14, 2002, and 60/388,920, filed Jun. 14, 2002, which are incorporated by reference herein in their entireties.

Anti-viral therapies and their dosages, routes of administration and recommended usage are known in the art and have been described in such literature as the Physicians' Desk Reference (59^{th} ed., 2005). Additional information on respiratory viral infections is available in Cecil Textbook of Medicine (18th ed., 1988).

### 5.2.6.4 Anti-Bacterial Agents

Anti-bacterial agents and therapies well known to one of skill in the art for the prevention, treatment, management, or amelioration of bacterial infections can be used in the compositions and methods of the invention. Non-limiting examples of anti-bacterial agents include proteins, polypeptides, peptides, fusion proteins, antibodies, nucleic acid molecules, organic molecules, inorganic molecules, and small molecules that inhibit or reduce a bacterial infection, inhibit or reduce the replication of bacteria, or inhibit or reduce the spread of bacteria to other subjects. In particular, examples of anti-bacterial agents include, but are not limited to, penicillin, cephalosporin, imipenem, axtreonam, vancomycin, cycloserine, bacitracin, chloramphenicol, erythromycin, clindamycin, tetracycline, streptomycin, tobramycin, gentamicin, amikacin, kanamycin, neomycin, spectinomycin, trimethoprim, norfloxacin, rifampin, polymyxin, amphotericin B, nystatin, ketocanazole, isoniazid, metronidazole, and pentamidine.

In a preferred embodiment, the anti-bacterial agent is an agent that inhibits or reduces a bacterial infection, inhibits or reduces the replication of a bacteria that causes an infection, or inhibits or reduces the spread of a bacteria that causes an infection to other subjects. In cases in which the bacterial infection is a mycoplasma infection (e.g., pharyngitis, tracheobronchitis, and pneumonia), the anti-bacterial agent is preferably a tetracycline, erythromycin, or spectinomycin. In cases in which the bacterial infection is tuberculosis, the anti-bacterial agent is preferably, rifampcin, isonaizid, pyranzinamide, ethambutol, and streptomycin.

Anti-bacterial therapies and their dosages, routes of administration and recommended usage are known in the art and have been described in such literature as the Physicians' Desk Reference (59^{th} ed., 2005). Additional information on respiratory infections and anti-bacterial therapies is available in Cecil Textbook of Medicine (18th ed., 1988).

### 5.2.6.5 Anti-Fungal Agents

Anti-fungal agents and therapies well known to one of skill in the art for prevention, management, treatment, and/or amelioration of a fungal infection or one or more symptoms thereof (e.g., a fungal respiratory infection) can be used in the compositions and methods of the invention. Non-limiting examples of anti-fungal agents include proteins, polypeptides, peptides, fusion proteins, antibodies, nucleic acid molecules, organic molecules, inorganic molecules, and small molecules that inhibit and/or reduce fungal infection, inhibit and/or reduce the replication of fungi, or inhibit and/or reduce the spread of fungi to other subjects. Specific examples of anti-fungal agents include, but are not limited to, azole drugs (e.g., miconazole, ketoconazole (NIZORAL®), caspofungin acetate (CANCIDAS®), imidazole, triazoles (e.g., fluconazole (DIFLUCAN®)), and itraconazole (SPORANOX®)), polyene (e.g., nystatin, amphotericin B (FUNGIZONE®), amphotericin B lipid complex (“ABLC”)(ABELCET®), amphotericin B colloidal dispersion (“ABCD”)(AMPHOTEC®), liposomal amphotericin B (AMBISONE®)), potassium iodide (KI), pyrimidine (e.g., flucytosine (ANCOBON®)), and voriconazole (VFEN®). See, e.g., Table 6, infra for a list of specific anti-fungal agents and their recommended dosages.

TABLE 6
Anti-fungal Agents

| Anti-fungal Agent | Dosage |
|---|---|
| Amphotericin B | |
| ABELCET( ®) | 5 mg/kg/day |
| (lipid complex injection) | |
| AMBISOME( ®) | 3-5 mg/kg/day |
| (liposome for injection) | |
| AMPHOTEC( ®) | 3-4 mg/kg/day |
| (complex for injection) | |
| Caspofungin acetate | 70 mg on day one |
| (CANCIDAS ®) | followed by 50 mg/day |
| Fluconazole | up to 400 mg/day (adults) |
| (DIFLUCAN ®) | up to 12 mg/kg/day (children) |
| Itraconazole | 200-400 mg/day |
| (SPORANOX ®) | |
| Flucytosine | 50-150 mg/kg/day in divided |
| (ANCOBON ®) | dose every 6 hours |
| Liposomal nystatin | 1-4 mg/kg |
| Ketoconazole | 200 mg single daily dose up to |
| (NIZORAL ®) | 400 mg/day in two divided doses |
| | (adults) |
| | 3.3-6.6 mg/kg/day for children |
| | 2 years old and older |
| Voriconazole | 6 mg/kg i.v. loading dose every 12 |
| (VFEND ®) | hours for two doses, followed by |
| | maintenance dose of 4 mg/kg i.v. |
| | every 12 hours, then oral maintenance |
| | dose of 200-100 mg tablet |

In certain embodiments, the anti-fungal agent is an agent that inhibits or reduces a fungal infection, inhibits or reduces the replication of a fungus that causes an infection, or inhibits or reduces the spread of a fungus that causes an infection to other subjects. In cases in which the fungal infection is Blastomyces dermatitidis, the anti-fungal agent is preferably itraconazole, amphotericin B, fluconazole, or ketoconazole. In cases in which the fungal infection is pulmonary aspergilloma, the anti-fungal agent is preferably amphotericin B, liposomal amphotericin B, itraconazole, or fluconazole. In cases in which the fungal infection is histoplasmosis, the anti-fungal agent is preferably amphotericin B, itraconazole, fluconazole, or ketoconazole. In cases in which the fungal infection is coccidioidomycosis, the anti-fungal agent is preferably fluconazole or amphotericin B. In cases in which the fungal infection is cryptococcosis, the anti-fungal agent is preferably amphotericin B, fluconazole, or combination of the two agents. In cases in which the infection is chromomycosis, the anti-fungal agent is preferably itraconazole, fluconazole, or flucytosine. In cases in which the fungal infection is mucormycosis, the anti-fungal agent is preferably amphotericin B or liposomal amphotericin B. In cases in which the pulmonary or respiratory fungal infection is pseudoallescheriasis, the anti-fungal agent is preferably itraconazole ore miconazole.

Anti-fungal therapies and their dosages, routes of administration, and recommended usage are known in the art and have been described in such literature as Dodds et al., 2000 Pharmacotherapy 20(11) 1335-1355, the Physicians' Desk Reference (59th ed., 2005) and the Merk Manual of Diagnosis and Therapy (17th ed., 1999).

### 5.2.6.6 Anti-Protozoan Agents

Anti-protozoan agents and therapies well known to one of skill in the art for prevention, management, treatment, and/or amelioration of a protozoa infection or one or more symptoms thereof (e.g., a respiratory infection associated with a protozoa infection) can be used in the compositions and methods of the invention. Non-limiting examples of anti-protozoan agents include proteins, polypeptides, peptides, fusion proteins, antibodies, nucleic acid molecules, organic molecules, inorganic molecules, and small molecules that inhibit and/or reduce a protozoa infection, inhibit and/or reduce the replication of protozoa, or inhibit and/or reduce the spread of protozoa to other subjects. Specific examples of anti-protozoan agents include, but are not limited to, chloroquine phosphate (Aralen™); quinine sulfate plus one of the following: doxycycline, tetracycline, or clindamycin; atovaquone-proguanil (Malarone™); Mefloquine (Lariam™); metronidazole (Flagyl); tinidazole (Tindamax); 5-nitroimidazole (omidazole), and agents described in U.S. Pat. No. 6,440,936.

In certain embodiments, the anti-protozoan agent is an agent that inhibits or reduces a protozoa infection, inhibits or reduces the replication of a protozoa that causes an infection, or inhibits or reduces the spread of a protozoa that causes an infection to other subjects. In cases in which the protozoan infection is Trichomoniasis, the anti-protozoan agent is preferably metronidazole (Flagyl), tinidazole (Tindamax), or 5-nitroimidazole (omidazole). In cases in which the protozoan infection is malaria, the anti-protozan agent is preferably chloroquine phosphate (Aralen™); quinine sulfate plus one of the following: doxycycline, tetracycline, or clindamycin; quinidine gluconate plus one of the following: docycycline, tetracycline, or clindamycin; Fansidar™; Malarone™ (atovaquone 250 mg plus proguanil 100 mg); or Mefloquine (Larium™).

Anti-protozoan therapies and their dosages, routes of administration, and recommended usage are known in the art and have been described in such literature as Dodds et al., 2000 Pharmacotherapy 20(11) 1335-1355, the Physicians' Desk Reference (59th ed., 2005); the Merk Manual of Diagnosis and Therapy (17th ed., 1999); and publications provided by the Centers for Disease Control and Prevention (CDC; http://www.cdc.gov) (Atlanta, Ga.).

### 5.3 Biological Assays

### 5.3.1 Immunospecificity of Antibodies

Antibodies of the present invention or fragments thereof may be characterized in a variety of ways well-known to one of skill in the art. In particular, antibodies of the invention or fragments thereof may be assayed for the ability to immunospecifically bind to EphA2 or EphrinA1. Such an assay may be performed in solution (e.g., Houghten, 1992, Bio/Techniques 13:412-421), on beads (Lam, 1991, Nature 354:82-84), on chips (Fodor, 1993, Nature 364:555-556), on bacteria (U.S. Pat. No. 5,223,409), on spores (U.S. Pat. Nos. 5,571,698; 5,403,484; and 5,223,409), on plasmids (Cull et al., 1992, Proc. Natl. Acad. Sci. USA 89:1865-1869) or on phage (Scott and Smith, 1990, Science 249:386-390; Cwirla et al., 1990, Proc. Natl. Acad. Sci. USA 87:6378-6382; and Felici, 1991, J. Mol. Biol. 222:301-310) (each of these references is incorporated herein in its entirety by reference). Antibodies or fragments thereof that have been identified to immunospecifically bind to EphA2 or Ephrin A1 can then be assayed for their specificity and affinity for an EphA2 or EphrinA1.

The antibodies of the invention or fragments thereof may be assayed for immunospecific binding to EphA2 or EphrinA1 and cross-reactivity with other antigens by any method known in the art. Immunoassays which can be used to analyze immunospecific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds., 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1 to 4 hours) at 40° C., adding protein A and/or protein G sepharose beads to the cell lysate, incubating for about an hour or more at 40° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al., eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, incubating the membrane in blocking solution (e.g., PBS with 3% BSA or non-fat milk), washing the membrane in washing buffer (e.g., PBS-Tween 20), incubating the membrane with primary antibody (the antibody of interest) diluted in blocking buffer, washing the membrane in washing buffer, incubating the membrane with a secondary antibody (which recognizes the primary antibody, e.g., an anti-human antibody) conjugated to an enzymatic substrate (e.g.,. horseradish peroxidase or alkaline phosphatase) or radioactive molecule (e.g., ³²P or ¹²⁵D diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of the present invention or a fragment thereof for EphA2 or EphrinA1 and the binding off-rates can be determined from the data by scatchard plot analysis. Competition with a second antibody can also be determined using radioimmunoassays. In this case, EphA2 or EphrinA1 is incubated with an antibody of the present invention conjugated to a labeled compound (e.g., ³H or ¹²⁵I) in the presence of increasing amounts of an unlabeled second antibody.

In a preferred embodiment, BIAcore kinetic analysis is used to determine the binding on and off rates of antibodies of the invention to EphA2 or EphrinA1. BIAcore kinetic analysis comprises analyzing the binding and dissociation of EphA2 or EphrinA1 from chips with immobilized antibodies of the invention on their surface. A typical BIAcore kinetic study involves the injection of 250 uL of an antibody reagent (mAb, Fab) at varying concentration in HBS buffer containing 0.005% Tween-20 over a sensor chip surface, onto which has been immobilized the antigen. The flow rate is maintained constant at 75 uL/min. Dissociation data is collected for 15 min. or longer as necessary. Following each injection/dissociation cycle, the bound mAb is removed from the antigen surface using brief, 1 min. pulses of dilute acid, typically 10-100 mM HCl, though other regenerants are employed as the circumstances warrant. More specifically, for measurement of the rates of association, kₒₙ, and dissociation, k_{off}, the antigen is directly immobilized onto the sensor chip surface through the use of standard amine coupling chemistries, namely the EDC/NHS method (EDC=N-diethylaminopropyl)-carbodiimide). Briefly, a 5-100 nM solution of the antigen in 10 mM NaOAc, pH4 or pH5 is prepared and passed over the EDC/NHS-activated surface until approximately 30-50 RU's worth of antigen are immobilized. Following this, the unreacted active esters are “capped” off with an injection of 1M Et-NH2. A blank surface, containing no antigen, is prepared under identical immobilization conditions for reference purposes. Once an appropriate surface has been prepared, a suitable dilution series of each one of the antibody reagents is prepared in HBS/Tween-20, and passed over both the antigen and reference cell surfaces, which are connected in series. The range of antibody concentrations that are prepared varies, depending on what the equilibrium binding constant, KD, is estimated to be. As described above, the bound antibody is removed after each injection/dissociation cycle using an appropriate regenerant.

The antibodies of the invention or fragments thereof can also be assayed for their ability to inhibit the binding of EphA2 or EphrinA1 to its host cell receptor or ligand, respectively, using techniques known to those of skill in the art. For example, cells expressing EphrinA1 can be contacted with EphA2 in the presence or absence of an antibody or fragment thereof and the ability of the antibody or fragment thereof to inhibit EphA2's binding can measured by, for example, flow cytometry or a scintillation assay. EphA2 or the antibody or antibody fragment can be labeled with a detectable compound such as a radioactive label (e.g., 32P, 35S, and 125I) or a fluorescent label (e.g., fluorescein isothiocyanate, rhodamine, phycoerythrin, phycocyanin, allophycocyanin, o-phthaldehyde and fluorescamine) to enable detection of an interaction between EphA2 and its host cell receptor. Alternatively, the ability of antibodies or fragments thereof to inhibit EphA2 from binding to its receptor can be determined in cell-free assays. For example, EphA2 can be contacted with an antibody or fragment thereof and the ability of the antibody or antibody fragment to inhibit the EphA2 from binding to its host cell receptor can be determined. Preferably, the antibody or the antibody fragment is immobilized on a solid support and EphA2 is labeled with a detectable compound. Alternatively, EphA2 is immobilized on a solid support and the antibody or fragment thereof is labeled with a detectable compound. EphA2 may be partially or completely purified (e.g., partially or completely free of other polypeptides) or part of a cell lysate. Further, EphA2 may be a fusion protein comprising EphA2, a derivative, analog or fragment thereof and a domain such as glutathionine-S-transferase. Alternatively, EphA2 can be biotinylated using techniques well known to those of skill in the art (e.g., biotinylation kit, Pierce Chemicals; Rockford, Ill.).

### 5.3.2 In Vitro Studies

The EphA2/EphrinA1 Modulators, compositions, or combination therapies of the invention can be tested in vitro and/or in vivo for their ability to modulate the biological activity of immune cells (e.g., T cells, neutrophils, and mast cells), endothelial cells, and epithelial cells. The ability of an EphA2/EphrinA1 Modulator, composition, or combination therapy of the invention to modulate the biological activity of immune cells (e.g., T cells, B cells, mast cells, macrophages, neutrophils, and eosinophils), endothelial cells, and epithelial cells can be assessed by: detecting the expression of antigens (e.g., activation of genes by EphA2) and genes involved in lymphocyte activation (e.g., Lgamma-6A/E)); detecting the proliferation of immune cells, endothelia cells and/or epithelial cells; detecting the activation of signaling molecules; detecting the effector function of immune cells (e.g., T cells, B cells, mast cells, macrophages, neutrophils, and eosinophils), endothelial cells, and/or epithelial cells; or detecting the differentiation of immune cells, endothelial cells, and/or epithelial cells. Techniques known to those of skill in the art can be used for measuring these activities. For example, cellular proliferation can be assayed by ³H-thymidine incorporation assays and trypan blue cell counts. Antigen expression can be assayed, for example, by immunoassays including, but are not limited to, competitive and non-competitive assay systems using techniques such as western blots, immunohistochemistry radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, and FACS analysis. The activation of signaling molecules can be assayed, for example, by kinase assays and electrophoretic shift assays (EMSAs). Mast cell degranulation can be assayed, for example by measuring serotonin (5-HT) release or histamine release with high-performance liquid chromatogoraphy (see, e.g., Taylor et al. 1995 Immunology 86(3): 427-433 and Kurosawa et al., 1998 Clin Exp Allergy 28(8): 1007-1012).

The EphA2/EphrinA1 Modulators, compositions, or combination therapies of the invention are preferably tested in vitro and then in vivo for the desired therapeutic or prophylactic activity prior to use in humans. For example, assays which can be used to determine whether administration of a specific pharmaceutical composition is indicated include cell culture assays in which a patient tissue sample is grown in culture and exposed to, or otherwise contacted with, a pharmaceutical composition, and the effect of such composition upon the tissue sample is observed. The tissue sample can be obtained by biopsy from the patient. This test allows the identification of the therapeutically most effective therapy (e.g., prophylactic or therapeutic agent) for each individual patient. In various specific embodiments, in vitro assays can be carried out with representative cells of cell types involved an infection (e.g., epithelial cells) to determine if a pharmaceutical composition of the invention has a desired effect upon such cell types.

The effect of an EphA2/EphrinA1 Modulator, a composition, or a combination therapy of the invention on peripheral blood lymphocyte counts can be monitored/assessed using standard techniques known to one of skill in the art. Peripheral blood lymphocytes counts in a subject can be determined by, e.g., obtaining a sample of peripheral blood from said subject, separating the lymphocytes from other components of peripheral blood such as plasma using, e.g., Ficoll-Hypaque (Pharmacia) gradient centrifugation, and counting the lymphocytes using trypan blue. Peripheral blood T-cell counts in subject can be determined by, e.g., separating the lymphocytes from other components of peripheral blood such as plasma using, e.g., a use of Ficoll-Hypaque (Pharmacia) gradient centrifugation, labeling the T-cells with an antibody directed to a T-cell antigen which is conjugated to FITC or phycoerythrin, and measuring the number of T-cells by FACS.

The methods of the invention for treating, managing, preventing, and/or ameliorating a viral infection or one or more symptoms thereof can be tested for their ability to inhibit viral replication or reduce viral load in in vitro assays. For example, viral replication can be assayed by a plaque assay such as described, e.g., by Johnson et al., 1997, Journal of Infectious Diseases 176:1215-1224 176:1215-1224. The EphA2/EphrinA1 Modulators, compositions, or combination therapies administered according to the methods of the invention can also be assayed for their ability to inhibit or downregulate the expression of viral polypeptides. Techniques known to those of skill in the art, including, but not limited to, western blot analysis, northern blot analysis, and RT-PCR can be used to measure the expression of viral polypeptides.

The methods of the invention for preventing, treating, managing, and/or ameliorating a bacterial infection or one or more symptoms thereof can be tested for activity against bacteria causing infections in in vitro assays well-known in the art. In vitro assays known in the art can also be used to test the existence or development of resistance of bacteria to a therapy (e.g., an EphA2/EphrinA1 Modulator, other prophylactic or therapeutic agent, a combination thereof, or a composition thereof) of the invention. Such in vitro assays are described in Gales et al., 2002, Diag. Nicrobiol. Infect. Dis. 44(3):301-311; Hicks et al., 2002, Clin. Microbiol. Infect. 8(11): 753-757; and Nicholson et al., 2002, Diagn. Microbiol. Infect. Dis. 44(1): 101-107.

The therapies (e.g., an EphA2/EphrinA1 Modulator alone or in combination with prophylactic or therapeutic agents, other than antibodies of the invention) of the invention for treating, managing, preventing, and/or ameliorating a fungal infection or one or more symptoms thereof can be tested for anti-fungal activity against different species of fungus. Any of the standard anti-fungal assays well-known in the art can be used to assess the anti-fungal activity of a therapy. The anti-fungal effect on different species of fungus can be tested. The tests recommended by the National Committee for Clinical Laboratories (NCCLS) (See National Committee for Clinical Laboratories Standards. 1995, Proposed Standard M27T. Villanova, Pa., all of which is incorporated herein by reference in its entirety) and other methods known to those skilled in the art (Pfaller et al., 1993, Infectious Dis. Clin. N. Am. 7: 435-444) can be used to assess the anti-fungal effect of a therapy. The antifungal properties of a therapy may also be determined from a fungal lysis assay, as well as by other methods, including, inter alia, growth inhibition assays, fluorescence-based fungal viability assays, flow cytometry analyses, and other standard assays known to those skilled in the art.

For example, the anti-fungal activity of a therapy can be tested using macrodilution methods and/or microdilution methods using protocols well-known to those skilled in the art (see, e.g., Clancy et al., 1997 Journal of Clinical Microbiology, 35(11): 2878-82; Ryder et al., 1998, Antimicrobial Agents and Chemotherapy, 42(5): 1057-61; U.S. Pat. No. 5,521,153; U.S. Pat. No. 5,883,120, U.S. Pat. No. 5,521,169, all of which are incorporated by reference in their entirety). Briefly, a fungal strain is cultured in an appropriate liquid media, and grown at an appropriate temperature, depending on the particular fungal strain used for a determined amount of time, which is also depends on the particular fungal strain used. An innoculum is then prepared photometrically and the turbidity of the suspension is matched to that of a standard, e.g., a McFarland standard. The effect of a therapy on the turbidity of the inoculum is determined visually or spectrophotometrically. The minimal inhibitory concentration (“MIC”) of the therapy is determined, which is defined as the lowest concentration of the lead compound which prevents visible growth of an inoculum as measured by determining the culture turbidity.

The anti-fungal activity of a therapy can also be determined utilizing colorimetric based assays well-known to one of skill in the art. One exemplary colorimetric assay that can be used to assess the anti-fungal activity of a therapy is described by Pfaller et al., 1994, Journal of Clinical Microbiology, 32(8): 1993-6, which is incorporated herein by reference in its entirety; also see Tiballi et al., 1995, Journal of Clinical Microbiology, 33(4): 915-7). This assay employs a colorimetric endpoint using an oxidation-reduction indicator (Alamar Biosciences, Inc., Sacramento, Calif.).

The anti-fungal activity of a therapy can also be determined utilizing photometric assays well-known to one of skill in the art (see, e.g., Clancy et al., 1997 Journal of Clinical Microbiology, 35(11): 2878-82; Jahn et al., 1995, Journal of Clinical Microbiology, 33(3): 661-667, each of which is incorporated herein by reference in its entirety). This photometric assay is based on quantifying mitochondrial respiration by viable fungi through the reduction of 3-(4,5-dimethyl-2thiazolyl)-2,5,-diphenyl-2H-tetrazolium bromide (MTT) to formazan. MIC's determined by this assay are defined as the highest concentration of the test therapy associated with the first precipitous drop in optical density. In some embodiments, the therapy is assayed for anti-fungal activity using macrodilution, microdilution and MTT assays in parallel.

Further, any in vitro assays known to those skilled in the art can be used to evaluate the prophylactic and/or therapeutic utility of an antibody, a composition, a combination therapy disclosed herein for a respiratory infection or one or more symptoms thereof.

### 5.3.3 In Vivo Assays

The EphA2/EphrinA1 Modulators, compositions, or combination therapies of the invention can be tested in suitable animal model systems prior to use in humans. Such animal model systems include, but are not limited to, rats, mice, chicken, cows, monkeys, pigs, dogs, rabbits, etc. Any animal system well-known in the art may be used. Several aspects of tile procedure may vary; said aspects include, but are not limited to, the temporal regime of administering the therapies (e.g., prophylactic and/or therapeutic agents), whether such therapies are administered separately or as an admixture, and the frequency of administration of the therapies.

Animal models for viral infections can also be used to assess the efficacy of an EphA2/EphrinA1 Modulator, a composition, or a combination therapy of the invention. Animal models for viral infections such as EBV-associated diseases, gammaherpesviruses, infectious mononucleosis, simian immunodeficiency virus (“SIV”), Borna disease virus infection, hepatitis, varicella virus infection, viral pneumonitis, Epstein-Barr virus pathogenesis, feline immunodeficiency virus (“FIV”), HTLV type 1 infection, human rotaviruses, and genital herpes have been developed (see, e.g., Hayashi et al., 2002, Histol Histopathol 17(4):1293-310; Arico et al., 2002, J Interferon Cytokine Res 22(11):1081-8; Flano et al., 2002, Immunol Res 25(3):201-17; Sauermann, 2001, Curr Mol Med 1(4):515-22; Pletnikov et al., 2002, Front Biosci 7:d593-607; Engler et al., 2001, Mol Immunol 38(6):457-65; White et al., 2001, Brain Pathol 11(4):475-9; Davis & Matalon, 2001, News Physiol Sci 16:185-90; Wang, 2001, Curr Top Microbiol Immunol. 258:201-19; Phillips et al., 2000, J Psychopharmacol. 14(3):244-50; Kazanji, 2000, AIDS Res Hum Retroviruses. 16(16):1741-6; Saif et al., 1996, Arch Virol Suppl. 12:153-61; and Hsiung et al., 1984, Rev Infect Dis. 6(1):33-50).

Animal models for viral respiratory infections such as, but not limited to, PIV (see, e.g., Shephard et al., 2003 Res Vet Sci 74(2): 187-190; Ottolini et al., 2002 J Infect Dis 186(12): 1713-1717), RSV (see, e.g., Culley et al., 2002 J Exp Med 196(10): 1381-1386; and Curtis et al., 2002 Exp Biol Med 227(9): 799-802) have been developed. In a specific embodiment, cotton rats are administered an antibody of the invention, a composition, or a combination therapy according to the methods of the invention, challenged with 10⁵ pfu of RSV, and four or more days later the rats are sacrificed and RSV titer and anti-RSV antibody serum titer is determined. Accordingly, a dosage that results in a 2 log decrease or a 99% reduction in RSV titer in the cotton rat challenged with 10⁵ pfU of RSV relative to the cotton rat challenged with 10⁵ pfU of RSV but not administered the formulation is the dosage of the formulation that can be administered to a human for the treatment, prevention or amelioration of one or more symptoms associated with RSV infection. Further, in accordance with this embodiment, the tissues (e.g., the lung tissues) from the sacrificed rats can be examined for histological changes.

The EphA2/EphrinA1 Modulators, compositions, or combination therapies of the invention can be tested for their ability to decrease the time course of viral infection. The EphA/EphrinA1 Modulators, compositions, or combination therapies of the invention can also be tested for their ability to increase the survival period of humans suffering from a viral infection by at least 25%, preferably at least 50%, at least 60%, at least 75%, at least 85%, at least 95%, or at least 99%. Further, antibodies, compositions, or combination therapies of the invention can be tested for their ability reduce the hospitalization period of humans suffering from viral infection by at least 60%, preferably at least 75%, at least 85%, at least 95%, or at least 99%. Techniques known to those of skill in the art can be used to analyze the function of the EphA2/EphrinA1 Modulators, compositions, or combination therapies of the invention in vivo.

Animal models for bacterial infections can also be used to assess the efficacy of an EphA2/EphrinA1 Modulator, a composition, or a combination therapy of the invention. Animal models for bacterial infections such as H. pylori-infection, genital mycoplasmosis, primary sclerosing cholangitis, cholera, chronic lung infection with Pseudomonas aeruginosa, Legionnaires' disease, gastroduodenal ulcer disease, bacterial meningitis, gastric Helicobacter infection, pneumococcal otitis media, experimental allergic neuritis, leprous neuropathy, mycobacterial infection, endocarditis, Aeromonas-associated enteritis, Bacteroides fragilis infection, syphilis, streptococcal endocarditis, acute hematogenous osteomyelitis, human scrub typhus, toxic shock syndrome, anaerobic infections, Escherichia coli infections, and Mycoplasma pneumoniae infections have been developed (see, e.g., Sugiyama et al., 2002, J Gastroenterol. 37 Suppl 13:6-9; Brown et al., 2001, Am J Reprod Immunol. 46(3):232-41; Vierling, 2001, Best Pract Res Clin Gastroenterol. 15(4):591-610; Klose, 2000, Trends Microbiol. 8(4):189-91; Stotland et al., 2000, Pediatr Pulmonol. 30(5):413-24; Brieland et al., 2000, Immunopharmacology 48(3):249-52; Lee, 2000, Baillieres Best Pract Res Clin Gastroenterol. 14(1):75-96; Koedel & Pfister, 1999, Infect Dis Clin North Am. 13(3):549-77; Nedrud, 1999, FEMS lmmunol Med Microbiol. 24(2):243-50; Prellner et al., 1999, Microb Drug Resist. 5(1):73-82; Vriesendorp, 1997, J Infect Dis. 176 Suppl 2:S164-8; Shetty & Antia, 1996, Indian J Lepr. 68(1):95-104; Balasubramanian et al., 1994, Immunobiology 191(4-5):395-401; Carbon et al., 1994, Int J Biomed Comput. 36(1-2):59-67; Haberberger et al., 1991, Experientia. 47(5):426-9; Onderdonk et al., 1990, Rev Infect Dis. 12 Suppl 2:S169-77; Wicher & Wicher, 1989, Crit Rev Microbiol. 16(3):181-234; Scheld, 1987, J Antimicrob Chemother. 20 Suppl A:71-85; Emslie & Nade, 1986, Rev Infect Dis. 8(6):841-9; Ridgway et al., 1986, Lab Anim Sci. 36(5):481-5; Quimby & Nguyen, 1985, Crit Rev Microbiol. 12(1):1-44; Onderdonk et al., 1979, Rev Infect Dis. 1(2):291-301; Smith, 1976, Ciba Found Symp. (42):45-72, and Taylor-Robinson, 1976, Infection, 4(1 Suppl):4-8).

The EphA2/EphrinA1 Modulators, compositions, or combination therapies of the invention can be tested for their ability to decrease the time course of bacterial infection, preferably bacterial respiratory infection by at least 25%, preferably at least 50%, at least 60%, at least 75%, at least 85%, at least 95%, or at least 99%. The EphA2/EphrinA1 Modulators, compositions, or combination therapies of the invention can also be tested for their ability to increase the survival period of humans suffering from a bacterial infection by at least 25%, preferably at least 50%, at least 60%, at least 75%, at least 85%, at least 95%, or at least 99%. Further, the EphA2/EphrinA1 Modulators, compositions, or combination therapies administered according to the methods of the invention can be tested for their ability reduce the hospitalization period of humans suffering from bacterial infection, by at least 60%, preferably at least 75%, at least 85%, at least 95%, or at least 99%. Techniques known to those of skill in the art can be used to analyze the finction of the EphA2/EphrinA1 Modulators, compositions, or combination therapies of the invention in vivo.

The efficacy of the EphA2/EphrinA1 Modulators, compositions, or combination therapies of the invention for the prevention, management, treatment, or amelioration of a fungal infection can be assessed in animal models for such infections. Animal models for fungal infections such as Candida infections, zygomycosis, Candida mastitis, progressive disseminated trichosporonosis with latent trichosporonemia, disseminated candidiasis, pulmonary paracoccidioidomycosis, pulmonary aspergillosis, Pneumocystis carinii pneumonia, cryptococcal meningitis, coccidioidal meningoencephalitis and cerebrospinal vasculitis, Aspergillus niger infection, Fusarium keratitis, paranasal sinus mycoses, Aspergillus fumigatus endocarditis, tibial dyschondroplasia, Candida glabrata vaginitis, oropharyngeal candidiasis, X-linked chronic granulomatous disease, tinea pedis, cutaneous candidiasis, mycotic placentitis, disseminated trichosporonosis, allergic bronchopulmonary aspergillosis, mycotic keratitis, Cryptococcus neoformans infection, fungal peritonitis, Curvularia geniculata infection, staphylococcal endophthalmitis, sporotrichosis, and dermatophytosis have been developed (see, e.g., Arendrup et al., 2002, Infection 30(5):286-91; Kamei, 2001, Mycopathologia 152(1):5-13; Guhad et al., 2000, FEMS Microbiol Lett. 192(1):27-31; Yamagata et al., 2000, J Clin Microbiol. 38(9):32606; Andrutis et al., 2000, J Clin Microbiol. 38(6):2317-23; Cock et al., 2000, Rev Inst Med Trop Sao Paulo 42(2):59-66; Shibuya et al., 1999, Microb Pathog. 27(3):123-31; Beers et al., 1999, J Lab Clin Med. 133(5):423-33; Najvar et al., 1999, Antimicrob Agents Chemother.43(2):413-4; Williams et al., 1988, J Infect Dis. 178(4):1217-21; Yoshida, 1998, Kansenshogaku Zasshi. June 1998;72(6):621-30; Alexaindrakis et al., 1998, Br J Ophthalmol. 82(3):306-11; Chakrabarti et al., 1997, J Med Vet Mycol. 35(4):295-7; Martin et al., 1997, Antimicrob Agents Chemother. 41(1):13-6; Chu et al., 1996, Avian Dis. 40(3):715-9; Fidel et al., 1996, J Infect Dis. 173(2):425-31; Cole et al., 1995, FEMS Microbiol Lett. 15;126(2):177-80; Pollock et al., 1995, Nat Genet. 9(2):202-9; Uchida et al., 1994, Jpn J Antibiot. 47(10):1407-12; : Maebashi et al., 1994, J Med Vet Mycol. 32(5):349-59; Jensen & Schonheyder, 1993, J Exp Anim Sci. 35(4):155-60; Gokaslan & Anaissie, 1992, Infect Immun. 60(8):3339-44; Kurup et al., 1992, J Immunol. 148(12):3783-8; Singh et al., 1990, Mycopathologia. 112(3):127-37; Salkowski & Balish, 1990, Infect Immun. 58(10):3300-6; Ahmad et al., 1986, Am J Kidney Dis. 7(2):153-6; Alture-Werber E, Edberg S C, 1985, Mycopathologia. 89(2):69-73; Kane et al., 1981, Antimicrob Agents Chemother. 20(5):595-9; Barbee et al., 1977, Am J Pathol. 86(1):281-4; and Maestrone et al., 1973, Am J Vet Res. 34(6):833-6). Animal models for fungal respiratory infections such as Candida albicans, Aspergillus fumigatus, invasive pulmonary aspergillosis, Pneumocystis carinii, pulmonary cryptococcosis, Pseudomonas aeruginosa, Cunninghamella bertholletia (see, e.g., Aratani et al., 2002 Med Mycol 40(6):557-563; Bozza et al., 2002 Microbes Infect 4(13): 1281-1290; Kurup et al., 2002 Int Arch Allergy Immunol 129(2):129-137; Hori et al., 2002 Eur J Immuno 32(5): 1282-1291; Rivera et al., 2002 J Immuno 168(7): 3419-3427; Vassallo et al., 2001, Am J Respir Cell Mol Biol 25(2): 203-211; Wilder et al., 2002 Am J Respir Cell Mol Biol 26(3): 304-314; Yonezawa et al., 2000 J Infect Chemother 6(3): 155-161; Cacciapuoti et al., 2000 Antimicrob Agents Chemother 44(8): 2017-2022; and Honda et al., 1998 Mycopathologia 144(3):141-146).

The EphA2/EphrinA1 Modulators, compositions, or combination therapies of the invention can be tested for their ability to decrease the time course of fungal infection by at least 25%, preferably at least 50%, at least 60%, at least 75%, at least 85%, at least 95%, or at least 99%. The EphA2/EphrinA1 Modulators compositions, or combination therapies of the invention can also be tested for their ability to increase the survival period of humans suffering from a fungal infection by at least 25%, preferably at least 50%, at least 60%, at least 75%, at least 85%, at least 95%, or at least 99%. Further, EphA2/EphrinA1 Modulators, compositions, or combination therapies administered according to the methods of the invention can be tested for their ability reduce the hospitalization period of humans suffering from fungal infection by at least 60%, preferably at least 75%, at least 85%, at least 95%, or at least 99%. Techniques known to those of skill in the art can be used to analyze the function of the EphA2/EphrinA1 Modulators, compositions, or combination therapies of the invention in vivo.

Further, any assays known to those skilled in the art can be used to evaluate the prophylactic and/or therapeutic utility of an EphA2/EphrinA1 Modulator, a composition, a combination therapy disclosed herein for prevention, treatment, management, and/or amelioration of an infection or one or more symptoms thereof.

### 5.3.4 Toxicity Assays

The toxicity and/or efficacy of the prophylactic and/or therapeutic protocols of the instant invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred. While therapies that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED50 with little or no toxicity. The dosage may vary within this range depending upon the dosage form employed and the route of administration utilized. For any therapy used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Further, any assays known to those skilled in the art can be used to evaluate the prophylactic and/or therapeutic utility of an EphA2/EphrinA1 Modulator, a composition, a combination therapy disclosed herein for an infection or one or more symptoms thereof.

### 5.4 Compositions & Methods of Administering EphA2/EphrinA1 Modulators

The invention provides for the prevention, treatment, management, and/or amelioration of an infection or one or more symptoms thereof. In a specific embodiment, a composition comprises one or more EphA2/EphrinA1 Modulators of the invention. In another embodiment, a composition comprises one or more EphA2/EphrinA1 Modulators of the invention and one or more prophylactic or therapeutic agents, other than the EphA2/EphrinA1 Modulators of the invention. Preferably, said agents are known to be useful for or having been or currently used for the prevention, treatment, management, and/or amelioration of an infection.

In a specific embodiment, a composition comprises one or more EphA2/EphrinA1 Modulators of the invention and one or more immunomodulatory agents. In another embodiment, a composition comprises one or more EphA2/EphrinA1 Modulators of the invention and one or more anti-inflammatory agents. In another embodiment, a composition comprising one or more EphA2/EphrinA1 Modulators of the invention and one or more anti-bacterial agents. In another embodiment, a composition comprises one or more EphA2/EphrinA1 Modulators of the invention and one or more anti-viral agents. In another embodiment, a composition comprising one or more EphA2/EphrinA1 Modulators of the invention and one or one or more anti-fungal agents. In another embodiment, a composition comprises one or more EphA2/EphrinA1 Modulators of the invention and any combination of one, two, three, or more of each of the following prophylactic or therapeutic agents: an immunomodulatory agent, an anti-inflammatory agent, an anti-viral agent, an anti-bacterial agent, an anti-fungal agent. In yet another embodiment, a composition comprises one or more EphA2/EphrinA1 Modulators of the invention and one or more integrin αᵥβ antagonists. In another embodiment, a composition comprises one or more EphA2/EphrinA1 Modulators of the invention and VITAXIN™, siplizumab, palivizumab, an anti-IL-9 antibody, or any combination thereof. In addition to prophylactic or therapeutic agents, the compositions of the invention may also comprise a carrier.

The compositions of the invention include bulk drug compositions useful in the manufacture of pharmaceutical compositions (e.g., compositions that are suitable for administration to a subject or patient) which can be used in the preparation of unit dosage forms. In a preferred embodiment, a composition of the invention is a pharmaceutical composition. Such compositions comprise a prophylactically or therapeutically effective amount of one or more prophylactic or therapeutic agents (e.g., an EphA2/EphrinA1 Modulator of the invention or other prophyilactic or therapeutic agent), and a pharmaceutically acceptable carrier. Preferably, the pharmaceutical compositions are formulated to be suitable for the route of administration to a subject.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Various delivery systems are known in the art and can be used to administer a prophylactic or therapeutic agent or composition of the invention to prevent, treat, manage, and/or ameliorate an infection, an inflammatory disorder, an autoimmune disorder, a proliferative disorder, or a infection (preferably, a respiratory infection) or one or more symptoms thereof, e.g., encapsulation in liposomes, microparticles, microcapsules, recombinant cells capable of expressing the antibody or antibody fragrnent, receptor-mediated endocytosis (see, e.g., Wu and Wu, J. Biol. Chem. 262:4429-4432 (1987)), construction of a nucleic acid as part of a retroviral or other vector, etc. Methods of administering a therapy (e.g., prophylactic or therapeutic agent) of the invention include, but are not limited to, parenteral administration (e.g., intradermal, intramuscular, intraperitoneal, intravenous and subcutaneous), epidurala administration, intratumoral administration, and mucosal adminsitration(e.g., intranasal and oral routes). In addition, pulmonary administration can also be employed, e.g., by use of an inhaler or nebulizer, and formulation with an aerosolizing agent. See, e.g., U.S. Pat. Nos. 6,019,968, 5,985, 320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety. In one embodiment, an anitbody, combination therapy, or a composition of the invention is administered using Alkermes AIR™ pulmonary drug delivery technology (Alkermes, Inc., Cambridge, Mass.). In a specific embodiment, prophylactic or therapeutic agents of the invention are administered intramuscularly, intravenously, intratumorally, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic agents of the invention locally to the area in need of treatment; this may be achieved by, for example, and not by way of limitation, local infusion, by injection, or by means of an implant, said implant being of a porous or non-porous material, including membranes and matrices, such as sialastic membranes, polymers, fibrous matrices (e.g., Tissuel®), or collagen matrices. In one embodiment, an effective amount of one or more EphA2/EphrinA1 Modulators of the invention is administered locally to the affected area to a subject at risk of or with an infection. In another embodiment, an effective amount of one or more EphA2/EphrinA1 Modulators of the invention is administered locally to the affected area in combination with an effective amount of one or more therapies (e.g. one or more prophylactic or therapeutic agents) other than an EphA2/EphrinA1 Modulator of the invention to a subject at risk of or with an infection.

In yet another embodiment, a therapy of the invention can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the therapies of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938, PCT publication WO 91/05548, PCT publication WO 96/20698, Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

In a specific embodiment, where the composition of the invention is a nucleic acid encoding a prophylactic or therapeutic agent, the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see, e.g., Joliot et al., 1991, Proc. Natl. Acad. Sci. USA 88:1864-1868). Alternatively, a nucleic acid can be introduced intracellularly and incorporated within host cell DNA for expression by homologous recombination.

A pharmaceutical composition of the invention is formulated to be compatible with its intended route of administration. Examples of routes of administration include, but are not limited to, parenteral, e.g., intravenous, intradermal, subcutaneous, oral, intranasal (e.g., inhalation), transdermal (e.g., topical), transmucosal, and rectal administration. In a specific embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

If the compositions of the invention are to be administered topically, the compositions can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the method of the invention comprises intranasal administration of a composition, the composition can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

If the method of the invention comprises oral administration, compositions can be formulated orally in the form of tablets, capsules, cachets, gelcaps, solutions, suspensions, and the like. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The method of the invention may comprise pulmonary administration, e.g., by use of an inhaler or nebulizer, of a composition formulated with an aerosolizing agent. See, e.g., U.S. Pat. Nos. 6,019,968, 5,985, 320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety. In a specific embodiment, an antibody of the invention, combination therapy, and/or composition of the invention is administered using Alkermes AIR™ pulmonary drug delivery technology (Alkermes, Inc., Cambridge, Mass.).

The method of the invention may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

The methods of the invention may additionally comprise of administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

The methods of the invention encompasses administration of compositions formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions are supplied either separately or mixed together in unit dosage form, for example, as a dry Iyophilized powder or water free concentrate in a hermetically sealed container such as an ampoule or sachette indicating the quantity of active agent. Where the mode of administration is infusion, composition can be dispensed with an infusion bottle containing sterile pharmaceutical grade water or saline. Where the mode of administration is by injection, an ampoule of sterile water for injection or saline can be provided so that the ingredients may be mixed prior to administration.

In particular, the invention also provides that one or more of the prophylactic or therapeutic agents, or pharmaceutical compositions of the invention is packaged in a hermetically sealed container such as an ampoule or sachette indicating the quantity of the agent. In one embodiment, one or more of the prophylactic or therapeutic agents, or pharmaceutical compositions of the invention is supplied as a dry sterilized lyophilized powder or water free concentrate in a hermetically sealed container and can be reconstituted (e.g., with water or saline) to the appropriate concentration for administration to a subject. Preferably, one or more of the prophylactic or therapeutic agents or pharmaceutical compositions of the invention is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized prophylactic or therapeutic agents or pharmaceutical compositions of the invention should be stored at between 2° C. and 8° C. in its original container and the prophylactic or therapeutic agents, or pharmaceutical compositions of the invention should be administered within 1 week, preferably within 5 days, within 72 hours, within 48 hours, within 24 hours, within 12 hours, within 6 hours, within 5 hours, within 3 hours, or within 1 hour after being reconstituted. In an alternative embodiment, one or more of the prophylactic or therapeutic agents or pharmaceutical compositions of the invention is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2° C. and 8° C. in its original container.

Generally, the ingredients of the compositions of the invention are derived from a subject that is the same species origin or species reactivity as recipient of such compositions. Thus, in a preferred embodiment, human or humanized antibodies are administered to a human patient for therapy or prophylaxis.

### 5.4.1 Gene Therapy

In specific embodiments, EphA2/EphrinA1 Modulators of the invention that are nucleotides are administered to treat, manage, or prevent an infection by way of gene therapy. Gene therapy refers to therapy performed by the administration to a subject of an expressed or expressible nucleic acid. In this embodiment of the invention, the antisense nucleic acids are produce and mediate a prophylactic or therapeutic effect. Gene therapy refers to therapy performed by the administration to a subject of an expressed or expressible nucleic acid. In a specific embodiment of the invention, the antisense nucleic acids are produced and mediate a prophylactic or therapeutic effect. In another specific embodiment of the invention, gene therapy is not an EphA2/EphrinA1 Modulator vaccine-based therapy (e.g., is not an EphA2- or EphrinA1 vaccine).

Any of the methods for gene therapy available in the art can be used according to the present invention. Exemplary methods are described below.

For general reviews of the methods of gene therapy, see Goldspiel et al., 1993, Clinical Pharmacy 12:488; Wu and Wu, 1991, Biotherapy 3:87; Tolstoshev, 1993, Ann. Rev. Pharmacol. Toxicol. 32:573; Mulligan, 1993, Science 260:926-932; and Morgan and Anderson, 1993, Ann. Rev. Biochem. 62:191; May, 1993, TIBTECH 11: 155. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In one aspect, a composition of the invention comprises EphA2 nucleic acids that decrease EphA2 expression, said nucleic acids being part of an expression vector that expresses the nucleic acid in a suitable host. In particular, such nucleic acids have promoters, preferably heterologous promoters, said promoter being inducible or constitutive, and, optionally, tissue-specific. In another particular embodiment, nucleic acid molecules are used in which the nucleic acid that decrease EphA2 expression and any other desired sequences are flanked by regions that promote homologous recombination at a desired site in the genome, thus providing for intrachromosomal expression of the nucleic acids that decrease EphA2 expression (Koller and Smithies, 1989, PNAS 86:8932; Zijlstra et al., 1989, Nature 342:435).

In another aspect, a composition of the invention comprises EphrinA1 nucleic acids that decrease EphrinA1 expression, said nucleic acids being part of an expression vector that expresses the nucleic acid in a suitable host. In particular, such nucleic acids have promoters, preferably heterologous promoters, said promoter being inducible or constitutive, and, optionally, tissue-specific. In another particular embodiment, nucleic acid molecules are used in which the nucleic acid that decrease EphrinA1 expression and any other desired sequences are flanked by regions that promote homologous recombination at a desired site in the genome, thus providing for intrachromosomal expression of the nucleic acids that decrease EphrinA1 expression (Koller and Smithies, 1989, PNAS 86:8932; Zijlstra et al., 1989, Nature 342:435).

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy. In a specific embodiment, the nucleic acid sequences are directly administered in vivo. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429) (which can be used to target cell types specifically expressing the receptors), etc. In another embodiment, nucleic acid-ligand complexes can be formed in which the ligand comprises a flisogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., International Patent Publication Nos. WO 92/06180; WO 92/22635; W092/203 16; W093/14188, WO 93/20221). Alternatively, the nucleic acid can be introduced intracellularly and incorporated within host cell DNA for expression, by homologous recombination (Koller and Smithies, 1989, PNAS 86:8932; and Zijlstra et al., 1989, Nature 342:435).

In a specific embodiment, viral vectors that contain the nucleic acid sequences that decrease EphrinA1 expression are used. For example, a retroviral vector can be used (see Miller et al., 1993, Meth. Enzymol. 217:581). These retroviraI vectors contain the components necessary for the correct packaging of the viral genome and integration into the host cell DNA. The nucleic acid sequences to be used in gene therapy are cloned into one or more vectors, which facilitates delivery of the nucleic acid into a subject. More detail about retroviral vectors can be found in Boesen et al., 1994, Biotherapy 6:291-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Adenoviruses have the advantage of being capable of infecting non-dividing cells. Kozarsky and Wilson, 1993, Current Opinion in Genetics Development 3:499 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431; Rosenfeld et al., 1992, Cell 68:143; Mastrangeli et al., 1993, J. Clin. Invest. 91:225; International Patent Publication No. W094/12649; and Wang et al., 1995, Gene Therapy 2:775. In a preferred embodiment, adenovirus vectors are used.

Adeno-associated virus (AAV) has also been proposed for use in gene therapy (Walsh et al., 1993, Proc. Soc. Exp. Biol. Med. 204:289-300; and U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell mediated gene transfer, spheroplast fusion, etc. Numerous techniques are known in the art for the introduction of foreign genes into cells (see, e.g., Loeffler and Behr, 1993, Meth. Enzymol. 217:599; Cohen et al., 1993, Meth. Enzymol. 217:618) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. The amount of cells envisioned for use depends on the desired effect, patient state, etc., and can be determined by one skilled in the art.

### 5.5 Dosages and Frequency of Administration

The amount of a prophylactic or therapeutic agent or a composition of the invention which will be effective in the prevention, treatment, management, and/or amelioration of an infection or one or more symptoms thereof can be determined by standard clinical methods. The frequency and dosage will vary also according to factors specific for each patient depending on the specific therapies (e.g., the specific therapeutic or prophylactic agent or agents) administered, the severity of the disorder, disease, or condition, the route of administration, as well as age, body, weight, response, and the past medical history of the patient. For example, the dosage of a prophylactic or therapeutic agent or a composition of the invention which will be effective in the treatment, prevention, management, and/or amelioration of an infection or one or more symptoms thereof can be determined by administering the composition to an animal model such as, e.g., the animal models disclosed herein or known in to those skilled in the art. In addition, in vitro assays may optionally be employed to help identify optimal dosage ranges. Suitable regimens can be selected by one skilled in the art by considering such factors and by following, for example, dosages are reported in literature and recommended in the Physicians' Desk Reference (59th ed., 2005).

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

For antibodies, proteins, polypeptides, peptides and fusion proteins encompassed by the invention, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention or fragments thereof may be reduced by enhancing uptake and tissue penetration of the antibodies by modifications such as, for example, lipidation.

In a specific embodiment, the dosage of EphA2/EphrinA1 Modulators (e.g., antibodies, compositions, or combination therapies of the invention) administered to prevent, treat, manage, and/or ameliorate an infection or one or more symptoms thereof in a patient is 150 μg/kg or less, preferably 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of a patient's body weight. In another embodiment, the dosage of the EphA2/EphrinA1 Modulators or combination therapies of the invention administered to prevent, treat, manage, and/or ameliorate an infection, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

In other embodiments, a subject is administered one or more doses of an effective amount of one or EphA2/EphrinA1 Modulators of the invention, wherein the dose of an effective amount achieves a serum titer of at least 0.1 μg/ml, at least 0.5 μg/ml, at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml of the EphA2/EphrinA1 Modulators of the invention. In yet other embodiments, a subject is administered a dose of an effective amount of one or more EphA2/EphrinA1 Modulators of the invention to achieve a serum titer of at least 0.1 μg/ml, at least 0.5 μg/ml, at least 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml of the antibodies and a subsequent dose of an effective amount of one or more EphA2/EphrinA1 Modulators of the invention is administered to maintain a serum titer of at least 0.1 μg/ml, 0.5 μg/ml, 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml. In accordance with these embodiments, a subject may be administered 1, 2, 3, 4, 5, 6, 7, 8, 9, 10, 11, 12 or more subsequent doses.

In a specific embodiment, the invention provides methods of preventing, treating, managing, or ameliorating an infection or one or more symptoms thereof, said method comprising administering to a subject in need thereof a dose of at least 10 μg, preferably at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg, at 100 μg, at least 105 μg, at least 110 μg, at least 115 μg, or at least 120 μg of one or more EphA2/EphrinA1 Modulators, combination therapies, or compositions of the invention. In another embodiment, the invention provides a method of preventing, treating, managing, and/or ameliorating an infection or one or more symptoms thereof, said methods comprising administering to a subject in need thereof a dose of at least 10 μg, preferably at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg, at least 105 μg, at least 110 μg, at least 115 μg, or at least 120 μg of one or more EphA2/EphrinA1 Modulators, combination therapies, or compositions of the invention once every 3 days, preferably, once every 4 days, once every 5 days, once every 6 days, once every 7 days, once every 8 days, once every 10 days, once every two weeks, once every three weeks, or once a month.

The present invention provides methods of preventing, treating, managing, or preventing an infection or one or more symptoms thereof, said method comprising: (a) administering to a subject in need thereof one or more doses of a prophylactically or therapeutically effective amount of one or more EphA2/EphrinA1 Modulators, combination therapies, or compositions of the invention; and (b) monitoring the plasma level/concentration of the said administered EphA2/EphrinA1, Modulators in said subject after administration of a certain number of doses of the said EphA2/EphrinA1 Modulators. Moreover, preferably, said certain number of doses is 1, 2, 3, 4, 5, 6, 7, 8, 9, 10, 11, or 12 doses of a prophylactically or therapeutically effective amount one or more EphA2/EphrinA1 Modulators, compositions, or combination therapies of the invention.

In a specific embodiment, the invention provides a method of preventing, treating, managing, and/or ameliorating an infection or one or more symptoms thereof, said method comprising: (a) administering to a subject in need thereof a dose of at least 10 μg (preferably at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg) of one or more EphA2/EphrinA1 Modulators of the invention; and (b) administering one or more subsequent doses to said subject when the plasma level of the EphA2/EphrinA1 Modulator administered in said subject is less than 0.1 μg/ml, preferably less than 0.25 μg/ml, less than 0.5 μg/ml, less than 0.75 μg/ml, or less than 1 μg/ml. In another embodiment, the invention provides a method of preventing, treating, managing, and/or ameliorating an infection or one or more symptoms thereof, said method comprising: (a) administering to a subject in need thereof one or more doses of at least 10 μg (preferably at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg) of one or more antibodies of the invention; (b) monitoring the plasma level of the administered EphA2/EphrinA1 Modulators of the invention in said subject after the administration of a certain number of doses; and (c) administering a subsequent dose of EphA2/EphrinA1 Modulators of the invention when the plasma level of the administered EphA2/EphrinA1 Modulator in said subject is less than 0.1 μg/ml, preferably less than 0.25 μg/ml, less than 0.5 μg/ml, less than 0.75 μg/ml, or less than 1 μg/ml. Preferably, said certain number of doses is 1, 2, 3, 4, 5, 6, 7, 8, 9, 10, 11, or 12 doses of an effective amount of one or more EphA2/EphrinA1 Modulators of the invention.

Therapies (e.g., prophylactic or therapeutic agents), other than the EphA2/EphrinA1 Modulators of the invention, which have been or are currently being used to prevent, treat, manage, and/or ameliorate an infection or one or more symptoms thereof can be administered in combination with one or more EphA2/EphrinA1 Modulators according to the methods of the invention to treat, manage, prevent, and/or ameliorate an infection or one or more symptoms thereof. Preferably, the dosages of prophylactic or therapeutic agents used in combination therapies of the invention are lower than those which have been or are currently being used to prevent, treat, manage, and/or ameliorate an infection or one or more symptoms thereof. The recommended dosages of agents currently used for the prevention, treatment, management, or amelioration of an infection or one or more symptoms thereof can be obtained from any reference in the art including, but not limited to, Hardman et al., eds., 2001, Goodman & Gilman's The Pharmacological Basis Of Basis Of Therapeutics, 10th ed., Mc-Graw-Hill, New York; Physicians' Desk Reference (59th ed., 2005), Medical Economics Co., Inc., Montvale, N.J., which are incorporated herein by reference in its entirety.

In various embodiments, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In preferred embodiments, two or more therapies are administered within the same patient visit.

In certain embodiments, one or more antibodies of the invention and one or more other therapies (e.g., prophylactic or therapeutic agents) are cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain embodiments, the administration of the same EphA2/EphrinA1 Modulators of the invention may be repeated and the administrations may be separated by at least 1 day, 2 days, 3 days, 5 days, 10 days, 15 days, 30 days, 45 days, 2 months, 75 days, 3 months, or at least 6 months. In other embodiments, the administration of the same therapy (e.g., prophylactic or therapeutic agent) other than an EphA2/EphrinA1 Modulator of the invention may be repeated and the administration may be separated by at least at least 1 day, 2 days, 3 days, 5 days, 10 days, 15 days, 30 days, 45 days, 2 months, 75 days, 3 months, or at least 6 months.

In certain embodiments, the EphA2- or EphrinA1 antigenic peptides and anti-idiotypic antibodies of the invention are formulated at 1 mg/ml, 5 mg/ml, 10 mg/ml, and 25 mg/ml for intravenous injections and at 5 mg/ml, 10 mg/ml, and 80 mg/ml for repeated subcutaneous administration and intramuscular injection.

Where the EphA2- or EphrinA1 vaccine is a bacterial vaccine, the vaccine can be formulated at amounts ranging between approximately 1×10² CFU/ml to approximately 1×10¹² CFU/ml, for example at 1×10² CFU/ml, 5×10² CFU/ml, 1×10³ CFU/ml, 5×10³ CFU/ml, 1×10⁴ CFU/ml, 5×10⁴ CFU/ml, 1×10⁵ CFU/ml, 5×10⁵ CFU/ml, 1×10⁶ CFU/ml, 5×10⁶ CFU/ml, 1×10⁷ CFU/ml, 5×10⁷ CFU/ml, 1×10⁸ CFU/ml, 5×10⁸ CFU/ml, 1×10⁹ CFU/ml, 5×10⁹ CFU/ml, 1×10¹⁰ CFU/ml, 5×10¹⁰ CFU/ml, 1×10¹¹ CFU/ml, 5×10¹¹ CFU/ml, or 1×10² CFU/ml.

For EphA2- and EphrinA1 antigenic peptides or anti-idiotypic antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight.

With respect to the dosage of bacterial EphA2- and EphrinA1 vaccines of the invention, the dosage is based on the amount colony forming units (c.f.u.). Generally, in various embodiments, the dosage ranges are from about 1.0 c.f.u./kg to about 1×10¹⁰ c.f.u./kg; from about 1.0 c.f.u./kg to about 1×10⁸ c.f.u./kg; from about 1×10² c.f.u./kg to about 1×10⁸ c.f.u./kg; and from about 1×10⁴ c.f.u./kg to about 1×10⁸ c.f.u./kg. Effective doses may be extrapolated from dose-response curves derived animal model test systems. In certain exemplary embodiments, the dosage ranges are 0.001-fold to 10,000-fold of the murine LD₅₀, 0.01-fold to 1,000-fold of the murine LD₅₀, 0.1-fold to 500-fold of the murine LD₅₀, 0.5-fold to 250-fold of the murine LD₅₀, 1-fold to 100-fold of the murine LD₅₀, and 5-fold to 50-fold of the murine LD₅₀. In certain specific embodiments, the dosage ranges are 0.00.1-fold, 0.01-fold, 0.1-fold, 0.5-fold, 1-fold, 5-fold, 10-fold, 50-fold, 100-fold, 200-fold, 500-fold, 1,000-fold, 5,000-fold or 10,000-fold of the murine LD₅₀.

### 5.6 Diagnostic Uses of EphA2/EphrinA1 Modulators

In specific embodiments, the Eph/EphrinA1 Modulators of the invention can be used for diagnostic purposes to detect, diagnose, prognose, or monitor an infection, in particular, an intracellular pathogen infection or one or more symptoms thereof. Such methods may be used in combination with other methods for detecting, diagnosing, monitoring or prognosing an infection. The invention also provides methods for prognosing and monitoring the efficacy of a therapy. The present invention also provides methods of detecting infected cells that overexpress EphA2 using the EphA2/EphrinA1 Modulators of the invention. In specific embodiments, the invention provides methods for detecting, diagnosing, monitoring or prognosing active and/or latent infections. The invention further provides for the detection of increased EphA2 expression in infected cells comprising: (a) assaying the expression of EphA2 in a biological sample from an individual using one or more EphA2/EphrinA1 Modulators of the invention (e.g., an EphA2 antibody or a soluble EphrinA1) that immunospecifically binds to an EphA2 polypeptide; and (b) comparing the level of EphA2 with a standard level of EphA2, e.g., in normal biological samples, whereby an increase in the assayed level of EphA2 compared to the standard level of EphA2 is indicative of an infection or one or more symptoms thereof.

In preferred embodiments, the labeled antibodies that immunospecifically bind to EphA2 are used for diagnostic purposes to detect, diagnose, prognose, or monitor an infection, preferably an intracellular pathogen infection caused by viruses, bacteria, fungi or protozoa. The invention provides methods for the detection of an infection, comprising: (a) assaying the expression of EphA2 in cells or a tissue sample of a subject using one or more antibodies that immunospecifically bind to EphA2; and (b) comparing the level of EphA2 with a control level, e.g., levels in normal tissue samples not infected, whereby an increase in the assayed level of EphA2 compared to the control level of EphA2 is indicative of an infection.

EphA2 antibodies can be used to assay EphA2 levels in a biological sample using classical immunohistological methods as described herein or as known to those of skill in the art e.g., see Jalkanren et al., 1985, J. Cell. Biol. 101:976-985; and Jalkanen et al., 1987, J. Cell. Biol. 105:3087-3096). The EphA2 antibodies used in the methods of the may have a low K_{off} rate (e.g., K_{off} less than 3×10⁻³s⁻¹). In one embodiment, the antibodies used in the methods of the invention are Eph099B-102.147, Eph099B-208.261, Eph099B-210.248, B233, EA2 or EA5. In a more preferred embodiment, the antibodies used in the methods of the invention are human or humanized Eph099B-102.147, Eph099B-208.261, Eph099B-210.248, B233, EA2 or EA5. In a specific embodiment, the antibodies used are not Eph099B-102.147, Eph099B-208.261, Eph099B-210.248, B233, EA2 or EA5.

Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹²¹ In), and technetium (⁹⁹Tc); luminescent labels, such as luminol; and fluorescent labels, such as fluorescein and rhodamine, and biotin.

One aspect of the invention is the detection and diagnosis of an infection in an animal, preferably a mammal, and most preferably a human. In one embodiment, diagnosis comprises: a) administering (for example, parenterally, subcutaneously, or intraperitoneally) to a subject an effective amount of a labeled EphA2/EphrinA1 Modulator of the invention (including molecules comprising, or alternatively consisting of, antibody fragments or variants thereof) that immunospecifically binds to EphA2; b) waiting for a time interval following the administering for permitting the labeled antibody to preferentially concentrate at sites in the subject where EphA2 is expressed (and for unbound labeled molecule to be cleared to background level); c) determining background level; and d) detecting the labeled EphA2/EphrinA1 Modulator in the subject, such that detection of labeled EphA2/EphrinA1 Modulator above the background level and above or below the level observed in a person without the infection. Background level can be determined by various methods including, comparing the amount of labeled molecule detected to a standard value previously determined for a particular system. Aberrant expression (i.e., increased) of EphA2 can occur particularly in epithelial cell types. In a specific embodiment, the methods of the invention are particularly useful for the treatment of latent intracellular pathogen infections.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹Tc. The labeled antibody will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds, Masson Publishing Inc. (1982). Depending on several variables, including the type of label used and the mode of administration, the time interval following the administration for permitting the labeled molecule to preferentially concentrate at sites in the subject and for unbound labeled molecule to be cleared to background level is 6 to 48 hours, 6 to 24 hours, or 6 to 12 hours. In another embodiment the time interval following administration is 5 to 20 days or 5 to 10 days.

In an embodiment, monitoring of the infection is carried out by repeating the method for diagnosing the infection, for example, one month after initial diagnosis, six months after initial diagnosis, one year after initial diagnosis, etc.

Presence of the labeled EphA2/EphrinA1 Modulator can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the EphA2/EphrinA1 Modulator is labeled with a radioisotope and is detected in the patient using a radiation responsive surgical instrument (Thurston et al., U.S. Pat. No. 5,441,050). In another embodiment, the EphA2/EphrinA1 Modulator is labeled with a fluorescent compound and is detected in the patient using a fluorescence responsive scanning instrument. In another embodiment, the EphA2/EphrinA1 Modulator is labeled with a positron emitting metal and is detected in the patient using positron emission-tomography. In yet another embodiment, the EphA2/EphrinA1 Modulator is labeled with a paramagnetic label and is detected in a patient using magnetic resonance imaging (MRI).

### 5.7 Kits

The invention provides a pharmaceutical pack or kit comprising one or more containers filled with an EphA2/EphrinA1 Modulator of the invention. Additionally, one or more other prophylactic or therapeutic agents useful for the treatment, management or prevention of an infection, or other relevant agents can also be included in the pharmaceutical pack or kit. In certain embodiments, the other prophylactic or therapeutic agent is an immunomodulatory agent (e.g., anti-IL-9 antibody). In other embodiments, the other prophylactic or therapeutic agent is an anti-viral agent. In a further embodiments, the the other prophylactic or therapeutic agent is an anti-bactieral agent. In yet further embodiments, the other prophylactic or therapeutic agent is an anti-fungal agent. In another embodiment, the other prophylactic or therapeutic agent is an anti-inflammatory agent. In yet another embodiment, the other prophylactic or therapeutic agent is an anti-protozoa agent. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### 5.8 Articles of Manufacture

The present invention also encompasses a finished packaged and labeled pharmaceutical product. This article of manufacture includes the appropriate unit dosage form in an appropriate vessel or container such as a glass vial or other container that is hermetically sealed. The invention encompasses both parenteral solutions and lyophilized powders, each being sterile, and the latter being suitable for reconstitution prior to injection. Alternatively, the unit dosage form may be a solid suitable for oral, transdermal, intransal, or topical delivery.

In a preferred embodiment, the unit dosage form is suitable for intravenous, intramuscular, intranasal, oral, topical or subcutaneous delivery. Thus, the invention encompasses solutions, preferably sterile, suitable for each delivery route.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures, total lymphocyte, mast cell counts, T cell counts, IgE production, and other monitoring information.

Specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material, wherein said pharmaceutical agent comprises EphA2/EphrinA1 Modulator and wherein said packaging material includes instruction means which indicate that said EphA2/EphrinA1 Modulator can be used to prevent, manage, treat, and/or ameliorate one or more symptoms associated with an infection or one or more symptoms thereof by administering specific doses and using specific dosing regimens as described herein. In specific embodiments, the infection causes and/or is associated by increased EphA2 expression.

The invention also provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material, wherein one pharmaceutical agent comprises an EphA2/EphrinA1 Modulator, a second pharmaceutical agent comprises a prophylactic or therapeutic agent other than an EphA2/EphrinA1 Modulator, and wherein said packaging material includes instruction means which indicate that said agents can be used to treat, prevent and/or ameliorate an infection or one or more symptoms thereof by administering specific doses and using specific dosing regimens as described herein.

The present invention provides that the adverse effects that may be reduced or avoided by the methods of the invention are indicated in informational material enclosed in an article of manufacture for use in preventing, treating and/or ameliorating one or more symptoms associated with an infection. Adverse effects that may be reduced or avoided by the methods of the invention include, but are not limited to, vital sign abnormalities (fever, tachycardia, bardycardia, hypertension, hypotension), hematological events (anemia, lymphopenia, leukopenia, thrombocytopenia), headache, chills, dizziness, nausea, asthenia, back pain, chest pain (chest pressure), diarrhea, myalgia, pain, pruritus, psoriasis, rhinitis, sweating, injection site reaction, and vasodilatation.

Further, the information material enclosed in an article of manufacture for use in preventing, treating, managing, and/or ameliorating an infection or one or more symptoms thereof can indicate that foreign proteins may also result in allergic reactions, including anaphylaxis, or cytosine release syndrome. The information material should indicate that allergic reactions may exhibit only as mild pruritic rashes or they may be severe such as erythroderma, Stevens-Johnson syndrome, vasculitis, or anaphylaxis. The information material should also indicate that anaphylactic reactions (anaphylaxis) are serious and occasionally fatal hypersensitivity reactions. Allergic reactions including anaphylaxis may occur when any foreign protein is injected into the body. They may range from mild manifestations such as urticaria or rash to lethal systemic reactions. Anaphylactic reactions occur soon after exposure, usually within 10 minutes. Patients may experience paresthesia, hypotension, laryngeal edema, mental status changes, facial or pharyngeal angioedema, airway obstruction, bronchospasm, urticaria and pruritus, serum sickness, arthritis, allergic nephritis, glomerulonephritis, temporal arthritis, or eosinophilia.

### 6. EXAMPLES

### 6.1 Materials

The following materials were used to perform the experiments described in Examples 1-8, infra:

RSV-A2 #10Y in-house stock (A. Brewah, MedImmune, Inc.)

BEAS-2B, normal human bronchial epithelial cell line (ATCC, Manassas, Va.)

HNBE, primary normal human bronchial epithelial cells (Cambrex, East Rutherford, N.J.)

Hep-2, epithelial carcinoma cell line (ATCC)

A549, lung epithelial carcinomna cell line (ATCC)

BEGM Bullet Kit, serum free growth medium (Cambrex)

Subculturing Reagent Pack (Cambrex)

Earles Minimal Essential Medium with GlutaMax (Invitrogen, Carlsbad, Calif.)

Fetal Bovine Serum, Qualified (Invitrogen)

Penicillin/Streptomycin (Invitrogen)

Phosphate Buffered Saline (PBS), pH 7.4 (Invitrogen)

Trypsin, 0.05%+EDTA, 0.48 mM (Invitrogen)

Cell Dissociation Buffer, enzyme free (Invitrogen)

Bovine Serum Albumin, Fraction V (Sigma, St. Louis, Mo.)

FACS Buffer: 1% BSA in PBS, pH 7.4

BCA Protein Assay Kit (Pierce Biotechnology, Inc., Rockford, Ill.)

Novex Xcell SureLock Cell (for SDS-PAGE) (Invitrogen)

Novex Xcell II Blot Module (Invitrogen)

Western Transfer Sponges (Invitrogen)

4-12% NuPage Bis Tris polyacrylamide gel (Invitrogen)

NuPage MES-SDS buffer (Invitrogen)

NuPage LDS Sample Buffer (Invitrogen)

NuPage Reducing Agent (Invitrogen)

NuPage Antioxidant (Invitrogen)

NuPage Western Transfer Buffer (Invitrogen)

Methanol, ACS grade (VWR, Bridgeport, N.J.)

MagicMark XP Western Protein MW Standards (Invitrogen)

0.2μ pore size nitrocellulose/filter paper sandwiches (Invitrogen)

Anti-Eck/EphA2 clone D7 mAb, (Upstate Biotechnology, Waltham, Mass.)

Goat anti-murine IgG, HRP conjugated (Jackson Immuno Research Labs, West Grove, Pa.)

Super Signal West Pico Chemiluminescent Substrate (Pierce)

Biomax XAR x-ray film, 13×18 cm (Kodak, Rochester, N.Y.)

X-ray film processor, Kodak X-OMAT 1000A (Kodak)

CO₂ incubator (VWR)

Laminar flow hood for cell culture (VWR)

10× Tris Buffered Saline (Biosource, Camarillo, Calif.)

EDTA (Sigma)

Aprotinin (Sigma)

Leupeptin (Sigma)

Sodium Vanadate (Sigma)

10× Tris Buffered Saline (Biosource)

Triton X 100 (Sigma)

Tween 20 (Sigma)

Fish Gelatin, 45% (Sigma)

Cell Lysis Buffer: 50 mM Tris, pH 7.5/150 mM NaCl/2 mM EDTA/1% TritonX100/0.1%

NaN₃/25 μg/ml Aprotinin/10 μg/ml Leupeptin/1 mM Na Vanadate

Western Blocking Buffer: Tris buffered saline/1% BSA/0.5% fish gelatin/0.1% Tween20

Western Wash Buffer (TBS-TB): Tris Buffered Saline/0.1% BSA/0.05% Tween20

Anti-RSV-F protein IgG, Synagis, clinical grade (Medhmmune, Inc., Gaithersburg, Md.)

Isotype Control human mAb, Vitaxin, clinical grade (Medimmune, Inc.)

Anti-EphA2 mAb, B233 (MedImmune, Inc.)

Isotype Control, murine IgG (BD Pharmingen, San Diego, Calif.)

Rabbit anti-human IgG, Aiexa488 conjugated (Molecular Probes, Inc., Eugene, Oreg.)

Goat anti-murine IgG, APC conjugated (BD Pharmingen)

ABI Prism 7000 Sequence Detection System (Applied Biosystems, ABI, Foster City, Calif.)

Microsoft Excel file qgene96 (Patrick Muller)

Total RNA Isolation Mini Kit (Agilent Technologies, Palo Alto, Calif.)

TaqMan One Step RT-PCR Mastermix Kit (ABI)

Assay on Demand for human EphA2 (ABI)

Eukaryotic 18S rRNA Endogenous Control (ABI)

96-well optical reaction plate (ABI)

Adhesive seal applicator kit (ABI)

Methyl cellulose (Sigma)

Crystal violet (Sigma)

### 6.2 Example 1

### Detection of EphA2 on BEAS-2B Cells Using Western Blot Analysis

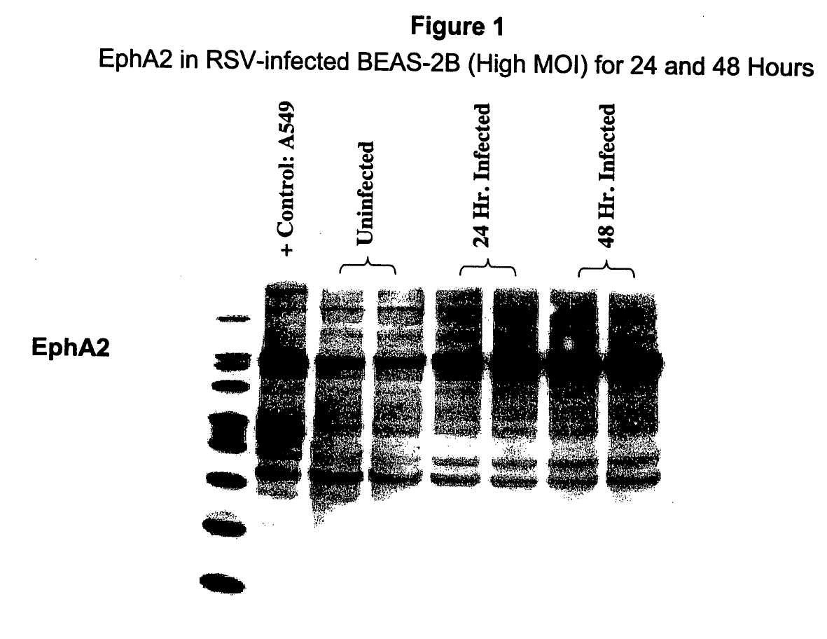

This example demonstrates that total EphA2 protein is increased following an infection with RSV using Western blot analysis (see FIG. 1).

Cell Culture

For cell culture, 60 mm plates were seeded with 10⁶ BEAS-2B cells in 5 ml BEGM. When the cells were -80% confluent, they were infected with RSV-A2.

RSV Infection

For infection of the cells, RSV-A2 stock, at a concentration of 1.8×10⁸ pfu/ml, was diluted in BEGM to 2.5×10⁷ pfu/ml, and BEAS-2B cells were infected with 1 ml of diluted virus. Plates were incubated at 37° C., 5% CO₂, for 2.5 hours with rocking every 30 minutes. After infection, the inoculum was removed and 5 ml fresh BEGM was added to the plate. Cells were incubated at 37° C. and 5% CO₂ for the indicated times.

Preparation of Cell Lysates

For preparation of the cell lysates, plates were chilled on ice during the lysis procedure. Medium was removed and cells were washed once with 5 ml ice cold PBS, pH 7.4. PBS was removed and 200 μl ice cold lysis buffer was added to each plate. Plates were rocked to distribute lysis buffer over the cells, and were then incubated on ice for 5 minutes. Plates were tilted and lysates collected from the edge of the monolayer, and then transferred to 1.5 ml tubes on ice.

Protein Determination

For protein determination, the protein concentration in each sample was determined by the BCA method. Volume of sample equaling 30 μg was calculated.

Western Blot Analysis of EphA2 in RSV-Infected Cells

Whole cell extracts were made from BEAS-2B cells infected for 0, 24, or 43 hours with RSV at a multiplicity of infection (MOI) of 10. At this MOI, virtually all the cells are infected immediately. Equal amounts of protein from each sample were run on SDS-PAGE. Thirty μg samples in reducing LDS sample buffer and Western blot standards were run on a 4-12% NuPage Bis Tris gel in SDS-MES buffer for 25 minutes at 100 V. Proteins were transferred to nitrocellulose using the Xcell II blot module for 1 hour at 30 V, according to the manufacturer's instructions. Proteins on the gel were transferred to a nitrocellulose membrane that was subsequently developed as a Western blot. Nonspecific protein binding sites on the blot were blocked by incubating the blot in 50 ml blocking buffer, rocking, for 1 hour at room temperature. Blocking buffer was discarded. The blot was treated with primary antibody, anti-EphA2 mAb D7 (which binds to human EphA2), 0.5 μg/ml in 20 ml TBS-TB, rocking for 1 hour at room temperature. Unbound primary antibody was washed off the blot by washing with 20-30 ml TBS-TB, 10 times over the course of 30 minutes, rocking at room temperature. The blot was treated with secondary antibody, goat anti-murine IgG, conjugated with peroxidase (80 ng/ml), 1:10,000 dilution in 20 ml TBS-TB, rocking for 30 minutes at room temperature. The blot was washed as before to remove unbound secondary antibody. The blot was washed again twice, briefly, with 20 ml TBS to prepare it for chemiluminescent development. Equal volumes of the two chemiluminescence reagents were combined just before use, and the drained blot was exposed to 2 ml of the substrate for 1-2 minutes. Substrate was drained off and the blot was placed on absorbent paper until it was damp (but not dripping). The blot was then placed between clear plastic sheets in a film cassette. X-ray film was exposed to the covered blot for various times, until an exposure was obtained that showed all standard and EphA2 bands.

As shown in FIG. 1, total EphA2 protein dramatically increases after RSV infection of BEAS-2B cells, and continues to increase from one day to two days after infection.

### 6.3 Example 2

### Detection of EphA2 on BEAS-2B Cells Using FACS Analysis

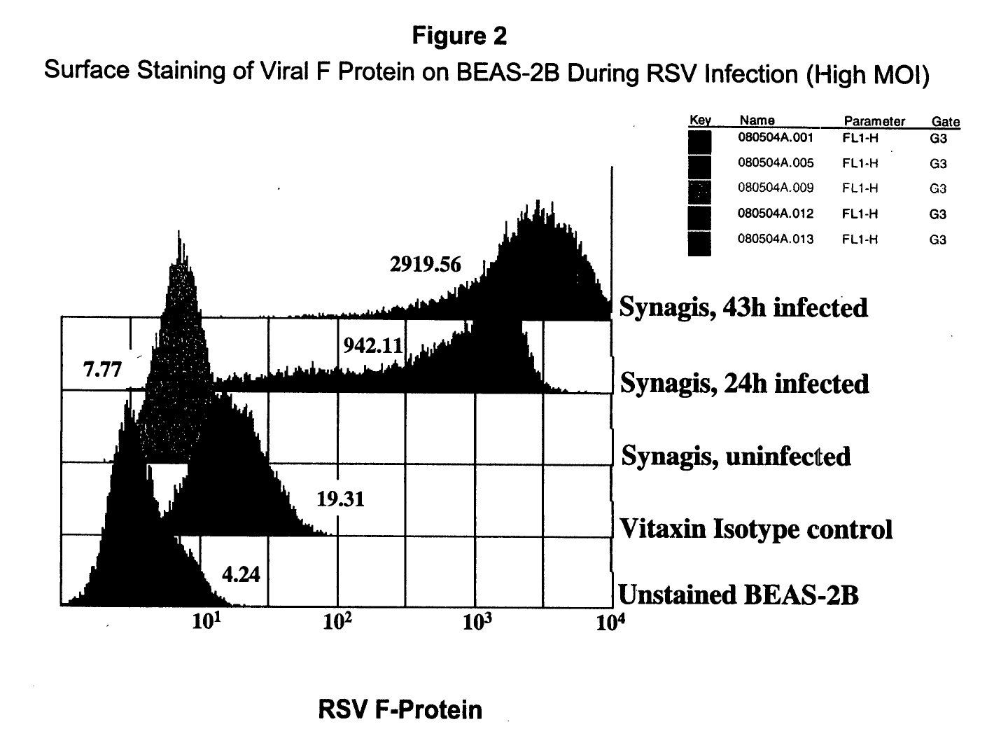

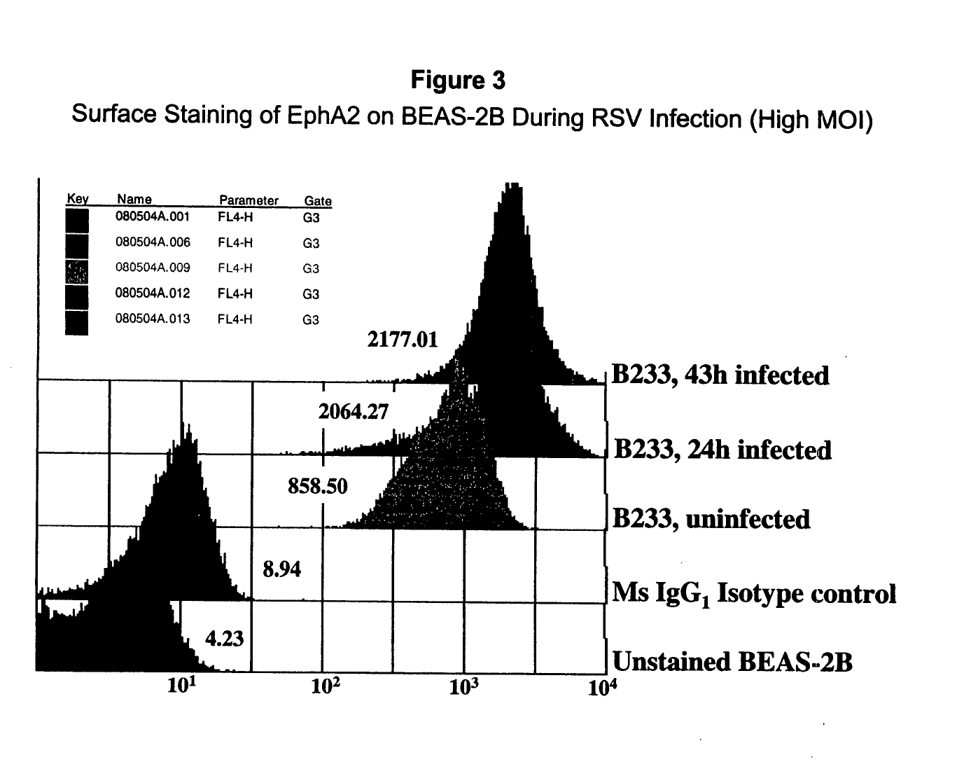

This example demonstrates the amount of RSV-F protein and EphA2 protein present on the surface of BEAS-2B cells infected with RSV increases, as measured by Fluorescence Activated Cell Sorting (FACS) (see FIGS. 2 and 3, respectively). FACS analysis measures the intensity of fluorescently labeled RSV-F protein or EphA2 protein on the cell surface and plots it as a histogram along the x-axis. The number of cells is plotted on the y-axis. The numbers beside each histogram are the mean fluorescence intensity (MFI). MFI is directly proportional to the amount of RSV-F protein or EphA2 protein on the cell surface. Thus, with respect to RSV-F protein, MFI is a measurement of the degree of infection of the cells.

Preparation of Cells

BEAS-2B cells were plated and infected as described in Example 1, supra.

At the indicated times after infection, the cells were washed once with PBS, then detached from the plates with a 1:1 mixture of Cell Dissociation Buffer and 0.05% trypsin/0.48mM EDTA, 2-3 min, 37° C. Cells (5-7×10⁵) were transferred to 5 ml FACS tubes, and the tubes were filled with cold FACS buffer. Cells were pelleted at 1100 rpm for 3 minutes at room temperature. Supernatants were decanted, and the cells were resuspended in 100 μl FACS buffer.

Nonspecific binding sites on the cells were blocked by adding 3 μg goat IgG/tube, and incubating for 10 minutes at room temperature. Primary antibody recognizing either RSV-F protein (Synagis), or EphA2 (B233), or their respective isotype control was added at a concentration of 1 μg/tube. Cells and antibody were mixed and incubated for 30 minutes on ice. After incubation, the tubes were filled with FACS buffer and centrifuged as described above.

Following centrifugation, the supernatants were decanted, and the cells were resuspended in 100 μl FACS buffer. Secondary antibody was added: 1 μg Rabbit anti-human IgG, Alexa 488 conjugated, for Synagis and its isotype control; 1 μg Goat anti-murine IgG, APC conjugated, for B233 and its isotype control. Secondary antibodies were allowed to bind for 30 minutes on ice, protected from light. Labeled cells were washed with FACS buffer again as before, resuspended in 500 μl FACS buffer, and then transferred to the FACS lab.

Data Acquisition and Analysis

Propidium iodide, which stains only dead cells, was added to each sample so that only live cells would be analyzed.

Flow Cytometry experiments were carried out using a FACSCalibur flow cytometry instrument (BD Biosciences; San Jose, Calif.) equipped with an argon-ion laser and a red diode laser. The instrument was Quality Control tested on a daily basis using the FACSComp™ system (BD Biosciences). Flow cytometry analyses were performed according to the instruction manual provided by BD (FACSCalibur™ User's System). FACS data were recorded and analyzed on Macintosh Power PCs G3 and G4 using BD CellQuest™ Software. Data were backed up daily to a server and recorded onto a CD monthly. One percent (w/v) albumin bovine fraction V in phosphate buffered saline (PBS), pH 7.4, free of calcium and magnesium, was used as buffer for antibody binding, cell washing, and resuspension prior to analysis. FACSFlow™ sheath fluid was used for the operation of the instrument according to the manufacturer's protocols.

FIG. 2 shows that RSV-F protein becomes highly expressed on the surface of RSV infected respiratory epithelial cells after one day, and continues to increase after two days. FIG. 3 shows that EphA2 expression also significantly increases on the surface of highly infected respiratory epithelial cells after one day, and increases slightly after the second day.

### 6.4 Example 3

### Detection of EphA2 mRNA Expression in BEAS-2B Cells During RSV Infection

This example illustrates EphA2 expression at the transcriptional level increases after RSV infection of respiratory epithelial cells, as analyzed by RT-PCR.

Preparation of Cells

BEAS-2B cells were plated and infected as described in Example 1. Total RNA was isolated with the Total RNA Isolation Kit (Agilent Technologies, Palo Alto, Calif.) according to the manufacturer's instructions. RNA concentration was determined by A260.

RT-PCR

For RT-PCR, total RNA was isolated from BEAS-2B cells infected at one or two days, and mRNA of EphA2 was reverse transcribed and amplified by real-time PCR. RT-PCR was performed with 100 ng RNA as template using the TaqMan One Step RT-PCR Mastermix Kit and the ABI Assay on Demand for human EphA2, according to the manufacturer's instructions (Applied Biosystems, Foster City, Calif.). 18S rRNA primers were used in separate reactions as normalization controls.

The instrument used was the ABI Prism 7000 Sequence Detection System and the software supplied by the manufacturer. The temperature cycles were as follows: one repeat each of 48° C., 30 min, and 95° C., 10 min, then 40 repeats of [95° C., 15 sec; 60° C., 1 min.] Threshold cycle (Ct) data were exported to qgene96, an Excel file with macros, created by Patrick Muller, and mean normalized expression levels were calculated.

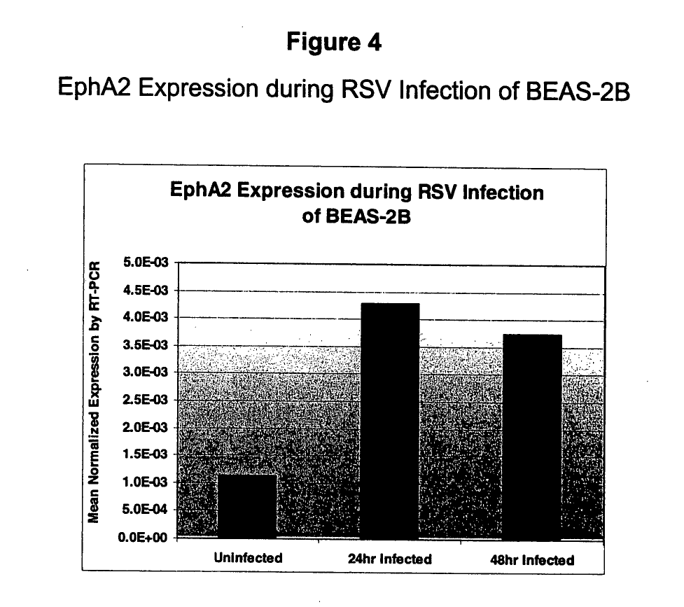

As depicted in FIG. 4, following RSV infection of respiratory epithelial cells, transcription of EphA2 increases about 4 fold after 24 hrs, and remains high at 48 hrs.

### 6.5 Example 4

### Detection of EphA2 on NHBE Cells Using Western Blot Analysis

This example demonstrates that total EphA2 protein is increased in primary human bronchial epithelial cells (NHBE) infected with RSV for one day.

Western Blot Analysis

Western blot analysis of EphA2 protein was performed as described in Example 1, supra.

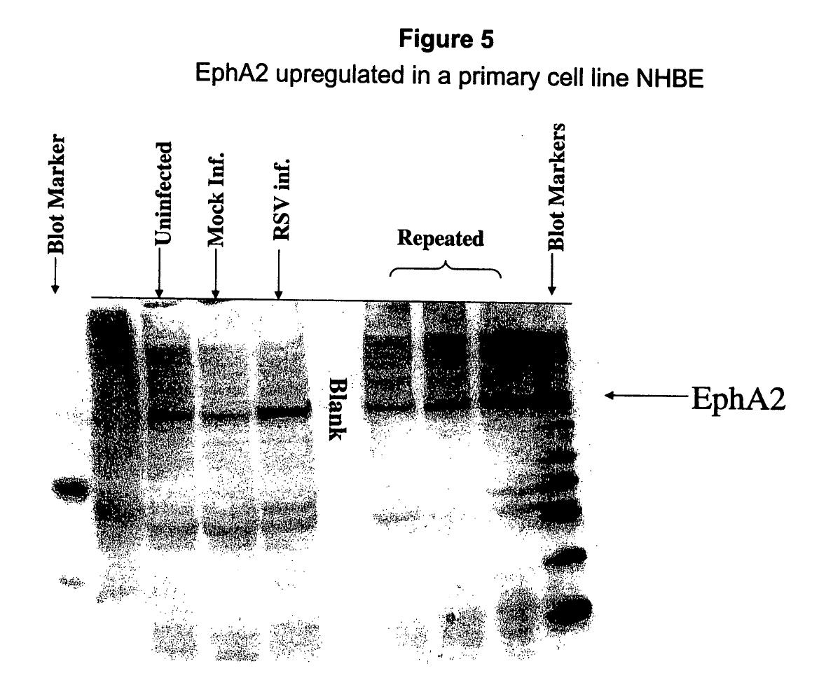

As shown in FIG. 5, EphA2 protein is significantly increased in primary human bronchial epithelium infected for 24 hours with RSV. Controls are no treatment or mock infection with cell lysate made from uninfected cells.

### 6.6 Example 5

### Detection of EphA2 on NHBE Cells Using FACS Analysis

This example shows the levels of RSV-F protein and EphA2 on the surface of primary human bronchial epithelium (NHBE cells) after 24 hours infection with lower amounts of RSV (MOI of 1 or 0.1).

FACS analysis was performed as described in Example 2, supra. In these experiments, the number of viral particles relative to number of cells (MOI) was 1 or 0.1 instead of 10.

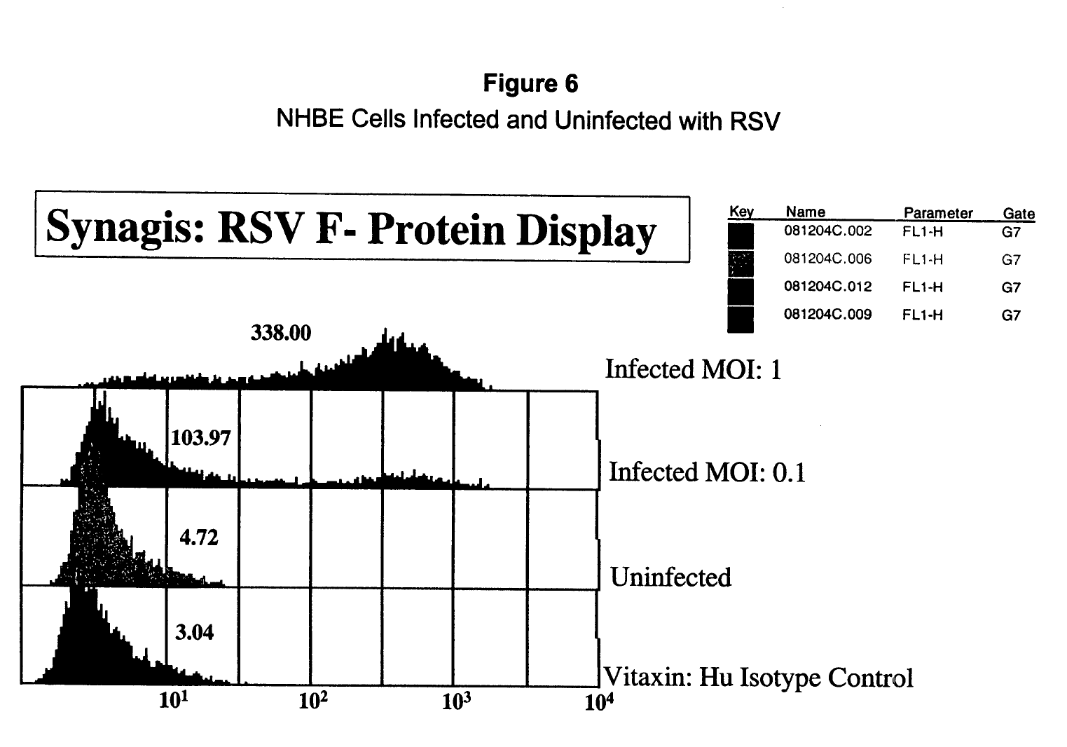

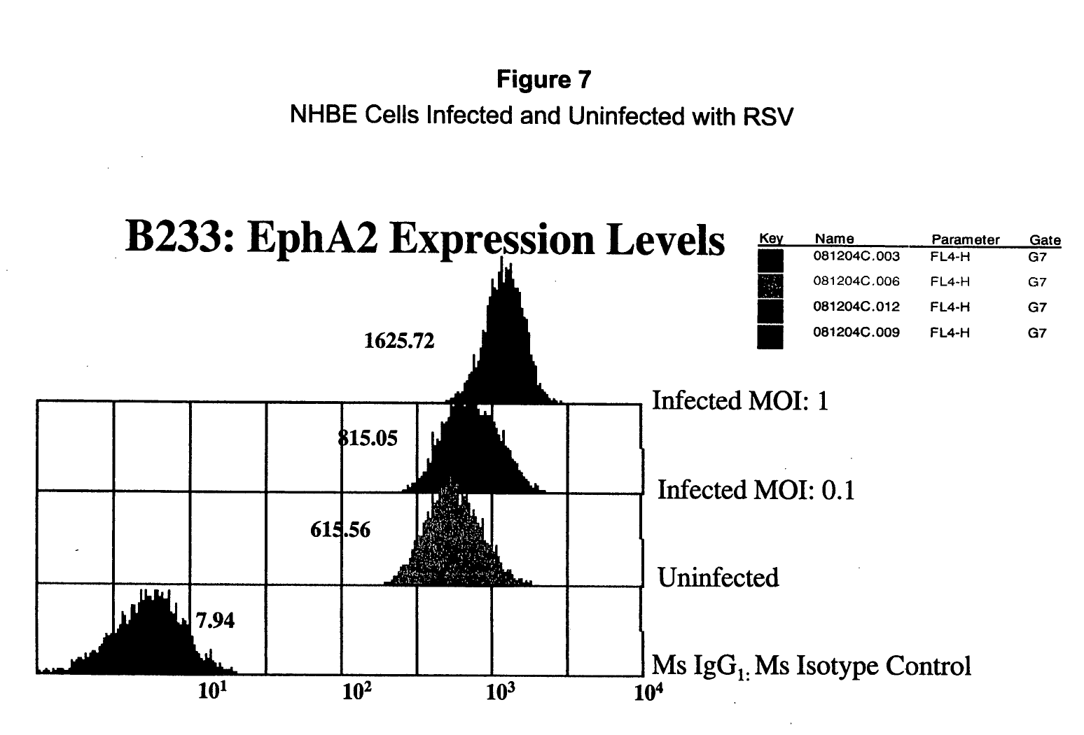

As depicted in FIGS. 6 and 7, primary human airway epithelium has a response to RSV similar to that of the cell line BEAS-2B. The number of cells expressing RSV-F protein on their surface is directly proportional to the degree of infection (MOI) at 24 hours (FIG. 6). EphA2 expression on the surface of infected cells is also increased with increasing MOI (FIG. 7).

### 6.7 Example 6

### Detection of EphA2 on NHBE and BEAS-2B Cells Using FACS Assay, Quadrant Analysis

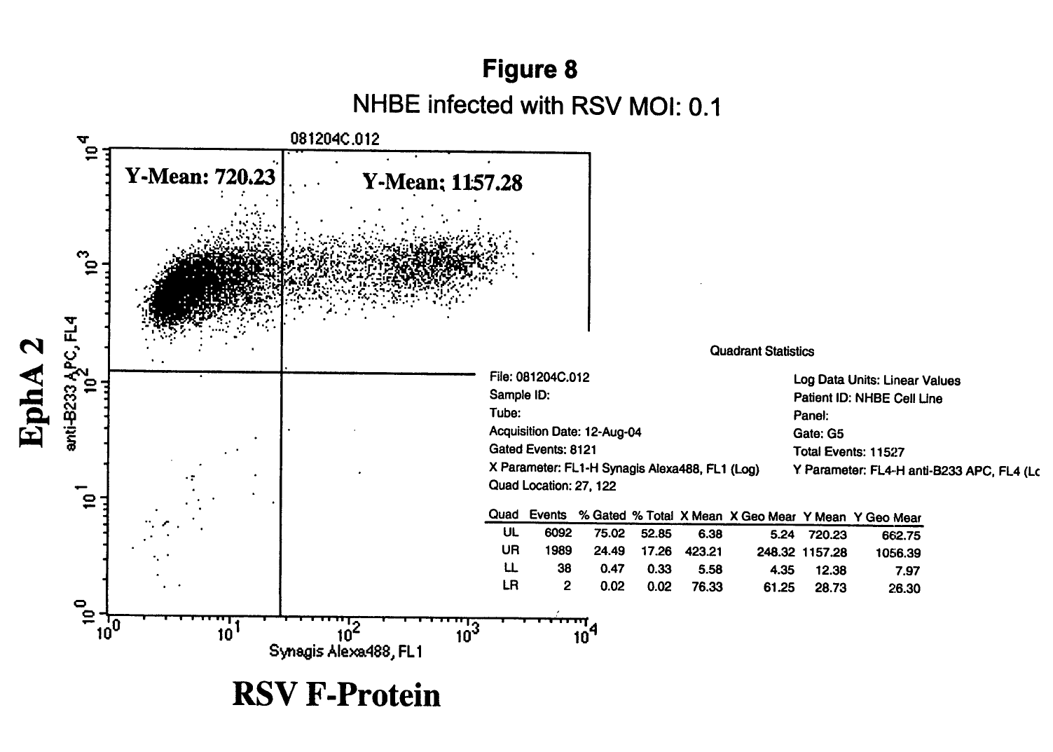

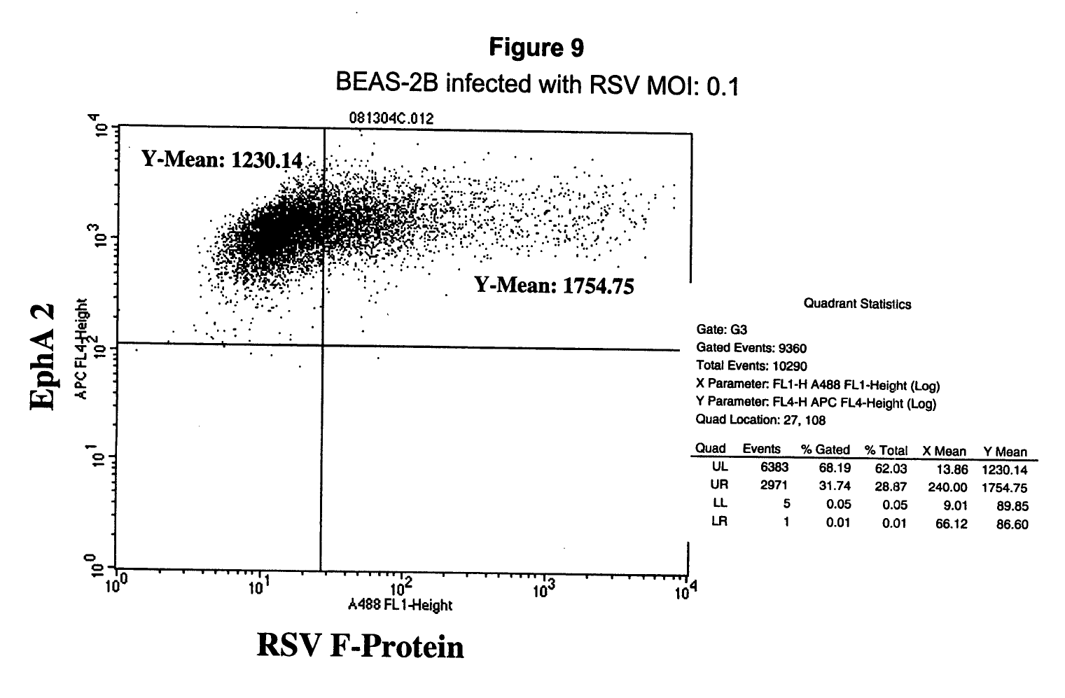

In this example, FACS assays and quadrant analysis were performed to determine which cells (e.g., infected cells or neighboring uninfected cells) up-regulate EphA2 after some of the cells have been infected with RSV (see FIGS. 8 and 9).

The low multiplicity infection with RSV was performed as described in Example 5, and after 24 hours, the cells were detached from the plates and labeled with both anti-RSV-F mAb and anti-EphA2 mAb before FACS analysis. Single labeled cells and isotype controls were included.

The data from double labeled cells were divided into quadrants, so that quantity of EphA2 could be compared between RSV-F negative and RSV-F positive cells. Because there is a continuum of RSV-F staining in the cell population, it is not possible to determine exactly which cells are uninfected and which are infected. Generally, however, cells in the upper left quadrant did not stain for RSV-F protein and were defined as uninfected, while cells in the upper right quadrant stained positive for RSV-F protein, and were defined as the infected population.

The data depicted in FIGS. 8 and 9 suggest that the amount of EphA2 on the surface of both NHBE cells (FIG. 8) and BEAS-2B cells (FIG. 9) is higher in the infected cells than in the uninfected cells. However, this is an estimate. It conceivable that some of the cells in the upper left quadrant were infected, but that insufficient time had passed for the RSV-F protein to appear on the cell surface.

### 6.8 Example 7

### Determination of the Mechanism of EphA2 Upregulation in NHBE and BEAS-2B Cells Using FACS Assay

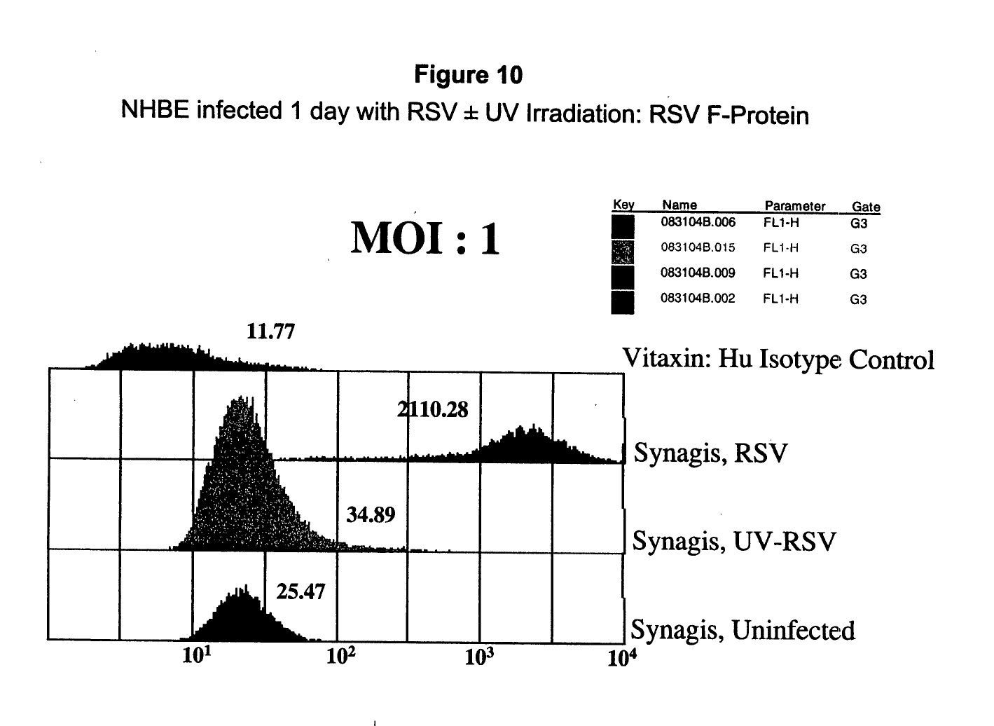

This example illustrates experiments performed to determine whether EphA2 is up-regulated by binding viral particles to the cell surface, or by an active infection process (see FIGS. 10-17).

Preparation of Viral Stocks

RSV was treated with UV irradiation to render it noninfectious but still intact.

10⁵ Hep-2 cells/well (1 ml) were seeded into 24 well plates for determining viral titer before and after UV irradiation. RSV-A2 #10 stock was divided into 4 ml flint glass vials, 380 μl/vial, and treated on a short wave UV light box, 30-60 minutes at room temperature.

Infection of NHBE or BEAS-2B Cells.

When the Hep-2 cells cells were 80-90% confluent (2 days growth), serial 10-fold dilutions of the viral stocks were made in (EMEM/10% FBS/PS), and 200 μl of each dilution was used to infect NHBE or BEAS-2B cells in duplicate. Infections were performed as described supra with untreated (1.2×10⁸ pfU/ml) or UV-treated (<50 pfu/ml) virus stocks, and FACS analysis was performed after 24 hours infection at a MOI of 1 or 0.1, using either NHBE or BEAS-2B cells. NHBE or BEAS-2B cells were infected for 1 hour at 37° C., and the plates were rocked by hand every 15 min. At the end of the inoculation time, 1 ml 0.75% Methyl cellulose in complete EMEM growth medium was added to each well, and the plates incubated at 37° C. for 4 days. Growth medium was removed and monolayers were fixed and stained by adding 0.5 ml/well of 20% methanol/0.1% crystal violet, and incubating for 30-60 minutes at room temperature Plaques appeared as holes or lighter circles in the dark purple monolayer. Before UV irradiation, the titer was 1.2×10⁸ pfu/ ml. After UV irradiation, no plaques were detected in the 10⁻¹ dilution, so less than 50 pfu/ml.

Preparation of FACS Samples

NHBE or BEAS-2B cells were plated and infected at a MOI of 1 or 0.1 as described above. After 24 hours, cells were detached and stained with either anti-RSV-F mAb or anti-EphA2 mAb, as described supra. FACS analysis was also performed as described supra.

Results

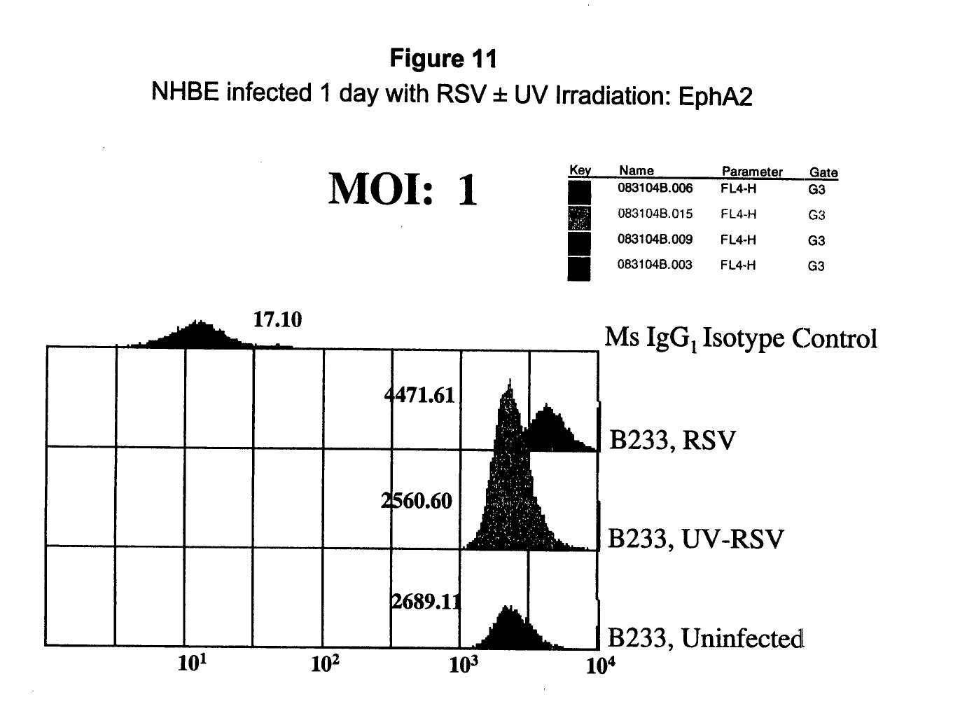

FIGS. 10 and 11 illustrate results showing that when NHBE cells were infected for one day with RSV at a MOI of 1, RSV-F protein was expressed on almost all the cells, and EphA2 increases approximately two-fold. When the cells were infected with UV-inactivated RSV under the same conditions, almost no RSV-F protein was expressed on the cells, and the level of EphA2 did not increase.

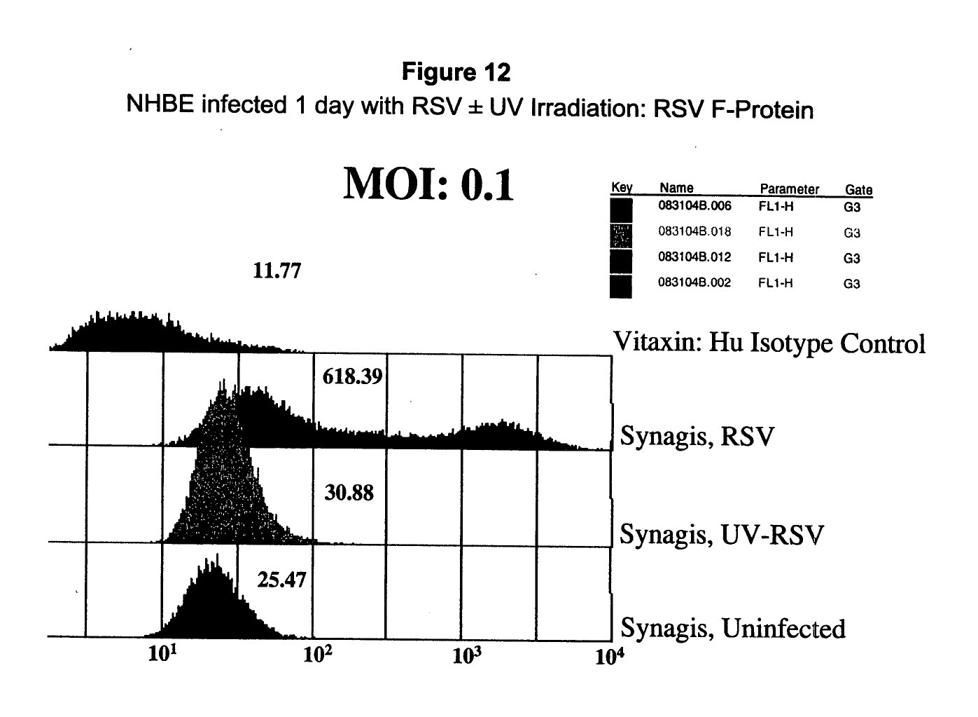

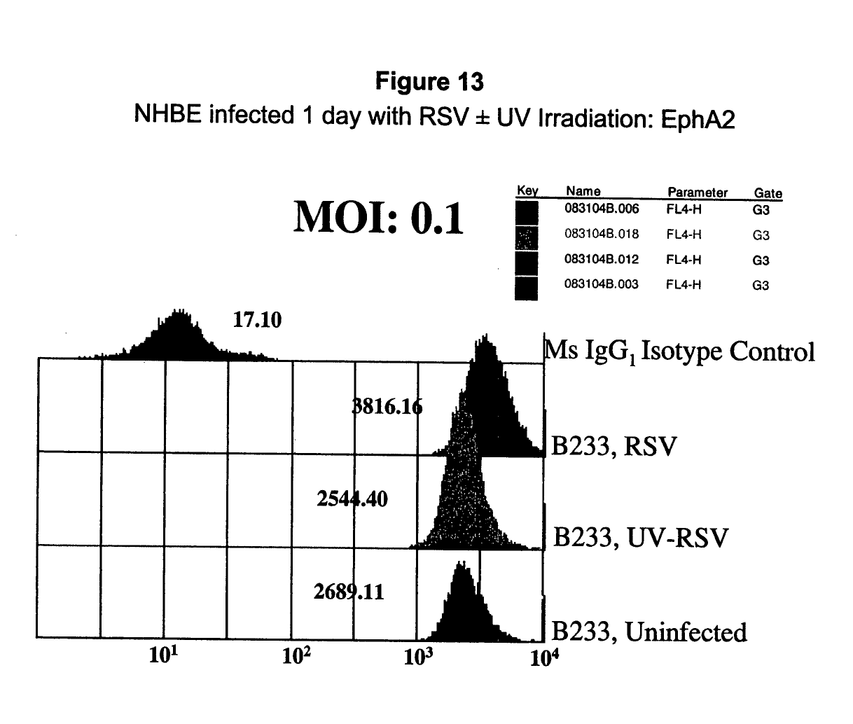

FIGS. 12 and 13 illustrate the results of the same experiment done at a MOI of 0.1. In this case, RSV-F protein was expressed on a smaller fraction of the cells, reflecting fewer cells infected after one day. The increase in EphA2, however, was almost as high as that for the MOI=1 experiment. When the virus was UV-inactivated, neither RSV-F protein nor any increase in EphA2 was observed in the cells.

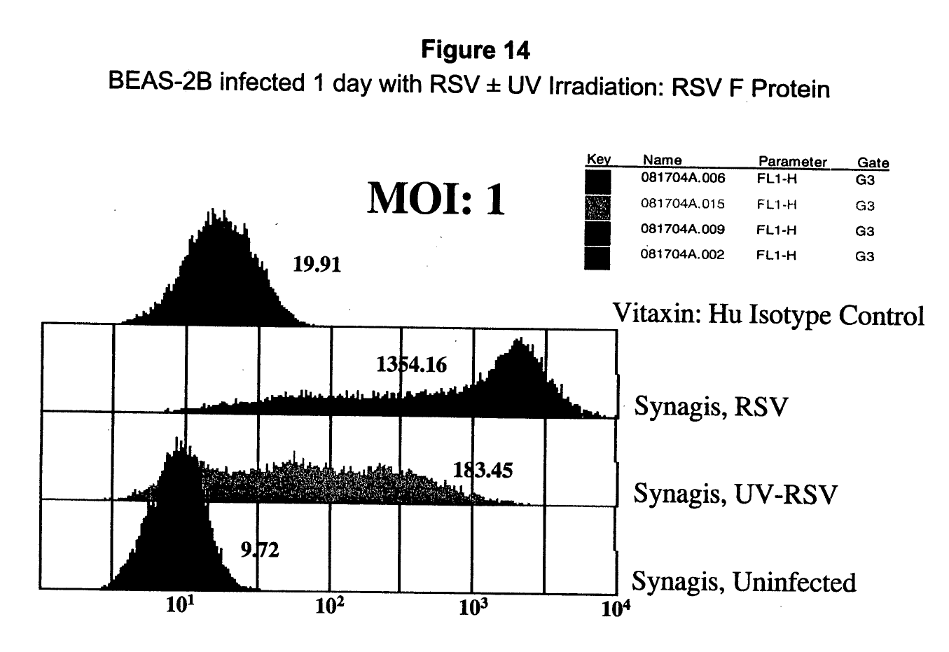

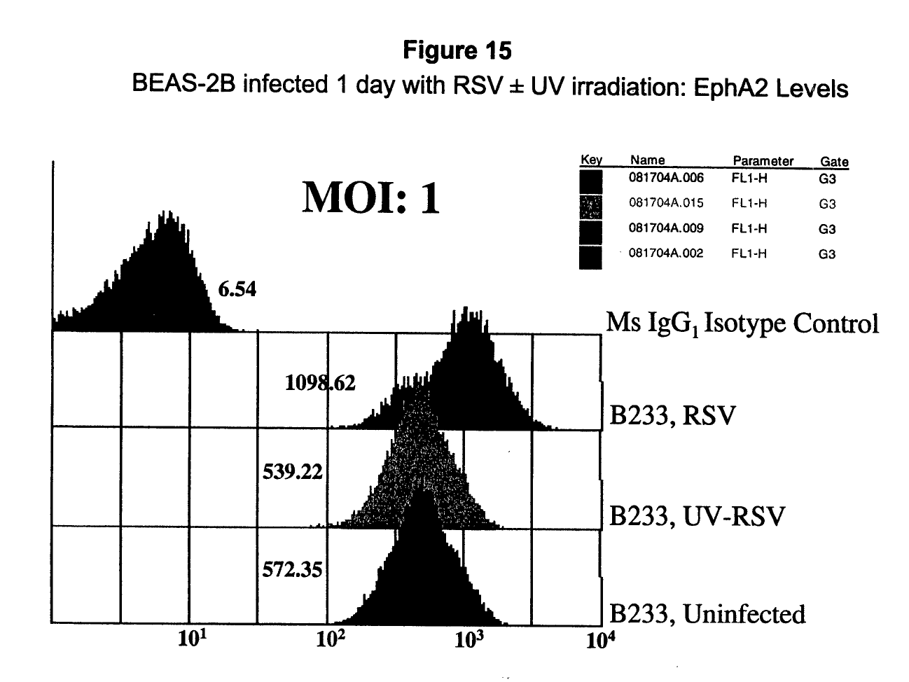

FIGS. 14 and 15 illustrate results from infecting BEAS-2B for one day at a MOI of 1 with untreated or UV-inactivated RSV. Results similar to those using NHBE cells were observed, although there was more expression of RSV-F protein on BEAS-2B infected with UV-inactivated RSV. No increase in EphA2 was observed when cells were infected with UV-inactivated RSV.

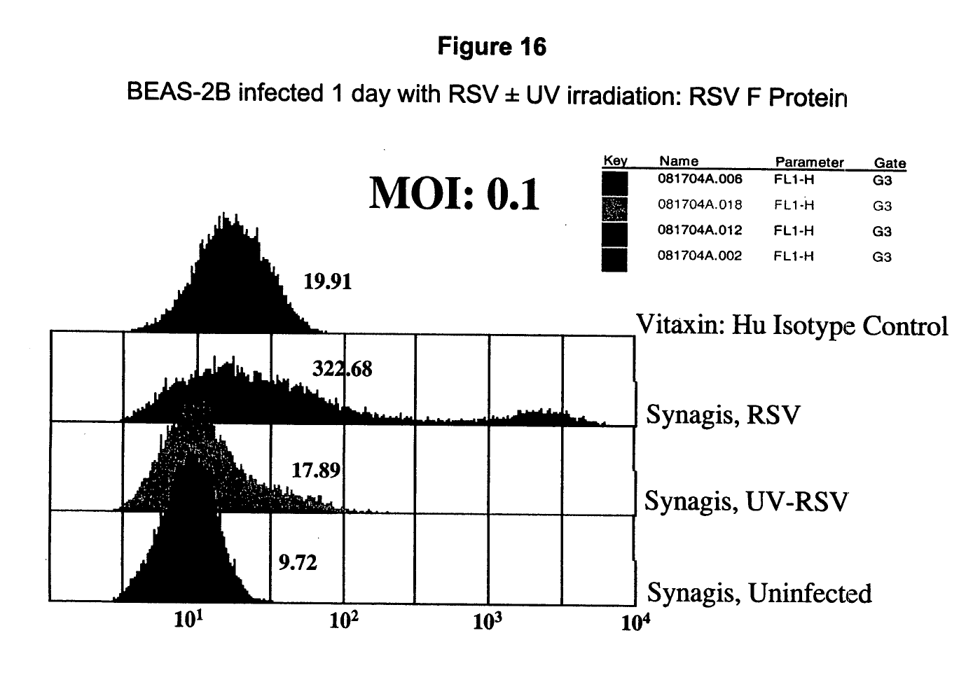

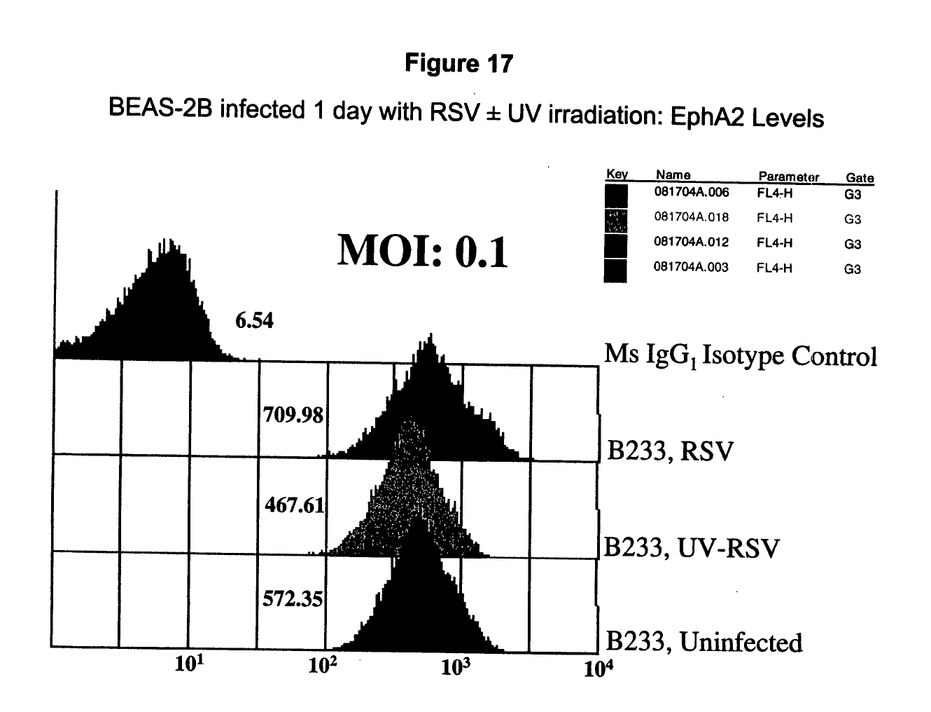

FIGS. 16 and 17 illustrate results from infecting BEAS-2B for one day at a MOI of 0.1 with untreated or UV-inactivated RSV. Similar to results using NHBE, a smaller fraction of the cells expressed RSV-F protein, and EphA2 increased slightly less than when the MOI of 1. The increase in EphA2 expression was observed only when cells were infected with untreated RSV, and not with UV-inactivated virus.

Thus, in either primary (NHBE cells) or an established cell line of bronchial epithelium (BEAS-2B cells), increases in cell surface EphA2 expression occured only during an active infection by RSV, and not during simple binding of viral particles to the cell membrane.

### 6.9 Example 8

### Detection of EphA2 in Other Cells Infected with RSV Using FACS Assay

To determine whether EphA2 upregulation occurs in response to RSV infection in types of cells other than NHBE and BEAS-2B, A549 or Hep-2 cells were infected with RSV at various MOI for 48 hours using methods described above. The cells were then detached and labeled with anti-EphA2 mAb, and analyzed by FACS for surface EphA2 using methods described above.

Results

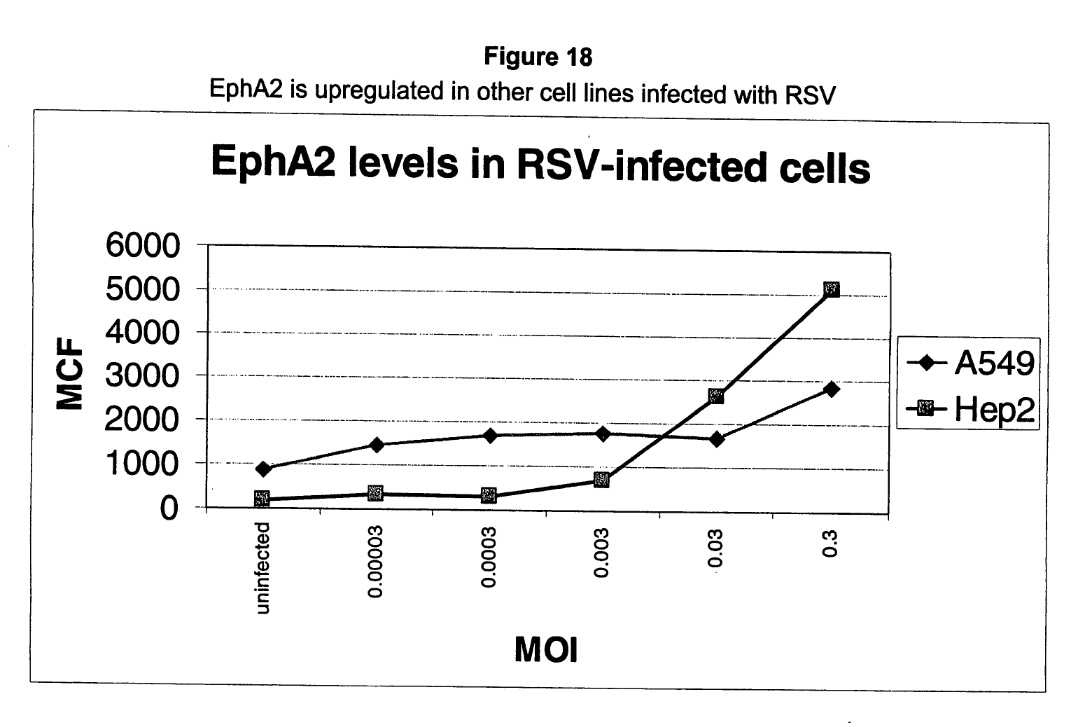

Besides NHBE and BEAS-2B, A549 and Hep-2 cells also displayed increased levels of EphA2 on their surface after infection with RSV (see FIG. 18).

### 6.10 Example 9

### Detection of EphA2 in Murine Lung

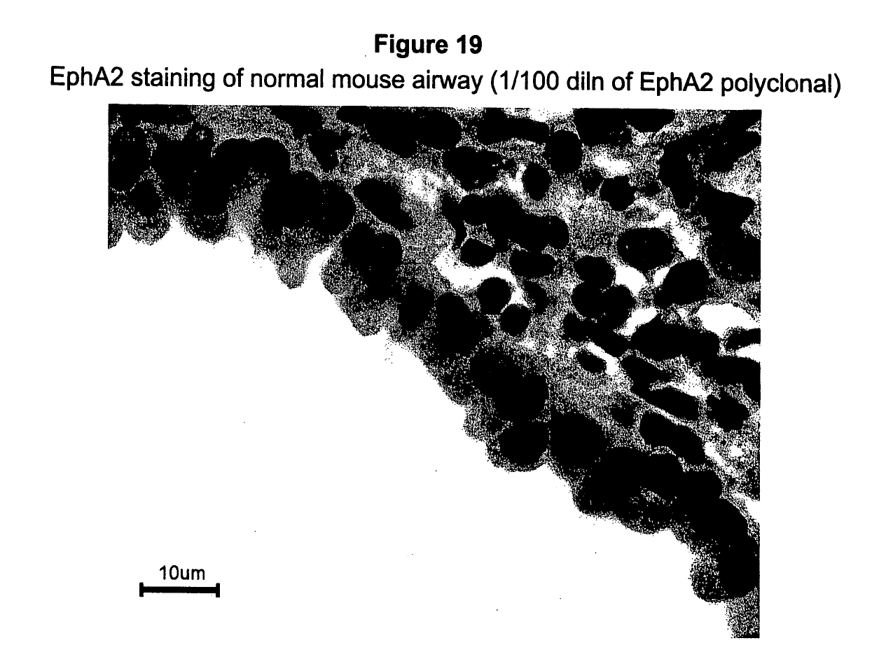

This example illustrates the presence of EphA2 in formalin-fixed paraffin-embedded normal, RSV-infected or bleomycin-treated murine lung tissue (see FIGS. 19-21).

The following materials were used to perform the immunohistochemistry (IHC) experiments described, infra:

Distilled water was obtained from a RODI system (Aztec, N. Mex.). 3% H2O2/methanol peroxidase block was prepared with 25 ml 30% H2O2 filled to 250 ml with methanol. A 5% bovine serum albumin solution (BSA) was made with 12.5 g BSA (Sigma 7906-SOOG Batch 103K1375) dissolved in TBS-tween (TBST). TBST was made with 60 ml Biofluids 10× TBS filled to 600 ml with distilled water plus 60 μl tween on the first incubation day and 400 ml Biofluids 10× TBS filled to 4 L with distilled water plus 400 ul tween on the second incubation day. A 1% BSA solution was prepared from 6 ml 5% BSA solution and filling to 30 ml with TBST. EphA2 (H-77) rabbit polyclonal IgG was obtained from Santa Cruz Biotechnology (cat. #SC-10746, Lot A311, 200 ug/ml); a 1:100 dilution was prepared by adding 90 μl to 9 ml 1% BSA solution. Purified rabbit IgG was (Control/RN: 1673.072, 1.18 mg/ml) and diluted to 1 μg/ml by adding 7.63 ul to 9 ml 1% BSA. A 1% BSA solution was used for the minus primary control. For the link antibody, goat anti-rabbit IgG biotinylated (Dako E0432, 0.99 mg/ml) was diluted to 2 μg/ml by adding 70.7 μl to 35 ml TBST. Streptavidin HRP (Dako P0397, Lot 00004379, 0.62 mg/ml) was diluted to 1.6 μg/ml by adding 90.3 μl to 35 ml TBST. A diaminobenzidine substrate (DAB) was obtained from Sigma and made by adding 540 μl Solution B (D5815 Lot 103K10302) to 18 ml Solution A (D5940 Lot 103K10301).

Staining Method

RSV-infected murine lung and normal murine lung tissue were formalin-fixed, then cut from paraffin-embedded blocks and mounted on positively-charged slides and stored at room temperature for several days. Immediately before immunohistochemistry analysis, slides were dewaxed by submersing them for 5 minutes each in the following solutions: 4 times in xylene, followed by 2 times in 100% reagent alcohol, followed by one time in 95% reagent alcohol, then one time in 70% alcohol. Slides were then immediately submersed in distilled water. Endogenous peroxidases in tissues were blocked by submersing slides in a 3% H₂O₂/methanol solution for 10 minutes (made immediately before use, after dewaxing slides). Slides were then rinsed in distilled water. Slides were then submersed for 30 minutes in a 5% BSA solution. Without rinsing, slides were prepared one at a time for incubation with primary antibody by wiping off excess liquid (5% BSA) from each slide, placing it flat on the incubator, then applying 1 ml of primary antibody (EphA2 H-77 rabbit polyclonal IgG, purified rabbit IgG, or minus primary solution). Slides were incubated in a humid environment at room temperature for 19.5 hours.

After rinsing in TBST, slides were transferred to a Dako Autostainer Plus, where the following incubations took place: incubated with TBST for 10 minutes; incubated with goat anti-rabbit IgG for 30 minutes; incubated with TBST for 5 minutes; incubated with Streptavidin HRP for 30 minutes, incubated with TBST for 5 minutes; incubated with TBST for 10 minutes; incubated with goat anti-rabbit IgG for 30 minutes; incubated with TBST for 5 minutes; incubated with Streptavidin HRP for 30 minutes, incubated with TBST for 5 minutes; incubated with DAB for 4 minutes, then rinsed with distilled water. Slides were removed from the autostaining machine and submersed in distilled water. Slides were then submersed in Mayer's hematoxylin for 2.5 minutes and then rinsed several times with distilled water. Slides were submersed for 30 seconds in Scott's Tap Water Substitute for “blueing,” then rinsed several times in distilled water. Slides were dehydrated by soaking them for 5 minutes in each of the following solutions: one time in 95% reagent alcohol, 3 times in 100% reagent alcohol, 4 times in xylene. Slides were removed from xylene and coverslips were adhered with DPX.

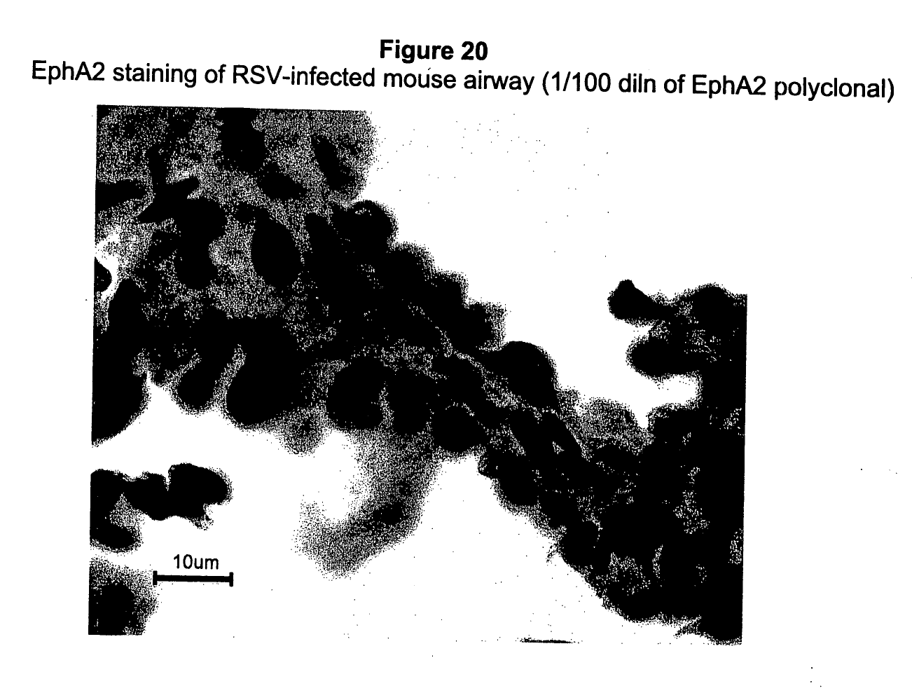

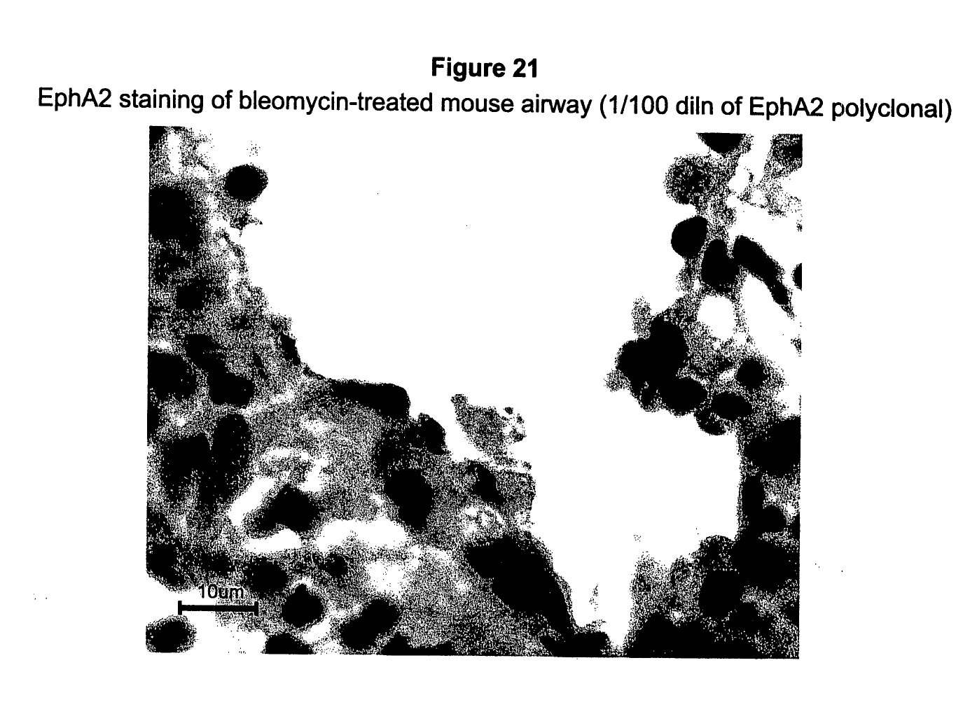

FIGS. 19-21 illustrate the results of IHC experiments staining for EphA2 in normal (FIG. 19), RSV-infected (FIG. 20) and bleomycin-treated (FIG. 21) mouse airway tissue.

### 7. EQUIVALENTS

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

What is claimed:

1. A method of treating an infection or a symptom thereof, said method comprising administering to a subject in need thereof a therapeutically effective amount of an EphA2/EphrinA1 Modulator.

2. The method of claim 1, wherein the infection is associated with an increase in EphA2 expression in the cells of said subject.

3. The method of claim 1, wherein the infection is a bacterial infection, a fungal infection or a protozoan infection.

4. The method of claim 1, wherein the infection is a viral infection.

5. The method of claim 4, wherein said viral infection is a RSV infection.

6. The method of claim 1, wherein the EphA2/EphrinA1 Modulator is an antibody that immunospecifically binds to EphA2.

7. The method of claim 6, wherein the antibody prevents binding of EphA2 to EphrinA1.

8. The method of claim 6, wherein the antibody induces EphA2 signal transduction.

9. The method of claim 6, wherein the antibody induces EphA2 degradation.

10. The method of claim 6, wherein the antibody is a monoclonal antibody.

11. The method of claim 6, wherein the antibody is a human or humanized antibody.

12. The method of claim 6, wherein the antibody is EA2 or EA5.

13. The method of claim 12, wherein said EA2 or EA5 antibody is humanized or chimerized.

14. The method of claim 1, wherein the EphA2/EphrinA1 Modulator is a soluble EphrinA1.

15. The method of claim 14, wherein the soluble EphrinA1 is EphrinA1 fused to the Fc protion of an IgG molecule.

16. The method of claim 1, wherein the EphA2/EphrinA1 Modulator is an EphA2 antisense molecule.

17. The method of claim 1, wherein the EphA2/EphrinA1 Modulator is an EphA2 vaccine.

18. The method of claim 1, further comprising the administration of an effective amount of a therapy other than an EphA2/EphrinA1 Modulator.

19. The method of claim 18, wherein the therapy is an anti-inflammatory agent, an immunomodulatory agent, an anti-viral agent, an anti-bacterial agent or an anti-fungal agent.

20. The method of claim 1, wherein the subject is a human subject.

21. The method of claim 4, wherein the EphA2/EphrinA1 Modulator is an antibody that immunospecifically binds to EphA2.

22. The method of claim 21, wherein the antibody prevents binding of EphA2 to EphrinA1.

23. The method of claim 21, wherein the antibody induces EphA2 signal transduction.

24. The method of claim 21, wherein the antibody induces EphA2 degradation.

25. The method of claim 21, wherein the antibody is a monoclonal antibody.

26. The method of claim 21, wherein the antibody is a human or humanized antibody.

27. The method of claim 21, wherein the antibody is EA2 or EA5.

28. The method of claim 27, wherein said EA2 or EA5 antibody is humanized or chimerized.

29. The method of claim 4, wherein the EphA2/EphrinA1 Modulator is a soluble EphrinA1.

30. The method of claim 29, wherein the soluble EphrinA1 is EphrinA1 fused to the Fc protion of an IgG molecule.

31. The method of claim 4, wherein the EphA2/EphrinA1 Modulator is an EphA2 antisense molecule.

32. The method of claim 4, wherein the EphA2/EphrinA1 Modulator is an EphA2 vaccine.

33. The method of claim 4, further comprising the administration of an effective amount of a therapy other than an EphA2/EphrinA1 Modulator.

34. The method of claim 33, wherein the therapy is an anti-inflammatory agent, an immunomodulatory agent, an anti-viral agent, an anti-bacterial agent or an anti-fungal agent.

35. The method of claim 4, wherein the subject is a human subject.

36. The method of claim 5, wherein the EphA2/EphrinA1 Modulator is an antibody that immunospecifically binds to EphA2.

37. The method of claim 36, wherein the antibody prevents binding of EphA2 to EphrinA1.

38. The method of claim 36, wherein the antibody induces EphA2 signal transduction.

39. The method of claim 36, wherein the antibody induces EphA2 degradation.

40. The method of claim 36, wherein the antibody is a monoclonal antibody.

41. The method of claim 36, wherein the antibody is a human or humanized antibody.

42. The method of claim 36, wherein the antibody is EA2 or EA5.

43. The method of claim 42, wherein said EA2 or EA5 antibody is humanized or chimerized.

44. The method of claim 5, wherein the EphA2/EphrinA1 Modulator is a soluble EphrinA1.

45. The method of claim 44, wherein the soluble EphrinA1 is EphrinA1 fused to the Fc protion of an IgG molecule.

46. The method of claim 5, wherein the EphA2/EphrinA1 Modulator is an EphA2 antisense molecule.

47. The method of claim 5, wherein the EphA2/EphrinA1 Modulator is an EphA2 vaccine.

48. The method of claim 5, further comprising the administration of an effective amount of a therapy other than an EphA2/EphrinA1 Modulator.

49. The method of claim 48, wherein the therapy is an anti-inflammatory agent, an immunomodulatory agent, an anti-viral agent, an anti-bacterial agent or an anti-fungal agent.

50. The method of claim 5, wherein the subject is a human subject.


SEQUENCE LISTING (43 sequences)

SEQ ID NO: 1 - protein, 15 aa, Homo sapiens
GGGGSGGGGSGGGGS

SEQ ID NO: 2 - protein, 15 aa, Homo sapiens
ESGRSGGGGSGGGGS

SEQ ID NO: 3 - protein, 14 aa, Homo sapiens
EGKSSGSGSESKST

SEQ ID NO: 4 - protein, 15 aa, Homo sapiens
EGKSSGSGSESKSTQ

SEQ ID NO: 5 - protein, 14 aa, Homo sapiens
EGKSSGSGSESKVD

SEQ ID NO: 6 - protein, 14 aa, Homo sapiens
GSTSGSGKSSEGKG

SEQ ID NO: 7 - protein, 18 aa, Homo sapiens
KESGSVSSEQLAQFRSLD

SEQ ID NO: 8 - protein, 16 aa, Homo sapiens
ESGSVSSEELAFRSLD

SEQ ID NO: 9 - protein, 4 aa, Homo sapiens
KDEL

SEQ ID NO: 10 - protein, 4 aa, Homo sapiens
DDEL

SEQ ID NO: 11 - protein, 4 aa, Homo sapiens
DEEL

SEQ ID NO: 12 - protein, 4 aa, Homo sapiens
QEDL

SEQ ID NO: 13 - protein, 4 aa, Homo sapiens
RDEL

SEQ ID NO: 14 - protein, 7 aa, Homo sapiens
PKKKRKV

SEQ ID NO: 15 - protein, 7 aa, Homo sapiens
PQKKIKS

SEQ ID NO: 16 - protein, 5 aa, Homo sapiens
QPKKP

SEQ ID NO: 17 - protein, 4 aa, Homo sapiens
RKKR

SEQ ID NO: 18 - protein, 5 aa, Homo sapiens
KKKRK

SEQ ID NO: 19 - protein, 12 aa, Homo sapiens
RKKRRQRRRAHQ

SEQ ID NO: 20 - protein, 16 aa, Homo sapiens
RQARRNRRRRWRERQR

SEQ ID NO: 21 - protein, 19 aa, Homo sapiens
MPLTRRRPAASQALAPPTP

SEQ ID NO: 22 - protein, 15 aa, Homo sapiens
MDDQRDLISNNEQLP

SEQ ID NO: 23 - protein, 32 aa, Homo sapiens
  misc_feature (7)..(8): Xaa can be any naturally occurring amino acid
MLFNLRXXLNNAAFRHGHNFMVRNFRCGQPLX

SEQ ID NO: 24 - protein, 3 aa, Homo sapiens
AKL

SEQ ID NO: 25 - protein, 6 aa, Homo sapiens
SDYQRL

SEQ ID NO: 26 - protein, 8 aa, Homo sapiens
GCVCSSNP

SEQ ID NO: 27 - protein, 8 aa, Homo sapiens
GQTVTTPL

SEQ ID NO: 28 - protein, 8 aa, Homo sapiens
GQELSQHE

SEQ ID NO: 29 - protein, 8 aa, Homo sapiens
GNSPSYNP

SEQ ID NO: 30 - protein, 8 aa, Homo sapiens
GVSGSKGQ

SEQ ID NO: 31 - protein, 8 aa, Homo sapiens
GQTITTPL

SEQ ID NO: 32 - protein, 8 aa, Homo sapiens
GQTLTTPL

SEQ ID NO: 33 - protein, 8 aa, Homo sapiens
GQIFSRSA

SEQ ID NO: 34 - protein, 8 aa, Homo sapiens
GQIHGLSP

SEQ ID NO: 35 - protein, 8 aa, Homo sapiens
GARASVLS

SEQ ID NO: 36 - protein, 8 aa, Homo sapiens
GCTLSAEE

SEQ ID NO: 37 - protein, 16 aa, Homo sapiens
AAVALLPAVLLALLAP

SEQ ID NO: 38 - protein, 12 aa, Homo sapiens
AAVLLPVLLAAP

SEQ ID NO: 39 - protein, 15 aa, Homo sapiens
VTVLALGALAGVGVG

SEQ ID NO: 40 - DNA, 30 bp, Homo sapiens
ccagcagtaccacttccttgccctgcgccg

SEQ ID NO: 41 - DNA, 30 bp, Homo sapiens
gccgcgtcccgttccttcaccatgacgacc

SEQ ID NO: 42 - DNA, 31 bp, Homo sapiens
ccagcagtaccgcttccttgccctgcggccg

SEQ ID NO: 43 - DNA, 30 bp, Homo sapiens; identical to SEQ ID NO: 41